(19) United States
(12) Patent Application Publication
(10) Pub. No.: US 2025/0288601 A1
(43) Pub. Date: Sep. 18, 2025

(54) METHODS FOR TREATING UROGENITAL HEALTH CONDITIONS

(71) Applicant: ONE BIO INC, Sacramento, CA (US)

(72) Inventors: Matthew Joseph AMICUCCI, Sacramento, CA (US); Angela Maria MARCOBAL-BARRANCO, Sacramento, CA (US); Cory Glen VIERRA, Sacramento, CA (US); Maria Ximena MALDONADO-GOMEZ, Sacramento, CA (US)

(73) Assignee: ONE BIO INC, Sacramento, CA (US)

(21) Appl. No.: 18/861,471

(22) PCT Filed: May 11, 2023
(86) PCT No.: PCT/US2023/021949

§ 371 Date: Oct. 29, 2024

Related U.S. Application Data
(60) Provisional application No. 63/341,385, filed on May 12, 2022.
(60) Provisional application No. 63/379,827, filed on Oct. 17, 2022.

(51) Int. Cl. A61K 31/702; A61K 35/00; A61K 35/74; A61K 35/745; A61K 35/747; A61K 36/185; A61K 36/21; A61K 36/48; A61K 36/49; A61K 36/899; A61K 36/8998; A61P 31/04

(52) U.S. Cl. CPC A61K 31/702; A61K 35/74; A61K 35/745; A61K 35/747; A61K 36/185; A61K 36/21; A61K 36/48; A61K 36/49; A61K 36/899; A61K 36/8998; A61P 31/04; A61K 2035/115

(57) ABSTRACT

Provided are methods for treating urogenital health conditions of a female subject. In some aspects, the methods comprise administering a composition comprising one or more oligosaccharides that are derived from one or more materials comprising at least one of galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane, or any combination thereof. In some aspects the methods comprise administering one or more oligosaccharides each of which independently contains 3 to 30 subunits and wherein at least 5% of the subunits comprise a beta-1,3 glucose residue, a beta-1,4 glucose residue, or a combination thereof.

### CROSS REFERENCE TO RELATED APPLICATIONS

This application claims the benefit of and priority to U.S. Provisional Patent Application No. 63/341,385, filed May 12, 2022 and U.S. Provisional Patent Application No. 63/379,827, filed Oct. 17, 2022, each of which are hereby incorporated by reference in its entirety.

### BACKGROUND OF INVENTION

In most healthy women of childbearing age, the vaginal microbiota is dominated by Lactobacillus. The species of Lactobacillus most commonly isolated from the reproductive tract of women worldwide include L. crispatus, L. jensenii, L. gasseri, L. iners, and L. vaginalis (Younes et al. 2018). These species are phylogenetically different from food and/or environmental Lactobacillus species, being better adapted to persistently colonize the female urogenital tract. Vaginal Lactobacillus metabolizes mucins to produce lactic acid and contributes to the maintenance of a low vaginal pH (generally 3.5-4.5) that accounts for a major part of the non-specific defense of the vagina, along with production of hydrogen peroxide and bacteriocins. During pregnancy, the dominance of Lactobacillus spp is even higher and very stable, reducing the risk for fetal inflammation responses to potential infections, influencing healthy term pregnancy outcomes (Di Giulio et al. 2015).

Depletion of vaginal Lactobacillus is associated with the development of clinical symptoms such as bacterial vaginosis (BV), characterized by an imbalanced vaginal flora deficient in Lactobacillus, and generally an increase in pH to around 4.5-7. Apart from reduction in abundance of Lactobacillus, BV is usually associated with increased propagation of conditionally pathogenic microorganisms such as Gardnerella vaginalis and other anaerobic bacteria, including Atopobium vaginaes, Prevotella spp., and Mobiluncus spp. BV affects about 15-50% of women of reproductive age worldwide (Allsworth et al. 2007). BV can be asymptomatic, and it has been associated with significant gynecologic and obstetric complications, such as pelvic inflammatory disease, endometritis, post-operative infections, including post-cesarean endometritis and post-hysterectomy vaginal cuff cellulitis. Strong associations have also been reported between BV and pre-term delivery, miscarriage, and amniotic fluid infections. Studies have linked BV to both female HIV-1 acquisition, and female-to-male HIV transmission (Cohen et al. 2012).

In addition to bacterial infections, imbalanced vaginal flora has also been associated with vaginal fungal infections. Vulvovaginal candidiasis is the most common vaginal fungal infection, characterized by an aggressive host response to Candida overgrowth (Fidel et al.). Some women can be prone to Candida colonization and candidiasis, being supported by the absence of specific beneficial Lactobacillus in the vaginal tract (Osset et al. 2011). Lactobacillus exert a protective effect by reducing the adhesion of the fungus to the vaginal mucosal, production of organic acids, as well as enhancing vaginal epithelial cell immune defense mechanism (Chew et al. 2015, Morais et al. 2017).

The traditional treatment for vaginal microbiome dysbiosis is the use of antibiotics, particularly Metronidazole, Clindamycin or Tinidazole which target anaerobes, for example Gardnerella vaginalis and Atopobioum vaginae. However, the treatment fails to permanently restore Lactobacillus in many women and in fact may decrease abundance. Therefore, it is common for BV to recur within three to 12 months, despite treatment. If this occurs, extended-use Metronidazole therapy is often attempted. Apart from the common side effects of antibiotic use, extended use may result in additional issues such as development of antibiotic resistant organisms and a dysbiotic microbiota profile in the intestine and urogenital tracts. A dysbiotic profile in the urogenital tract may lead to increased colonization opportunity for detrimental bacteria such as E. coli and the possibility of urinary tract infections.

Some approaches have involved use of combinations of lactic acid and antibiotics carried in intravaginal rings, use of vaginal acidifiers, use of activated charcoal, and use of bacteriophages targeted to vaginal pathogens. However, there is insufficient evidence of clinical efficacy and these approaches have not entered into clinical practice.

To provide an alternative approach without the disadvantages of antibiotics, probiotics have been proposed. Probiotics are defined by the Food and Agriculture Organization (FAO) and the World Health Organization (WHO) as living microorganisms which, when administered in adequate amounts, confer a beneficial health effect on the host. The probiotics may be administered orally or intravaginally. Probiotics such as Lactobacillus reuteri RC-14, Lactobacillus rhamnosus GR-1 and Lactobacillus fermentum LF15 have been explored (Macklaim et al. 2015); although there may be others. However, many clinical studies have failed to reveal significant results and many probiotic strains fail to grow and survive in the female vagina. Currently there is insufficient evidence for or against recommending probiotics for the treatment of BV (Mastromarino et al, 2013).

To overcome the inability of many industrially produced probiotics to colonize, it has been suggested to administer a prebiotic in combination with the probiotic, or to administer a beneficial micro-organism isolated from the subject (US Patent Application Publication 2003/0118571). A prebiotic is a complex carbohydrate, such as an oligosaccharide, which is not absorbed by the human host, but which selectively enhances the growth or metabolic activity of certain beneficial bacteria.

Oligosaccharides are short chains of carbohydrates that have been shown to have a variety of functions (e.g., bioactive functions, etc.) that are influenced by a number of structural attributes such as stereochemistry, branching, degree of polymerization, monosaccharide composition, and glycosidic bond positions (Amicucci, Nandita et al. 2019). Oligosaccharides from human milk (HMOs), for example, promote the growth of certain microbes that are nascent to the infant gut, while also modulating the immune system, reducing instances of diarrhea, and protecting the host from pathogen adhesion (Morrow, Ruiz-Palacios et al. 2004, LoCascio, Ninonuevo et al. 2007, Smilowitz, Lebrilla et al. 2014).

However, oligosaccharides are not universally utilized by all microbiota, and even the same type of oligosaccharide, depending on chain length and structure, can have different effects. For example, one study reported that, while oligofructose was utilized by certain Lactobacillus species, the oligofructose did not increase the proportion of lactobacilli (a favorable urogenital organism) and actually stimulated growth of Candida albicans (an unfavorable pathogen) (Collins et al. 2018). On the other hand, a different study found that vaginal Lactobacillus isolates were able to utilize shorter chain oligofructose, whereas C. albicans did not (Rosseau et al. 2005).

Different oligosaccharide prebiotics, including gluco-oligosaccharides (GOS), galacto-oligosaccharides (GaOS), fructo-oligosaccharides (FOS), Jactulose, and glucomannan hydrolysates (Al-Ghazzewi et al. 2016), have been used to modulate vaginal microbiome, but with limited success. These prebiotics do not reflect the genomic and physiological links between the vaginal Lactobacillus and their carbohydrate structures. Overall, there is also insufficient clinical evidence and these approaches have not been adopted into clinical practice.

While oligosaccharides show promise for modulating certain microbiota and their associated metabolic intermediates and products, the ability to access testable oligosaccharides has been limited by the few available methods of production. For example, biological synthesis is currently the primary tool for producing oligosaccharides at scale, including human milk oligosaccharides (Merighi, McCoy et al. 2016, Yu, Liu et al. 2018). However, little work has been done to expand the availability of oligosaccharides beyond HMOs or other common oligosaccharides, such as galacto-oligosaccharides (GaOS) and fructo-oligosaccharides (FOS) (Gosling, Stevens et al. 2010, Dominguez, Rodrigues et al. 2014). Chemical methods for the depolymerization of polysaccharides, while known in the art, are not routinely employed, but show promise to provide a more robust and broader path to polysaccharide depolymerization to produce oligosaccharides. For example, a method for polysaccharide depolymerization using Fenton's chemistry followed by cleavage using a strong-Arrhenius base (Na⁺OH⁻, K⁺OH⁻, or Ca²⁺(OH⁻)₂) has recently been described (Amicucci, Park et al. 2018, Amicucci, Nandita et al. 2020). Methods based on Fenton chemistry have also been created that employ volatile nitrogen-based bases that can be evaporated off and also actively quench hydrogen peroxide (see WO 2021/097138).

While such methods in some cases provide access to unique oligosaccharides and oligosaccharide compositions, depending on the depolymerization conditions and source of polysaccharide, it is unknown whether a given oligosaccharide or oligosaccharide composition can be utilized by a given microorganism or even if specific oligosaccharide structures can modulate communities of bacteria and metabolites in various microbiomes, including the gut, skin, urogenital, respiratory, oral, and so forth. Thus, there is a need in the art for oligosaccharides that can modulate microbiomes and/or their metabolic products in targeted directions, particularly in the urogenital health context, and associated methods of such modulation.

### SUMMARY OF THE INVENTION

In some aspects, disclosed is a method of improving urogenital health in a female subject, the method comprising: administering to the female subject a therapeutically effective amount of a composition comprising one or more oligosaccharides; wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; and wherein the administering step results in improving urogenital health in the female subject.

In another aspect, disclosed is a method of improving urogenital health in a female subject, the method comprising: administering to the female subject a therapeutically effective amount of a composition comprising one or more oligosaccharides; wherein each of the one or more oligosaccharides independently contains 3 to 50 subunits; wherein at least 5% of the subunits comprise a beta-1,3 glucose residue, a beta-1,4 glucose residue, or a combination thereof; and wherein the administering step results in improving urogenital health in the female subject. In some aspects, at least 5% of the subunits comprise a beta-1,4 glucose residue. In some aspects, at least 5% of the subunits comprise a beta 1,3 glucose residue. In some aspects, the one or more oligosaccharides having subunits comprising at least 5% beta-1,4 glucose are derived from one or more materials disclosed herein. In some aspects, the one or more oligosaccharides having subunits comprising at least 5% beta-1,3 glucose are derived from one or more materials disclosed herein.

The present invention further includes a method of treating or preventing a vaginal infection, such as a bacterial or fungal infection, in a female subject, the method comprising: administering to the female subject a therapeutically effective amount of a composition comprising one or more oligosaccharides; wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; and wherein the administering step results in treating or preventing a vaginal infection in the female subject.

Further disclosed herein is a method of modulating the microbiota of the urogenital tract of a female subject, the method comprising: administering to the female subject a therapeutically effective amount of a composition comprising one or more oligosaccharides; wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; wherein the administering step comprises contacting said microbiota of the urogenital tract with said one or more oligosaccharides; and wherein the administering step results in modulating the microbiota of the urogenital tract of the female subject.

In some aspects, the invention provides a method of lowering the pH of the urogenital tract of a female subject, the method comprising: administering to the female subject a therapeutically effective amount of a composition comprising one or more oligosaccharides; wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; wherein the administering step results in lowering the pH of the urogenital tract.

In some aspects, the method enhances microbial production of lactic acid in a urogenital tract of the female subject. In some aspects, the method results in a vaginal pH of 3.5-4.5. In some aspects, the method lowers vaginal pH from a starting pH of 4.5-7 to a final pH of 3.5-4.5. In some aspects, the method increases abundance of Lactobacillus species in the female subject urogenital tract. In some aspects, the one or more oligosaccharides are preferentially metabolized by beneficial vaginal Lactobacillus species but not preferentially metabolized by vaginal dysbiosis-associated bacteria. In some aspects, the Lactobacillus species comprise L. crispatus, L. gasseri, L. jensenii, L. iners, L. vaginalis, or any combination thereof.

The present invention further provides a formulation comprising any one of the one or more oligosaccharides disclosed herein. In some aspects, the formulation further comprises a pharmaceutically acceptable carrier. In some aspects, the formulation comprises the synthetic composition, or the one or more oligosaccharides and at least one microorganism.

Further disclosed herein is a use of a composition for improving urogenital health in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof.

In some aspects, disclosed is a use of a composition for treating or preventing a vaginal infection in a female subject, such as a bacterial or fungal infection, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof.

In further aspects, the invention provides a use of a composition for modulating the microbiota of the urogenital tract in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof.

The present invention further includes a use of a composition for lowering the pH of the urogenital tract in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof.

Further disclosed herein is a medicament, for use in for improving urogenital health in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof.

In some aspects, the invention provides a medicament, for use in treating or preventing a vaginal infection in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, soy, pea, sugar cane or any combination thereof.

In another aspect, provided herein is a medicament, for use in modulating the microbiota of the urogenital tract in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof.

In some aspects, the invention provides a medicament, for use in lowering the pH of the urogenital tract in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof.

In other aspects, disclosed herein is a method of making a medicament for use in for improving urogenital health in a female subject, the method comprising combining: an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof, and an optional carrier.

In some aspects, the invention provides a method of making a medicament for use in treating or preventing bacterial vaginosis in a female subject, the method comprising combining: an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof, and an optional carrier.

Further disclosed herein is a method of making a medicament for use in modulating the microbiota of the urogenital tract in a female subject, the method comprising combining: an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof, and an optional carrier.

In some aspects, the invention provides a method of making a medicament for use in lowering the pH of the urogenital tract in a female subject, the method comprising combining: an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a part of a moringa plant, a part of a Cucurbita species, spent distillers' grains, gellan gum, xanthan gum, legumes, soy, pea, sugar cane or any combination thereof, and an optional carrier.

Without wishing to be bound by any particular theory, there may be discussion herein of beliefs or understandings of underlying principles relating to the uses, compositions, and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 illustrates growth of Lactobacillus rhamnosus on glucose, CLX109, and CLX108, measured by absorbance at 600 nm (n=3). Basal media is minimum media without carbohydrate.

FIG. 2 illustrates a comparison of lactic acid production in Bif. longum subsp. longum.

FIG. 3 illustrates a comparison of succinic acid production in Bif. longum subsp. longum.

FIG. 4 illustrates a comparison of lactic acid production in Bif. longum subsp. infantis.

FIG. 5 illustrates a comparison of succinic acid production in Bif. longum subsp. infantis.

FIG. 6 illustrates a comparison of lactic acid production in Bif. pseudocatenulatum.

FIG. 7 illustrates a comparison of succinic acid production in Bif. pseudocatenulatum.

FIG. 8 illustrates growth of Lactobacillus crispatus ATCC 33820 on glucose, lactose and 6 oligosaccharides, measured by absorbance at 600 nm (n=3). No carbohydrate is minimum media without carbohydrate.

FIG. 9 illustrates a comparison of lactic acid production in Lactobacillus crispatus ATCC 33820.

FIGS. 10A-F show ¹H-13C HSQC NMR spectra of CLX109 (FIG. 10E), CLX115 (FIG. 10B), CLX123 (FIG. 10A), CLX125 (FIG. 10C), CLX126 (FIG. 10D), and CLX128 (FIG. 10F) recorded on a Brucker 600 MHz NMR spectrometer in DMSO-d₆ with 0.03% TMS. The x-axis provides the parts per million (ppm) for F2 at intervals of 0.1 ppm.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

As used herein, the terms “about” and “approximately,” when used to modify an amount specified in a numeric value or range, means that slight variations from a stated value may be used to achieve substantially the same results as the stated value. Unless otherwise defined herein, in circumstances where this definition cannot be applied or is exceedingly difficult to apply, then the terms “about” and “approximately” mean±10% of the recited value.

As used herein, a “base” refers to a compound or collection of compounds that can accept hydrogen ions from a peroxyl oxidized carbohydrate, water, or non-aqueous solvent. In some aspects, a base includes Lewis bases, non-Arrhenius bases, weak-Arrhenius bases, other molecules that produce hydroxide ions through their decomposition, or other compounds that can accept hydrogen ions from a hydroperoxyl oxidized carbohydrate. Unless otherwise specified, base does not mean a strong-Arrhenius base.

As used herein, a “strong-Arrhenius base” as applied to the disclosed methods refers to a compound that completely dissociates in water to release one or more hydroxide ions into solution. As used herein, a “strong-Arrhenius base” as applied to the disclosed methods refers explicitly to KOH, NaOH, Ba(OH)₂, CsOH, Sr(OH)₂, Ca(OH)₂, LiOH, and RbOH.

As used herein, a “weak-Arrhenius base” as applied to the disclosed methods refers to a compound that incompletely dissociates in water to release one or more hydroxide ions into solution, e.g. ammonium hydroxide, H₂O, etc. As “weak-Arrhenius base” is used herein, there are no compounds which meet both the definition of strong-Arrhenius base and weak-Arrhenius base.

As used herein, a “non-Arrhenius base” as applied to the disclosed methods refers to a compound or atom that can donate electrons (e.g., Lewis Bases), accept protons (e.g., Bronsted-Lowry Bases), or releases hydroxide ions through its decomposition (NH₄HCO₃), but explicitly does not qualify as an Arrhenius base.

As used herein, a “Lewis base” as applied to the disclosed methods refers to a compound or atom that can donate electron pairs (e.g., F⁻, benzene, H⁻, pyridine, acetonitrile, acetone, urea, etc.).

As used herein, a “Bronsted-Lowry base” as applied to the disclosed methods refers to a compound or atom that can accept or bond to a hydrogen ion (e.g., methanol, formaldehyde, ammonia, etc.).

As used herein, a “cleavage agent” or “cleavage reagent” as applied to the disclosed methods preferably refers to a single, or collection of non-Arrhenius and/or weak-Arrhenius bases used to cleave polysaccharides after hydroperoxyl oxidation thereof. In certain aspects, a cleavage agent or cleavage reagent breaks glycosidic bonds in the polysaccharide, which bonds may be present between any two saccharides of the polysaccharide. In embodiments, the cleavage reagent (cleavage initiator) may also be, and preferably is a peroxide-quenching reagent, and in either case may be used in combination with an additional compatible peroxide-quenching agent that may or may not also be a cleavage agent. In some aspects, a cleavage reagent may be an enzyme. In some aspects, the cleavage reagent enzyme may be a glycosyl hydrolase, a lytic polysaccharide monooxygenase, a glycosyl transferase, transglycosidase, polysaccharide lyase, carbohydrate binding module, glycoysl transferase, carbohydrate esterase, a cocktail containing two or more of the aforementioned enzymes, or any enzyme that is carbohydrate active. In some aspects, a cleavage reagent may be a solid-phase acid catalyst or a solid-phase base catalyst.

As used herein “Fenton's reagent” refers to a reagent comprising a peroxide agent and a metal. In certain aspects, the peroxide agent is hydrogen peroxide. In certain aspects, the metal is Fe(II), Fe(III), Cu(I), Cu(II), Mn(II), Zn(II), Ni(II), and Co(II), alkaline earth metals Ca(II) and Mg(II), the lanthanide Ce(IV) or any combination thereof.

As used herein, “monosaccharide ratio”, “monosaccharide peak area ratio”, “ratio of monosaccharide” (sometimes referred to as “monosaccharide:monosaccharide ratio”) or similar terms refer to any number of the comparisons dependent upon the relationships observed in the hydrolytic monosaccharide compositional analysis. Absolute concentrations of each monosaccharide are calculated on a relative percent basis in relation to the summation of all other monosaccharides observed. Monosaccharide ratios are calculated by dividing one contributing monosaccharide by any other monosaccharide within the composition. Monosaccharide ratios are not intended to limit the composition to the listed monosaccharides. For example, a glucose:galactose ratio of 1:1 means that there are roughly equal amounts of glucose subunits and galactose subunits in the composition, but the composition may also comprise mannose subunits, rhamnose subunits, or any other subunit.

As used herein, the term “hydrolytic monosaccharide compositional analysis” refers to the method described in Amicucci, Galermo et al. 2019, hereby incorporated by reference in its entirety for all purposes to the extent not inconsistent with the description herein, with some modifications. The hydrolysis reaction to produce monosaccharides was performed at the optimized condition of 100° C. for 2 hours. Samples were ran on an Agilent 1290 Infinity II ultra-high performance liquid chromatography (UHPLC) system couple to an Agilent 6490A triple quadrupole (QqQ) mass spectrometer. Separation was carried out on an Agilent InfinityLab Poroshell HPH-C18 column (2.1 mm×50 mm, 1.9 μm particle size) plus a guard column (5 mm) with the same solvent system described in the paper. With a constant flow rate of 1.2 mL/min, an isocratic gradient of 8.5% B was used for the first 4-min elution period, followed by 15% B for 0.4 min. For the flush period, 97% B was held for 1 min. The column thermostat was set at 35° C. For the mass spectrometry parameters, the only change from the method described in the paper is that the fragmentor voltage was set at 380V. For data analysis, the hydrolysis correction factor was not applied since the samples herein contains oligosaccharides instead of polysaccharides. In this analysis method, monosaccharide composition is calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves. For example, 30% glucose, as measured by the herein hydrolytic monosaccharide compositional analysis, refers to containing 30 g of glucose per 100 g of the sum of all 14 monosaccharides described above.

As used herein, “linkage ratio”, “linkage peak area ratio”, “ratio of linkage” or other similar terms refer to any number of comparisons dependent upon the relationships observed in the glycosidic linkage composition analysis. For example, an oligosaccharide having a ratio of beta-1,3 linked to beta-1,4 linked residues ranging from 1:1 to 1:5 refers to a linkage ratio as measured by glycosidic linkage composition analysis. Peak area for each linkage is calculated on a relative percent basis of the peak area in relationship to the summation of all other linkage peaks areas observed. Peak area ratios are calculated by dividing one contributing linkage by any other linkage of the same monosaccharide within the composition.

As used herein, the terms “glycosidic linkage composition”, “glycosidic linkage composition analysis”, “glycosidic linkage analysis”, “permethylated linkage composition analysis” or similar terms, refer to a method described in Galermo, Nandita et al. 2018, hereby incorporated by reference in its entirety for all purposes to the extent not inconsistent with the description herein, with some modifications. The permethylation reaction time was 30 min instead. Samples were ran on an Agilent 1290 Infinity II UHPLC system couple to an Agilent 6490A QqQ mass spectrometer. Separation was carried out on an Agilent InfinityLab Poroshell HPH-C18 column (2.1 mm×100 mm, 1.9 μm particle size) plus a guard column (5 mm) with the same solvent system described in the paper. With a constant flow rate of 0.8 mL/min, an isocratic gradient of 14% B was used for the 16-min elution period, followed by a 2-min 99% B flush period. The column thermostat was set at 35° C. The short-hand glycosidic linkage annotation used herein is also described in this paper. In this method, glycosidic linkage composition is calculated by integrating the chromatographic peak area of all peaks with the following m/z values: 481.2, 495.2, 509.2, 523.3, 525.2, 537.3, 539.3, 553.3, 567.3, 581.3. For example, 20% 4-galactose, as measured by the herein described method permethylated linkage composition analysis, refers to the peak area of 4-galactose being 20% of the sum of the peak area of all linkage peaks with the m/z values listed above.

As used herein, the term “other minor linkages” refers to the sum of linkages which are either not entirely annotated or constitute less than 2% of any samples. Therefore, the contributions of these linkages to the sample glycosidic linkage composition are summed into this “other minor linkages” category.

As used herein, the term “oligosaccharide analysis” refers to a HPLC-quadrupole time-of-flight (Q-TOF) method described in Amicucci, Nandita et al. 2020, hereby incorporated by reference in its entirety for all purposes to the extent not inconsistent with the description herein, with some modifications. For sample prep, oligosaccharides were reduced by incubation with 2.0 M NaBH4 for 1 h at 65° C. Oligosaccharides were purified using C-18 cartridge 96-well plates: plates were washed with 100% ACN, and the oligosaccharides were loaded and eluted with water. Oligosaccharides were subsequently purified using porous graphitized carbon (PGC) 96-well plates: PCG plates were washed with 80% acetonitrile and 0.1% (v/v) TFA in water, and the oligosaccharides from C-18 purification were loaded and washed with water. The oligosaccharides were eluted with 40% acetonitrile with 0.05% (v/v) TFA. Samples were completely dried by evaporative centrifugation and reconstituted for mass spectrometry analysis. Instrumentation was performed on an Agilent 1260 Infinity II HPLC coupled to an Agilent 6530 Q-TOF mass spectrometer. Using the same stationary (plus a 5 mm guard column) and mobile phase as described in the paper, separation was carried out using the following gradient: 2-15% B, 0-20 min; 15-60% B; 20-45 min. The column thermostat was set at 35° C. The fragmentor voltage was set at 75V. In this method, “oligosaccharide weight %” is calculated by dividing the chromatographic peak area of a particular oligosaccharide by the total peak area of all oligosaccharides identified in that sample during the defined chromatographic period.

As used herein, “retention factor” refers to the ratio obtained by dividing the retention time of a given peak observed in an oligosaccharide analysis (e.g., HPLC spectrum) by the first oligosaccharide peak (i.e., the lowest retention time) observed in the oligosaccharide analysis.

As used herein, “enhances microbial production” refers to a biologically relevant increase in production of a particular metabolite or group of metabolites. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1,000% increase or more. In some aspects, the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “decreases microbial production” refers to a biologically relevant decrease in the production of a particular metabolite or group of metabolites. In some aspects, a biologically relevant decrease is a statistically significant change as measured by parametric or non-parametric tests. In some aspects, a biologically relevant decrease can be measured in feces, serum, urine, or organ tissue. In some aspects, a biologically relevant decrease is measured through a host metabolic product (choline can be measured as TMA or TMAO). In some aspects, a biologically relevant decrease is a 10% decrease or a 20% decrease or a 50% decrease or a 75% decrease or a 90% decrease or more. In some aspects, the biologically relevant decrease can be in the absolute amount of a metabolite or group of metabolites. In some aspects, the biologically relevant decrease can be the rate that a metabolite or group of metabolites are produced. In some aspects, the biologically relevant decrease can be in the relative amount of a metabolite or group of metabolites.

As used herein, “decreases microbial utilization” refers to a biologically relevant increase in the amount of a particular metabolite or group of metabolites due to lower microbial utilization. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1,000% increase or more. In some aspects the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “slows microbial utilization” refers to a biologically relevant increase in the amount or buildup of a particular metabolite or group of metabolites due to slowed microbial utilization. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1,000% increase or more. In some aspects the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “increases abundance of” refers to a biologically relevant increase in the population of a certain bacterial taxa. In some aspects, a biologically relevant increase is a statistically significant change as measured by parametric or non-parametric tests. In some aspects, a biologically relevant increase can be measured in feces, jejunum, cecum, ileum, stomach, large intestines, duodenum, mouth, respiratory tract, skin, urogenital tract, vaginal tract, or other microbial community. In some aspects, a biologically relevant increase is a 10% increase or a 5× increase or a 10× increase or a 50× increase or a 100× or 1,000× increase or 10,000× or more. In some aspects the biologically relevant increase can be in the absolute amount of a taxa or group of taxa. In some aspects, the biologically relevant increase can be the rate that a taxa or group of taxa increases in the microbial community. In some aspects, the biologically relevant increase can be in the relative amount of a taxa or group of taxa in a microbial community. In some aspects, an increase in abundance refers to the presence of one microbial taxa as compared to another microbial taxa.

“Oligosaccharide preferentially metabolized by beneficial vaginal Lactobacillus species but not preferentially metabolized by vaginal dysbiosis-associated bacteria” (and similarly worded phrases) means an oligosaccharide which has a monosaccharide structure including glycosidic bonds which can be depolymerized by glycosyl hydrolases of vaginal Lactobacillus species but not those of bacteria associated with vaginal dysbiosis, such as Gardnerella vaginalis, and Atopobium vaginae (or at least such vaginal dysbiosis bacteria cannot depolymerize and utilize such oligosaccharides as effectively or efficiently as Lactobacillus species).

In one aspect of the invention, it is believed that Lactobacillus species preferentially metabolize oligosaccharides having beta 1,4 glucose linkages. In keeping with this aspect, oligosaccharides having beta 1,4 glucose linkages at a concentration of 5% or more (e.g., between 5-100%, between 10-100%, between 15-100%, between 20-100%, between 30-100%, between 40-100%, between 50-100%, between 75-100%, between 85-100%, or between 95-100% beta 1,4 glucose linkages), such as beta-glucan-, xanthan-, gellan gum-, and cello-derived oligosaccharides, are expected to result in higher rates of consumption by Lactobacillus species than oligosaccharides without beta 1,4 glucose linkages. In another aspect of the invention, it is believed that Lactobacillus species preferentially metabolize oligosaccharides having beta 1,3 glucose linkages. For example, oligosaccharides having beta 1,3 glucose linkages at a concentration of 5% or more (e.g., between 5-100%, between 10-100%, between 15-100%, between 20-100%, between 30-100%, between 40-100%, between 50-100%, between 75-100%, between 85-100%, or between 95-100% beta 1,3 glucose linkages), such as oligosaccharides derived from beta-glucan and curdlan, are expected to result in higher rates of consumption by Lactobacillus species than oligosaccharides without beta 1,3 glucose linkages. In further aspects, it is believed that Lactobacillus species preferentially metabolize oligosaccharides having both beta 1,4 glucose linkages and beta 1,3 glucose linkages. In keeping with this aspect, oligosaccharides having beta 1,4 and beta 1,3 glucose linkages at a total concentration of 5% or more (e.g., between 5-100%, between 10-100%, between 15-100%, between 20-100%, between 30-100%, between 40-100%, between 50-100%, between 75-100%, between 85-100%, or between 95-100% beta 1,4 glucose linkages) are expected to result in higher rates of consumption by Lactobacillus species than oligosaccharides without beta 1,4 or beta 1,3 glucose linkages. In some embodiments, Lactobacillus species preferentially metabolize short-chain oligosaccharides (e.g., oligosaccharides having a degree of polymerization between 2-10, 2-8, or 2-6) than long-chain oligosaccharides.

As used herein “oligosaccharide” refers to an oligomer of saccharides, in which the mean DP of the oligomer is between 2 and 30 monosaccharide units, such as between 3-30, 3-20, 3-20, 3-15, 3-10, 3-8, 3-6, 5-30, 5-20, or 5-10 monosaccharide units. An oligosaccharide can be linear, branched, primarily linear with pendant saccharide monomers, or any combination thereof. An “oligosaccharide” refers to an individual oligomer chain.

As used herein, “oligosaccharide composition”, “oligosaccharide pool”, or “oligosaccharide mixture” refers to a mixture of two or more oligosaccharides, each of which can be the same or different from one another. Although efforts have been made herein to consistently use the terms “oligosaccharide” and “oligosaccharide composition” according to their preceding definitions, the intended meaning will be clear from context when such terms are used herein. In embodiments, “one or more oligosaccharides” refers to an oligosaccharide mixture when more than one oligosaccharide is present.

As used herein, the term “synthetic oligosaccharide” refers to an oligosaccharide produced by the depolymerization of one or more polysaccharides. In certain aspects, the term synthetic oligosaccharide refers to compositions of oligosaccharides produced by the methods disclosed herein and/or methods incorporated by reference. In some aspects, the term synthetic oligosaccharide refers to oligosaccharides prepared by synthesizing the oligosaccharide from monosaccharides.

As used herein, the term “synthetic composition” means a composition which is artificially prepared and preferably means a composition containing at least one compound that is produced ex vivo chemically and/or biologically, e.g., by means of chemical reaction, enzymatic reaction, recombinantly, or any combination thereof. The synthetic composition typically comprises one or more compounds, including one or more of the oligosaccharides described herein.

As used herein, the term “polysaccharide” refers to a polysaccharide or a material comprising a polysaccharide. Additionally, as used herein, the term “polysaccharide” refers to any carbohydrate polymer and, in some aspects, is also linked to other non-carbohydrate moieties (e.g., glycoproteins, proteoglycans, glycopeptides, glycolipids, glycoconjugates, glycosides, or any combination thereof). Moreover, as used herein “polysaccharide” refers to a polymer having greater than 30 monosaccharide units, and, in some aspects, generally contains up to 500,000 monosaccharides, or a material comprising such a polymer. A polysaccharide can be a linear polymer, branched polymer, primarily linear polymer with pendant saccharide monomers, or any combination thereof. Polysaccharides are complex carbohydrates found ubiquitously in various organisms including, e.g., plants, mammals, insects, fungi, bacteria, diatoms, and algae. Polysaccharides are generally used for their rheological properties but more recently have been explored for their prebiotic and immunomodulation potential.

As used herein, the term “subunit” (sometimes referred to as “unit” or “residue”) means a species that is covalently bonded to or within an oligomer (e.g., oligosaccharide) or polymer (e.g., polysaccharide). In some aspects, such species generally include saccharides (e.g., glucose, galactose, mannose, etc.). For example, when an oligosaccharide composition comprises a glucose subunit, it means that the composition comprises a glucose molecule that is bound to or within an oligomer or polymer. Therefore a composition that contains only free monomeric glucose would not contain a glucose subunit. Similarly, when an oligosaccharide composition comprises a sum of glucose, galactose, and mannose subunits in an amount of at least 60 wt. % based on total weight of saccharide subunits, this means that the mass of all of the glucose subunits, galactose subunits, and mannose subunits are summed, and the subunits of all saccharides are summed, and then the first sum is divided by the second sum. Additionally, when an oligosaccharide composition comprises non-terminal galactose subunits, and at least 70 wt. % of the non-terminal galactose subunits are specified to have at least one 4-linkage, this feature is calculated by summing the mass of all non-terminal galactose subunits having at least one 4-linkage (and this can include, for example, galactose subunits with 4,6-linkages and 4,3-linkages), and then dividing by the total mass of non-terminal galactose subunits regardless of linkage type. The same concept is applicable to any feature where reference to “at least one X-linkage,” in which X is an integer (e.g., such as “a weight ratio of glucose subunits having at least one 4-linkage to glucose subunits having at least one 3-linkage is between 2:1 to 4:1” and other such features). Moreover, in such calculations the actual mass of the subunit is used (i.e., in bound form) rather than the mass of the unit as if it was hydrolyzed (which would add the mass of water). Other features can be calculated similarly. These features can be determined with the aid of the various analytical techniques, such as hydrolytic monosaccharide compositional analysis, oligosaccharide analysis, glycosidic linkage analysis, NMR HSQC Analysis, and so forth, as well as other techniques known in the art.

The “degree of polymerization” or “DP” of an oligosaccharide or polysaccharide refers to the total number of sugar monomer units that are part of a particular carbohydrate. For example, a tetra galacto-oligosaccharide has a DP of 4, having 3 galactose moieties and one glucose moiety. When used to describe a group of oligosaccharides (e.g., an oligosaccharide composition), DP generally refers to the mean DP of the oligosaccharides in the composition. In some aspects, the DP of an oligosaccharide is referred to as “DP #”, where “#” corresponds to an integer representing the total number (or average number if used to describe a group of oligosaccharides) of sugar monomer units (e.g., “DP3” means a degree of polymerization of 3). For oligosaccharides or oligosaccharide compositions discussed herein having a DP between the range of 3-9, the recited DP includes a variance of up to ±2 monomer units of the recited value. For oligosaccharides or oligosaccharide compositions having a DP of 10 or greater, the recited DP includes a variance of up to ±20% of the recited value.

Disclosed herein are examples of starting materials (sometimes referred to herein as “one or more materials,” “materials,” “parent,” “parent polysaccharides,” or similar terms) from which certain oligosaccharides and compositions thereof are derived. It will be appreciated by one having skill in the art that any toxic portions of said starting materials will be minimized or excluded from the methods, compositions, medicaments, and formulations herein.

As used herein, “glucuronoxylan” is a polysaccharide with a glycosidic linkage composition comprising a β-1,4 xylose backbone with about 13% α-1,2 Glucose-4-OMe.

As used herein, “homo-xylan” is a polysaccharide with a glycosidic linkage composition comprising at least 90% β-1,4 xylose backbone.

As used herein, “arabinan” is a polysaccharide with a glycosidic linkage composition comprising at least 75% arabinose sub-units comprised of α-1,3, α-1,5, and α-1,3,5 linkages. In some aspects, arabinan contains 10-20% galactose subunits, 10-20% xylose subunits, or 10-20% of a combination of galactose and xylose subunits. In some aspects arabinan is “de-branched” and contains only α-1,5 arabinose linkages.

As used herein, “arabinan oligosaccharide” means an oligosaccharide that contains alpha-linked arabinan residues and that optionally resembles a beetroot arabinan and/or a legume arabinan, such as, a pea arabinan, and/or a soy arabinan. In some aspects, the oligosaccharide comprises alpha 1-5, alpha 1-3, or alpha 1-2 glycosidic linkages. Arabinan oligosaccharides can be linear or branched. In some aspects, the molecular weight distribution of arabinan oligosaccharides is such that at least 50% of the mass is smaller than 50 kDa. Arabinan oligosaccharides can be made through Fenton-type depolymerizations as described in WO2021097138A1, WO2018236917A1, WO2020247389A1, and WO2022241163A1, which are each incorporated by reference herein in its entirety, and more specifically for methodologies of synthesis, to the extent not inconsistent with the description herein. Oligosaccharides from the compositions CLX122 and CLX122DSF are examples of arabinan oligosaccharides.

In embodiments herein glycosidic linkages, particularly those in arabinan oligosaccharides, are described as trisecting and in particular as trisecting in the 2, 3 and 5 position. Glycosidic linkages representing terminal, linear, bisecting, and trisecting monomers of glucose, galactose, mannose, xylose, arabinose, ribose, fucose, rhamnose, glucuronic acid and galacturonic acid are known in the art. [Galermo et al., 2018; Galermo et al., 2019]. Methods for characterization of glycosidic linkages, particularly with respect to bisecting or trisecting linkages are provided, for example, in Galermo et al., 2018, Galermo et al., 2019 and Amicucci et al., 2019 (Analytical Chemistry), each of which is incorporated by reference herein in its entirety, and more specifically for methods of characterizing glycosidic linkages in general and for characterizing glycosidic linkages as bisecting or trisecting, to the extent not inconsistent with the description herein.

As used herein, “barley” comprises a polysaccharide comprising beta glucan with a glycosidic linkage composition comprising a glucose backbone comprising β-1,4 and β-1,3 in about a 4 to 1 ratio.

As used herein, “lichenan” is a polysaccharide (comprising beta glucan) with a glycosidic linkage composition comprising a β-1,4 glucose backbone with alternating β-1,3 glucose about 33% of the time.

As used herein, “galactomannan” is a polysaccharide with a glycosidic linkage composition comprising a β-1,4 mannose backbone, with about 22% α-1,3 galactose branching.

As used herein, “beet” or “beet plant” refers to any part of the plant in the genus Beta. “Beets” may refer to Beta vulgarisor or other distinct species and subspecies, adanesisi, maritima, vulgaris, altissima, circla, flavescens, conditiva and crassa. “Beet” may refer to by-products of the plant during harvest or food processing, non-limiting examples include Beet tap roots, beet stems, beet leaves, beet powder, beet fiber, and beetroots. “Beets” may refer to the solid material after roasting, fermentation, hot-water, enzymatic, chemical, alkaline, super critical fluid, sun drying, organic solvent, acidic, mechanical pressure, candied, pickling or pressure based extractions. “Beets” may refer to other non-beta genus, which are colloquially known as sugar beets, sea beets, spinach beets, Swiss chard, beet root, table beets, garden beet, red beet, dinner beat golden beet or mangel-wurzel.

As used herein, “β-glucan” (also called “beta glucan”) is a polysaccharide that contains β-linked glucose residues. Beta glucan includes cereal beta glucans, yeast beta glucans, and fungal beta glucans. In some aspects, beta glucan polymers comprises beta 1-3, beta 1-4, or beta 1-6 glycosidic linkages. In some aspects, beta glucan polymers comprises a glycosidic linkage composition comprising a glucose backbone comprising β-1,4 and β-1,3 in about a 4 to 1 ratio. Beta glucans can be linear or branched. In some aspects, within each class of beta glucans, the distribution of polymers is such that at least 80% of the mass is larger than 50 kDa. “Beta glucan” can refer to the solid material after roasting, fermentation, hot-water, enzymatic, chemical, alkaline, super critical fluid, sun drying, organic solvent, acidic, mechanical pressure or pressure based extractions.

As used herein, “β-glucan oligosaccharide” (also called “beta glucan oligosaccharide”) means an oligosaccharide that contains beta-linked glucose residues and that resembles a cereal beta glucan, a yeast beta glucan, and/or a fungal beta glucan. In some aspects, the oligosaccharide comprises beta 1-3, beta 1-4, and/or beta 1-6 glycosidic linkages. Beta glucan oligosaccharides can be linear or branched. In some aspects, the molecular weight distribution of beta glucan oligosaccharides is such that at least 50% of the mass is smaller than 5 kDa. In some aspects, beta glucan oligosaccharides is created through enzymatic, chemical, or biological synthesis or through the depolymerization of beta glucans via enzymatic, chemical, physical, or biological processes. In some aspects, beta glucan oligosaccharides is made through Fenton-type depolymerizations as described in WO2021097138A1, WO2018236917A1, WO2020247389A1, and WO2022241163A1, which are each incorporated by reference herein in its entirety, and more specifically for methodologies of synthesis, to the extent not inconsistent with the description herein. Oligosaccharide compositions CLX112, CLX115, and CLX115Cu are all examples of beta glucan oligosaccharides.

“Cereal beta glucan” means a beta glucan found in the cell walls of cereals and which contains beta-linked glucose units that are in the beta-3 position and beta-4 positions. In cereals, the cereal beta glucan may be found alongside other polymers such as cellulose, starch, and arabinoxylans. In an embodiment, cereal beta glucan has a structure in which the linear polymer is comprised of beta-4 linked glucose residues with beta-3 linked residues interspersed at a ratio of about 1:1 to 5:1 beta-4:beta-3 linked glucose residues. In an embodiment, cereal beta glucan has a structure in which the linear polymer is comprised of beta-4 linked glucose residues with beta-3 linked residues interspersed at a ratio of about 3:1 to 5:1 beta-4:beta-3 linked glucose residues. Cereal beta glucan can be obtained from cereals and grains such as oats, barley, wheat, rye, and rice, for example. Cereal beta glucans can be extracted from the bran or the endosperm of cereals and grains, and can be the solid material after roasting, fermentation, hot-water, enzymatic, chemical, alkaline, super critical fluid, sun drying, organic solvent, acidic, mechanical pressure or pressure-based extractions. In some aspects, the distribution of polymers in cereal beta glucan is such that at least 80% of the mass is larger than 50 kDa.

“Cereal beta glucan oligosaccharide” means an oligosaccharide that resembles beta glucan found in the cell walls of cereals and contains beta-linked glucose units that are in the beta-3 position and beta-4 positions. Cereal beta glucan oligosaccharides may be found alongside polysaccharides or oligosaccharides such as cellulose, starch, and arabinoxylans in their polysaccharide or oligosaccharide forms. Cereal beta glucan oligosaccharides can have a structure in which the linear polymer is comprised of beta-4 linked glucose residues with beta-3 linked residues interspersed at a ratio of about 1:1 to 5:1 beta-4:beta-3 linked glucose residues. In some aspects, cereal beta glucan oligosaccharides have a structure in which the linear polymer is comprised of beta-4 linked glucose residues with beta-3 linked residues interspersed at a ratio of about 3:1 to 5:1 beta-4:beta-3 linked glucose residues. In some aspects, cereal beta glucan oligosaccharides are derived from cereal beta glucan. In some aspects, the molecular weight distribution of cereal beta glucan oligosaccharides is such that at least 50% of the mass is smaller than 5 kDa. In some aspects, cereal beta glucan oligosaccharides refer to oligosaccharides created through enzymatic, chemical, or biological synthesis or through the depolymerization of beta glucans via enzymatic, chemical, physical, or biological processes. In some aspects, cereal beta glucan oligosaccharides are made through Fenton-type depolymerizations as described in WO2021097138A1, WO2018236917A1, WO2020247389A1, WO2022241163A1, which are each incorporated by reference herein it its entirety, and more specifically for methodologies of synthesis, to the extent not inconsistent with the description herein. Oligosaccharides CLX112, CLX115, and CLX115Cu are all examples of “cereal beta glucan oligosaccharides”.

As used herein, “legume” refers to any part of a plant in the family Fabaceae (or Leguminosae), or the fruit or seed of such a plant. Examples of legumes include peas, beans, soy, chickpeas, peanuts, lentils, lupins, mesquite, carob, tamarind, alfalfa, and clover. In some aspects, legume refers to by-products of the plant during harvest or food processing. Non-limiting examples include powders, pods, flowers, stems, roots, seeds, fiber, or crude protein. Legume may refer to the solid material after roasting, fermentation, hot-water, enzymatic, chemical, alkaline, super critical fluid, sun drying, organic solvent, acidic, mechanical pressure or pressure-based extractions.

As used herein, “pea” refers to any part of the plant in the genus Pisum, Cajanus, lathyrus or Vigina. In some aspects, “pea” refers to Pisum sativum, Cajanus cajanor, Vigna unguiculata, Lathyrus aphaca or other species. In some aspects, “pea” refers to by-products of the plant during harvest or food processing, non-limiting examples include pea powder, pea pods, pea flower, pea stem, pea stipules, pea root, pea seeds, pea fiber, or crude pea protein. In some aspects, “pea” refers to the solid material after roasting, fermentation, hot-water, enzymatic, chemical, alkaline, super critical fluid, sun drying, organic solvent, acidic, mechanical pressure or pressure based extractions. “Pea” may refer to other non-Pisum, Cajanus, lathyrus or Vigina genus, which are colloquially known as pea, snow pea, split pea, snap pea, field pea or sugar pea.

As used herein, “soy” refers to any part of the plant in the genus Glycine or Soja. “Soy” may refer to Dolichos soja L., Glycine angustifolia Miq., Glycine gracilis Skvortsov, Glycine hispida (Moench) maxim., Glycine soja, Phaseolus max L., Soja angustifolia, Soja hispida Moench, Soja japonica savi, Soja max, Soja H., Soja viridis or other species. In some aspects, “soy” refers to by-products of the plant during harvest or food processing, non-limiting examples include, Soy root, soy stem, soy leaves, soy flowers, soy fruiting pods, soy bean, soy protein, soy okra (pulp or curd), soy fiber or soy bean testa. In some aspects, “soy” refers to the solid material after roasting, fermentation, hot-water, enzymatic, chemical, alkaline, super critical fluid, sun drying, organic solvent, acidic, mechanical pressure or pressure based extractions. “Soy” may refer to other non-Glycine or Soja genus, which are colloquially known as soy bean, kongbiji or soya.

As used herein, “Sphingomonas elodea extract” refers to any part of the bacteria in the genus Sphingomonas. “Sphingomonas elodea extract” may refer to Pseudomonas elodea or other species. “Sphingomonas elodea” may refer to by-products of the bacteria during harvest or food processing, non-limiting examples include, extracellular polysaccharides, intracellular polysaccharides, Sphingomonas elodea cell wall, Spingomonas elodea carbohydrate membrane, or purified Sphingomonas elodea gellan gum polysaccharides. “Sphingomonas elodea extract” may refer to the solid material after roasting, fermentation, hot-water, enzymatic, chemical, alkaline, super critical fluid, sun drying, organic solvent, acidic, mechanical pressure or pressure based extractions. “Sphingomonas elodea extract” may refer to other non-Sphingomonas which are colloquially known as gellan gum, bacteria extract or gelling agent.

As used herein, “sugar cane” refers to any part of the plant in the genus Saccharum. “Sugar Cane” may refer to Saccharum officinarum, Saccharum sinense, Saccharum barberi, Saccharum arundinaceum, Saccharum bengalense, Saccharum edule, Saccharum procerum, Saccharum ravennae, Saccharum robustum, Saccharum spontaneum, hybrids of two, three or more species, or other species. In some aspects, “sugar cane” refers to by-products of the plant during harvest or food processing, non-limiting examples include, Sugar cane Leaf (barbojo), Sugar cane stalks (cane), raw sugarcane cylinders or cubes, sugar cane bagasse, fresh sugar cane juice, Sugar cane molasses, Sugar cane rapadura, Sugar cane flour or Processed sugar cane. “Sugar Cane” may refer to the solid material after roasting, fermentation, hot-water, enzymatic, chemical, alkaline, super critical fluid, sun drying, organic solvent, acidic, mechanical pressure or pressure based extractions. “Sugar Cane” may also refer to “Power Cane”. In some aspects, “sugar Cane” refers to other non-Saccharum genus, which are colloquially known as sugar cane.

As used herein, “Xanthomonas campestris extract” refers to any part of the bacteria in the genus Xanthomonas. “Xanthomonas campestris extract” may refer to extracts from Xanthomonas campestris pv. armoraciae, Xanthomonas campestris pv. begoniae A, Xanthomonas campestris pv. begoniae B, Xanthomonas campestris pv. campestris, Xanthomonas campestris pv. cannabis, Xanthomonas campestris pv. carota, Xanthomonas campestris pv. corylina, Xanthomonas campestris pv. dieffenbachiae, Xanthomonas campestris pv. glycines syn. Xanthomonas axonopodis pv. glycines, Xanthomonas campestris pv. graminis, Xanthomonas campestris pv. hederae, Xanthomonas campestris pv. hyacinthi, Xanthomonas campestris pv. juglandis, Xanthomonas campestris pv. malvacearum or Xanthomonas citri subsp. malvacearum, Xanthomonas campestris pv. musacearum, Xanthomonas campestris pv. mangiferaeindicae, Xanthomonas campestris pv. mori, Xanthomonas campestris pv. nigromaculans, Xanthomonas campestris pv. pelargonii, Xanthomonas campestris pv. phaseoli, Xanthomonas campestris pv. poinsettiicola, Xanthomonas campestris pv. pruni, Xanthomonas campestris pv. raphani, Xanthomonas campestris pv. sesami, Xanthomonas campestris pv. tardicrescens, Xanthomonas campestris pv. translucens, Xanthomonas campestris pv. vesicatoria, Xanthomonas campestris pv. viticola or other species. “Xanthomonas campestris extract” may refer to by-products of the bacteria during harvest or food processing, non-limiting examples include, Xanthomonas campestris extracellular polysaccharides, Xanthomonas campestris intracellular polysaccharides, Xanthomonas campestris cell wall, Xanthomonas campestris carbohydrate membrane, or purified Xanthomonas campestris xanthan gum polysaccharides. “Xanthomonas campestris extract” may refer to the solid material after roasting, fermentation, hot-water, enzymatic, chemical, alkaline, super critical fluid, sun drying, organic solvent, acidic, mechanical pressure or pressure based extractions. “Xanthomonas campestris extract” may refer to other non-Xanthomonas which are colloquially known as xanthan gum, bacteria extract or gelling agent.

As used herein, “xanthan gum” refers to a polysaccharide with a β-1,4 glucose backbone with alternating α-1,2 mannose. Xanthan gum can be derived from Xanthomonas campestris. In some aspects, the oligosaccharides derived from xanthan gum (“xanthan gum oligosaccharide”) possess various structural features similar to those in the parent polysaccharide. In some aspects, xanthan gum oligosaccharides comprise a composition having 63.86% glucose and 30.64% mannose. In some aspects, xanthan gum oligosaccharides comprise a glycosidic linkage composition of 29.88% 4-glucose, 16.02% 2-mannose, 12.24% 3,6-galactose, 11.54% terminal glucose and 6.09% 4,6-mannose. In some aspects, xanthan gum oligosaccharides are created through enzymatic, chemical, or biological synthesis or through the depolymerization of beta glucans via enzymatic, chemical, physical, or biological processes. In some aspects, xanthan gum oligosaccharides are made through Fenton-type depolymerizations as described in WO2021097138A1, WO2018236917A1, WO2020247389A1, and WO2022241163A1, which are each incorporated by reference herein it its entirety, specifically for methodologies of synthesis, to the extent not inconsistent with the description herein. Oligosaccharide CLX123 provides examples of xanthan gum oligosaccharides.

As used herein, a “therapeutically effective amount” of the disclosed compounds (e.g., oligosaccharide or oligosaccharide composition) is a dosage of the compound that is sufficient to achieve a desired therapeutic effect, such as an anti-inflammatory effect, stimulation of growth of specified microbiota, and so forth. For example, a therapeutically effective amount of a compound may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day. A therapeutically effective amount can be administered in one or more doses to achieve the desired treatment outcome. The compositions herein (e.g., pharmaceutical compositions) may administered on a regular basis, such as once-a-day, twice-a-day, every two days, weekly, or bi-weekly for a specified time period in order to achieve and/or maintain the desired therapeutic effect.

As used herein, “improving urogenital health in a female subject” refers to one or more of modulating the microbiota of the urogenital tract of a female subject, treating or preventing a vaginal infection, maintaining and/or lowering the pH of the urogenital tract of a female subject, selectively enhancing the growth or metabolic activity of certain beneficial bacteria, ameliorating one or more symptoms of a disease, condition, or pathology (such as pain, itching, tenderness, odor, discomfort, dryness, etc.), any other effect believed to support the overall health of the urogenital tract of a female subject, or any combination thereof. Additionally, in embodiments where a female subject is at risk of, currently has, or has had but no longer has, bacterial vaginosis, “improving urogenital health in a female subject” includes preventing, treating, or ameliorating bacterial vaginosis or a symptom thereof, or any combination thereof, such as reducing the risk or recurrence of a urinary tract infection. Moreover, in embodiments where a female subject is at risk of, currently has, or has had but no longer has, a fungal infection, “improving urogenital health in a female subject” includes preventing, treating, or ameliorating a fungal infection or a symptom thereof, or any combination thereof, such as reducing the risk or recurrence of a yeast infection.

As used herein, “treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease or condition in a susceptible subject, a reduction in severity of some or all clinical symptoms of the disease or condition (e.g., pain and/or discomfort), a slower progression of the disease or condition, an improvement in the overall health or well-being of the subject, or by other parameters well known in the art that are specific to the particular disease or condition. The phrases “treating a disease” and “treating a condition” are inclusive of inhibiting the full development of a disease or condition, for example, in a subject who is at risk for a disease or condition, or who has a disease or condition, such as inflammatory bowel disease (IBD), bacterial vaginosis, vulvovaginal candidiasis, and so forth. Consistent with these definitions, “treatment” also includes addressing an underlying nutritional need. “Treating,” “treatment,” and “therapy” are used interchangeably.

“Preventive treatment” or “prevention” means treatment given or action taken to diminish the risk of onset or recurrence of a disease, such as prophylactically administering a composition to a subject who does not exhibit signs of a disease or condition, or exhibits only early signs of the disease or condition, for the purpose of decreasing the risk of developing a pathology or condition, or diminishing the severity of a pathology or condition. Although in some aspects the disease or condition is avoided, either permanently or subject to re-treatment, in alternative aspects the onset of the disease or condition is delayed. “Preventing,” “prevention,” “preventative treatment,” and “prevent” are used interchangeably.

As used herein, “reduction” refers to any measureable decrease to achieve a desired effect. Any variation of the term, “reduction”, such as “reducing” or “reduce” are used interchangeably.

“Primary prevention” means prevention of the initial onset of a condition in a subject.

“Secondary prevention” means, in a subject who has a condition or who has had a condition, (i) prevention of reoccurrence of the condition, (ii) increase in the duration of remission of the condition, and/or (iii) reduction in severity of symptoms of the condition.

As used herein, “live biotherapeutic” refers to a therapeutic or medicinal product that comprises a living microorganism, such as an archaeon, a bacterium, an algae, a fungus (e.g., a yeast), or any combination thereof, as an active ingredient. In some aspects, the live biotherapeutic may comprise an engineered (e.g., genetically modified) or un-engineered living organism, or a mixture of both engineered and un-engineered living organisms.

A “prebiotic” or “prebiotic nutrient” is generally a non-digestible or partially-digestible (i.e., digestible by the subject/human/animal, and does not include digestion by microbes) food ingredient that beneficially affects a host when ingested by selectively stimulating the growth and/or the activity of one or a limited number of microbes in the gastrointestinal tract, urogenital system, or other portion of the host. As used herein, the term “prebiotic” refers to the above described non-digestible or partially-digestible food ingredients in their non-naturally occurring states, e.g., after purification, chemical or enzymatic synthesis as opposed to, for instance, in whole human milk.

A “probiotic” refers to live microorganisms that when administered in adequate amounts confer a health benefit on the host.

As used herein, the term “microbiota”, “microflora” and “microbiome” mean a community of living microorganisms that typically inhabits a bodily organ or part, for example, the gastro-intestinal or urogenital organs of complex organisms, such as mammals and humans. Dominant members of the urogenital microbiota, for example, include Lactobacillus crispatus, Lactobacillus jensenii, Lactobacillus gasseri, Lactobacillus iners, and Lactobacillus vaginalis.

The term “Bifidobacterium” and its synonyms refer to a genus of anaerobic bacteria having beneficial properties for humans. Members of the Bifidobacterium genus are some of the major strains that make up the gut microbiome, the bacteria that reside in the gastrointestinal tract and have health benefits for their hosts (Guarner and Malagelada 2003).

As used herein, the term “modulate” refers to the ability of a disclosed compound (e.g., oligosaccharide or oligosaccharide composition) to alter the amount, degree, or rate of a biological function, the progression of a disease, or amelioration of a condition. For example, modulating can refer to the ability of a compound to elicit a decrease in pain, discomfort, itching, odor or other conditions associated with bacterial vaginosis and/or vulvovaginal candidiasis.

As used herein, the term “modulation of microbiota” (or “modulating microbiota,” or similar terms) means exerting a modifying or controlling influence on microbiota, for example, an influence leading to an increase in the indigenous intestinal abundance of Bifidobacterium, and/or butyrate producing bacteria. In another example, the influence may lead to a reduction of the intestinal abundance of Ruminococcus gnavus and/or Proteobacteria. In yet another example, the influence involves increasing the urogenital (e.g., vaginal) abundance of certain Lactobacillus species, including Lactobacillus crispatus, Lactobacillus jensenii, Lactobacillus gasseri, Lactobacillus vaginalis, or any combination thereof, or, in some aspects, not increasing (and sometimes actively decreasing) the abundance of microbiota associated with vaginal dysbiosis, including Gardnerella vaginalis, Atopobium vaginae, Prevotella spp., Mobiluncus spp., Lactobacillus iners, or any combination thereof. In some aspects, the influence involves a combination of increasing urogenital (e.g., vaginal) abundance of such Lactobacillus species and not increasing (or actively decreasing) the abundance of such microbiota associated with vaginal dysbiosis.

As used herein, the term “relative abundance of a bacteria” means the abundance of that bacteria relative to other bacteria in the microbiota in or on the particular organ of a complex organism, such as a human or mammal.

As used herein, the term “oral administration” means any form for the delivery of a composition to a subject through the mouth. Accordingly, oral administration is a form of enteral administration.

As used herein, the term “enteral administration” means any form for delivery of a composition to a subject that causes the deposition of the composition in the gastrointestinal tract (including the stomach). Methods of enteral administration include feeding through a naso-gastric tube or jejunum tube, oral, sublingual and rectal.

As used herein, the term “topical administration” means delivery of a composition to a body surface such as the skin or mucous membrane, for example the skin or mucous membrane in the urogenital area.

As used herein, the term “intra-vaginal administration” means any form for delivery of a composition into the vagina.

“Absorbent article” means devices which absorb and contains body exudates, and, more specifically, refers to devices which are placed against or in proximity to the body of the wearer to absorb and contain the various exudates discharged from the body. Typical absorbent articles include diapers, adult incontinence briefs, training pants, diaper holders and liners, absorbent inserts, feminine hygiene products such as sanitary napkins and panty liners. Absorbent articles also include wipes, such as household cleaning wipes and feminine wipes.

As used herein, the term “feminine hygiene product” means a personal care product used by women during menstruation, vaginal discharge, and other bodily functions related to the vulva. Feminine hygiene product may include sanitary napkins or towels, panty liners, tampons, menstrual cups, and feminine wipes.

As used herein, the term “absorbent insert” means a component of an absorbent article that is adapted to contain and/or absorb body exudates such as urine, feces, and/or menses. It usually forms an outer cover or chassis of the absorbent article.

As used herein, the term “wipe” as a noun means an article used to cleanse body parts. Preferred wipes are those used for the cleaning of the peri-anal area after defecation or for the cleansing of other body parts after a change of an absorbent article.

As used herein, the term “disposable” means, in relation to an absorbent article, intended to be discarded after a single use.

As used herein, the term “urogenital” means the vulva, vagina, urinary tract, bladder, and surrounding areas of the body.

As used herein, the term “female subject” generally means a complex organism that has a female sex organ, such as a vagina. In some embodiments, a female subject is a human female subject. As used in relation to a human, the term “female subject” includes those assigned a gender of female at birth and who have a vagina, as well as those that were assigned male at birth but have undergone sex reassignment surgery to have a neovagina. In this respect, a “neovagina” is synonymous with a “vagina” herein, particularly where a neovagina contains beneficial and/or pathogenic bacteria disclosed elsewhere herein that can be affected (e.g., treated or modulated) by the methods disclosed herein.

As used herein, when a table, spectrum, or other data is referred to as representing the features or properties possessed by a particular composition, oligosaccharide, or other compound or mixture, unless specified otherwise, the same analysis method and procedure used to obtain the table, spectrum, or other data is to be used to determine the properties of the particular composition, oligosaccharide, or other compound or mixture.

Various oligosaccharide compositions disclosed herein are identified by a “CLX” designation. Such CLX compositions can be prepared in any suitable manner and by any suitable method, including ground up synthetic methods (e.g., oligomerizing monomeric or shorter chain oligosaccharides into the indicated oligosaccharides), or by depolymerization methods (e.g., by depolymerizing polysaccharides or longer chain oligosaccharides into shorter chain oligosaccharides). For example, in some aspects, the CLX compositions disclosed herein can be prepared by a depolymerization method disclosed in WO 2018/236917 (Amicucci et al., “Production of bioactive oligosaccharides”) or WO 2021/097138 (Amicucci et al., “High-yield peroxide quench-controlled polysaccharide depolymerization and compositions thereof”), both of which are hereby incorporated by reference in their entireties for all purposes, and more specifically for methodologies of preparation, to the extent not inconsistent with the description herein. In some embodiments, CLX compositions disclosed herein were prepared by a method comprising dissolving the indicated source polysaccharide(s) (e.g., lichenan, xylan, etc.) in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and iron (II) sulfate or copper (II) sulfate (in either case, 2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH and sample was reacted at 45° C. in a shaker-incubator for 1 hour at 20 RPM, the cap was left loose to allow oxygen, ammonia, and carbon dioxide gases to be released. The sample was then frozen and lyophilized, then stored at −80° C. The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

The oligosaccharide compositions disclosed herein, including CLX compositions, are characterized, in part, by the relative amounts of monosaccharide subunits present in each composition. The amount of each subunit is represented as a percentage as defined herein under “hydrolytic monosaccharide compositional analysis” and/or as a “monosaccharide ratio” as defined herein. It will be appreciated by one having skill in the art that hydrolytic monosaccharide compositional analysis is subject to random, experimental error, and the percentages and ratios should therefore be read to encompass reasonable variations from the stated value. Specifically, in some aspects, percentages and ratios associated with monosaccharide subunits of oligosaccharide compositions include variations of ±20% of the stated percentage or ratio. In keeping with this aspect, for a CLX composition recited as having 50% of its mass comprising glucose, it will be appreciated by a skilled artisan that said CLX composition may have anywhere between 40%-60% of its mass comprising glucose. As a further example of this aspect, a glucose:galactose ratio of 2:1 includes variations glucose:galactose ratios of 1.6:1 to 2.4:1 (e.g., 1.6:1, 1.8:1, 2:1, 2.2:1, 2.4:1) and 2:0.8 to 2:1.2 (e.g., 2:0.8, 2:0.9, 2:1, 2:1.1, 2:1.2). In some aspects, percentages and ratios associated with monosaccharide subunits of oligosaccharide compositions include variations of ±10% of the stated percentage or ratio. In some aspects, percentages and ratios associated with monosaccharide subunits of oligosaccharide compositions include variations of ±5% of the stated percentage or ratio. In some aspects, percentages and ratios associated with monosaccharide subunits of oligosaccharide compositions include variations of ±1% of the stated percentage or ratio.

Relatedly, the oligosaccharide compositions, including CLX compositions, are also characterized by the relative amounts of glycosidic linkages present in each composition. The amount of each linkage is represented as a percentage as defined herein under “glycosidic linkage composition” and/or as a “linkage ratio” as defined herein. It will appreciated by one having skill in the art that glycosidic linkage composition analysis is subject to random, experimental error, and the percentages and ratios should therefore be read to encompass reasonable variations from the stated value. Specifically, in some aspects, percentages and ratios associated with glycosidic linkages of oligosaccharide compositions include variations of ±20% of the stated percentage or ratio. In some aspects, percentages and ratios associated with glycosidic linkages of oligosaccharide compositions include variations of ±10% of the stated percentage or ratio. In some aspects, percentages and ratios associated with glycosidic linkages of oligosaccharide compositions include variations of ±5% of the stated percentage or ratio. In some aspects, percentages and ratios associated with glycosidic linkages of oligosaccharide compositions include variations of ±1% of the stated percentage or ratio.

For 1H-13C HSQC NMR analysis of the CLX compositions, oligosaccharides were dissolved in D₂O at a concentration of 50 mg/ml and were analyzed on a 600 MHz Bruker NMR spectrometer for their HSQC spectra.

As used herein, the term “CLX096” refers to an oligosaccharide composition wherein 76% of the mass comprises arabinose, 13% of the mass comprises galactose, 4% of the mass comprises xylose, 3% of the mass comprises glucose, and 2% of the mass comprises other, as measured by hydrolytic monosaccharide compositional analysis. The glycosidic linkage composition of the material comprises 36% terminal arabinose (T-Arab-f), 27% 3,5-branched arabinose, 20% 5-linked arabinose, 5% 4-linked galactose, 2% terminal galactose (T-Gal-p), and 5% other. In some aspects, the CLX096 composition generally is derived from sugar beet arabinan. In some aspects, the CLX096 composition is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) hydrolytic monosaccharide composition and glycosidic linkage composition as CLX096. For clarity, the hydrolytic monosaccharide compositional analysis and glycosidic linkage composition for CLX096 is performed on the material used to prepare the oligosaccharide composition, whereas such analyses for other CLX compositions typically are performed on the oligosaccharide composition itself.

As used herein, the term “CLX097” refers to an oligosaccharide composition wherein 48% of the mass comprises glucose, 26% of the mass comprises galactose, 10% of the mass comprises arabinose, and 9% of the mass comprises rhamnose, as measured by hydrolytic monosaccharide compositional analysis. In this composition, the glycosidic linkage composition comprises 10% 4-linked glucose, 16% terminal glucose, 12% 4-linked galactose, 6% as 6-linked galactose, 9% as terminal galactose, 4% 5-linked arabinose, 10% as terminal arabinose, and 5% terminal rhamnose. The CLX097 composition is defined by the oligosaccharide weight % in Table B, as measured by oligosaccharide analysis. In some aspects, CLX097 generally is derived from moringa leaf. In some aspects, CLX097 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) hydrolytic monosaccharide composition, glycosidic linkage composition, and oligosaccharide analysis as CLX097.

As used herein, the term “CLX098” refers to an oligosaccharide composition wherein 72% of the mass comprises glucose, 11% of the mass comprises galactose, and 6% of the mass comprises arabinose, as measured by hydrolytic monosaccharide compositional analysis. In this composition, the glycosidic linkage composition comprises 2% 3-linked glucose, 91% terminal glucose, and 2% 3-linked galactose. The CLX098 composition is defined by the oligosaccharide weight % in Table C, as measured by oligosaccharide analysis. In some aspects, CLX098 generally is derived from butternut squash skin. In some aspects, CLX098 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) hydrolytic monosaccharide composition, glycosidic linkage composition, and oligosaccharide analysis as CLX098.

As used herein, the term “CLX099” refers to an oligosaccharide composition wherein 73% of the mass comprises glucose, 4% of the mass comprises galactose, 6% of the mass comprises mannose, 9% of the mass comprises arabinose, and 6% of the mass comprises xylose, as measured by hydrolytic monosaccharide compositional analysis. In this composition, the glycosidic linkage composition comprises 53% 4-linked glucose, 2% 4,6-linked glucose, 15% terminal glucose, 4% 4-linked xylose, 4% 3-linked glucose, and 8% terminal arabinose. The CLX099 composition is defined by the oligosaccharide weight % in Table D, as measured by oligosaccharide analysis. In some aspects, CLX099 generally is derived from spent distillers' grain. In some aspects, CLX099 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) hydrolytic monosaccharide composition, glycosidic linkage composition, and oligosaccharide analysis as CLX099.

As used herein, the term “CLX101” refers to an oligosaccharide composition wherein 99% of the mass comprises glucose, as measured by hydrolytic monosaccharide compositional analysis. In this composition, the glycosidic linkage composition comprises 75% 3-linked glucose, 9% terminal glucose, and 15% other minor linkages. The CLX101 composition is defined by the 1H-13C HSQC NMR correlations described in Table A. The CLX101 composition is defined by the oligosaccharide weight % in Table I, as measured by oligosaccharide analysis. CLX101 has a dynamic viscosity at 25° C. at 100 mg/ml of 1.306 mPa*s. In some aspects, CLX101 generally is derived from microbial curdlan. In some aspects, CLX101 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX101.

As used herein, the term “CLX102” refers to an oligosaccharide composition wherein 37% of the mass comprises glucose and 60% of the mass comprises mannose, as measured by hydrolytic monosaccharide compositional analysis. In this composition, the glycosidic linkage composition comprises 32% 4-linked glucose, 8% terminal glucose, 48% 4-linked mannose, and 13% terminal mannose. The CLX102 composition is defined by the 1H-13C HSQC NMR correlations described in Table A. The CLX102 composition is defined by the oligosaccharide weight % in Table J, as measured by oligosaccharide analysis. CLX102 has a dynamic viscosity at 25° C. at 100 mg/ml of 1.392 mPa*s. In some aspects, CLX102 generally is derived from glucomannan. In some aspects, CLX102 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX102.

As used herein, the term “CLX103” refers to an oligosaccharide composition wherein 85% of the mass comprises xylose, 5% of the mass comprises glucose, 5% of the mass comprises mannose, and 2% of the mass comprises galactose, as measured by hydrolytic monosaccharide compositional analysis. In this composition, the glycosidic linkage composition comprises 14% 4-linked glucose, 5% terminal glucose, 55% 4-linked xylose, 7% terminal xylose, and 15% 4-mannose. CLX103 composition is defined by the 1H-13C HSQC NMR correlations described in Table A. The CLX103 composition is defined by the oligosaccharide weight % in Table E, as measured by oligosaccharide analysis. CLX103 has a dynamic viscosity at 25° C. at 100 mg/ml of 1.093 mPa*s. In some aspects, CLX103 generally is derived from beech wood xylan. In some aspects, CLX103 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX103.

As used herein, the term “CLX105” refers to an oligosaccharide composition wherein 87% of the mass comprises galactose and 6% of the mass comprises arabinose, as measured by hydrolytic monosaccharide compositional analysis. In this composition, the glycosidic linkage composition comprises 17% 3-linked galactose, 14% 3,6-linked galactose, 12% 6-linked galactose, 51% terminal galactose, and 3% terminal arabinose. The CLX105 composition is defined by the 1H-13C HSQC NMR correlations described in Table A. The CLX105 composition is defined by the oligosaccharide weight % in Table K, as measured by oligosaccharide analysis. CLX105 has a dynamic viscosity at 25° C. at 100 mg/ml of 1.210 mPa*s. In some aspects, CLX105 generally is derived from arabinogalactan. In some aspects, CLX105 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX105.

As used herein, the term “CLX108” refers to an oligosaccharide composition wherein 73% of the mass comprises mannose and 23% of the mass comprises galactose, as measured by hydrolytic monosaccharide compositional analysis. In this composition, the glycosidic linkage composition comprises 20% terminal galactose, 62% 4-linked mannose, 9% 4,6-linked mannose, and 7% terminal mannose. The CLX108 composition is defined by the 1H-13C HSQC NMR correlations described in Table A. The CLX108 composition is defined by the oligosaccharide weight % in Table L, as measured by oligosaccharide analysis. CLX108 has a dynamic viscosity at 25° C. at 100 mg/ml of 6.447 mPa*s. In some aspects, CLX108 generally is derived from locust bean gum. In some aspects, CLX108 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX108.

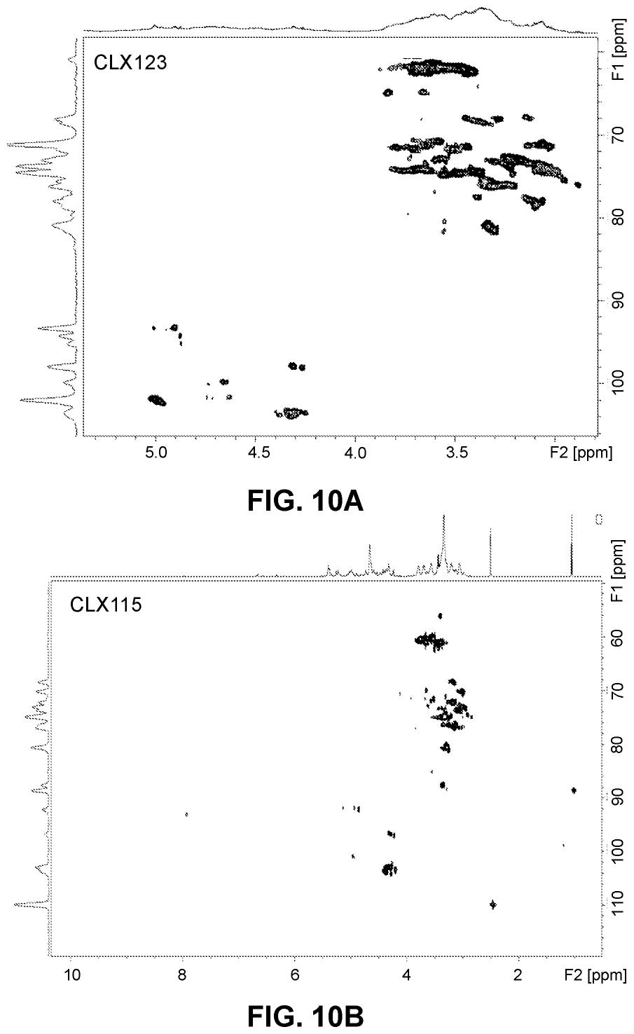

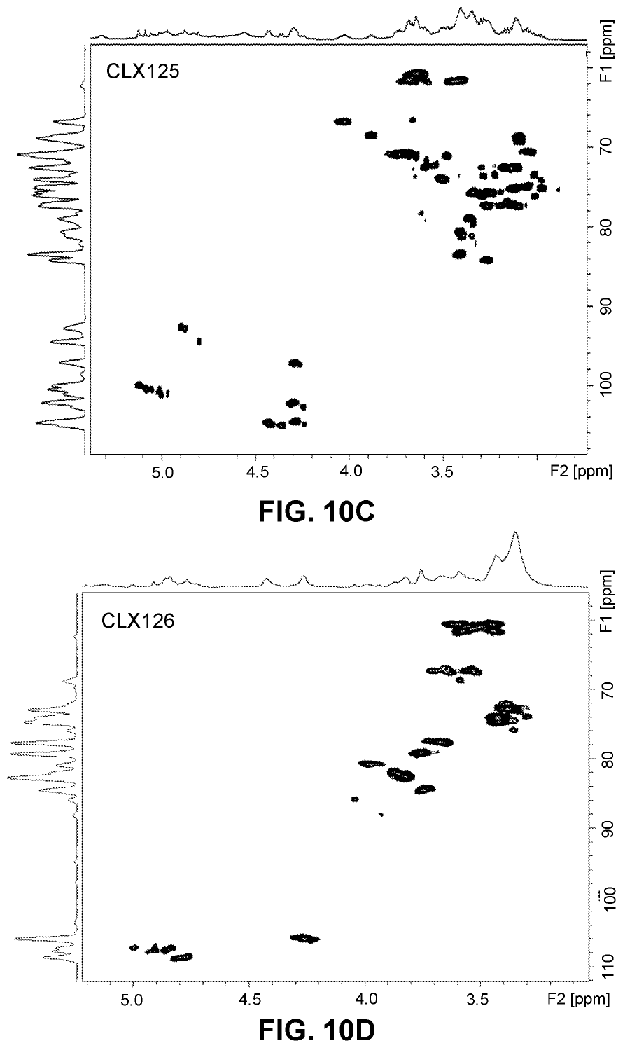

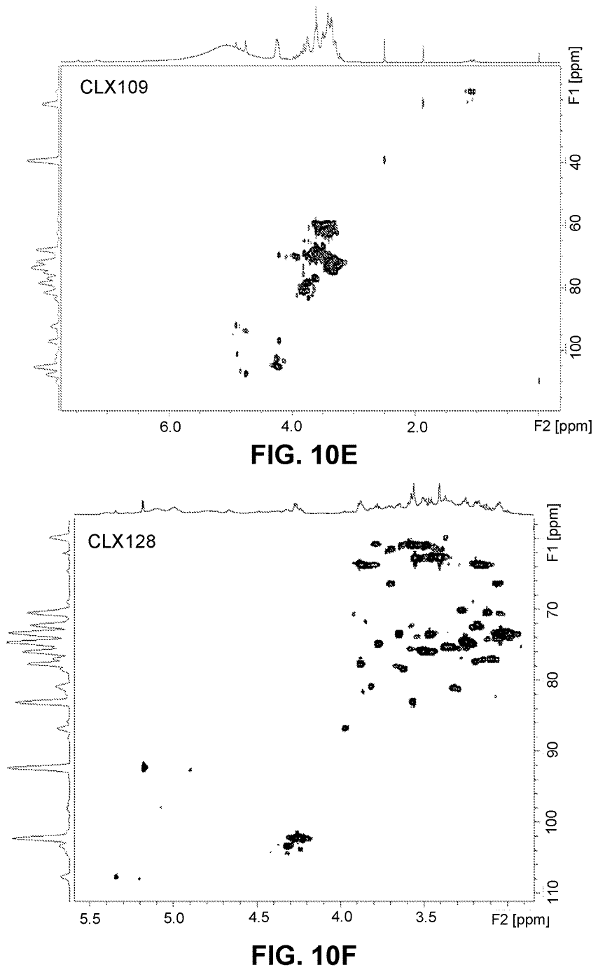

As used herein, the term “CLX109” refers to an oligosaccharide composition wherein 80% of the mass comprises galactose, 9% of the mass comprises arabinose, 5% of the mass comprises rhamnose, and 3% of the mass comprises galacturonic acid, as measured by hydrolytic monosaccharide compositional analysis. In this composition, the glycosidic linkage composition comprises 62% 4-linked galactose, 34% terminal galactose, and 2% terminal arabinose. The CLX109 composition comprises, approximately, the 1H-13C HSQC NMR correlations set forth in Table A for CLX109 (see also the spectrum in FIG. 10E). The CLX109 composition comprises, approximately, the values set forth in Table M, as measured by oligosaccharide analysis. CLX109 has a dynamic viscosity at 25° C. at 100 mg/ml of 0.830 mPa*s. In some aspects, CLX109 generally is derived from pectic galactan. In some aspects, CLX109 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX109.

As used herein, the term “CLX110” refers to an oligosaccharide composition wherein 80% of the mass comprises glucose, 9% of the mass comprises mannose, and 9% of the mass comprises galactose, as measured by hydrolytic monosaccharide compositional analysis. In this composition, the glycosidic linkage composition comprises 26% 3-linked glucose, 43% 4-linked glucose, 23% terminal glucose, and 7% terminal galactose. CLX110 composition is defined by the 1H-13C HSQC NMR correlations described in Table A. The CLX110 composition is defined by the oligosaccharide weight % in Table F, as measured by oligosaccharide analysis. CLX110 has a dynamic viscosity at 25° C. at 100 mg/ml of 1.250 mPa*s. In some aspects, CLX110 generally is derived from lichenan. In some aspects, CLX110 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX110.

As used herein, the term “CLX111” refers to an oligosaccharide composition wherein 78% of the mass comprises mannose and 19% of the mass comprises galactose, as measured by hydrolytic monosaccharide compositional analysis. In this composition, the glycosidic linkage composition comprises 2% 4-linked glucose, 18% terminal galactose, 47% 4-linked mannose, 7% 4,6-linked mannose, and 20% terminal mannose. CLX111 composition is defined by the 1H-13C HSQC NMR correlations described in Table A. The CLX111 composition is defined by the oligosaccharide weight % in Table G, as measured by oligosaccharide analysis. CLX 111 has a dynamic viscosity at 25° C. at 100 mg/ml of 1.683 mPa*s. In some aspects, CLX111 generally is derived from carob galactomannan. In some aspects, CLX111 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX111.

As used herein, the term “CLX112” refers to an oligosaccharide composition wherein 97% of the mass comprises glucose, as measured by hydrolytic monosaccharide compositional analysis. In this composition, the glycosidic linkage composition comprises 17% 3-linked glucose, 49% 4-linked glucose, and 31% terminal glucose. CLX112 composition is defined by the 1H-13C HSQC NMR correlations described in Table A. The CLX112 composition is defined by the oligosaccharide weight % in Table H, as measured by oligosaccharide analysis. CLX112 has a dynamic viscosity at 25° C. at 100 mg/ml of 1.248 mPa*s. In some aspects, CLX 112 generally is derived from barley beta glucan. In some aspects, CLX112 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX112.

As used herein, the term “CLX113” refers to an oligosaccharide composition wherein 49% of the mass comprises glucose, 36% of the mass comprises xylose, and 14% of the mass comprises galactose, as measured by hydrolytic monosaccharide compositional analysis. In this composition, the glycosidic linkage composition comprises 28% 4-linked glucose, 6% 6-linked glucose, 20% 4,6-linked glucose, 4% terminal glucose, 21% terminal galactose, 6% 2-linked xylose, and 11% terminal xylose. The CLX113 composition is defined by the 1H-13C HSQC NMR correlations described in Table A. The CLX113 composition is defined by the oligosaccharide weight % in Table N, as measured by oligosaccharide analysis. CLX113 has a dynamic viscosity at 25° C. at 100 mg/ml of 1.209 mPa*s. In some aspects, CLX113 generally is derived from tamarind seed xyloglucan. In some aspects, CLX113 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX113.

As used herein, the term “CLX114” refers to an oligosaccharide composition wherein 60% of the mass comprises xylose, 37% of the mass comprises arabinose, and 2% of the mass comprises galactose, as measured by hydrolytic monosaccharide compositional analysis. In this composition, the glycosidic linkage composition comprises 31% 4-linked xylose, 22% 3,4-linked xylose, 3% terminal xylose, 31% terminal arabinose, and 11% other minor linkages. The CLX114 composition is defined by the 1H-13C HSQC NMR correlations described in Table A. The CLX114 composition is defined by the oligosaccharide weight % in Table O, as measured by oligosaccharide analysis. CLX114 has a dynamic viscosity at 25° C. at 100 mg/ml of 1.877 mPa*s. In some aspects, CLX114 generally is derived from Rye arabinoxylan. In some aspects, CLX114 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX114.

As used herein, the term “CLX115” refers to an oligosaccharide composition wherein 95% of the mass comprises glucose and 2% of the mass comprises arabinose, as measured by hydrolytic monosaccharide compositional analysis. For composition CLX115, the glycosidic linkage composition comprises 64% 4-linked glucose, 23% 3-linked glucose, and 13% terminal glucose. The CLX115 composition comprises, approximately, the 1H-13C HSQC NMR correlations set forth in Table A for CLX115 (see also the spectrum in FIG. 10B). The CLX115 composition comprises, approximately, the values set forth in Table P, as measured by oligosaccharide analysis. CLX115 has a dynamic viscosity of 1.382 mPa*s at 100 mg/ml at 25° C. In some aspects, CLX115 generally is derived from oat beta glucan. In some aspects, CLX115 is derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX115. CLX115 was produced in accordance with the depolymerization described in Example 5.

As used herein, the term “CLX115Cu” refers to an oligosaccharide composition wherein 87.5% of the mass comprises glucose and 4.5% of the mass comprises arabinose, as measured by hydrolytic monosaccharide compositional analysis. For composition CLX115Cu, the glycosidic linkage composition comprises, approximately, the amount set forth in Table A, for CLX 115Cu. The CLX 115Cu oligosaccharide composition comprises, approximately, the values set forth in Table V, as measured by oligosaccharide analysis. CLX115Cu was produced in accordance with the depolymerization described in Example 4.

As used herein, the term “CLX122” refers to an oligosaccharide composition wherein 80% of the mass comprises arabinose, 10% of the mass comprises galactose, 4% of the mass comprises glucose, 4% of the mass comprises galacturonic acid, and 2% of the mass comprises rhamnose, as measured by hydrolytic monosaccharide compositional analysis. The CLX122 composition comprises, approximately, the 1H-13C HSQC NMR correlations set forth in Table A for CLX122. The CLX122 oligosaccharide composition comprises, approximately, the values set forth in Table U, as measured by oligosaccharide analysis. CLX122 has a dynamic viscosity at 25° C. at 100 mg/ml of 2.913 mPa*s. In some aspects, CLX122 generally is derived from Pea. In some aspects, CLX122 is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX122. CLX122 was produced in accordance with the depolymerization described in Example 5.

As used herein, the term “CLX122DSF” refers to an oligosaccharide composition wherein 77% of the mass comprises arabinose, 10% of the mass comprises glucose, 6% of the mass comprises galactose, 3% of the mass comprises galacturonic acid, and 3% of the mass comprises rhamnose, as measured by hydrolytic monosaccharide compositional analysis. The CLX122DSF composition comprises, approximately, the ¹H-¹³C HSQC NMR correlations set forth in Table A for CLX122DSF. The CLX122DSF oligosaccharide composition comprises, approximately, the values set forth in Table W, as measured by oligosaccharide analysis. CLX122DSF has a dynamic viscosity at 25° C. at 100 mg/ml of 1.555 mPa*s. In some aspects, CLX122DSF generally is derived from Pea. In some aspects, CLX122DSF is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, ¹H-¹³C HSQC NMR analysis and glycosidic linkage composition as CLX122DSF. CLX122DSF was produced in accordance with the depolymerization described in Example 5, with the exception that CLX122DSF was treated with an alpha-amylase prior to depolymerization to remove starch.

As used herein, the term “CLX123” refers to an oligosaccharide composition wherein 64% of the mass comprises glucose, 31% of the mass comprises mannose, and 3% of the mass comprises glucuronic acid, as measured by hydrolytic monosaccharide compositional analysis. For composition CLX123, the glycosidic linkage composition comprises 30% 4-linked glucose, 3% 3-linked glucose, 12% terminal glucose, 12% 3,6-linked galactose, 16% 2-linked mannose, 6% 4,6-linked mannose, 6% terminal mannose, and 4% 4-linked glucuronic acid. The CLX123 composition comprises, approximately, the 1H-13C HSQC NMR correlations set forth in Table A for CLX123 (see also the spectrum in FIG. 10A). The CLX123 oligosaccharide composition comprises, approximately, the values set forth in Table Q, as measured by oligosaccharide analysis. CLX123 has a dynamic viscosity at 25° C. at 100 mg/ml of 1.658 mPa*s. In some aspects, CLX123 generally is derived from Xanthomonas Campestris extract. In some aspects, CLX123 is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX123.

As used herein, the term “CLX125” refers to an oligosaccharide composition wherein 44% of the mass comprises glucose, 43% of the mass comprises rhamnose, and 8% of the mass comprises glucuronic acid, as measured by hydrolytic monosaccharide compositional analysis. For composition CLX125, the glycosidic linkage composition comprise 24% 4-linked glucose, 21% 3-linked glucose, 10% terminal glucose, 22% 4-linked rhamnose, 7% terminal rhamnose, 5% 4-linked glucuronic acid, and 3% terminal glucuronic acid. CLX 125 composition comprises, approximately, the 1H-13C HSQC NMR correlations set forth in Table A for CLX125 (see also the spectrum in FIG. 10C). The CLX125 oligosaccharide composition comprises, approximately, the values set forth in Table R, as measured by oligosaccharide analysis. CLX125 has a dynamic viscosity at 25° C. at 100 mg/ml of 1.525 mPa*s. In some aspects, CLX125 generally is derived from Sphingomonas elodea extract. In some aspects, CLX125 is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX125.

As used herein, the term “CLX126” refers to an oligosaccharide composition wherein 50% of the mass comprises galactose, 34% of the mass comprises arabinose, 5% of the mass comprises xylose, 3% of the mass comprises galacturonic acid, 3% of the mass comprises glucose, 2% of the mass comprises rhamnose, and 2% of the mass comprises fucose, as measured by hydrolytic monosaccharide compositional analysis. For composition CLX126, the glycosidic linkage composition comprises 42% 4-linked galactose, 5% terminal galactose, 3% 4-linked glucose, 11% 5-linked arabinose, 14% terminal arabinose, 5% 3,5-linked arabinose, and 2% 2,3,5-linked arabinose. The CLX126 composition comprises, approximately, the 1H-13C HSQC NMR correlations set forth in Table A for CLX126 (see also the spectrum in FIG. 10D). The CLX126 oligosaccharide composition comprises, approximately, the values set forth in Table S, as measured by oligosaccharide analysis. CLX126 has a dynamic viscosity at 25° C. at 100 mg/ml of 4.425 mPa*s. In some aspects, CLX126 generally is derived from Soy. In some aspects, CLX126 is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX126.

As used herein, the term “CLX128” refers to an oligosaccharide composition wherein 47% of the mass comprises xylose, 35% of the mass comprises glucose, 12% of the mass comprises arabinose, and 2% of the mass comprises galactose, as measured by hydrolytic monosaccharide compositional analysis. For composition CLX128, the glycosidic linkage composition comprises 3% 3-linked glucose, 16% 4-linked glucose, 9% terminal glucose, 27% 4-linked xylose, 6% terminal xylose, 5% 3,4-linked xylose, 7% 5-linked arabinose, 8% terminal arabinose, 6% 2-linked fucose, and 2% terminal glucuronic acid. The CLX128 composition comprises, approximately, the 1H-13C HSQC NMR correlations set forth in Table A for CLX128 (see also the spectrum in FIG. 10F). The CLX128 oligosaccharide composition comprises, approximately, the values set forth in Table T, as measured by oligosaccharide analysis. CLX128 has a dynamic viscosity at 25° C. at 100 mg/ml of about 1.818 mPa*s. In some aspects, CLX128 generally is derived from Sugar Cane. In some aspects, CLX128 is derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX128.

TABLE A
1H-13C HSQC NMR correlations from oligosaccharide compositions used herein.
The listed pairs correspond to those major peaks in the anomeric region.

| CLX101 | | CLX102 | | CLX111 | | CLX112 | | CLX105 | | CLX110 | |
|---|---|---|---|---|---|---|---|---|---|---|---|
| 1H δ | 13C δ | 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] | [ppm] | [ppm] |
| 3.37 | 103.44 | 4.52 | 102.44 | 4.50 | 96.76 | 4.64 | 95.88 | 3.54 | 100.74 | 4.51 | 102.45 |
| 3.42 | 99.57 | 4.55 | 96.58 | 4.51 | 96.70 | 4.75 | 102.73 | 3.64 | 99.80 | 4.54 | 102.32 |
| 3.52 | 105.61 | 4.56 | 96.52 | 4.55 | 96.52 | 4.67 | 95.70 | 3.65 | 99.80 | 4.64 | 95.82 |
| 3.52 | 98.05 | 4.62 | 95.88 | 4.57 | 96.46 | 4.76 | 102.68 | 3.66 | 102.68 | 4.66 | 95.75 |
| 3.57 | 103.32 | 4.65 | 95.76 | 4.58 | 96.41 | 4.78 | 102.50 | 3.70 | 93.24 | 4.67 | 95.75 |
| 3.74 | 90.66 | 4.66 | 95.76 | 4.85 | 96.82 | 4.79 | 102.50 | 3.71 | 105.14 | 4.75 | 102.73 |
| 3.76 | 90.61 | 4.76 | 99.98 | 4.87 | 96.76 | 5.23 | 91.89 | 3.71 | 105.08 | 4.79 | 102.52 |
| 3.80 | 84.04 | 4.84 | 96.82 | 4.89 | 93.65 | 4.65 | 95.88 | 3.77 | 90.96 | 5.23 | 91.92 |
| 3.92 | 90.66 | 4.86 | 96.76 | 4.91 | 93.65 | 4.52 | 102.50 | 3.78 | 100.16 | 5.37 | 99.03 |
| 3.95 | 90.66 | 4.88 | 93.65 | 5.00 | 98.34 | 4.51 | 102.50 | 3.86 | 81.93 | 5.42 | 99.72 |
| 4.64 | 95.88 | 4.90 | 93.65 | 5.00 | 94.00 | 4.54 | 102.32 | 3.91 | 103.61 | | |
| 4.65 | 95.88 | 5.17 | 93.77 | 5.03 | 98.69 | 4.66 | 95.70 | 3.91 | 99.22 | | |
| 4.67 | 95.64 | 5.21 | 91.84 | 5.18 | 93.83 | | | 3.93 | 98.63 | | |
| 4.69 | 95.64 | 5.27 | 92.30 | 5.23 | 92.54 | | | 3.96 | 98.57 | | |
| 4.76 | 102.73 | 5.39 | 99.57 | 5.23 | 88.55 | | | 4.05 | 99.22 | | |
| 4.77 | 102.68 | | | 5.24 | 88.55 | | | 4.20 | 98.40 | | |
| 4.80 | 102.50 | | | 5.26 | 92.25 | | | 4.24 | 98.46 | | |
| 4.81 | 102.50 | | | 5.28 | 92.30 | | | 4.45 | 103.44 | | |
| 5.24 | 92.01 | | | 5.29 | 101.39 | | | 4.69 | 103.85 | | |
| CLX103 | | CLX113 | | CLX108 | | CLX114 | | CLX122 | | CLX123 | |
| 1H δ | 13C δ | 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] | [ppm] | [ppm] |
| 4.16 | 101.09 | 4.56 | 104.43 | 4.52 | 102.52 | 4.46 | 101.27 | 5.00 | 107.17 | 5.01 | 93.22 |
| 4.31 | 101.15 | 4.56 | 102.45 | 4.53 | 102.52 | 4.48 | 101.56 | 4.93 | 107.83 | 5.00 | 101.92 |
| 4.35 | 109.59 | 4.57 | 96.57 | 4.55 | 96.50 | 4.49 | 101.56 | 4.90 | 105.88 | 4.97 | 102.02 |
| 4.39 | 100.10 | 4.67 | 103.00 | 4.57 | 96.50 | 4.51 | 102.91 | 4.90 | 106.90 | 4.97 | 102.29 |
| 4.40 | 100.74 | 4.67 | 95.68 | 4.64 | 104.30 | 4.51 | 101.56 | 4.86 | 107.67 | 4.91 | 93.19 |
| 4.46 | 101.80 | 4.68 | 95.75 | 4.76 | 100.13 | 4.55 | 102.91 | 4.83 | 107.22 | 4.88 | 94.14 |
| 4.48 | 101.62 | 4.69 | 95.75 | 4.87 | 96.78 | 4.58 | 102.27 | 4.81 | 108.63 | 4.87 | 95.07 |
| 4.48 | 100.86 | 4.70 | 95.68 | 4.91 | 93.63 | 4.58 | 99.92 | 4.77 | 107.44 | 4.74 | 101.59 |
| 4.49 | 102.79 | 4.96 | 98.83 | 5.03 | 99.38 | 4.58 | 96.46 | 4.77 | 108.55 | 4.73 | 99.99 |
| 4.49 | 101.62 | 5.18 | 98.62 | 5.03 | 98.69 | 4.60 | 96.41 | 4.26 | 105.83 | 4.71 | 101.77 |
| 4.49 | 100.86 | 5.24 | 91.79 | 5.03 | 97.80 | 4.61 | 102.27 | 4.04 | 85.79 | 4.66 | 99.74 |
| 4.50 | 102.79 | 5.41 | 92.06 | 5.09 | 107.44 | 4.62 | 96.35 | 3.92 | 87.99 | 4.64 | 99.82 |
| 4.53 | 102.91 | | | 5.18 | 93.77 | 4.63 | 99.86 | 4.63 | 101.54 | 3.42 | 99.83 |
| 4.54 | 102.91 | | | 5.19 | 107.10 | 4.63 | 96.35 | 4.38 | 103.77 | 3.22 | 97.84 |
| 4.55 | 100.21 | | | 5.28 | 92.27 | 5.05 | 108.18 | 4.34 | 103.32 | | |
| 4.57 | 102.21 | | | 5.41 | 92.13 | 5.19 | 92.01 | 4.31 | 97.82 | | |
| 4.57 | 100.21 | | | 5.43 | 92.06 | 5.23 | 108.59 | 4.31 | 103.77 | | |
| 4.58 | 102.21 | | | | | 5.28 | 108.01 | 4.27 | 98.04 | | |
| 4.58 | 96.46 | | | | | 5.33 | 108.01 | 4.25 | 103.47 | | |
| 4.59 | 100.27 | | | | | 5.35 | 91.72 | 3.84 | 64.91 | | |
| 4.59 | 96.46 | | | | | 5.39 | 107.60 | 3.66 | 64.86 | | |
| 4.63 | 101.27 | | | | | | | | | | |
| 4.64 | 101.27 | | | | | | | | | | |
| 4.71 | 101.68 | | | | | | | | | | |
| 4.72 | 101.74 | | | | | | | | | | |
| 4.73 | 96.64 | | | | | | | | | | |
| 4.74 | 96.64 | | | | | | | | | | |
| 5.06 | 101.39 | | | | | | | | | | |
| 5.06 | 98.46 | | | | | | | | | | |
| 5.09 | 96.93 | | | | | | | | | | |
| 5.12 | 98.05 | | | | | | | | | | |
| 5.19 | 91.95 | | | | | | | | | | |
| 5.29 | 97.75 | | | | | | | | | | |
| 5.30 | 97.75 | | | | | | | | | | |
| 5.40 | 99.57 | | | | | | | | | | |
| 5.41 | 97.93 | | | | | | | | | | |
| 5.41 | 92.13 | | | | | | | | | | |
| CLX115 | | CLX125 | | CLX109 | | CLX126 | | CLX128 | | CLX122DSF | |
| 1H δ | 13C δ | 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] | [ppm] | [ppm] |
| 4.96 | 100.76 | 5.12 | 99.92 | 4.91 | 92.33 | 4.99 | 107.2 | 5.34 | 107.75 | 5.17 | 100.60 |
| 4.93 | 91.79 | 5.08 | 100.49 | 4.89 | 101.49 | 4.93 | 107.81 | 5.20 | 108.06 | 5.08 | 95.20 |
| 4.86 | 92.07 | 5.05 | 100.46 | 4.85 | 92.53 | 4.90 | 106.86 | 5.18 | 92.33 | 5.07 | 100.12 |
| 4.38 | 103.66 | 5.01 | 100.49 | 4.84 | 106.89 | 4.91 | 107.59 | 5.08 | 97.86 | 5.02 | 108.48 |
| 4.33 | 102.65 | 5.00 | 101.01 | 4.80 | 93.82 | 4.86 | 107.64 | 4.90 | 92.64 | 5.02 | 100.21 |
| 4.32 | 96.44 | 4.97 | 100.95 | 4.75 | 107.74 | 4.83 | 107.22 | 4.42 | 104.18 | 5.01 | 92.27 |
| 4.28 | 102.79 | 4.90 | 92.50 | 4.74 | 94.09 | 4.82 | 108.8 | 4.37 | 103.14 | 4.99 | 100.39 |
| 4.28 | 96.71 | 4.88 | 92.79 | 4.26 | 103.12 | 4.79 | 108.68 | 4.36 | 103.22 | 4.99 | 95.51 |
| 4.27 | 104.10 | 4.80 | 94.54 | 4.24 | 105.38 | 4.78 | 108.53 | 4.32 | 104.29 | 4.99 | 92.13 |
| 4.22 | 96.94 | 4.43 | 104.69 | 4.21 | 102.89 | 4.26 | 105.84 | 4.32 | 103.30 | 4.97 | 106.14 |
| 4.20 | 103.24 | 4.36 | 105.04 | 4.21 | 97.19 | 4.23 | 105.98 | 4.31 | 102.08 | 4.96 | 101.79 |
| | | 4.30 | 102.1 | 4.13 | 103.63 | 4.04 | 85.82 | 4.31 | 104.34 | 4.92 | 107.05 |
| | | 3.93 | 88.01 | | | 4.23 | 106.25 | 4.52 | 104.19 | 4.90 | 101.62 |
| | | 3.75 | 79.18 | | | 4.04 | 85.87 | 4.40 | 103.84 | 4.90 | 98.46 |
| | | 3.70 | 67.26 | | | 3.93 | 88.07 | 4.38 | 104.56 | 4.90 | 92.05 |
| | | 3.65 | 66.74 | | | 3.75 | 79.21 | 4.27 | 104.25 | 4.89 | 106.52 |
| | | 3.63 | 67.51 | | | 3.7 | 67.35 | 4.23 | 104.33 | 4.87 | 101.19 |
| | | 3.54 | 67.16 | | | 3.67 | 66.73 | 4.22 | 104.41 | 4.85 | 107.00 |
| | | 3.54 | 66.61 | | | 3.65 | 66.83 | 4.21 | 104.44 | 4.85 | 92.48 |
| | | 3.52 | 67.48 | | | 3.62 | 67.35 | 4.19 | 98.44 | 4.82 | 106.57 |
| | | | | | | 3.57 | 67.20 | 4.16 | 104.63 | 4.80 | 93.82 |
| | | | | | | 3.55 | 67.17 | 4.15 | 104.15 | 4.76 | 107.75 |
| | | | | | | 3.53 | 67.14 | 4.07 | 104.75 | 4.72 | 94.18 |
| | | | | | | 3.51 | 67.45 | | | 4.63 | 98.84 |
| | | | | | | | | | | 4.32 | 102.54 |
| | | | | | | | | | | 4.31 | 96.51 |
| | | | | | | | | | | 4.26 | 102.05 |
| | | | | | | | | | | 4.26 | 96.68 |
| | | | | | | | | | | 4.24 | 105.55 |
| | | | | | | | | | | 4.23 | 102.75 |
| | | | | | | | | | | 4.21 | 97.16 |

TABLE B
CLX097 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 3Hex | 504.19 | 4.119 | 30.38 | 1 |
| 2 | 4Hex | 666.24 | 9.243 | 45.8 | 2.244 |
| 3 | 5Hex | 828.29 | 12.216 | 23.82 | 2.966 |

TABLE C
CLX098 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars. “NR”
refers to non-reducing oligosaccharides, e.g., those with a
fructose on the reducing end or where the pseudo-reducing end
contains sugars linked through their anomeric carbons.

| | | | RT | Oligo | Retention |
|---|---|---|---|---|---|
| Compound | Identity | Mass | (min) | Wt % | Factor |
| 1 | 3Hex (NR) | 504.17 | 1.764 | 8.32 | 1 |
| 2 | 3Hex (NR) | 504.17 | 3.6 | 2.46 | 2.041 |
| 3 | 3Hex (NR) | 504.17 | 4.904 | 4.63 | 2.78 |
| 4 | 3Hex (NR) | 504.17 | 10.952 | 59.66 | 6.209 |
| 5 | 4Hex (NR) | 666.22 | 9.623 | 6.23 | 5.455 |
| 6 | 4Hex (NR) | 666.22 | 14.113 | 18.69 | 8.001 |

TABLE D
CLX099 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 2Hex1Pent | 474.17 | 5.513 | 2.73 | 2.126 |
| 2 | 2Hex1Pent | 474.17 | 7.884 | 1.05 | 3.04 |
| 3 | 2Hex1Pent | 474.17 | 9.314 | 3.14 | 3.592 |
| 4 | 3Hex | 504.18 | 2.593 | 2.31 | 1 |
| 5 | 3Hex | 504.19 | 4.128 | 14.95 | 1.592 |
| 6 | 3Hex | 504.19 | 6.208 | 5.53 | 2.394 |
| 7 | 3Hex | 504.19 | 8.294 | 1.47 | 3.199 |
| 8 | 3Hex | 504.18 | 14.159 | 1.48 | 5.46 |
| 9 | 4Hex | 666.24 | 9.26 | 32.53 | 3.571 |
| 10 | 5Hex | 828.29 | 10.114 | 0.99 | 3.901 |
| 11 | 5Hex | 828.29 | 10.555 | 0.88 | 4.071 |
| 12 | 5Hex | 828.29 | 11.519 | 1.08 | 4.442 |
| 13 | 5Hex | 828.29 | 12.269 | 30.74 | 4.732 |
| 14 | 6Hex | 990.34 | 13.463 | 1.14 | 5.192 |

TABLE E
CLX103 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 2Pent | 282.11 | 6.692 | 1.60 | 1 |
| 2 | 3Pent | 414.16 | 8.429 | 14.82 | 1.26 |
| 3 | 3Pent1HexAOMe | 604.2 | 18.034 | 2.79 | 2.695 |
| 4 | 4Pent | 546.2 | 16.521 | 18.68 | 2.469 |
| 5 | 4Pent1HexAOMe | 736.24 | 20.205 | 2.61 | 3.019 |
| 6 | 4Pent1HexAOMe | 736.24 | 21.15 | 6.91 | 3.16 |
| 7 | 4Pent1HexAOMe | 736.24 | 23.844 | 4.67 | 3.563 |
| 8 | 4Pent1HexAOMe | 736.24 | 25.394 | 2.61 | 3.795 |
| 9 | 4Pent1HexAOMe | 736.24 | 26.435 | 2.91 | 3.95 |
| 10 | 5Hex2Pent | 1092.42 | 8.109 | 1.07 | 1.212 |
| 11 | 5Pent | 678.24 | 8.432 | 1.07 | 1.26 |
| 12 | 5Pent | 678.24 | 23.199 | 19.00 | 3.467 |
| 13 | 6Hex1Pent | 1122.35 | 18.442 | 7.75 | 2.756 |
| 14 | 6Pent | 810.28 | 26.735 | 9.99 | 3.995 |
| 15 | 7Pent | 942.32 | 28.974 | 3.51 | 4.33 |

TABLE F
CLX110 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 1Hex2Pent | 444.17 | 2.988 | 0.50 | 2.008 |
| 2 | 2Hex | 342.13 | 2.925 | 0.46 | 1.966 |
| 3 | 2Hex1Pent | 474.18 | 2.211 | 0.62 | 1.486 |
| 4 | 2Hex2Pent | 606.22 | 8.398 | 2.08 | 5.644 |
| 5 | 3Hex | 504.19 | 1.488 | 0.99 | 1 |
| 6 | 3Hex | 504.18 | 6.593 | 0.81 | 4.431 |
| 7 | 3Hex1Deoxyhex | 650.24 | 5.754 | 0.30 | 3.867 |
| 8 | 3Hex1HexA | 680.22 | 5.977 | 1.65 | 4.017 |
| 9 | 3Hex1Pent | 636.23 | 6.275 | 7.59 | 4.217 |
| 10 | 3Hex1Pent | 636.23 | 6.83 | 1.29 | 4.59 |
| 11 | 3Hex1Pent | 636.23 | 8.396 | 0.29 | 5.642 |
| 12 | 3Hex2Pent | 768.27 | 10.68 | 1.43 | 7.177 |
| 13 | 4Hex | 666.24 | 4.016 | 17.74 | 2.699 |
| 14 | 4Hex | 666.24 | 4.726 | 0.99 | 3.176 |
| 15 | 4Hex | 666.24 | 5.06 | 0.58 | 3.401 |
| 16 | 4Hex | 666.24 | 5.439 | 0.93 | 3.655 |
| 17 | 4Hex | 666.24 | 9.279 | 0.88 | 6.236 |
| 18 | 4Hex | 666.24 | 10.087 | 0.35 | 6.779 |
| 19 | 4Hex | 666.24 | 16.429 | 1.54 | 11.041 |
| 20 | 4Hex1Deoxyhex | 812.26 | 10.22 | 0.78 | 6.868 |
| 21 | 4Hex1HexA | 842.27 | 9.907 | 4.28 | 6.658 |
| 22 | 4Hex1HexA | 842.27 | 10.504 | 0.59 | 7.059 |
| 23 | 4Hex1Pent | 798.28 | 9.459 | 6.96 | 6.357 |
| 24 | 5Hex | 828.29 | 7.567 | 15.88 | 5.085 |
| 25 | 5Hex | 828.29 | 8.882 | 0.70 | 5.969 |
| 26 | 5Hex | 828.29 | 9.089 | 0.61 | 6.108 |
| 27 | 5Hex | 828.29 | 9.783 | 1.38 | 6.575 |
| 28 | 5Hex | 828.29 | 17.217 | 0.47 | 11.571 |
| 29 | 5Hex | 828.29 | 18.075 | 0.52 | 12.147 |
| 30 | 5Hex1Deoxyhex | 974.31 | 13.623 | 0.88 | 9.155 |
| 31 | 5Hex1HexA | 1004.33 | 12.747 | 4.44 | 8.567 |
| 32 | 5Hex1HexA | 1004.32 | 13.358 | 0.79 | 8.977 |
| 33 | 5Hex1Pent | 960.34 | 12.139 | 2.06 | 8.158 |
| 34 | 6Hex | 990.35 | 10.877 | 8.71 | 7.31 |
| 35 | 6Hex | 990.35 | 11.152 | 0.34 | 7.495 |
| 36 | 6Hex | 990.34 | 11.405 | 0.55 | 7.665 |
| 37 | 6Hex | 990.35 | 11.76 | 0.64 | 7.903 |
| 38 | 6Hex | 990.34 | 14.094 | 0.59 | 9.472 |
| 39 | 6Hex1HexA | 1166.38 | 15.181 | 1.68 | 10.202 |
| 40 | 6Hex1Pent | 1122.39 | 14.439 | 0.96 | 9.704 |
| 41 | 7Hex | 1152.4 | 13.039 | 4.18 | 8.763 |
| 42 | 8Hex | 1314.45 | 14.906 | 0.98 | 10.017 |

TABLE G
CLX111 comprises the oligosaccharides shown in this table, as
easured by oligosaccharide analysis. Hex refers to hexose sugars,
Pent refers to pentose sugars, HexA refers to hexuronic acid
sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 3Hex | 504.19 | 1.489 | 2.72 | 1 |
| 2 | 3Hex | 504.19 | 1.845 | 1.04 | 1.2391 |
| 3 | 3Hex | 504.19 | 2.978 | 1.54 | 2 |
| 4 | 3Hex1Pent | 636.23 | 6.404 | 1.79 | 4.3009 |
| 5 | 3Hex1Pent | 636.23 | 6.968 | 1.16 | 4.6797 |
| 6 | 3Hex1Pent | 636.23 | 8.198 | 0.38 | 5.5057 |
| 7 | 3Hex1Pent | 636.23 | 8.566 | 0.55 | 5.7529 |
| 8 | 3Hex2Pent | 768.27 | 10.827 | 0.28 | 7.2713 |
| 9 | 3Hex2Pent | 768.27 | 12.342 | 0.23 | 8.2888 |
| 10 | 3Hex2Pent | 768.27 | 13.372 | 0.43 | 8.9805 |
| 11 | 4Hex | 666.24 | 4.109 | 12.41 | 2.7596 |
| 12 | 4Hex | 666.24 | 4.802 | 4.74 | 3.225 |
| 13 | 4Hex | 666.24 | 5.122 | 5.89 | 3.4399 |
| 14 | 4Hex | 666.24 | 5.502 | 6.4 | 3.6951 |
| 15 | 4Hex | 666.24 | 7.239 | 3.54 | 4.8617 |
| 16 | 4Hex1Deoxyhex | 812.26 | 10.495 | 0.58 | 7.0484 |
| 17 | 4Hex1Deoxyhex | 812.26 | 14.627 | 0.42 | 9.8234 |
| 18 | 4Hex1HexA | 842.27 | 9.515 | 0.39 | 6.3902 |
| 19 | 4Hex1HexA | 842.27 | 10.301 | 1.26 | 6.9181 |
| 20 | 4Hex1HexA | 842.27 | 11.02 | 0.71 | 7.4009 |
| 21 | 4Hex1HexA | 842.27 | 11.725 | 0.52 | 7.8744 |
| 22 | 4Hex1HexA | 842.27 | 12.33 | 0.98 | 8.2807 |
| 23 | 4Hex1HexA | 842.27 | 14.866 | 0.43 | 9.9839 |
| 24 | 4Hex1HexA | 842.27 | 16.706 | 0.33 | 11.2196 |
| 25 | 4Hex1Pent | 798.28 | 9.614 | 1.3 | 6.4567 |
| 26 | 4Hex1Pent | 798.28 | 10.996 | 0.83 | 7.3848 |
| 27 | 4Hex1Pent | 798.28 | 11.525 | 0.31 | 7.7401 |
| 28 | 4Hex1Pent | 798.28 | 14.191 | 0.44 | 9.5306 |
| 29 | 5Hex | 828.29 | 7.65 | 7.46 | 5.1377 |
| 30 | 5Hex | 828.29 | 7.896 | 3.61 | 5.3029 |
| 31 | 5Hex | 828.29 | 8.52 | 2.77 | 5.722 |
| 32 | 5Hex | 828.29 | 9.013 | 3.16 | 6.0531 |
| 33 | 5Hex | 828.29 | 9.737 | 2.53 | 6.5393 |
| 34 | 5Hex | 828.29 | 9.999 | 4.5 | 6.7152 |
| 35 | 5Hex | 828.29 | 11.477 | 0.62 | 7.7079 |
| 36 | 5Hex | 828.29 | 13.038 | 3.41 | 8.7562 |
| 37 | 6Hex | 990.34 | 11.077 | 3.58 | 7.4392 |
| 38 | 6Hex | 990.34 | 11.342 | 1.11 | 7.6172 |
| 39 | 6Hex | 990.34 | 11.581 | 1.58 | 7.7777 |
| 40 | 6Hex | 990.34 | 11.96 | 1.27 | 8.0322 |
| 41 | 6Hex | 990.34 | 12.288 | 1.28 | 8.2525 |
| 42 | 6Hex | 990.34 | 12.746 | 0.79 | 8.5601 |
| 43 | 6Hex | 990.34 | 13.842 | 1.74 | 9.2962 |
| 44 | 6Hex | 990.34 | 14.332 | 2.32 | 9.6253 |
| 45 | 6Hex | 990.34 | 15.227 | 1.21 | 10.2263 |
| 46 | 6Hex | 990.34 | 17.443 | 0.31 | 11.7146 |
| 47 | 7Hex | 1152.4 | 13.245 | 1.35 | 8.8952 |
| 48 | 7Hex | 1152.4 | 14.057 | 0.86 | 9.4406 |
| 49 | 7Hex | 1152.39 | 14.302 | 0.48 | 9.6051 |
| 50 | 7Hex | 1152.39 | 14.551 | 0.39 | 9.7723 |
| 51 | 7Hex | 1152.4 | 15.271 | 0.49 | 10.2559 |
| 52 | 7Hex | 1152.39 | 15.516 | 0.31 | 10.4204 |
| 53 | 7Hex | 1152.4 | 16.316 | 0.79 | 10.9577 |
| 54 | 7Hex | 1152.39 | 17.823 | 0.51 | 11.9698 |

TABLE H
CLX112 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 3Hex | 504.19 | 10.431 | 7.9 | 1 |
| 2 | 3Hex | 504.19 | 14.158 | 13.53 | 1.357 |
| 3 | 3Hex | 504.19 | 17.074 | 5.47 | 1.637 |
| 4 | 4Hex | 666.24 | 21.248 | 11.27 | 2.037 |
| 5 | 4Hex | 666.24 | 24.78 | 6.29 | 2.376 |
| 6 | 4Hex | 666.24 | 25.028 | 8.82 | 2.399 |
| 7 | 4Hex | 666.24 | 25.857 | 9.81 | 2.479 |
| 8 | 5Hex | 828.29 | 27.544 | 2.08 | 2.641 |
| 9 | 5Hex | 828.29 | 28.172 | 3.8 | 2.701 |
| 10 | 5Hex | 828.29 | 28.914 | 1.18 | 2.772 |
| 11 | 5Hex | 828.29 | 29.507 | 7.33 | 2.829 |
| 12 | 5Hex | 828.29 | 30.241 | 11.24 | 2.899 |
| 13 | 6Hex | 990.34 | 32.369 | 2.62 | 3.103 |
| 14 | 6Hex | 990.34 | 34.032 | 6.74 | 3.263 |
| 15 | 6Hex | 990.34 | 37.05 | 1.95 | 3.552 |

TABLE I
CLX101 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 1Hex1Pent | 312.12 | 1.663 | 0.75 | 1.14 |
| 2 | 2Hex | 342.13 | 1.456 | 3.34 | 1.00 |
| 3 | 2Hex1Pent | 474.17 | 12.672 | 4.45 | 8.70 |
| 4 | 2Hex1Pent | 474.17 | 13.644 | 1.57 | 9.37 |
| 5 | 3Hex | 504.18 | 7.423 | 0.85 | 5.10 |
| 6 | 3Hex | 504.19 | 11.902 | 38.62 | 8.17 |
| 7 | 3Hex1Pent | 636.23 | 24.761 | 0.98 | 17.01 |
| 8 | 4Hex | 666.24 | 24.35 | 20.24 | 16.72 |
| 9 | 5Hex | 828.29 | 30.063 | 18.87 | 20.65 |
| 10 | 6Hex | 990.34 | 36.833 | 10.32 | 25.30 |

TABLE J
CLX102 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex1Pent | 474.17 | 2.263 | 0.17 | 1.518 |
| 2 | 2Hex1Pent | 474.17 | 9.187 | 0.88 | 6.162 |
| 3 | 2Hex1Pent | 474.17 | 10.653 | 0.54 | 7.145 |
| 4 | 2Hex1Pent | 474.17 | 12.713 | 0.37 | 8.526 |
| 5 | 3Hex | 504.18 | 1.491 | 0.84 | 1 |
| 6 | 3Hex | 504.18 | 2.193 | 0.35 | 1.471 |
| 7 | 3Hex | 504.19 | 6.695 | 4.31 | 4.49 |
| 8 | 3Hex | 504.19 | 7.637 | 2.06 | 5.122 |
| 9 | 3Hex | 504.18 | 8.885 | 1.94 | 5.959 |
| 10 | 3Hex | 504.19 | 9.605 | 1.74 | 6.442 |
| 11 | 3Hex | 504.19 | 12.868 | 1.45 | 8.63 |
| 12 | 3Hex | 504.18 | 14.159 | 0.4 | 9.496 |
| 13 | 3Hex1Pent | 636.23 | 6.413 | 0.92 | 4.301 |
| 14 | 3Hex1Pent | 636.23 | 12.074 | 1.13 | 8.098 |
| 15 | 3Hex1Pent | 636.23 | 13.068 | 0.77 | 8.765 |
| 16 | 3Hex1Pent | 636.23 | 18.947 | 1.52 | 12.708 |
| 17 | 3Hex1Pent | 636.23 | 19.573 | 0.75 | 13.127 |
| 18 | 3Hex1Pent | 636.23 | 20.258 | 0.81 | 13.587 |
| 19 | 4Hex | 666.24 | 4.124 | 2.33 | 2.766 |
| 20 | 4Hex | 666.24 | 6.206 | 2.04 | 4.162 |
| 21 | 4Hex | 666.24 | 9.373 | 2.99 | 6.286 |
| 22 | 4Hex | 666.24 | 10.25 | 2.66 | 6.875 |
| 23 | 4Hex | 666.24 | 10.97 | 3.94 | 7.357 |
| 24 | 4Hex | 666.24 | 14.977 | 1.75 | 10.045 |
| 25 | 4Hex | 666.24 | 16.802 | 4.87 | 11.269 |
| 26 | 4Hex | 666.24 | 17.38 | 5.13 | 11.657 |
| 27 | 4Hex | 666.24 | 19.079 | 1.69 | 12.796 |
| 28 | 4Hex | 666.24 | 19.911 | 0.58 | 13.354 |
| 29 | 4Hex | 666.24 | 20.691 | 0.36 | 13.877 |
| 30 | 4Hex | 666.24 | 24.147 | 1.15 | 16.195 |
| 31 | 4Hex1Pent | 798.28 | 9.604 | 0.45 | 6.441 |
| 32 | 4Hex1Pent | 798.28 | 15.28 | 0.48 | 10.248 |
| 33 | 4Hex1Pent | 798.28 | 17.219 | 0.45 | 11.549 |
| 34 | 4Hex1Pent | 798.28 | 18.793 | 0.64 | 12.604 |
| 35 | 4Hex1Pent | 798.28 | 20.328 | 1.41 | 13.634 |
| 36 | 4Hex1Pent | 798.28 | 21.012 | 0.36 | 14.093 |
| 37 | 4Hex1Pent | 798.28 | 21.923 | 0.44 | 14.704 |
| 38 | 4Hex1Pent | 798.28 | 22.714 | 0.55 | 15.234 |
| 39 | 4Hex1Pent | 798.28 | 26.723 | 0.35 | 17.923 |
| 40 | 4Hex1Pent | 798.28 | 27.138 | 0.22 | 18.201 |
| 41 | 4Hex1Pent | 798.28 | 27.347 | 0.26 | 18.341 |
| 42 | 4Hex1Pent | 798.28 | 27.565 | 0.32 | 18.488 |
| 43 | 5Hex | 828.29 | 7.648 | 1.05 | 5.129 |
| 44 | 5Hex | 828.29 | 9.213 | 1.28 | 6.179 |
| 45 | 5Hex | 828.29 | 12.21 | 0.37 | 8.189 |
| 46 | 5Hex | 828.29 | 14.335 | 1.28 | 9.614 |
| 47 | 5Hex | 828.29 | 14.932 | 0.75 | 10.015 |
| 48 | 5Hex | 828.29 | 16.24 | 2.01 | 10.892 |
| 49 | 5Hex | 828.29 | 17.527 | 1.3 | 11.755 |
| 50 | 5Hex | 828.29 | 18.549 | 5.98 | 12.441 |
| 51 | 5Hex | 828.29 | 18.882 | 0.36 | 12.664 |
| 52 | 5Hex | 828.29 | 19.204 | 1.29 | 12.88 |
| 53 | 5Hex | 828.29 | 19.522 | 0.69 | 13.093 |
| 54 | 5Hex | 828.29 | 20.062 | 1.8 | 13.455 |
| 55 | 5Hex | 828.29 | 20.313 | 1.44 | 13.624 |
| 56 | 5Hex | 828.29 | 21.885 | 1.43 | 14.678 |
| 57 | 5Hex | 828.29 | 25.896 | 3.46 | 17.368 |
| 58 | 5Hex | 828.29 | 27.822 | 0.56 | 18.66 |
| 59 | 5Hex | 828.29 | 28.03 | 0.64 | 18.799 |
| 60 | 6Hex | 990.34 | 11.067 | 0.31 | 7.423 |
| 61 | 6Hex | 990.34 | 11.999 | 0.4 | 8.048 |
| 62 | 6Hex | 990.34 | 15.579 | 0.48 | 10.449 |
| 63 | 6Hex | 990.34 | 17.343 | 0.5 | 11.632 |
| 64 | 6Hex | 990.34 | 18.939 | 0.55 | 12.702 |
| 65 | 6Hex | 990.34 | 19.881 | 1.01 | 13.334 |
| 66 | 6Hex | 990.34 | 21.075 | 0.83 | 14.135 |
| 67 | 6Hex | 990.34 | 21.86 | 0.73 | 14.661 |
| 68 | 6Hex | 990.34 | 22.178 | 0.72 | 14.875 |
| 69 | 6Hex | 990.34 | 22.854 | 2.01 | 15.328 |
| 70 | 6Hex | 990.34 | 23.366 | 1.15 | 15.671 |
| 71 | 6Hex | 990.34 | 24.012 | 1.16 | 16.105 |
| 72 | 6Hex | 990.34 | 25.898 | 0.92 | 17.37 |
| 73 | 6Hex | 990.34 | 26.344 | 0.71 | 17.669 |
| 74 | 6Hex | 990.34 | 28.735 | 0.53 | 19.272 |
| 75 | 6Hex | 990.34 | 28.984 | 0.59 | 19.439 |
| 76 | 7Hex | 1152.39 | 17.924 | 0.18 | 12.021 |
| 77 | 7Hex | 1152.4 | 22.096 | 0.58 | 14.82 |
| 78 | 7Hex | 1152.39 | 22.626 | 0.44 | 15.175 |
| 79 | 7Hex | 1152.4 | 22.915 | 0.27 | 15.369 |
| 80 | 7Hex | 1152.39 | 23.686 | 0.42 | 15.886 |
| 81 | 7Hex | 1152.39 | 24.151 | 0.37 | 16.198 |
| 82 | 7Hex | 1152.39 | 24.537 | 0.74 | 16.457 |
| 83 | 7Hex | 1152.39 | 25.769 | 0.59 | 17.283 |
| 84 | 7Hex | 1152.39 | 27.136 | 0.51 | 18.2 |
| 85 | 7Hex | 1152.39 | 28.252 | 0.34 | 18.948 |
| 86 | 7Hex | 1152.4 | 28.633 | 0.57 | 19.204 |
| 87 | 8Hex | 1314.45 | 24.955 | 0.37 | 16.737 |

TABLE K
CLX105 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex | 342.13 | 1.233 | 0.58 | 1 |
| 2 | 2Hex | 342.13 | 1.43 | 1.79 | 1.16 |
| 3 | 2Hex1Pent | 474.18 | 2.505 | 0.47 | 2.032 |
| 4 | 2Hex1Pent | 474.18 | 3.087 | 0.77 | 2.504 |
| 5 | 2Hex1Pent | 474.18 | 5.487 | 0.38 | 4.45 |
| 6 | 2Hex1Pent | 474.18 | 5.912 | 0.19 | 4.795 |
| 7 | 2Hex1Pent | 474.18 | 6.635 | 0.18 | 5.381 |
| 8 | 2Hex1Pent | 474.18 | 9.208 | 0.61 | 7.468 |
| 9 | 2Hex1Pent | 474.18 | 9.452 | 0.28 | 7.666 |
| 10 | 2Hex2Pent | 606.22 | 3.233 | 0.29 | 2.622 |
| 11 | 2Hex2Pent | 606.22 | 4.847 | 0.75 | 3.931 |
| 12 | 2Hex2Pent | 606.22 | 7.968 | 0.27 | 6.462 |
| 13 | 3Hex | 504.19 | 1.679 | 1.59 | 1.362 |
| 14 | 3Hex | 504.19 | 2.53 | 3.99 | 2.052 |
| 15 | 3Hex | 504.19 | 2.732 | 1.91 | 2.216 |
| 16 | 3Hex | 504.19 | 5.552 | 4.44 | 4.503 |
| 17 | 3Hex1Pent | 636.23 | 6.836 | 0.85 | 5.544 |
| 18 | 3Hex1Pent | 636.23 | 8.019 | 0.25 | 6.504 |
| 19 | 3Hex1Pent | 636.23 | 9.729 | 0.23 | 7.891 |
| 20 | 3Hex1Pent | 636.23 | 9.949 | 0.63 | 8.069 |
| 21 | 3Hex1Pent | 636.23 | 11.439 | 1.27 | 9.277 |
| 22 | 3Hex2Pent | 768.27 | 8.577 | 0.20 | 6.956 |
| 23 | 3Hex3Pent | 900.31 | 12.393 | 0.29 | 10.051 |
| 24 | 4Hex | 666.24 | 3.534 | 6.47 | 2.866 |
| 25 | 4Hex | 666.24 | 4.2 | 5.10 | 3.406 |
| 26 | 4Hex | 666.24 | 5.381 | 4.02 | 4.364 |
| 27 | 4Hex | 666.24 | 6.643 | 5.20 | 5.388 |
| 28 | 4Hex | 666.24 | 8.843 | 7.15 | 7.172 |
| 29 | 4Hex | 666.24 | 9.955 | 2.70 | 8.074 |
| 30 | 4Hex | 666.24 | 11.036 | 0.91 | 8.951 |
| 31 | 4Hex | 666.24 | 14.586 | 1.15 | 11.83 |
| 32 | 4Hex1Pent | 798.28 | 12.257 | 0.21 | 9.941 |
| 33 | 4Hex1Pent | 798.28 | 12.449 | 0.23 | 10.097 |
| 34 | 4Hex1Pent | 798.28 | 12.783 | 0.63 | 10.367 |
| 35 | 4Hex1Pent | 798.28 | 14.336 | 0.50 | 11.627 |
| 36 | 4Hex1Pent | 798.28 | 18.353 | 0.52 | 14.885 |
| 37 | 5Hex | 828.29 | 6.433 | 2.57 | 5.217 |
| 38 | 5Hex | 828.29 | 6.983 | 2.91 | 5.663 |
| 39 | 5Hex | 828.29 | 9.107 | 2.45 | 7.386 |
| 40 | 5Hex | 828.29 | 10.555 | 4.78 | 8.56 |
| 41 | 5Hex | 828.29 | 11.023 | 2.10 | 8.94 |
| 42 | 5Hex | 828.29 | 11.7 | 5.43 | 9.489 |
| 43 | 5Hex | 828.29 | 14.088 | 0.77 | 11.426 |
| 44 | 5Hex | 828.29 | 15.047 | 3.80 | 12.204 |
| 45 | 6Hex | 990.34 | 8.723 | 0.94 | 7.075 |
| 46 | 6Hex | 990.34 | 11.052 | 0.76 | 8.964 |
| 47 | 6Hex | 990.35 | 11.731 | 1.68 | 9.514 |
| 48 | 6Hex | 990.34 | 11.938 | 1.53 | 9.682 |
| 49 | 6Hex | 990.34 | 12.269 | 2.86 | 9.951 |
| 50 | 6Hex | 990.34 | 14.213 | 0.73 | 11.527 |
| 51 | 6Hex | 990.35 | 14.432 | 0.24 | 11.705 |
| 52 | 6Hex | 990.35 | 15.11 | 1.94 | 12.255 |
| 53 | 6Hex | 990.34 | 16.676 | 1.33 | 13.525 |
| 54 | 6Hex | 990.35 | 17.193 | 0.92 | 13.944 |
| 55 | 6Hex | 990.34 | 17.84 | 0.56 | 14.469 |
| 56 | 6Hex | 990.34 | 21.269 | 1.03 | 17.25 |
| 57 | 7Hex | 1152.4 | 12.943 | 0.51 | 10.497 |
| 58 | 7Hex | 1152.39 | 13.193 | 0.47 | 10.7 |
| 59 | 7Hex | 1152.39 | 13.639 | 0.53 | 11.062 |
| 60 | 7Hex | 1152.4 | 16.038 | 0.26 | 13.007 |
| 61 | 7Hex | 1152.4 | 16.731 | 0.50 | 13.569 |
| 62 | 7Hex | 1152.4 | 17.67 | 1.40 | 14.331 |

TABLE L
CLX108 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 3Hex | 504.19 | 1.492 | 2.27 | 1 |
| 2 | 3Hex | 504.19 | 1.855 | 0.85 | 1.243 |
| 3 | 3Hex | 504.18 | 3.002 | 0.75 | 2.012 |
| 4 | 3Hex | 504.18 | 4.461 | 1.28 | 2.99 |
| 5 | 3Hex (NR) | 504.17 | 11.02 | 9.48 | 7.386 |
| 6 | 3Hex1Pent | 636.23 | 6.511 | 4.42 | 4.364 |
| 7 | 3Hex1Pent | 636.23 | 7.066 | 2.09 | 4.736 |
| 8 | 3Hex1Pent | 636.23 | 8.646 | 1.15 | 5.795 |
| 9 | 4Hex | 666.24 | 4.188 | 7.94 | 2.807 |
| 10 | 4Hex | 666.24 | 4.911 | 2.47 | 3.292 |
| 11 | 4Hex | 666.24 | 5.225 | 3.70 | 3.502 |
| 12 | 4Hex | 666.24 | 5.608 | 3.28 | 3.759 |
| 13 | 4Hex | 666.24 | 7.33 | 1.86 | 4.913 |
| 14 | 4Hex | 666.24 | 15.185 | 1.00 | 10.178 |
| 15 | 4Hex1Deoxyhex | 812.26 | 10.546 | 0.99 | 7.068 |
| 16 | 4Hex1HexA | 842.27 | 10.31 | 2.49 | 6.91 |
| 17 | 4Hex1Pent | 798.28 | 9.688 | 4.29 | 6.493 |
| 18 | 4Hex1Pent | 798.28 | 11.09 | 2.19 | 7.433 |
| 19 | 4Hex1Pent | 798.28 | 11.592 | 1.11 | 7.769 |
| 20 | 4Hex1Pent | 798.28 | 14.207 | 0.98 | 9.522 |
| 21 | 5Hex | 828.29 | 7.755 | 6.79 | 5.198 |
| 22 | 5Hex | 828.29 | 7.992 | 2.61 | 5.357 |
| 23 | 5Hex | 828.29 | 8.599 | 2.52 | 5.763 |
| 24 | 5Hex | 828.29 | 9.09 | 2.45 | 6.092 |
| 25 | 5Hex | 828.29 | 9.802 | 1.88 | 6.57 |
| 26 | 5Hex | 828.29 | 10.062 | 3.12 | 6.744 |
| 27 | 5Hex | 828.29 | 13.089 | 2.17 | 8.773 |
| 28 | 6Hex | 990.34 | 11.153 | 5.21 | 7.475 |
| 29 | 6Hex | 990.34 | 12.344 | 1.34 | |
| 30 | 5Hex | 828.29 | 13.089 | 2.17 | |
| 31 | 7Hex | 1152.39 | 13.293 | 3.21 | |
| 32 | 6Hex | 990.34 | 13.877 | 2.18 | |
| 33 | 7Hex | 1152.39 | 14.078 | 1.24 | |
| 34 | 4Hex1Pent | 798.28 | 14.207 | 0.98 | |
| 35 | 6Hex | 990.34 | 14.341 | 2.19 | |
| 36 | 4Hex | 666.24 | 15.185 | 1.00 | |
| 37 | 6Hex | 990.34 | 15.221 | 1.50 | |
| 38 | 7Hex | 1152.39 | 15.273 | 1.03 | |
| 39 | 7Hex | 1152.39 | 16.279 | 1.25 | |

TABLE M
CLX109 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex1Pent | 474.18 | 2.658 | 0.59 | 1 |
| 2 | 2Hex1Pent | 474.17 | 3.038 | 5.45 | 1.143 |
| 3 | 3Hex | 504.19 | 2.69 | 6.74 | 1.012 |
| 4 | 3Hex1HexA | 680.22 | 8.937 | 9.12 | 3.362 |
| 5 | 3Hex1Pent | 636.23 | 6.014 | 0.60 | 2.263 |
| 6 | 3Hex1Pent | 636.23 | 7.292 | 10.79 | 2.743 |
| 7 | 4Hex | 666.24 | 6.614 | 15.53 | 2.488 |
| 8 | 4Hex1Deoxyhex | 812.26 | 13.126 | 3.46 | 4.938 |
| 9 | 4Hex1HexA | 842.27 | 11.857 | 11.47 | 4.461 |
| 10 | 4Hex1Pent | 798.28 | 9.508 | 0.62 | 3.577 |
| 11 | 4Hex1Pent | 798.28 | 10.112 | 7.58 | 3.804 |
| 12 | 5Hex | 828.29 | 9.652 | 9.27 | 3.631 |
| 13 | 5Hex1Deoxyhex | 974.31 | 14.753 | 2.85 | 5.55 |
| 14 | 5Hex1HexA | 1004.32 | 13.573 | 7.20 | 5.106 |
| 15 | 6Hex | 990.34 | 11.515 | 4.50 | 4.332 |
| 16 | 6Hex1HexA | 1166.37 | 14.673 | 3.11 | 5.52 |
| 17 | 7Hex | 1152.39 | 12.782 | 1.15 | 4.809 |

TABLE N
CLX113 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex1Pent | 474.17 | 6.596 | 8.71 | 1 |
| 2 | 2Hex1Pent | 474.17 | 7.182 | 1.54 | 1.089 |
| 3 | 2Hex1Pent | 474.17 | 9.915 | 6.7 | 1.503 |
| 4 | 2Hex2Pent | 606.22 | 10.489 | 0.41 | 1.59 |
| 5 | 2Hex2Pent | 606.22 | 14.055 | 8.77 | 2.131 |
| 6 | 3Hex1Pent | 636.23 | 11.569 | 2.3 | 1.754 |
| 7 | 3Hex1Pent | 636.23 | 12.735 | 4.64 | 1.931 |
| 8 | 3Hex1Pent | 636.23 | 17.858 | 2.32 | 2.707 |
| 9 | 3Hex1Pent | 636.23 | 19.639 | 2.6 | 2.977 |
| 10 | 3Hex1Pent | 636.23 | 21.047 | 2.57 | 3.191 |
| 11 | 3Hex2Pent | 768.27 | 16.236 | 1.77 | 2.461 |
| 12 | 3Hex2Pent | 768.27 | 16.913 | 7.56 | 2.564 |
| 13 | 3Hex2Pent | 768.27 | 22.6 | 4.71 | 3.426 |
| 14 | 3Hex2Pent | 768.27 | 23.712 | 4.79 | 3.595 |
| 15 | 3Hex2Pent | 768.27 | 24.427 | 2.85 | 3.703 |
| 16 | 3Hex3Pent | 900.31 | 25.675 | 1.94 | 3.893 |
| 17 | 4Hex1Pent | 798.28 | 19.976 | 2.44 | 3.029 |
| 18 | 4Hex1Pent | 798.28 | 22.29 | 1.57 | 3.379 |
| 19 | 4Hex1Pent | 798.28 | 26.972 | 0.62 | 4.089 |
| 20 | 4Hex1Pent | 798.28 | 27.808 | 0.91 | 4.216 |
| 21 | 4Hex1Pent | 798.28 | 28.061 | 0.79 | 4.254 |
| 22 | 4Hex2Pent | 930.32 | 18.582 | 2.65 | 2.817 |
| 23 | 4Hex2Pent | 930.32 | 22.74 | 1.7 | 3.448 |
| 24 | 4Hex2Pent | 930.32 | 23.905 | 1.69 | 3.624 |
| 25 | 4Hex2Pent | 930.32 | 24.564 | 3.31 | 3.724 |
| 26 | 4Hex2Pent | 930.32 | 24.966 | 3.86 | 3.785 |
| 27 | 4Hex2Pent | 930.32 | 27.736 | 1.18 | 4.205 |
| 28 | 4Hex2Pent | 930.32 | 28.44 | 0.98 | 4.312 |
| 29 | 4Hex2Pent | 930.32 | 28.778 | 1.97 | 4.363 |
| 30 | 4Hex2Pent | 930.32 | 29.18 | 1.24 | 4.424 |
| 31 | 4Hex2Pent | 930.32 | 30.836 | 0.75 | 4.675 |
| 32 | 4Hex3Pent | 1062.36 | 26.017 | 1.49 | 3.944 |
| 33 | 4Hex3Pent | 1062.36 | 26.225 | 1.2 | 3.976 |
| 34 | 4Hex3Pent | 1062.36 | 29.365 | 0.99 | 4.452 |
| 35 | 4Hex3Pent | 1062.36 | 29.76 | 0.96 | 4.512 |
| 36 | 4Hex3Pent | 1062.36 | 31.172 | 0.58 | 4.726 |
| 37 | 4Hex3Pent | 1062.36 | 31.49 | 1.02 | 4.774 |
| 38 | 5Hex3Pent | 1224.42 | 26.484 | 1.27 | 4.015 |
| 39 | 5Hex3Pent | 1224.42 | 28.91 | 0.54 | 4.383 |
| 40 | 5Hex3Pent | 1224.42 | 29.282 | 0.74 | 4.439 |
| 41 | 5Hex3Pent | 1224.42 | 30.009 | 0.89 | 4.55 |
| 42 | 5Hex3Pent | 1224.42 | 31.349 | 0.47 | 4.753 |

TABLE O
CLX114 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 3Pent | 414.15 | 5.215 | 2.15 | 1 |
| 2 | 3Pent | 414.15 | 8.612 | 5.60 | 1.651 |
| 3 | 3Pent | 414.15 | 11.556 | 5.01 | 2.216 |
| 4 | 3Pent | 414.15 | 14.346 | 8.99 | 2.751 |
| 5 | 3Pent | 414.15 | 21.696 | 3.32 | 4.16 |
| 6 | 4Pent | 546.2 | 7.539 | 2.29 | 1.446 |
| 7 | 4Pent | 546.2 | 8.925 | 5.02 | 1.711 |
| 8 | 4Pent | 546.19 | 16.952 | 4.99 | 3.251 |
| 9 | 4Pent | 546.2 | 20.455 | 5.68 | 3.922 |
| 10 | 5Pent | 678.24 | 11.806 | 3.97 | 2.264 |
| 11 | 5Pent | 678.24 | 16.229 | 4.00 | 3.112 |
| 12 | 5Pent | 678.24 | 19.91 | 5.20 | 3.818 |
| 13 | 5Pent | 678.24 | 20.812 | 8.23 | 3.991 |
| 14 | 5Pent | 678.24 | 23.691 | 3.63 | 4.543 |
| 15 | 5Pent | 678.24 | 25.947 | 6.89 | 4.975 |
| 16 | 6Pent | 810.28 | 17.177 | 2.16 | 3.294 |
| 17 | 6Pent | 810.28 | 21.326 | 2.88 | 4.089 |
| 18 | 6Pent | 810.28 | 24.969 | 4.62 | 4.788 |
| 19 | 6Pent | 810.28 | 25.26 | 3.51 | 4.844 |
| 20 | 6Pent | 810.28 | 26.913 | 3.30 | 5.161 |
| 21 | 7Pent | 942.32 | 25.618 | 2.90 | 4.912 |
| 22 | 7Pent | 942.32 | 27.697 | 5.66 | 5.311 |

TABLE P
CLX115 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| Com- | | | RT | Oligo | Retention |
|---|---|---|---|---|---|
| pound | Identity | Mass | (min) | Wt. % | Factor |
| 1 | 1Hex1Pent | 314.1214 | 1.839 | 0.09 | 1.196 |
| 2 | 1Hex2Pent | 446.1638 | 14.618 | 0.46 | 9.511 |
| 3 | 1Hex2Pent | 446.1641 | 14.73 | 0.17 | 9.584 |
| 4 | 1Hex2Pent | 446.1635 | 15.648 | 0.28 | 10.181 |
| 5 | 1Hex2Pent | 446.1643 | 17.222 | 0.11 | 11.205 |
| 6 | 2Hex | 344.1321 | 1.537 | 1.05 | 1 |
| 7 | 2Hex | 344.1329 | 2.878 | 2.41 | 1.872 |
| 8 | 2Hex1Pent | 476.1744 | 12.931 | 1.06 | 8.413 |
| 9 | 2Hex1Pent | 476.174 | 13.229 | 0.49 | 8.607 |
| 10 | 2Hex1Pent | 476.1753 | 13.732 | 1.53 | 8.934 |
| 11 | 2Hex1Pent | 476.1742 | 15.112 | 0.33 | 9.832 |
| 12 | 2Hex1Pent | 476.1748 | 16.113 | 0.84 | 10.483 |
| 13 | 2Hex1Pent | 476.1742 | 16.527 | 0.33 | 10.753 |
| 14 | 2Hex2Pent | 608.2157 | 24.944 | 0.45 | 16.229 |
| 15 | 2Hex2Pent | 608.2161 | 26.779 | 0.19 | 17.423 |
| 16 | 3Hex | 506.1856 | 3.295 | 0.63 | 2.144 |
| 17 | 3Hex | 506.1856 | 4.549 | 0.8 | 2.96 |
| 18 | 3Hex | 506.1843 | 10.5 | 0.08 | 6.831 |
| 19 | 3Hex | 506.1855 | 11.335 | 7.55 | 7.375 |
| 20 | 3Hex | 506.1855 | 14.01 | 0.23 | 9.115 |
| 21 | 3Hex | 506.1857 | 15.193 | 9.74 | 9.885 |
| 22 | 3Hex | 506.1857 | 15.445 | 0.05 | 10.049 |
| 23 | 3Hex | 506.184 | 15.874 | 0.08 | 10.328 |
| 24 | 3Hex | 506.1856 | 17.831 | 6.41 | 11.601 |
| 25 | 3Hex1HexA | 682.2171 | 25.124 | 0.13 | 16.346 |
| 26 | 3Hex1HexA | 682.2176 | 27.182 | 0.58 | 17.685 |
| 27 | 3Hex1HexA | 682.2177 | 27.272 | 0.19 | 17.744 |
| 28 | 3Hex1HexA | 682.2169 | 28.058 | 0.67 | 18.255 |
| 29 | 3Hex1Pent | 638.2261 | 9.335 | 0.1 | 6.074 |
| 30 | 3Hex1Pent | 638.2263 | 10.131 | 0.07 | 6.591 |
| 31 | 3Hex1Pent | 638.2276 | 23.161 | 0.93 | 15.069 |
| 32 | 3Hex1Pent | 638.2275 | 23.637 | 0.4 | 15.379 |
| 33 | 3Hex1Pent | 638.2273 | 24.298 | 0.44 | 15.809 |
| 34 | 3Hex1Pent | 638.2271 | 25.161 | 0.72 | 16.37 |
| 35 | 3Hex1Pent | 638.2282 | 25.768 | 0.84 | 16.765 |
| 36 | 3Hex1Pent | 638.2271 | 26.311 | 0.23 | 17.118 |
| 37 | 3Hex2Pent | 770.2696 | 29.593 | 0.06 | 19.254 |
| 38 | 3Hex2Pent | 770.2686 | 30.152 | 0.19 | 19.617 |
| 39 | 3Hex2Pent | 770.2681 | 30.838 | 0.07 | 20.064 |
| 40 | 3Hex2Pent | 770.2695 | 30.903 | 0.12 | 20.106 |
| 41 | 4Hex | 668.237 | 7.62 | 0.19 | 4.958 |
| 42 | 4Hex | 668.238 | 8.72 | 0.1 | 5.673 |
| 43 | 4Hex | 668.237 | 9.276 | 1.35 | 6.035 |
| 44 | 4Hex | 668.2351 | 9.819 | 0.06 | 6.388 |
| 45 | 4Hex | 668.2378 | 10.269 | 1.06 | 6.681 |
| 46 | 4Hex | 668.2379 | 10.63 | 0.34 | 6.916 |
| 47 | 4Hex | 668.2388 | 21.557 | 8.22 | 14.025 |
| 48 | 4Hex | 668.2386 | 24.211 | 0.1 | 15.752 |
| 49 | 4Hex | 668.239 | 25.244 | 4.57 | 16.424 |
| 50 | 4Hex | 668.239 | 25.507 | 5.92 | 16.595 |
| 51 | 4Hex | 668.2387 | 26.403 | 6.18 | 17.178 |
| 52 | 4Hex | 668.2343 | 26.607 | 0.04 | 17.311 |
| 53 | 4Hex1Deoxyhex | 814.2585 | 31.081 | 0.31 | 20.222 |
| 54 | 4Hex1HexA | 844.247 | 29.807 | 0.04 | 19.393 |
| 55 | 4Hex1Pent | 800.2787 | 28.827 | 0.23 | 18.755 |
| 56 | 4Hex1Pent | 800.2797 | 29.062 | 0.41 | 18.908 |
| 57 | 4Hex1Pent | 800.2787 | 29.552 | 0.11 | 19.227 |
| 58 | 4Hex1Pent | 800.2799 | 29.615 | 0.56 | 19.268 |
| 59 | 4Hex1Pent | 800.2788 | 30.227 | 1.2 | 19.666 |
| 60 | 5Hex | 830.2911 | 11.751 | 0.2 | 7.645 |
| 61 | 5Hex | 830.2895 | 13.261 | 0.48 | 8.628 |
| 62 | 5Hex | 830.2893 | 13.518 | 0.46 | 8.795 |
| 63 | 5Hex | 830.2899 | 13.84 | 0.71 | 9.005 |
| 64 | 5Hex | 830.2907 | 27.932 | 2.29 | 18.173 |
| 65 | 5Hex | 830.2922 | 28.423 | 3.57 | 18.493 |
| 66 | 5Hex | 830.2907 | 29.322 | 1.02 | 19.077 |
| 67 | 5Hex | 830.2908 | 29.82 | 4.98 | 19.401 |
| 68 | 5Hex | 830.291 | 30.469 | 6.13 | 19.824 |
| 69 | 6Hex | 992.3433 | 14.683 | 0.1 | 9.553 |
| 70 | 6Hex | 992.342 | 15.905 | 0.98 | 10.348 |
| 71 | 6Hex | 992.3433 | 31.896 | 1.52 | 20.752 |
| 72 | 6Hex | 992.3427 | 31.937 | 1.02 | 20.779 |
| 73 | 6Hex | 992.3434 | 33.337 | 0.52 | 21.69 |
| 74 | 6Hex | 992.3429 | 33.37 | 1.9 | 21.711 |
| 75 | 6Hex | 992.3429 | 33.763 | 0.91 | 21.967 |
| 76 | 6Hex | 992.3421 | 35.487 | 1.12 | 23.088 |

TABLE Q
CLX123 comprises the oligosaccharides shown in this
table, as measured by oligosaccharide analysis.
Hex refers to hexose sugars, Pent refers to pentose
sugars, and HexA refers to hexuronic acid sugars.

| | | | RT | Oligo | Retention |
|---|---|---|---|---|---|
| Compound | Identity | Mass | (min) | Wt. % | Factor |
| 1 | 2Hex1HexA | 518.16 | 8.325 | 0.62 | 4.73 |
| 2 | 2Hex1HexA | 518.16 | 8.947 | 3.64 | 5.084 |
| 3 | 2Hex1Pent | 474.17 | 2.959 | 0.17 | 1.681 |
| 4 | 2Hex1Pent | 474.17 | 3.194 | 1.02 | 1.815 |
| 5 | 2Hex1Pent | 474.17 | 4.957 | 3.69 | 2.816 |
| 6 | 2Hex1Pent | 474.17 | 6.228 | 0.45 | 3.539 |
| 7 | 2Hex1Pent | 474.17 | 7.324 | 0.47 | 4.161 |
| 8 | 2Hex1Pent | 474.17 | 8.744 | 0.56 | 4.968 |
| 9 | 3Hex | 504.18 | 1.76 | 0.23 | 1 |
| 10 | 3Hex | 504.18 | 2.979 | 1.35 | 1.693 |
| 11 | 3Hex | 504.18 | 3.493 | 3.1 | 1.985 |
| 12 | 3Hex | 504.18 | 4.368 | 0.71 | 2.482 |
| 13 | 3Hex | 504.18 | 4.621 | 5.04 | 2.626 |
| 14 | 3Hex | 504.19 | 4.969 | 10.34 | 2.823 |
| 15 | 3Hex | 504.18 | 6.283 | 1.47 | 3.57 |
| 16 | 3Hex | 504.18 | 7.081 | 1.01 | 4.023 |
| 17 | 3Hex | 504.18 | 9.241 | 1.5 | 5.251 |
| 18 | 3Hex | 504.18 | 9.776 | 2.12 | 5.555 |
| 19 | 3Hex | 504.18 | 15.365 | 1.46 | 8.73 |
| 20 | 3Hex1HexA | 680.22 | 4.082 | 0.5 | 2.319 |
| 21 | 3Hex1HexA | 680.22 | 6.239 | 0.48 | 3.545 |
| 22 | 3Hex1HexA | 680.22 | 19.397 | 2.04 | 11.021 |
| 23 | 3Hex1Pent | 636.23 | 7.418 | 0.22 | 4.215 |
| 24 | 3Hex1Pent | 636.23 | 8.142 | 3.1 | 4.626 |
| 25 | 3Hex1Pent | 636.23 | 9.592 | 1.44 | 5.45 |
| 26 | 3Hex1Pent | 636.23 | 10.399 | 1.06 | 5.909 |
| 27 | 3Hex1Pent | 636.23 | 10.963 | 1.67 | 6.229 |
| 28 | 3Hex1Pent | 636.23 | 16.021 | 0.65 | 9.103 |
| 29 | 4Hex | 666.24 | 7.534 | 1.63 | 4.281 |
| 30 | 4Hex | 666.24 | 7.918 | 0.6 | 4.499 |
| 31 | 4Hex | 666.24 | 8.781 | 7.65 | 4.989 |
| 32 | 4Hex | 666.24 | 9.533 | 3.13 | 5.416 |
| 33 | 4Hex | 666.24 | 10.45 | 4.42 | 5.938 |
| 34 | 4Hex | 666.24 | 10.889 | 3.98 | 6.187 |
| 35 | 4Hex | 666.24 | 11.44 | 0.5 | 6.5 |
| 36 | 4Hex | 666.24 | 11.7 | 0.56 | 6.648 |
| 37 | 4Hex | 666.24 | 12.426 | 1.56 | 7.06 |
| 38 | 4Hex | 666.24 | 16.009 | 0.45 | 9.096 |
| 39 | 4Hex | 666.24 | 16.252 | 1.35 | 9.234 |
| 40 | 4Hex | 666.24 | 25.297 | 0.23 | 14.373 |
| 41 | 4Hex1HexA | 842.27 | 8.233 | 0.65 | 4.678 |
| 42 | 4Hex1HexA | 842.27 | 13.575 | 1.24 | 7.713 |
| 43 | 4Hex1Pent | 798.28 | 11.859 | 0.58 | 6.738 |
| 44 | 4Hex1Pent | 798.28 | 12.178 | 0.74 | 6.919 |
| 45 | 4Hex1Pent | 798.28 | 13.095 | 2.49 | 7.44 |
| 46 | 4Hex1Pent | 798.28 | 14.144 | 0.79 | 8.036 |
| 47 | 4Hex1Pent | 798.28 | 19.536 | 0.42 | 11.1 |
| 48 | 5Hex | 828.29 | 11.298 | 0.55 | 6.419 |
| 49 | 5Hex | 828.29 | 11.761 | 0.51 | 6.682 |
| 50 | 5Hex | 828.29 | 11.962 | 2.04 | 6.797 |
| 51 | 5Hex | 828.29 | 12.608 | 1.79 | 7.164 |
| 52 | 5Hex | 828.29 | 12.853 | 5.08 | 7.303 |
| 53 | 5Hex | 828.29 | 13.744 | 0.63 | 7.809 |
| 54 | 5Hex | 828.29 | 14.038 | 2.43 | 7.976 |
| 55 | 5Hex | 828.29 | 15.144 | 0.39 | 8.605 |
| 56 | 5Hex | 828.29 | 17.108 | 0.41 | 9.72 |
| 57 | 5Hex | 828.29 | 19.354 | 0.82 | 10.997 |
| 58 | 5Hex | 828.29 | 21.535 | 0.24 | 12.236 |
| 59 | 5Hex1Pent | 960.33 | 15.57 | 0.28 | 8.847 |
| 60 | 6Hex | 990.34 | 15.31 | 0.93 | 8.699 |
| 61 | 7Hex | 1152.39 | 16.851 | 0.77 | 9.574 |

TABLE R
CLX125 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | | | Reten- |
|---|---|---|---|---|---|
| Com- | | | RT | Oligo | tion |
| pound | Identity | Mass | (min) | Wt. % | Factor |
| 1 | 1Hex1HexA1Deoxyhex | 502.17 | 19.054 | 10.75 | 7.46 |
| 2 | 2Hex1Deoxyhex | 488.19 | 2.554 | 5.22 | 1 |
| 3 | 2Hex1HexA | 518.16 | 18.245 | 6.09 | 7.144 |
| 4 | 2Hex1HexA1Deoxyhex | 664.22 | 17.01 | 20.06 | 6.66 |
| 5 | 2Hex1HexA1Deoxyhex | 664.22 | 26.202 | 11.78 | 10.259 |
| 6 | 2Hex1HexA1Deoxyhex | 664.22 | 30.461 | 6.73 | 11.927 |
| 7 | 2Hex1HexA2Deoxyhex | 810.28 | 34.83 | 7.10 | 13.637 |
| 8 | 3Hex | 504.19 | 3.133 | 0.21 | 1.227 |
| 9 | 3Hex | 504.19 | 3.384 | 0.24 | 1.325 |
| 10 | 3Hex | 504.18 | 4.804 | 2.33 | 1.881 |
| 11 | 3Hex1HexA1Deoxyhex | 826.28 | 28.498 | 11.38 | 11.158 |
| 12 | 3Hex1HexA1Deoxyhex | 826.27 | 28.523 | 3.44 | 11.168 |
| 13 | 3Hex1HexA2Deoxyhex | 972.33 | 32.07 | 8.64 | 12.557 |
| 14 | 4Hex | 666.24 | 9.489 | 0.27 | 3.715 |
| 15 | 4Hex | 666.24 | 10.444 | 0.32 | 4.089 |
| 16 | 4Hex | 666.24 | 10.847 | 2.00 | 4.247 |
| 17 | 4Hex | 666.23 | 16.998 | 1.52 | 6.655 |
| 18 | 5Hex | 828.29 | 13.948 | 1.36 | 5.461 |
| 19 | 8Hex | 1314.46 | 26.197 | 0.55 | 10.257 |

TABLE S
CLX126 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex1HexA | 518.16 | 5.195 | 2.97 | 1.612 |
| 2 | 2Hex1Pent | 474.17 | 3.223 | 2.41 | 1 |
| 3 | 2Hex1Pent | 474.17 | 3.759 | 8.13 | 1.166 |
| 4 | 2Hex2Pent | 606.22 | 8.304 | 1.36 | 2.576 |
| 5 | 2Hex2Pent | 606.22 | 9.147 | 2.01 | 2.838 |
| 6 | 3Hex | 504.18 | 3.24 | 8.55 | 1.005 |
| 7 | 3Hex | 504.18 | 5.024 | 1.54 | 1.559 |
| 8 | 3Hex1HexA | 680.22 | 10.517 | 5.15 | 3.263 |
| 9 | 3Hex1Pent | 636.23 | 7.456 | 0.70 | 2.313 |
| 10 | 3Hex1Pent | 636.22 | 8.003 | 0.83 | 2.483 |
| 11 | 3Hex1Pent | 636.23 | 8.309 | 2.00 | 2.578 |
| 12 | 3Hex1Pent | 636.23 | 9.023 | 9.78 | 2.8 |
| 13 | 3Hex2Pent | 768.27 | 11.553 | 0.98 | 3.585 |
| 14 | 3Hex2Pent | 768.27 | 11.749 | 0.64 | 3.645 |
| 15 | 3Hex2Pent | 768.27 | 12.553 | 1.84 | 3.895 |
| 16 | 4Hex | 666.24 | 8.283 | 9.35 | 2.57 |
| 17 | 4Hex | 666.24 | 10.913 | 1.47 | 3.386 |
| 18 | 4Hex | 666.24 | 19.608 | 0.38 | 6.084 |
| 19 | 4Hex1HexA | 842.27 | 13.588 | 4.66 | 4.216 |
| 20 | 4Hex1Pent | 798.28 | 10.957 | 0.49 | 3.4 |
| 21 | 4Hex1Pent | 798.28 | 11.271 | 1.01 | 3.497 |
| 22 | 4Hex1Pent | 798.28 | 12.045 | 9.22 | 3.737 |
| 23 | 4Hex2Pent | 930.32 | 13.503 | 0.42 | 4.19 |
| 24 | 5Hex | 828.29 | 11.557 | 6.93 | 3.586 |
| 25 | 5Hex1HexA | 1004.32 | 15.435 | 3.39 | 4.789 |
| 26 | 5Hex1Pent | 960.33 | 13.319 | 0.89 | 4.132 |
| 27 | 5Hex1Pent | 960.33 | 13.904 | 5.94 | 4.314 |
| 28 | 6Hex | 990.34 | 13.511 | 4.43 | 4.192 |
| 29 | 6Hex1HexA | 1166.38 | 16.633 | 1.45 | 5.161 |
| 30 | 7Hex1Pent | 1284.44 | 16.31 | 1.09 | 5.061 |

TABLE T
CLX128 comprises the oligosaccharides shown in this table, as
measured by oligosaccharide analysis. Hex refers to hexose
sugars, Pent refers to pentose sugars, HexA refers to hexuronic
acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex1Pent | 474.18 | 13.955 | 3.91 | 7.131 |
| 2 | 3Hex | 504.18 | 11.599 | 1.85 | 5.927 |
| 3 | 3Hex | 504.19 | 15.414 | 18.53 | 7.876 |
| 4 | 3Hex | 504.18 | 18.158 | 2.07 | 9.278 |
| 5 | 3Hex (NR) | 504.17 | 1.957 | 1.30 | 1 |
| 6 | 3Hex (NR) | 504.17 | 3.84 | 0.30 | 1.962 |
| 7 | 3Hex (NR) | 504.17 | 5.404 | 1.29 | 2.761 |
| 8 | 3Hex1Pent | 636.23 | 24.526 | 2.88 | 12.532 |
| 9 | 3Hex1Pent | 636.23 | 25.874 | 0.31 | 13.221 |
| 10 | 3Pent | 414.15 | 2.398 | 0.39 | 1.225 |
| 11 | 3Pent | 414.15 | 9.736 | 8.48 | 4.975 |
| 12 | 3Pent | 414.15 | 13.843 | 0.82 | 7.074 |
| 13 | 4Hex | 666.24 | 21.972 | 1.80 | 11.227 |
| 14 | 4Hex | 666.24 | 25.37 | 13.78 | 12.964 |
| 15 | 4Hex | 666.24 | 25.62 | 2.34 | 13.091 |
| 16 | 4Hex | 666.24 | 26.463 | 2.28 | 13.522 |
| 17 | 4Hex1Pent | 798.28 | 29.11 | 1.70 | 14.875 |
| 18 | 4Pent | 546.19 | 10.029 | 1.13 | 5.125 |
| 19 | 4Pent | 546.2 | 15.641 | 1.48 | 7.992 |
| 20 | 4Pent | 546.19 | 18.132 | 7.52 | 9.265 |
| 21 | 4Pent | 546.2 | 21.384 | 0.76 | 10.927 |
| 22 | 5Hex | 828.29 | 28.523 | 0.74 | 14.575 |
| 23 | 5Hex | 828.29 | 29.329 | 8.52 | 14.987 |
| 24 | 5Hex | 828.29 | 29.834 | 1.76 | 15.245 |
| 25 | 5Hex | 828.29 | 30.473 | 2.21 | 15.571 |
| 26 | 5Pent | 678.24 | 17.349 | 0.70 | 8.865 |
| 27 | 5Pent | 678.24 | 20.908 | 0.79 | 10.684 |
| 28 | 5Pent | 678.24 | 21.722 | 1.17 | 11.1 |
| 29 | 5Pent | 678.24 | 24.34 | 5.23 | 12.437 |
| 30 | 5Pent | 678.24 | 26.576 | 0.50 | 13.58 |
| 31 | 6Hex | 990.34 | 33.38 | 0.47 | 17.057 |
| 32 | 6Pent | 810.28 | 27.583 | 1.49 | 14.095 |
| 33 | 7Pent | 942.32 | 29.641 | 1.51 | 15.146 |

TABLE U
CLX122 comprises the oligosaccharides shown in this
table, as measured by oligosaccharide analysis.
(Hex = hexose, generally galactose or mannose).

| | | | RT | Oligo | Retention |
|---|---|---|---|---|---|
| Compound | Identity | Mass | (min) | Wt. % | Factor |
| 1 | 2Hex1Pent | 474.17 | 3.789 | 3.20 | 1.157 |
| 2 | 2Hex1Pent | 474.17 | 5.019 | 1.67 | 1.532 |
| 3 | 3Hex | 504.18 | 3.276 | 3.64 | 1 |
| 4 | 3Hex | 504.18 | 5.023 | 5.46 | 1.533 |
| 5 | 3Hex | 504.18 | 15.334 | 2.97 | 4.681 |
| 6 | 3Hex (NR) | 504.17 | 3.911 | 1.52 | 1.194 |
| 7 | 3Hex (NR) | 504.17 | 5.613 | 3.36 | 1.713 |
| 8 | 3Hex (NR) | 504.17 | 10.914 | 3.05 | 3.332 |
| 9 | 3Hex (NR) | 504.17 | 12.221 | 8.03 | 3.73 |
| 10 | 3Hex1Pent | 636.23 | 8.958 | 3.67 | 2.734 |
| 11 | 3Hex1Pent | 636.23 | 10.904 | 1.65 | 3.328 |
| 12 | 3Pent | 414.15 | 3.867 | 0.98 | 1.18 |
| 13 | 3Pent | 414.15 | 6.269 | 9.92 | 1.914 |
| 14 | 3Pent | 414.16 | 7.057 | 0.81 | 2.154 |
| 15 | 4Hex | 666.24 | 8.187 | 3.64 | 2.499 |
| 16 | 4Hex | 666.24 | 10.829 | 5.60 | 3.306 |
| 17 | 4Hex | 666.24 | 19.604 | 0.58 | 5.984 |
| 18 | 4Hex | 666.24 | 25.305 | 2.23 | 7.724 |
| 19 | 4Hex (NR) | 666.22 | 9.171 | 3.30 | 2.799 |
| 20 | 4Hex (NR) | 666.22 | 12.226 | 0.95 | 3.732 |
| 21 | 4Hex (NR) | 666.22 | 12.227 | 4.67 | 3.732 |
| 22 | 4Hex (NR) | 666.22 | 15.283 | 3.31 | 4.665 |
| 23 | 4Hex1Pent | 798.28 | 11.988 | 3.05 | 3.659 |
| 24 | 4Hex1Pent | 798.28 | 14.07 | 1.23 | 4.295 |
| 25 | 4Pent | 546.19 | 10.644 | 1.79 | 3.249 |
| 26 | 4Pent | 546.2 | 11.358 | 3.33 | 3.467 |
| 27 | 4Pent | 546.2 | 13.022 | 0.89 | 3.975 |
| 28 | 5Hex | 828.29 | 11.467 | 2.50 | 3.5 |
| 29 | 5Hex | 828.29 | 13.946 | 4.75 | 4.257 |
| 30 | 5Hex | 828.29 | 29.244 | 1.39 | 8.927 |
| 31 | 5Pent | 678.24 | 15.834 | 2.26 | 4.833 |
| 32 | 5Pent | 678.23 | 17.759 | 1.00 | 5.421 |
| 33 | 6Hex | 990.34 | 15.785 | 2.77 | 4.818 |
| 34 | 6Hex1Pent | 1122.39 | 15.078 | 0.83 | 4.603 |

TABLE V
CLX115Cu comprises the oligosaccharides shown in this
table. Hex refers to hexose sugars, Pent refers to
pentose sugars, HexA refers to hexuronic acid sugars,
and Deoxyhex refers to deoxyhexose sugars.

| | | | RT | Oligo | Retention |
|---|---|---|---|---|---|
| Compound | Identity | Mass | (min) | Wt. % | Factor |
| 1 | 1Hex1Pent | 312.12 | 2.063 | 3.5 | 1.158 |
| 2 | 1Hex1Pent | 312.12 | 11.288 | 0.31 | 6.334 |
| 3 | 1Hex1Pent | 312.12 | 12.054 | 0.44 | 6.764 |
| 4 | 1Hex1Pent | 312.12 | 14.528 | 0.4 | 8.153 |
| 5 | 1Hex2Pent | 444.17 | 13.12 | 0.95 | 7.363 |
| 6 | 1Hex2Pent | 444.17 | 14.019 | 0.9 | 7.867 |
| 7 | 1Hex2Pent | 444.17 | 15.704 | 0.28 | 8.813 |
| 8 | 2Hex | 342.13 | 2.189 | 0.2 | 1.228 |
| 9 | 2Hex | 342.13 | 2.916 | 0.82 | 1.636 |
| 10 | 2Hex | 342.13 | 3.01 | 2.45 | 1.689 |
| 11 | 2Hex | 342.13 | 9.733 | 1.32 | 5.462 |
| 12 | 2Hex | 342.13 | 13.479 | 1.89 | 7.564 |
| 13 | 2Hex1Pent | 474.18 | 10.122 | 0.05 | 5.68 |
| 14 | 2Hex1Pent | 474.17 | 11.287 | 1.96 | 6.334 |
| 15 | 2Hex1Pent | 474.17 | 11.594 | 0.43 | 6.506 |
| 16 | 2Hex1Pent | 474.17 | 12.054 | 2.65 | 6.764 |
| 17 | 2Hex1Pent | 474.18 | 14.528 | 1.12 | 8.153 |
| 18 | 2Hex1Pent | 474.18 | 14.944 | 0.44 | 8.386 |
| 19 | 2Hex2Pent | 606.22 | 23.54 | 0.32 | 13.21 |
| 20 | 2Hex2Pent | 606.22 | 24.287 | 0.38 | 13.629 |
| 21 | 2Hex2Pent | 606.22 | 25.041 | 0.61 | 14.052 |
| 22 | 3Hex | 504.19 | 1.782 | 0.09 | 1 |
| 23 | 3Hex | 504.19 | 3.333 | 0.43 | 1.87 |
| 24 | 3Hex | 504.18 | 4.242 | 0.15 | 2.38 |
| 25 | 3Hex | 504.18 | 5.108 | 0.12 | 2.866 |
| 26 | 3Hex | 504.18 | 6.804 | 0.31 | 3.818 |
| 27 | 3Hex | 504.18 | 8.412 | 0.76 | 4.721 |
| 28 | 3Hex | 504.18 | 8.669 | 0.39 | 4.865 |
| 29 | 3Hex | 504.19 | 9.734 | 9.22 | 5.462 |
| 30 | 3Hex | 504.19 | 12.287 | 1.16 | 6.895 |
| 31 | 3Hex | 504.19 | 23.908 | 0.43 | 13.416 |
| 32 | 3Hex | 504.19 | 24.73 | 0.32 | 13.878 |
| 33 | 3Hex | 504.19 | 27.971 | 0.46 | 15.696 |
| 34 | 3Hex1Deoxyhex | 650.24 | 1.903 | 0.06 | 1.068 |
| 35 | 3Hex1HexA | 680.22 | 23.207 | 1.06 | 13.023 |
| 36 | 3Hex1HexA | 680.22 | 25.112 | 0.94 | 14.092 |
| 37 | 3Hex1HexA | 680.22 | 25.972 | 1.07 | 14.575 |
| 38 | 3Hex1Pent | 636.23 | 19.245 | 0.03 | 10.8 |
| 39 | 3Hex1Pent | 636.23 | 21.862 | 1.58 | 12.268 |
| 40 | 3Hex1Pent | 636.23 | 22.377 | 0.32 | 12.557 |
| 41 | 3Hex1Pent | 636.23 | 22.939 | 0.71 | 12.873 |
| 42 | 3Hex1Pent | 636.23 | 23.624 | 0.76 | 13.257 |
| 43 | 3Hex1Pent | 636.23 | 24.175 | 1.49 | 13.566 |
| 44 | 3Hex1Pent | 636.22 | 24.268 | 0.1 | 13.618 |
| 45 | 3Hex1Pent | 636.23 | 24.438 | 0.31 | 13.714 |
| 46 | 3Hex1Pent | 636.23 | 24.656 | 0.09 | 13.836 |
| 47 | 3Hex2Pent | 768.27 | 28.4 | 0.2 | 15.937 |
| 48 | 3Hex2Pent | 768.27 | 29.119 | 0.16 | 16.341 |
| 49 | 4Hex | 666.24 | 7.554 | 0.18 | 4.239 |
| 50 | 4Hex | 666.24 | 8.467 | 0.12 | 4.751 |
| 51 | 4Hex | 666.24 | 17.617 | 0.24 | 9.886 |
| 52 | 4Hex | 666.24 | 17.98 | 0.14 | 10.09 |
| 53 | 4Hex | 666.24 | 18.202 | 0.71 | 10.214 |
| 54 | 4Hex | 666.24 | 18.74 | 0.21 | 10.516 |
| 55 | 4Hex | 666.24 | 19.121 | 0.3 | 10.73 |
| 56 | 4Hex | 666.24 | 20.19 | 9.67 | 11.33 |
| 57 | 4Hex | 666.24 | 21.297 | 0.4 | 11.951 |
| 58 | 4Hex | 666.24 | 22.289 | 0.21 | 12.508 |
| 59 | 4Hex | 666.24 | 23.248 | 0.13 | 13.046 |
| 60 | 4Hex | 666.24 | 23.697 | 4.91 | 13.298 |
| 61 | 4Hex | 666.24 | 23.84 | 2.45 | 13.378 |
| 62 | 4Hex | 666.24 | 23.869 | 0.25 | 13.395 |
| 63 | 4Hex | 666.24 | 24.452 | 0.28 | 13.722 |
| 64 | 4Hex | 666.24 | 24.73 | 7.14 | 13.878 |
| 65 | 4Hex1HexA | 842.27 | 30.693 | 1.13 | 17.224 |
| 66 | 4Hex1Pent | 798.28 | 27.188 | 0.57 | 15.257 |
| 67 | 4Hex1Pent | 798.28 | 27.761 | 0.48 | 15.579 |
| 68 | 4Hex1Pent | 798.28 | 28.4 | 1.13 | 15.937 |
| 69 | 5Hex | 828.29 | 22.477 | 0.05 | 12.613 |
| 70 | 5Hex | 828.29 | 22.735 | 0.05 | 12.758 |
| 71 | 5Hex | 828.29 | 23.539 | 0.1 | 13.209 |
| 72 | 5Hex | 828.29 | 24.626 | 0.09 | 13.819 |
| 73 | 5Hex | 828.29 | 24.857 | 0.29 | 13.949 |
| 74 | 5Hex | 828.29 | 26.032 | 2.76 | 14.608 |
| 75 | 5Hex | 828.29 | 26.525 | 0.07 | 14.885 |
| 76 | 5Hex | 828.29 | 26.537 | 3.33 | 14.892 |
| 77 | 5Hex | 828.29 | 27.442 | 1.4 | 15.4 |
| 78 | 5Hex | 828.29 | 27.974 | 4.82 | 15.698 |
| 79 | 5Hex | 828.29 | 28.668 | 0.12 | 16.088 |
| 80 | 5Hex | 828.29 | 28.674 | 7.11 | 16.091 |
| 81 | 5Hex | 828.29 | 28.697 | 0.04 | 16.104 |
| 82 | 5Hex | 828.29 | 41.967 | 0.32 | 23.551 |
| 83 | 5Hex1Deoxyhex | 974.35 | 11.313 | 0.12 | 6.348 |
| 84 | 5Hex1Deoxyhex | 974.35 | 12.165 | 0.08 | 6.827 |
| 85 | 5Hex1Deoxyhex | 974.35 | 13.385 | 0.03 | 7.511 |
| 86 | 5Hex1Pent | 960.34 | 30.951 | 0.07 | 17.369 |
| 87 | 5Hex1Pent | 960.33 | 31.583 | 0.15 | 17.723 |
| 88 | 6Hex | 990.34 | 26.403 | 0.06 | 14.816 |
| 89 | 6Hex | 990.34 | 27.633 | 0.05 | 15.507 |
| 90 | 6Hex | 990.34 | 30.321 | 0.51 | 17.015 |
| 91 | 6Hex | 990.34 | 31.531 | 1.44 | 17.694 |
| 92 | 6Hex | 990.34 | 31.983 | 1.21 | 17.948 |
| 93 | 6Hex | 990.34 | 33.875 | 0.94 | 19.01 |

TABLE W
CLX122DSF comprises the oligosaccharides shown in
this table (Hex = hexose, generally galactose
or glucose and Pent=Pentose generally Arabinose).

| | | | RT | Oligo | Retention |
|---|---|---|---|---|---|
| Compound | Identity | Mass | (min) | Wt. % | Factor |
| 1 | 2Hex1Pent | 474.18 | 3.738 | 1.46 | 1.133 |
| 2 | 2Hex1Pent | 474.18 | 4.894 | 0.53 | 1.483 |
| 3 | 2Hex1Pent | 474.17 | 7.865 | 0.43 | 2.383 |
| 4 | 2Hex1Pent | 474.17 | 8.657 | 0.82 | 2.623 |
| 5 | 2Hex1Pent | 474.18 | 11.39 | 1 | 3.452 |
| 6 | 2Hex1Pent | 474.17 | 14.152 | 0.38 | 4.288 |
| 7 | 3Hex | 504.18 | 3.3 | 2.62 | 1 |
| 8 | 3Hex | 504.19 | 3.711 | 0.48 | 1.125 |
| 9 | 3Hex | 504.19 | 4.89 | 1.58 | 1.482 |
| 10 | 3Hex | 504.19 | 7.959 | 0.83 | 2.412 |
| 11 | 3Hex | 504.18 | 15.53 | 2.25 | 4.706 |
| 12 | 3Hex | 504.19 | 20.938 | 0.64 | 6.345 |
| 13 | 3Hex | 504.19 | 22.266 | 1.23 | 6.747 |
| 14 | 3Hex | 504.19 | 25.228 | 0.62 | 7.645 |
| 15 | 3Hex | 504.19 | 26.138 | 0.82 | 7.921 |
| 16 | 3Pent | 414.16 | 4.045 | 5.2 | 1.226 |
| 17 | 3Pent | 414.15 | 5.625 | 3.51 | 1.705 |
| 18 | 3Pent | 414.15 | 6.668 | 10.74 | 2.021 |
| 19 | 3Pent | 414.15 | 7.368 | 2.49 | 2.233 |
| 20 | 3Pent | 414.15 | 10.065 | 0.49 | 3.05 |
| 21 | 3Pent | 414.15 | 10.977 | 4.44 | 3.326 |
| 22 | 3Pent | 414.15 | 11.713 | 4.51 | 3.549 |
| 23 | 3Pent | 414.15 | 12.482 | 2.1 | 3.782 |
| 24 | 3Pent | 414.15 | 12.785 | 1.97 | 3.874 |
| 25 | 3Pent | 414.15 | 13.3 | 2.24 | 4.03 |
| 26 | 3Pent | 414.15 | 14.031 | 1.95 | 4.252 |
| 27 | 3Pent | 414.15 | 16.631 | 1.15 | 5.04 |
| 28 | 4Hex | 666.24 | 7.958 | 1.86 | 2.412 |
| 29 | 4Hex | 666.24 | 10.667 | 1.69 | 3.232 |
| 30 | 4Hex | 666.24 | 25.519 | 1.97 | 7.733 |
| 31 | 4Hex1Pent | 798.28 | 11.491 | 0.58 | 3.482 |
| 32 | 4Pent | 546.2 | 4.044 | 0.67 | 1.225 |
| 33 | 4Pent | 546.2 | 6.632 | 1.38 | 2.01 |
| 34 | 4Pent | 546.2 | 10.927 | 0.55 | 3.311 |
| 35 | 4Pent | 546.2 | 10.982 | 3.69 | 3.328 |
| 36 | 4Pent | 546.2 | 11.359 | 1.02 | 3.442 |
| 37 | 4Pent | 546.2 | 11.712 | 3.62 | 3.549 |
| 38 | 4Pent | 546.2 | 12.493 | 2.06 | 3.786 |
| 39 | 4Pent | 546.2 | 12.778 | 1.86 | 3.872 |
| 40 | 4Pent | 546.2 | 13.301 | 2.3 | 4.031 |
| 41 | 4Pent | 546.2 | 14.03 | 2.07 | 4.252 |
| 42 | 4Pent | 546.2 | 16.028 | 3.61 | 4.857 |
| 43 | 4Pent | 546.2 | 16.64 | 1.37 | 5.042 |
| 44 | 5Hex | 828.29 | 11.06 | 0.88 | 3.352 |
| 45 | 5Pent | 678.24 | 15.339 | 0.92 | 4.648 |
| 46 | 5Pent | 678.24 | 15.787 | 1.36 | 4.784 |
| 47 | 5Pent | 678.24 | 16.082 | 2.71 | 4.873 |
| 48 | 5Pent | 678.24 | 16.642 | 1.43 | 5.043 |
| 49 | 5Pent | 678.24 | 17.207 | 0.69 | 5.214 |
| 50 | 5Pent | 678.24 | 17.957 | 1.83 | 5.442 |
| 51 | 5Pent | 678.24 | 20.845 | 0.48 | 6.317 |
| 52 | 6Hex | 990.34 | 15.36 | 0.57 | 4.655 |
| 53 | 6Pent | 810.28 | 17.546 | 0.42 | 5.317 |
| 54 | 6Pent | 810.28 | 19.118 | 0.93 | 5.793 |
| 55 | 6Pent | 810.28 | 19.47 | 0.99 | 5.9 |

Table X. Table X1 provides glyosidic linkage composition analysis of oligosaccharide compositions CLX115, CLX115Cu, CLX112, and CLX122. Table X2 provides glyosidic linkage composition analysis of oligosaccharide compositions CLX123, CLX125, CLX126, and CLX128. Data are presented in units of peak area %. “Other” refers to linkages making up less than 2%. The notation “--” represents a linkage that exists in an amount less than 2% (which can be 0%) of the total oligosaccharide weight. If linkage is not fully described it will be denoted by the monosaccharide, when known, or the type of monosaccharide, either pentose or hexose, followed by multiple or a single “x” denoting the number of branch points and finally the retention time, in parentheses, in the units of minutes. “T” refers to terminal units. “P” refers to terminal units in the pyranose form. “f” refers to terminal units in the furanose form.

TABLE X1

| CLX No. | 3-Glc | 4-Glc | 6-Glc | 4,6-Glc | T-Glc | 4-Gal | T-Gal-P | T-Xyl | 4-Xyl | 2,3,4-Xyl |
|---|---|---|---|---|---|---|---|---|---|---|
| CLX115 | 23 | 64 | — | — | 13 | — | — | — | — | — |
| CLX115Cu | — | 64.41 | — | — | 16.42 | 9.27 | — | — | — | — |
| CLX112 | 17 | 49 | — | — | 31 | — | — | — | — | — |
| CLX122 | — | 3.4 | — | — | — | 4.54 | 2.89 | — | — | — |
| | | | | | | | x-Hex | 2-Pent | X-Pent | |
| CLX No. | 5-Arab | 3,5-Arab | 2,5-Arab | 2,3,5-Arab | T-Arab-f | 2-Rham | (2.108 min) | (2.769 min) | (5.289 min) | Other |
| CLX115 | — | — | — | — | — | — | — | — | — | — |
| CLX115Cu | — | — | — | — | — | — | — | — | — | 5.96 |
| CLX112 | — | — | — | — | — | — | — | — | — | — |
| CLX122 | 23.16 | 4.2 | 9.25 | 4.38 | 34.99 | — | — | — | — | 0.45 |

TABLE X2

| CLX No. | 3-Glc | 4-Glc | T-Glc | T-Man | 4-Gal | 6-Gal | 3,6-Gal | T-Gal-p |
|---|---|---|---|---|---|---|---|---|
| CLX126 | — | 3.28 | — | — | 41.54 | — | — | 5.28 |
| CLX123 | 3.28 | 29.88 | 11.54 | 5.53 | — | — | 12.24 | — |
| CLX128 | 3.44 | 15.54 | 8.94 | — | — | — | — | — |
| CLX125 | 20.96 | 24.24 | 10.25 | — | — | — | — | — |
| CLX No. | 2-Man | 4,6-Man | T-Xyl | 4-Xyl | 2,3,4-Xyl | 3,4-Xyl | 5-Arab | 3,5-Arab |
| CLX126 | — | — | — | — | — | — | 11.11 | 4.81 |
| CLX123 | 16.02 | 6.09 | — | — | — | — | — | — |
| CLX128 | — | — | 5.97 | 27.17 | — | 4.81 | 7 | — |
| CLX125 | — | — | — | — | — | — | — | — |
| | | | | | | | 2,x,x-Rham | |
| CLX No. | 2,5-Arab | 2,3,5-Arab | T-Arab-f | 2-Fuc | 4-Rham | T-Rham | (1.626 min) | 4-GlcA |
| CLX126 | — | 2.2 | 13.98 | — | — | — | — | — |
| CLX123 | — | — | — | — | — | — | — | 4.12 |
| CLX128 | — | — | 8.31 | 5.97 | — | — | — | — |
| CLX125 | — | — | — | — | 21.57 | 7.33 | — | 4.91 |

Any viscosity measurement or property reported herein employs water as the solvent, unless specified otherwise.

In some aspects, a composition or compound of the invention, such as an oligosaccharide or composition comprising an oligosaccharide, is isolated or substantially purified. In an embodiment, an isolated or purified oligosaccharide or oligosaccharide composition is at least partially isolated or substantially purified as would be understood in the art. In an embodiment, a substantially purified composition, oligosaccharide or formulation of the invention has a chemical purity of 95%, optionally for some applications 99%, optionally for some applications 99.9%, optionally for some applications 99.99%, and optionally for some applications 99.999% pure.

When disclosing numerical values herein, for example, 1, 2, 3, 4, 5, 6, 7, 8, 9, 10, the following sentence typically follows such numerical values: “Each of the foregoing numbers can be preceded by the term ‘about,’ ‘at least,’ ‘at least about,’ ‘less than,’ or ‘less than about,’ and any of the foregoing numbers can be used singly to describe a single point or an open-ended range, or can be used in combination to describe multiple single points or a close-ended range.” This sentence means that each of the aforementioned numbers can be used alone (e.g., 4), can be prefaced with the word “about” (e.g., about 8), prefaced with the phrase “at least about” (e.g., at least about 2), prefaced with the phrase “at least” (e.g., at least 10), prefaced with the phrase “less than” (e.g., less than 1), prefaced with the phrase “less than about” (e.g., less than about 7), or used in any combination with or without any of the prefatory words or phrases to define a range (e.g., 2 to 9, about 1 to 4, at least 3, 8 to about 9, 8 to less than 10, about 1 to about 10, and so on). Moreover, when a range is described as “about X or less,” this phrase is the same as a range that is a combination of “about X” and “less than about X” in the alternative. For example, “about 10 or less” is the same as “about 10, or less than about 10.” Such interchangeable range descriptions are contemplated herein. In addition, the phrases “at least about” and “about X or more” have the same meaning and can be used interchangeably. Other range formats may be disclosed herein, but the difference in formats should not be construed to imply that there is a difference in substance.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

It has now been surprisingly found that the urogenital health of a female subject may be improved by administration of one or more oligosaccharides which are preferentially metabolized by beneficial vaginal Lactobacillus species and, in some aspects, not preferentially metabolized by vaginal dysbiosis-associated bacteria. It is believed that the one or more oligosaccharides improve urogenital health by restoring at least partially the composition or functioning of beneficial Lactobacillus species in the urogenital tracts. As an outcome in some aspects, vaginal pH decreases, opportunistic bacteria are reduced, or a combination thereof.

Disclosed herein are novel therapeutic strategies for the treatment or prevention of diseases or conditions associated with the female subject urogenital tract, including bacterial vaginosis and vulvovaginal candidiasis. In some aspects, disclosed is a therapeutic method comprising contacting one or more microbes (e.g., bacteria, fungi, yeast) with a composition comprising one or a mixture of CLX096, CLX097, CLX098, CLX099, CLX101, CLX102, CLX103, CLX109, CLX110, CLX111, CLX112, CLX115, CLX115Cu, CLX122, CLX122DSF, CLX123, CLX125, CLX126, or CLX128 (i) to selectively stimulate growth of one or more microbes, (ii), that are not metabolized by one or more microbes, and/or (iii) to increase the production of beneficial metabolites such as lactic acid, which lowers the pH in an area of interest, such as a vagina, to inhibit growth of undesired microbes. In some aspects, the microbe stimulated by the oligosaccharide(s) described herein is a probiotic microbe generally located in a healthy vagina, comprising at least one of L. crispatus, L. jensenii, L. gasseri, L. iners, or any combination thereof and/or the probiotic microbe comprising Lactobacillus reuteri (e.g., L. reuteri RC-14), Lactobacillus rhamnosus (e.g., L. rhamnosus GR-1), Lactobacillus fermentum (e.g., L. fermentum LF15) Lactobacillus crispatus (e.g., L. crispatus LV88 and/or CTV-05), Lactobacillus gasseri, Lactobacillus acidophilus, Lactobacillus plantarum, or any other probiotic disclosed herein, or a combination thereof. In some aspects, one or more of CLX096, CLX097, CLX098, CLX099, CLX101, CLX102, CLX103, CLX109, CLX110, CLX111, CLX112, CLX115, CLX115Cu, CLX122, CLX122DSF, CLX123, CLX125, CLX126, or CLX128 is combined with other ingredients to produce foodstuffs and supplements including infant formula, geriatric supplements, drinks, nutritional supplements, baking flours, and snack foods. In some aspects, one or more of these oligosaccharides is also used as a pharmaceutical product.

In some aspects, administration of a pharmaceutically acceptable composition of one or more of CLX096, CLX097, CLX098, CLX099, CLX101, CLX102, CLX103, CLX109, CLX110, CLX111, CLX112, CLX115, CLX115Cu, CLX122, CLX122DSF, CLX123, CLX125, CLX126, or CLX128 is employed to stimulate the growth of Lactobacillus species in the vagina, which in some aspects, treats or prevents diseases or conditions of the vagina, including bacterial vaginosis and vulvovaginal candidiasis. In some aspects, administration of a pharmaceutically acceptable composition of one or more of CLX096, CLX097, CLX098, CLX099, CLX101, CLX102, CLX103, CLX109, CLX110, CLX111, CLX112, CLX115, CLX115Cu, CLX122, CLX122DSF, CLX123, CLX125, CLX126, or CLX128 is employed to increase the production of lactic acid, hydrogen peroxide, or a combination thereof. Pharmaceutically acceptable salts, stereoisomers, and metabolites of one or more of the oligosaccharides described herein also are contemplated.

Administration of and administering a compound or composition should be understood to mean providing a compound or salt thereof or a pharmaceutical composition comprising a compound, in which the compound is an oligosaccharide or an oligosaccharide composition, optionally including additional active ingredients, such as antibiotics, probiotics, and/or prebiotics. In some aspects, the compound or composition is administered by another person to the subject (e.g., orally, intravenously, and/or topically) or, in some aspects, it is self-administered by the subject (e.g., orally, such as via tablets or capsules, and/or topically via a cream, ointment, or gel). The term “subject” refers to mammals (for example, humans and veterinary animals such as dogs, cats, pigs, horses, sheep, and cattle). Administration of one or more of CLX096, CLX097, CLX098, CLX099, CLX101, CLX102, CLX103, CLX109, CLX110, CLX111, CLX112, CLX115, CLX115Cu, CLX122, CLX122DSF, CLX123, CLX125, CLX126, or CLX128 in combination with other compounds, such as excipients, colorants, fillers, binders, diluents, buffering agents, moistening agents, preservatives, flavoring agents, dyes, disintegrating agents, pharmaceutically compatible carriers, or any combination thereof, is contemplated.

In some aspects, disclosed is a composition for improving urogenital health in a female subject, the composition comprising a therapeutically effective amount of one or more oligosaccharides preferentially metabolized by beneficial vaginal Lactobacillus species but not preferentially metabolized by vaginal dysbiosis-associated bacteria.

In some aspects, the one or more oligosaccharides are preferentially metabolized by L. crispatus, L. gasseri, L. jensenii, L. vaginalis, L. iners, or any combination thereof. In some aspects, the one or more oligosaccharides is not preferentially metabolized by L. iners, Gardenerella vaginalis, Atopobium vaginae, Prevotella species, Mobiluncus species, or any combination thereof. In some aspects, the one or more oligosaccharides include glycosidic bonds which can be depolymerized via bacteria glycosyl hydrolases in the vagina, for example, bacteria glycosyl hydrolases produced by L. crispatus, L. gasseri, L. jensenii, or any combination thereof. In some aspects, although Lactobacillus iners is sometimes observed in women who have, or are recovering from, bacterial vaginosis, it is hypothesized that Lactobacillus iners may nevertheless play a role in reestablishing a healthy vaginal flora. Accordingly, in some aspects, the one or more oligosaccharides are preferentially metabolized by Lactobacillus iners, and in other aspects, the one or more oligosaccharides are not preferentially metabolized by Lactobacillus iners.

In some aspects, provided is a pack for improving urogenital health in a female subject, the pack comprising at least 4, e.g., at least 7, at least 10, at least 12, at least 14, at least 18, at least 21, at least 24, or at least 28 individual daily doses of an effective amount of one or more oligosaccharides preferentially metabolized by beneficial vaginal Lactobacillus species but not preferentially metabolized by vaginal dysbiosis-associated bacteria.

In some aspects, provided is a method for improving urogenital health in a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for reducing the risk of bacterial vaginosis or recurrence of bacterial vaginosis in a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for reducing the severity of bacterial vaginosis in a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for treating bacterial vaginosis in a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for reducing the risk of urinary tract infection or recurrence of urinary tract infection in a female subject having bacterial vaginosis or having had bacterial vaginosis, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for reducing the risk of a vaginal fungal infection or recurrence of a vaginal fungal infection in a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for reducing the severity of a vaginal fungal infection in a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for treating a vaginal fungal infection in a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, provided is a method for lowering the pH of the urogenital tract of a female subject, the method comprising administering to the female subject a therapeutically effective amount of one or more oligosaccharides preferentially metabolized by beneficial vaginal Lactobacillus species but not preferentially metabolized by vaginal dysbiosis-associated bacteria.

In some aspects, the oligosaccharides and oligosaccharide compositions useful e.g., for the purposes and methods described herein, are generally those CLX compositions defined elsewhere herein (or combinations thereof), which in some aspects are derived from the polysaccharides (or sources thereof) described elsewhere herein, as well as those oligosaccharides that have the same (or substantially the same, e.g., values within 10%, or within 15%, or within 20%, or within 25%, or within 30% of the values for) hydrolytic monosaccharide composition, glycosidic linkage composition, oligosaccharide analysis, and 1H-13C HSQC NMR analysis (if such analyses are provided herein) as CLX096, CLX097, CLX098, CLX099, CLX101, CLX102, CLX103, CLX109, CLX110, CLX111, CLX112, CLX115, CLX115Cu, CLX122, CLX122DSF, CLX123, CLX125, CLX126, or CLX128 or any combination thereof.

In some aspects, sources of the oligosaccharides described herein include, for example, starting materials comprising a polysaccharide containing at least one of the following glycosidic bonds: galactose-mannose (α1-6), xylose-xylose (β1-4), arabinose-xylose (α1-2/3), xylose-glucose (α1-6), galactose-xylose (β1-2), glucose-glucose (β1-4), or any combination thereof. In some aspects, these materials comprise polysaccharides comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, gellan gum, xanthan gum, or any combination thereof. Sources of galactomannan include seed mucilage, such as guar, tara, cassia, carob, fenugreek, or any combination thereof. In some aspects, galactomannan is derived from the cell walls of molds such as Aspergillus, optionally combined with one or more seed mucilage sources above. Sources of β-glucan include cereals such as barley, wheat, rye, oat and/or non-cereal sources such as lichenan, or any combination thereof. Sources of homo-xylan, glucuronoxylan, and glucuronoarabinoxylan include soft and hard woods such as beech wood; fruit and vegetable seeds, such as the cell walls of seeds such as apple, pear, pepper, tomato, peach, and cherry; Poaceae; corn cob; sugar cane; bamboo; cereal brans; switchgrass; grasses; or any combination thereof. Sources of arabinoxylan include cereal grains and bran such as oat, wheat, corn, rice, rye, and flax. Sources of xyloglucan include the cell walls of vascular plants such as tamarind, cranberries, and brassica. Sources of arabinan includes sugar beet, spent sugar beet, legumes (e.g., soy, and/or tree nuts), or any combination thereof. Sources of arabinogalactan include both bacterial (arabinogalactan type I) and plant sources (arabinogalactan type II). The plant sources of arabinogalactan include larch, acacia, and Angeissus latifolia. Bacterial sources of arabinogalactan are actinobacteria, preferably mycobacterium. Bacterial sources of gellan gum include Sphingomonas elodea. Bacterial sources of xanthan gum include Xanthomonas campestris. Additional sources of oligos include other natural products like plants (or parts thereof, such as flesh, skin, seed, etc.) from the Cucurbita species (e.g., squash (e.g., butternut, acorn, winter, summer, zucchini), gourd (e.g., fingerleaf, fig leaf, buffalo, stinking), pumpkin (e.g., cushaw, winter, “Dickinson,” field), etc.), moringa (e.g., Moringa oleifera or a part thereof, including the leaves, branches, trunks, bark, roots, pods, seeds, flowers, fruit, etc.), spent distillers' grains (e.g., cereal byproducts of the distillation process, such as corn, rice, or other grains, or any combination thereof), soy, pea, sugar cane, or any combination thereof. In some aspects, any combination of the above listed sources can be used to provide an oligosaccharide composition.

In some aspects, oligosaccharides or oligosaccharide compositions are derived from barley, carob, spent distillers' grain, lichenan, butternut squash (e.g., skin, flesh, etc.), moringa leaf, or any combination thereof. In some aspects, the oligosaccharides contain disaccharides, trisaccharides, tetrasaccharides, and/or pentasaccharides that are the same or similar to those found in barley, carob, spent distillers' grain, lichenan, butternut squash, or moringa leaf oligosaccharides and polysaccharides. In some aspects, the oligosaccharides and oligosaccharide compositions can contain glycosidic linkages that are the same or similar to those found in barley, carob, spent distillers' grain, lichenan, butternut squash or moringa leaf oligosaccharides. In some aspects, the oligosaccharides are or resemble those described in Tables A-W.

Beneficial vaginal microbes produce glycoside hydrolases that, without wishing to be bound by theory, are believed to hydrolyze polysaccharides and/or oligosaccharides depending on the types of linkages present in such saccharides. GH activity information is available in the CAZy database (http://www.cazy.org). By assessing which GHs are present in higher abundance in the beneficial vaginal microbes as opposed to the pathogenic microbes, the types of carbohydrate linkages that may be hydrolyzed preferentially by certain microbes may be rationalized. Beneficial vaginal microbes produce the glycosyl hydrolase families: GH 36 (preferably with EC 3.2.1.22 activity), GH8 (preferably with EC 3.2.1.4 activity), GH 10 (preferably with EC 3.2.1.8 activity), GH 31 (preferably with EC 3.2.1. 117 activity), GH 51 (preferably with EC 3.2.1.55 activity), and GH 2 (preferably with EC 3.2.1. 23 activity).

In some aspects, suitable oligosaccharide or oligosaccharide compositions are those that increase the abundance of Lactobacillus species compared to the abundance of vaginal dysbiosis-associated bacterial, as measured by methods disclosed herein, including culturing such microbes on the oligosaccharides or oligosaccharide compositions of interest and measuring optical density, a marker of growth, or both.

In some aspects, the oligosaccharides and oligosaccharide compositions disclosed herein are produced by any suitable method.

In some aspects, the oligosaccharides and oligosaccharide compositions are produced by depolymerizing a suitable polysaccharide using a chemical method, such as oxidative chemistry.

In some aspects, the oligosaccharides and oligosaccharide compositions disclosed herein are produced from polysaccharides by high-yield peroxide-quench-controlled methods (Controlled Oligosaccharide Generation (“COG”) methods), as disclosed in WO 2021/097138, hereby incorporated by reference in its entirety for all purposes to the extent not inconsistent with the description herein, and more specifically for the generation of oligosaccharides from polysaccharides by COG methods. Such methods comprise a multi-step reaction (e.g., two-step, three-step, etc., reaction) that includes an initial oxidative step using a Fenton's system/reagent and a subsequent peroxide-quenching/PS-cleavage step using either: a PS-cleavage agent that also functions as a peroxide-quenching agent; or using a PS-cleavage agent in combination with a compatible peroxide-quenching reagent that does not interfere with the PS-cleavage reaction. In the methods, the PS-cleavage agent may be, for example, a weak-Arrhenius base or non-Arrhenius base. In the methods, the PS-cleavage initiator preferably also functions as a peroxide-quencher to quench (e.g., sufficiently reduce or eliminate) residual hydrogen peroxide and/or radicals thereof to minimize or eliminate off-target side reactions. The methods, for example, comprise reacting polysaccharides with hydrogen peroxide and a suitable metal or metal ion (e.g., Fe(II), Fe(III), Cu(I), Cu(II), Ca(II), Mg(II), Mn(II), Zn(II), Ni(II), Ce(IV), Co(II) or other metal ions), followed by cleaving glycosidic linkages in the hydroperoxyl-treated polysaccharides with a high-yield peroxide-quenching/cleavage agent such as ammonium bicarbonate, ammonium hydroxide, ammonia, urea, sodium amide, dimethyl amine, trimethylamine, pyridine, N,N-diisopropylethylamine, other ammonium-based reagents, a weak Arrhenius base, a non-Arrhenius base, a Lewis base, a Bronsted-Lowry base, or any combination thereof, thereby generating high yields of oligosaccharides, and lower molecular weight polysaccharides (polysaccharide cleavage products that are yet polysaccharides) from the parent (starting material) polysaccharides, while reducing or eliminating peeling (sequential alkaline degradation of carbohydrates through a mechanism that releases monomeric units from the reducing end of the polymer) and unwanted side-reactions.

In some aspects, the oligosaccharides and oligosaccharide compositions disclosed herein are produced from polysaccharides by a method known as Fenton's Initiation Toward Defined Oligosaccharide Groups (FITDOG), as disclosed in WO 2018/236917, hereby incorporated by reference herein in its entirety for all purposes, and more specifically for methodologies of production, to the extent not inconsistent with the description herein. Such methods comprise reacting a polysaccharide using Fenton's reagent composed of iron (Fe⁺, Fe²⁺) or other transition metal (including but not limited to, Cu¹⁺, Co²⁺, etc., including those disclosed herein for the COG method) and hydrogen peroxide. In some aspects, the reaction is allowed to proceed for 30 minutes (or for example, between 10 minutes and 4 hours, e.g., 15 minutes to 2 hours or 10 minutes to one hour). In some aspects, the transition metal or alkaline earth metal in the reaction mixture is at a concentration of at least 0.65 mM, or 0.65 mM to 20 mM, or 10 μM to 20 mM. The reaction is subsequently quenched with base (e.g., an Arrhenius base or strong Arrhenius base, such as aqueous sodium hydroxide calcium hydroxide, potassium hydroxide, etc., or any combination thereof). In some aspects, prior to the reacting, the method comprises contacting polysaccharides with one or more polysaccharide degrading enzyme, such as an amylase, isoamylase, cellulase, maltase, glucanase, or a combination thereof.

In some aspects, the oligosaccharides and oligosaccharide compositions are produced by a method comprising ground-up synthesis (e.g., biological synthesis or resin polymerization) by polymerizing monosaccharides and/or lower molecular weight oligosaccharides.

In some aspects, the oligosaccharides and oligosaccharide compositions are created by elevated time, temperature, pressure processes.

In some aspects, the oligosaccharides and oligosaccharide compositions are created by either depolymerization, polymerization, or transglycosylation by the use of enzymes. In some aspects, the one or more polysaccharide degrading enzyme(s) comprises, for example, an amylase, isoamylase, cellulase, maltase, glucanase, xylanase, lactase, or a combination thereof.

In some aspects, the oligosaccharides and oligosaccharide compositions are created by chemical synthesis. In some aspects, the oligosaccharides can be synthesized in microorganisms such as yeast, algae, and bacteria. In some aspects, the oligosaccharides can be synthesized in eukaryotic cells.

In some aspects, the oligosaccharide composition are created by depolymerization or polymerization by negative and/or positive solid state or soluble catalysts.

In some aspects, the oligosaccharide composition are created from depolymerized from different natural products (e.g., polysaccharides found in nature). In some aspects, the natural product that the oligosaccharide composition was produced from does not matter, so long as the carbohydrate structure is similar. In some aspects, whether a carbohydrate composition was produced via depolymerization, polymerization, or transglycosylation does not matter, so long as the carbohydrate structure is similar.

In some aspects, the oligosaccharides in an oligosaccharide composition have the same or different ranges of DP. In some aspects, certain DPs have enhanced functions over other DPs. In some aspects, the mean DP is 2, 3, 4, 5, 6, 7, 8, 9, 10, 11, 12, 13, 14, 15, 16, 17, 18, 19, 20, 21, 22, 23, 24, 25, 26, 27, 28, 29, or 30. Each of the foregoing numbers can be preceded by the word “about,” “at least,” “at least about,” “less than,” or “less than about,” and any of the foregoing numbers can be used singly to describe a single point or an open-ended range, or can be used in combination to describe multiple single points or a close-ended range. For example, in some aspects, the mean DP is 2 to 6, 3 to 6, at least 2, 2 to 30, 3 to 30, less than 10, 2 to about 14, and the like.

In some aspects, each oligosaccharide with a particular DP or DP range is present in an oligosaccharide composition at any suitable amount, based on the total weight of the composition. For example, in some aspects an oligosaccharide with a particular mean DP or mean DP range is present in an amount (wt. %) of 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 96, 97, 98, 99, or 100, based on the total weight of the oligosaccharide composition. For example, in some aspects, an oligosaccharide with a particular mean DP or mean DP range is present in an amount (wt. %) of 3 to 10, 5 to 14, less than 26, and the like, based on the total weight of the composition. In some aspects, these weight percents apply to any of the mean DPs or mean DP ranges disclosed elsewhere herein.

In some aspects, each oligosaccharide with a particular mean DP or mean DP range can be present in an oligosaccharide composition at any suitable amount, based on the total weight of oligosaccharides having a mean DP of 2 to 100. For example, in some aspects an oligosaccharide with a particular mean DP or mean DP range can be present in an amount (wt. %) of 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 96, 97, 98, 99, or 100, based on the total weight of oligosaccharides having a DP of 2 to 100. For example, in some aspects, an oligosaccharide with a particular mean DP or mean DP range is present in an amount (wt. %) of 20 to 50, 64 to 80, less than 30, at least 24, and the like, based on the total weight of the oligosaccharides having a mean DP of 2 to 100. In some aspects, these weight percents apply to any of the mean DPs or mean DP ranges disclosed elsewhere herein.

In some aspects, the oligosaccharide refers to a single oligosaccharide, several oligosaccharides (e.g., an oligosaccharide composition), or a pool of oligosaccharides (e.g., also an oligosaccharide composition) obtained by depolymerizing a polysaccharide. In some aspects, the oligosaccharides is formulated with their parent polysaccharides (e.g., CLX112, which is generally derived from beta-glucan barley, can further comprise beta-glucan from barley). The mean degree of polymerization (DP) of the composition is preferably 3 to 100, for example 3 to about 30, or about to 10 to about 100, which spans the definition of oligosaccharides (mean DP of 2 to 30) and polysaccharides (mean DP of over 30 up to about 500,000). In some aspects, the composition preferably has a mean degree of polymerization of 3 to 30; for example, 3 to about 20. In some aspects, the oligosaccharide or oligosaccharide composition is formulated in a mixture with monosaccharides, disaccharides, trisaccharides, tetrasaccharides, pentasaccharides, and so forth, and/or polysaccharides (for example, polysaccharides of chain length larger than 30). In some aspects, the oligosaccharides are mixed with oligosaccharides of mean DP >20 and/or polysaccharides of mean DP >30 to increase stability and or efficacy. In some aspects, oligosaccharides of mean degree of polymerization of 3 to 30 comprise at least 50% by mass of the total mixture, more preferably at least about 60% by mass, for example at least about 70% by mass, in each case the mass expressed relative to the total mass of saccharides in the composition. In some aspects, monosaccharides and/or disaccharides comprise less than about 20% of the mixture by mass, preferably less than about 10% by mass.

In some aspects, the oligosaccharides comprise 2, 3, 4, 5, or 6 hexose residues. In some aspects, the oligosaccharides contain 1, 2, 3, or more pentose residues. In some aspects, the oligosaccharides contain an equal number of hexose and pentose residues. In some aspects the oligosaccharides contain fewer pentose residues than hexose residues.

In some aspects, disclosed are mixtures containing two or more different synthetic oligosaccharides as described herein. In some aspects, unpurified or semi-purified depolymerization products are used for preparation of oligosaccharide mixtures or, alternatively, oligosaccharides are purified to produce specially formulated pools. In some aspects, the synthetic oligosaccharides in the mixtures are obtained, for example, by depolymerizing one or more polysaccharides. In some aspects, the at least one synthetic oligosaccharide is present in a mixture at any suitable amount, or is present relative to all other oligosaccharides at any suitable amount. For example, in some aspects, the amount of at least one of the synthetic oligosaccharides in the mixture is at least 1%. In some aspects, the synthetic oligosaccharide is present, for example, in an amount ranging from about 1% to about 99%, or from about 5% to about 95%, or from about 10% to about 90%, or from about 20% to about 80%, or from about 30% to about 70%. The synthetic oligosaccharide may be present, for example, in an amount ranging from about 1% to about 10%, or from about 10% to about 20%, or from about 20% to about 30%, or from about 30% to about 40%, or from about 40% to about 50%, or from about 50% to about 60%, or from about 60% to about 70%, or from about 70% to about 80%, or from about 80% to about 90%, or from about 90% to about 99%. In some aspects, the percentage refers to a mol % (i.e., based on the total number of moles of oligosaccharides in the mixture), or a weight % (i.e., based on the total weight of oligosaccharides in the mixture or based on the total weight of the mixture including all excipients, solvents, etc.). In some aspects, the amount of at least one of synthetic oligosaccharides is at least 5 mol %.

In some aspects, the oligosaccharide compositions as described herein have different, enhanced, and unexpected properties when compared to its parent polysaccharide.

In some aspects, the oligosaccharide compositions disclosed herein are in the form of an enterally administered composition, a topically administered composition, an intra-vaginally administered composition (each of which can be a pharmaceutical composition), an intra-vaginal device, or absorbent article (e.g., disposable absorbent article) such as a diaper, a pant, an undergarment, an adult incontinence product, an absorbent insert for a diaper or undergarment (or pant), a wipe, or a feminine hygiene product, such as a sanitary napkin, a tampon, or a panty liner. During prevention or treatment, a combination of any such compositions, articles, or devices may be employed.

In some aspects, the oligosaccharides or oligosaccharide compositions or formulations (e.g., pharmaceutical compositions, nutritional composition, food, etc.) are administered to any subject/individual in need thereof. In some aspects, the individual is an infant or toddler. For example, in some aspects, the individual is less than, e.g., 3 months, 6 months, 9 months, one year, two years or three years old. In some aspects, the individual is between 3-18 years old. In some aspects, the individual is an adult (e.g., 18 years or older). In some aspects, the individual is over 50, 55, 60, 65, 70, or 75 years old. In some aspects, the individual is between three years old and 18 years old. In some aspects, the subject is a female subject. In some aspects, the subject is a male subject (e.g., assigned male at birth but has been reassigned as female with surgery and/or hormone treatment).

In some aspects, the oligosaccharides is formulated into products for oral hygiene. Without wishing to be bound by theory, it is believed that oral sex with a female subject as the receiving partner can increase the risk of an infection, such as bacterial vaginosis, in the female subject. Accordingly, prior to providing oral sex to a female subject partner, a decrease in risk of an infection may result if the giving partner uses an oral hygiene product comprising the oligosaccharides or oligosaccharides disclosed herein, which can reduce the incidence of harmful bacteria in the giving partner's mouth, and also provide beneficial oligosaccharides directly to the vagina of the receiving partner. Additionally, an amount of the oligosaccharides or oligosaccharide composition may benefit the individual brushing her teeth, since the oral hygiene product may be absorbed into the bloodstream either through the oral membranes or blood vessels, or via ingestion. In some aspects oral hygiene products are tooth paste, mouth wash, chewing gum, mints, candies, lozenges, and floss. In some aspects, the oligosaccharides are formulated at approximately 10 mg/application. In some aspects, the oligosaccharides are formulated at approximately 100 mg/application. In some aspects, the oligosaccharides are formulated at approximately 200 mg or more/application.

In some aspects, oligosaccharide compositions as described herein are used to supplement a beverage. Examples of such beverages include, without limitation, infant formula, follow-on formula, toddler's beverage, milk, fermented milk, fruit juice, fruit-based drinks, and sports drinks. Many infant and toddler formulas are known in the art and are commercially available, including, for example, Carnation Good Start™ (Nestle Nutrition Division; Glendale, Calif.) and Nutrish AB™ produced by Mayfield Dairy Farms (Athens, Tenn.). Other examples of infant or baby formula include those disclosed in U.S. Pat. No. 5,902,617, hereby incorporated by reference in its entirety for all purposes, and more specifically for examples of formula, to the extent not inconsistent with the description herein. Other beneficial formulations of the compositions include the supplementation of animal milks, such as cow's milk.

In some aspects, the oligosaccharide or oligosaccharide compositions described herein are used to generate a prebiotic for food supplementation. In some aspects, the oligosaccharide or oligosaccharide composition are used to modulate appetite control and/or control of energy (caloric) intake in subject in need thereof (e.g., children, or other subjects, with excess weight and obesity).

In some aspects, the oligosaccharide or oligosaccharide compositions are administered as a prebiotic formulation (i.e., without bacteria) or as a probiotic formulation (i.e., with one or more desirable bacteria such as Bifidobacteria or Lactobacillus as described elsewhere herein). In general, any food or beverage that can be consumed by humans or animals, or otherwise suitably administered or topically applied, may be used to make formulations containing the prebiotic and probiotic oligosaccharide containing compositions. Exemplary foods include those with a semi-liquid consistency to allow easy and uniform dispersal of the prebiotic and probiotic compositions described herein. However, other consistencies (e.g., powders, liquids, etc.) can also be used without limitation. Accordingly, such food items include, without limitation, dairy-based products such as cheese, cottage cheese, yogurt, and ice cream. Processed fruits and vegetables, including those targeted for infants/toddlers, such as apple sauce or strained peas and carrots, are also suitable for use in combination with the oligosaccharides of the present invention. Both infant cereals such as rice- or oat-based cereals and adult cereals such as Cream of Wheat™, etc., are also suitable for use in combination with the oligosaccharides. In some aspects, the oligosaccharide or oligosaccharide composition is used in medical foods, for example, such as Pedialyte™, Ensure™, etc. In addition to foods targeted for human consumption, in some aspects, animal feeds are supplemented with the prebiotic and probiotic oligosaccharide containing compositions.

In some aspects, the oligosaccharides or oligosaccharide compositions disclosed herein are formulated as a pharmaceutical composition. In some aspects, the pharmaceutical composition contains a pharmaceutically acceptable carrier, e.g. phosphate buffered saline solution, mixtures of ethanol in water, water and emulsions such as an oil/water or water/oil emulsion, as well as various wetting agents or excipients. In some aspects, the pharmaceutical composition also contains excipient and/or carriers such as solvents, dispersants, coatings, absorption promoting agents, controlled release agents, and inert excipients, such as starches, granulating agents, microcrystalline cellulose, diluents, lubricants, binders, and disintegrating agents. In some aspects, such pharmaceutical compositions are prepared by standard pharmaceutical formulation techniques such as those disclosed in Remington's Pharmaceutical Sciences, Mack Publishing Co., Easton, Pa. (19th Edition).

In some aspects, the oligosaccharides or oligosaccharide compositions as described herein are formulated into pills or tablets or encapsulated in capsules, such as gelatin capsules. In some aspects, tablet forms optionally include, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. In some aspects, lozenge or candy forms comprise the compositions in a flavor, e.g., sucrose, as well as pastilles comprising the compositions in an inert base, such as gelatin and glycerin or sucrose and acacia emulsions, gels, and the like containing, in addition to the active ingredient, carriers known in the art. In some aspects, the prebiotic or probiotic oligosaccharide containing formulations also contain conventional food supplement fillers and extenders such as, for example, rice flour. In some aspects, the products are also used to help the absorption of other nutrients and minerals.

Pharmaceutically acceptable carriers are those carriers that are compatible with the other ingredients in the formulation and are biologically acceptable. In some aspects, carriers can be solid or liquid. It is currently contemplated that preferred carriers are liquid carriers. In some aspects, carriers include one or more substances that also act as solubilizers, suspending agents, fillers, glidants, compression aids, binders, tablet-disintegrating agents, or encapsulating materials. In some aspects, liquid carriers are used in preparing solutions, suspensions, emulsions, syrups and elixirs. In some aspects, the active ingredient is dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. In some aspects, the liquid carrier contains other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. In some aspects, compositions for oral administration are in either liquid or solid form.

Suitable examples of liquid carriers for oral and parenteral administration include water of appropriate purity, aqueous solutions (particularly containing additives, e.g. cellulose derivatives, sodium carboxymethyl cellulose solution), alcohols (including monohydric alcohols and polyhydric alcohols e.g. glycols) and their derivatives, and oils. Sterile liquid carriers are used in sterile liquid form compositions for parenteral administration. In some aspects, the liquid carrier for pressurized compositions is halogenated hydrocarbon or other pharmaceutically acceptable propellant. In some aspects, liquid pharmaceutical compositions that are sterile solutions or suspensions are administered by, for example, intramuscular, intraperitoneal or subcutaneous injection. In some aspects, sterile solutions are be administered intravenously. In some aspects, compositions for oral administration are in either liquid or solid form. In some aspects, the carrier is also in the form of creams and ointments, pastes, and gels. In some aspects, the creams and ointments are viscous liquid or semisolid emulsions of either the oil-in-water or water-in-oil type.

In some aspects, the pharmaceutical compositions are also formulated as rectal suppositories, vaginal suppositories, intra-vaginal devices, or topically applied formulations such as lotions, sprays, creams, gels, emulsions, etc.

In some aspects, the oligosaccharides or oligosaccharide compositions as described herein will comprise or further comprise a non-human protein, non-human lipid, non-human carbohydrate, or other non-human component. For example, in some aspects, the compositions comprise a bovine (or other non-human) milk protein, a soy protein, a pea protein, a rice protein, beta-lactoglobulin, whey, soybean oil or starch. In some aspects, the oligosaccharides are combined with polysaccharides. In some aspects, the oligosaccharides are combined with their parent polysaccharide.

In some aspects, oligosaccharide compositions as described herein are in the form of a nutritional composition. In some aspects, the nutritional composition comprises a food, a beverage, a rehydration solution, a medical food or food for special medical purposes, a nutritional supplement, and the like. In some aspects, the nutritional composition contains sources of protein, lipids and/or digestible carbohydrates and, in some aspects, is in solid, powdered, or liquid forms. In some aspects, the synthetic composition is designed to be the sole source of nutrition, or as a food or nutritional supplement which forms part of the diet.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. In some aspects, the protein comprises whole protein or hydrolyzed protein, either partially hydrolyzed or extensively hydrolyzed. Hydrolyzed protein offers the advantage of easier digestion which can be important for humans with inflamed or compromised GI tracts. In some aspects, the protein is also provided in the form of free amino acids. In some aspects, the protein comprises about 5% to about 30% of the energy of the nutritional composition, normally about 10% to 20%.

In some aspects, the protein source is a source of glutamine, threonine, cysteine, serine, proline, or a combination of these amino acids. In aspects, the glutamine source is a glutamine dipeptide and/or a glutamine enriched protein. In some aspects, glutamine is included due to the use of glutamine by enterocytes as an energy source. Threonine, serine, and proline are important amino acids for the production of mucin. Mucin coats the gastrointestinal tract and can improve intestinal barrier function and mucosal healing. Cysteine is a major precursor of glutathione, which is key for the antioxidant defenses of the body.

Suitable digestible carbohydrates include maltodextrin, hydrolyzed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, lactose, honey, sugar alcohols (e.g., maltitol, erythritol, sorbitol), or mixtures thereof. Preferably the composition is reduced in or free from added lactose or other FODMAP carbohydrates (i.e., fermentable oligosaccharides, disaccharides, monosaccharides, and polyols). Generally digestible carbohydrates provide about 35% to about 55% of the energy of the nutritional composition. A particularly suitable digestible carbohydrate is a low dextrose equivalent (DE) maltodextrin.

Suitable lipids include medium chain triglycerides (MCT) and long chain triglycerides (LCT). Preferably the lipid is a mixture of MCTs and LCTs. For example, in some aspects, MCTs comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. In some aspects, the lipids contain essential fatty acids (omega-3 and omega-6 fatty acids). Preferably these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1.

In some aspects, a nutritional composition, pharmaceutical composition, or absorbent article (described elsewhere herein) includes vitamins and/or minerals. In some aspects, particularly in the case of a nutritional composition intended to be a sole source of nutrition, the composition (e.g., nutritional, pharmaceutical, or for an absorbent article) includes a complete vitamin and mineral profile. Examples of vitamins include vitamins A, B-complex (such as B1, B2, B6 and B12), C, D, E and K, niacin, and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium, and boron. In some aspects, the nutritional composition includes all of the aforementioned vitamins and minerals. In some aspects, the nutritional composition includes any of the aforementioned vitamins and minerals individually, or any combination thereof.

In some aspects, a nutritional composition, pharmaceutical composition, or absorbent article (described elsewhere herein) includes a carotenoid such as lutein, lycopene, zeaxanthin, and beta-carotene. In some aspects, the total amount of carotenoid included varies from about 0.001 μg/ml to about 10 μg/ml. In some aspects, lutein is included in an amount of from about 0.001 μg/ml to about 10 μg/ml, preferably from about 0.044 μg/ml to about 5 μg/ml of lutein. In some aspects, lycopene is included in an amount from about 0.001 μg/ml to about 10 μg/ml, preferably about 0.0185 μg/ml to about 5 μg/ml of lycopene. In aspects, beta-carotene comprises from about 0.001 μg/ml to about 10 mg/ml, for example about 0.034 μg/ml to about 5 μg/ml of beta-carotene.

In some aspects, a nutritional composition, pharmaceutical composition, or absorbent article (described elsewhere herein) contains reduced concentrations of sodium; for example, from about 300 mg/l to about 400 mg/l. In the case of a nutritional or pharmaceutical composition, in the some aspects, the remaining electrolytes are present in concentrations set to meet needs without providing an undue renal solute burden on kidney function. For example, potassium is preferably present in a range of about 1180 to about 1300 mg/l; and chloride is preferably present in a range of about 680 to about 800 mg/l.

In some aspects, a nutritional composition, pharmaceutical composition, or absorbent article (described elsewhere herein) contains various other ingredients such as preservatives; emulsifying agents; thickening agents; buffers; fiber prebiotics (e.g. fructo-oligosaccharides, galacto-oligosaccharides, or a combination thereof); probiotics (e.g., B. animalis subsp. lactis BB-12, B. lactis HN019, B. lactis Bi07, B. infantis ATCC 15697, Lactobacillus rhamnosus (e.g., L. rhamnosus GG, L. rhamnosus HNOOl, L. rhamnosus GR-1), Lactobacillus acidophilus (e.g., L. acidophilus LA-5, L. acidophilus NCFM), Lactobacillus fermentum (e.g., L. fermentum CECT5716, L. fermentum LF15), Bifidobacterium longum (e.g., B. longum BB536, B. longum AH1205, B. longum AH1206), B. breve M-16V, B. adolescentis, B. pseudocatenulatum, Lactobacillus reuteri (e.g., L. reuteri DSM 17938, L. reuteri RC-14, L. reuteri ATCC 55730, L. reuteri ATCC PTA-6485), Lactobacillus crispatus (e.g., L. crispatus LV88, L. crispatus CTV-05 (a live bacterial therapeutic), L. crispatus ATCC 33820), Lactobacillus jensenii (e.g., L. jensenii PVAS25), Lactobacillus gasseri, Lactobacillus iners, Lactobacillus plantarum, Lactobacillus vaginalis, or any combination thereof); antioxidant/anti-inflammatory compounds including tocopherols, carotenoids, ascorbate/vitamin C, ascorbyl palmitate, polyphenols, glutathione, superoxide dismutase (melon), or any combination thereof; other bioactive factors (e.g. growth hormones, cytokines, TFG-β, or any combination thereof); antibiotics (e.g., metronidazole, clindamycin, tinidazole, or any combination thereof); colorants; flavors; stabilizers; lubricants; or any combination thereof. In some aspects, for example, the nutritional composition, pharmaceutical composition, or absorbent article (described elsewhere herein) comprises an oligosaccharide or oligosaccharide composition disclosed herein in combination with an antibiotic (e.g., metronidazole, clindamycin, tinidazole, or any combination thereof); a probiotic (e.g., Lactobacillus crispatus, Lactobacillus jensenii, Lactobacillus gasseri, Lactobacillus iners, Lactobacillus plantarum, Lactobacillus vaginalis, or any combination thereof); or a combination thereof. In some aspects, any composition disclosed herein (e.g., nutritional, pharmaceutical, etc.) does not include a probiotic. In some aspects, any composition disclosed herein does not include lactulose. In some aspects, any composition disclosed herein does not include fructo-oligosaccharides, galacto-oligosaccharides, gluco-oligosaccharides, or any combination thereof.

In some aspects, the nutritional composition, pharmaceutical composition, or absorbent article (described elsewhere herein) includes, for example, fructo-oligosaccharides such as RAFFINOSE (Rhone-Poulenc, Cranbury, New Jersey), inulin (Imperial Holly Corp., Sugar Land, Texas), and NUTRAFLORA (Golden Technologies, Westminister, Colorado), as well as lactose, xylo-oligosaccharides, soy-oligosaccharides, lactulose/lactitol and galacto-oligosaccharides among others. In some applications, other beneficial bacteria, such as Lactobacillus, Rumminococcus, Akkermansia, Bacteroides, Faecalibacterium are included in the compositions.

In some aspects, a nutritional composition, pharmaceutical composition, or absorbent article (described elsewhere herein) is formulated as a soluble powder, a liquid concentrate, or a ready-to-use formulation. In the case of a nutritional or pharmaceutical composition, in some aspects, it is fed to a human in need via a nasogastric tube or orally. In some aspects, various flavors, fibers and other additives are also present.

In some aspects, nutritional compositions are prepared by any commonly used manufacturing techniques for preparing nutritional compositions in solid or liquid form. For example, in some aspects, the composition is prepared by combining various feed solutions. In some aspects, a protein-in-fat feed solution is prepared by heating and mixing the lipid source and then adding an emulsifier (e.g., lecithin), fat soluble vitamins, and at least a portion of the protein source while heating and stirring. A carbohydrate feed solution is then prepared by adding minerals, trace, and ultra-trace minerals, thickening or suspending agents to water while heating and stirring. The resulting solution is held for 10 minutes with continued heat and agitation before adding carbohydrates (e.g., the oligosaccharides described herein and digestible carbohydrate sources). The resulting feed solutions are then blended while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavors are added, and water is added to achieve the desired total solid level. In some aspects, for a liquid product, the resulting solution is then aseptically packed to form an aseptically packaged nutritional composition. In this form, in some aspects, the nutritional composition is in ready-to-feed or concentrated liquid form. Alternatively, in some aspects, the composition is spray-dried and processed and packaged as a reconstitutable powder.

In some aspects when the nutritional product is a ready-to-feed nutritional liquid, it may be preferred that the total concentration of the oligosaccharides or oligosaccharide compositions in the liquid, by weight of the liquid, is from about 0.1% to about 1.5%, including from about 0.2% to about 1.0%, for example from about 0.3% to about 0.7%. In some aspects, when the nutritional product is a concentrated nutritional liquid, it is preferred that the total concentration of oligosaccharide compositions in the liquid, by weight of the liquid, is from about 0.2% to about 3.0%, including from about 0.4% to about 2.0%, for example from about 0.6% to about 1.5%.

In some aspects, a nutritional composition or pharmaceutical composition, is in a unit dosage form. In aspects, the unit dosage form contains an acceptable food- or pharmaceutical-grade carrier, e.g., phosphate buffered saline solution, mixtures of ethanol in water, water, and emulsions, such as an oil/water or water/oil emulsion, as well as various wetting agents or excipients. In some aspects, the unit dosage form contains other materials that do not produce an adverse, allergic, or otherwise unwanted reaction when administered to a subject. In some aspects, the carriers and other materials include solvents, dispersants, coatings, absorption promoting agents, controlled release agents, and one or more inert excipients, such as starches, granulating agents, microcrystalline cellulose, diluents, lubricants, binders, and disintegrating agents. Preferably carriers and other materials are low in FODMAPs or contain no FODMAPs.

In some aspects, the unit dosage form is administered orally, e.g., as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. In some aspects, an orally administered composition includes one or more binders, lubricants, inert diluents, flavoring agents, and humectants. In some aspects, an orally administered composition such as a tablet is coated and formulated to provide sustained, delayed, or controlled release of the oligosaccharide compositions. In aspects, the unit dosage form is administered by naso-gastric tube or direct infusion into the GI tract or stomach. In some aspects, the unit dosage form includes agents such as antibiotics, probiotics, analgesics, and anti-inflammatory agents (e.g., omega 3 poly-unsaturated fatty acids, ibuprofen, or a combination thereof).

In some aspects, the proper dosage of the nutritional or pharmaceutical composition is determined in a conventional manner, based upon factors such as the subject's condition, immune status, body weight and age. For example, the amount of oligosaccharide required to be administered for the improving urogenital health of a female subject will vary depending upon factors such as the risk and severity of the underlying condition(s), any other medical conditions or diseases, age, the form of the composition, and other medications being administered. Further the amount may vary depending upon whether the oligosaccharide is being used to treat (when the dose may be higher) or whether the oligosaccharide are being used as a secondary prevention/maintenance (when the dose may be lower). However, the required amount can be readily set by a medical practitioner. For example, and in general, when administered enterally, the female subject is preferably administered an amount of 0.5 g to 15 g per day of the oligosaccharide, more preferably 1 g to 10 g per day. For example, and in general, the female subject may be administered 2 g to 7.5 g per day. When administered topically or intra-vaginally, the female subject is preferably administered an amount of 0.1 g to 10 g of the oligosaccharide, more preferably 0.2 g to 7.5 g. For example, the topically or intra-vaginally administered composition may contain 0.5 g to 5 g of the oligosaccharide. In aspects when administered in the form of a disposable absorbent article, at least a portion of the article is coated or impregnated with the oligosaccharide in an amount of 0.2 g to 200 g per square meter, preferably between 5.0 g and 100 g per square meter, more preferably between 8.0 g and 50 g per square meter. In aspects when administered orally, the dosage of an oligosaccharide composition of the present invention ranges from about 1 micrograms/L to about 25 grams/L of oligosaccharides, or about 100 micrograms/L to about 15 grams/L of oligosaccharides, or about 1-10 g/L, 5-15 g/L, 10-50 g/L, or as high as 200 g/L. In some aspects, the dosage is 50-70 g/day, 10 g/day, between 1 and 10 g/day, over 100 g/day, or 0.25-3 g/day. Exemplary dosages of probiotic (e.g., Lactobacillus species and/or Bifidobacteria species) include, but are not limited to, about 10⁴ to about 10¹² colony forming units (CFU) per dose, or about 10⁶ to about 10¹⁰ CFU. Other bacterium can also be dosed at similar concentrations, but are not limited to, about 10⁴ to about 10¹² colony forming units (CFU) per dose or about 10⁶ to about 10¹⁰ CFU. In some aspects, appropriate dose regimes are determined by methods known to those skilled in the art, including medical professionals. During an initial treatment phase, the dosing can be higher. During a maintenance phase, the dosing can be reduced.

In some aspects, the oligosaccharide or oligosaccharide composition is used in conjunction with an absorbent article (e.g., disposable absorbent article). For example, in some aspects, the oligosaccharide or oligosaccharide composition is applied to, coated on, infused into, or saturated in an absorbent article, such as a disposable absorbent article. When the absorbent article (e.g., disposable absorbent article) is a diaper, a pant, an adult incontinence product, an absorbent insert for a diaper or undergarment (or pant), or a feminine hygiene product, such as a sanitary napkin and a panty liner, the absorbent article typically comprises a liquid pervious top-sheet, a liquid impervious back-sheet, and/or an absorbent core positioned between the top-sheet and the back-sheet. The absorbent core is preferably compressible, conformable, and non-irritating to the wearer's skin.

In some aspects, the absorbent core is manufactured in a wide variety of sizes and shapes (e.g., rectangular, oval, hourglass, “T” shaped, dog bone, asymmetric, etc.). In some aspects, the absorbent core includes any of a wide variety of liquid-absorbent materials commonly used in absorbent articles, such as comminuted wood pulp. In some aspects, the absorbent core includes other absorbent components that are often used in absorbent articles, for example, an acquisition system, or a secondary top-sheet for increasing the wearer's comfort.

In some aspects, the back-sheet is impervious to liquids (e.g., menses and/or urine) and preferably comprises a thin plastic film, although other flexible liquid impervious materials may also be used. The back-sheet prevents the exudates absorbed and contained in the absorbent core from wetting articles which contact the absorbent article such as bedsheets, pants, pajamas and undergarments. In some aspects, the back-sheet comprises a woven or nonwoven material, polymeric films such as thermoplastic films of polyethylene or polypropylene, or composite materials such as a film-coated nonwoven material.

The top-sheet is preferably compliant, soft feeling, and non-irritating to the wearer's skin. Further, the top-sheet typically is liquid pervious, permitting liquids (e.g., menses and/or urine) to readily penetrate through its thickness. A suitable top-sheet may be manufactured from a wide range of materials such as woven and nonwoven materials (e.g., a nonwoven web of fibers), including apertured nonwovens; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. When the top-sheet comprises a nonwoven web, the web may be manufactured by a wide number of known techniques. For example, the web may be spunbonded, spunlace carded, wet-laid, melt-blown, hydroentangled, hydroformed, hydroapertured, combinations of the above, or the like.

In some aspects, the oligosaccharide or oligosaccharide composition is coated, sprayed, infused, saturated, or otherwise applied onto the top-sheet and/or a substrate beneath the top-sheet. Suitable substrates include a nonwoven web, a film, or tissue, or other such substrates disclosed herein. In use in some aspects, the oligosaccharide or oligosaccharide composition are transferable to the wearer's skin by normal contact, wearer motion, urine, menses, other bodily fluids, body heat, and/or force due to wiping.

Suitable methods for producing disposable absorbent articles are well known in the art, for example, as described in WO 2013/163075, hereby incorporated by reference in its entirety for all purposes, and more specifically for methods of production, to the extent not inconsistent with the description herein.

In some aspects, the oligosaccharide or oligosaccharide composition is in the form of an enterally administered composition. In some aspects, the enterally administered composition comprises an amount of 0.5 g to 15 g of an oligosaccharide or oligosaccharide composition, more preferably 1 g to 10 g. For example, in some aspects, the enterally administered composition contains 2 g to 7.5 g of an oligosaccharide or oligosaccharide composition. The topically administered oligosaccharide or oligosaccharide composition and the intra-vaginally administered oligosaccharide or oligosaccharide composition preferably contain an amount of 0.1 g to 10 g of the oligosaccharide or oligosaccharide composition, more preferably 0.2 g to 7.5 g. For example, in some aspects, the topically or intra-vaginally administered oligosaccharide or oligosaccharide composition contains 0.5 g to 5 g of the oligosaccharide or oligosaccharide composition. In some aspects, when in the form of a disposable absorbent article, at least a portion of the article is coated or impregnated with an oligosaccharide or oligosaccharide composition in an amount of 0.2 g to 200 g per square meter, preferably between 5.0 g and 100 g per square meter, more preferably between 8.0 g and 50 g per square meter. In some aspects, in the case of a female subject requiring improvement in urogenital health or treatment, the female subject is administered a higher dose initially followed by a lower dose. The higher dose is preferably administered for up to 14 days, for example up to 7 days. The lower dose may be administered over an extended period of time. In some aspects, in the case of a female subject requiring management to reduce the risk of bacterial vaginosis, recurrence of bacterial vaginosis, urinary tract infection or recurrence of urinary tract infection, the female subject is administered a lower maintenance dose over an extended period of time.

The invention can be further understood by the following non-limiting examples.

### Example 1

Example 1 demonstrates a method of production of oligosaccharides and oligosaccharide compositions from certain source materials.

Oligosaccharides were produced using a three-step pathway from the following polysaccharide sources: fenugreek galactomannan, carob galactomannan (to produce CLX111), sugar beet arabinan (to produce CLX096), and beech wood xylan (to produce CLX103). Each polysaccharide source was lyophilized, finely ground, hydrated in deionized water (500 mg per 20 ml H₂O), and the pH adjusted to 5.2. A Fenton metal catalyst such as Fe(II), Cu(II), or similar metal (0.247 μmolar) and 5 mL hydrogen peroxide was added. The reaction mixture was then held at 55° C. for 2 hours. Through Fenton catalysis, oxidation of the polysaccharide occurred, thus making the glycosidic bonds more labile. Once complete the pH of the reaction was raised to 10 by the addition of ammonium hydroxide, at which time the charge state of the Fenton catalyst was altered, causing immediate precipitation, and the remaining hydrogen peroxide was degraded. The mixture was then subjected to a second incubation at 45° C. for 1 hour, during which the labile glycosidic bonds were cleaved, forming multiple unique oligosaccharides.

The reaction mixture was then fully lyophilized to remove volatile components and water. The dried reaction mixture was then rehydrated in 10 ml per gram of material using deionized water. The non-water-soluble components were then separated via centrifugation at 6000 rpm for 20 min, the resulting pellet was discarded whereas the supernatant was carried forward to purification via size exclusion filtration. Separation was conducted using 0.03M Ammonium bicarbonate buffer (pH 7.2) run on a BioRad Bio-Scale™ Mini Bio-Gel® P-6 Desalting Cartridges, 50 ml. Purification was conducted using a 10 mL injection volume, at a flowrate of 5 ml/min with an immediate 60 mL collection window. The collected P-6 purification fraction was then subjected to lyophilization.

The dried oligosaccharide sample was then subjected to selective alcohol precipitation to further select for small oligosaccharides. To accomplish this, the dried oligosaccharide sample was hydrated in 70% ethanol and mixed thoroughly for 20 min. The resulting mixture was subjected to centrifugation at 6000 rpm for 20 min, the resulting pellet was discarded whereas the supernatant was dried via rotary evaporation under reduced pressure, thereby providing the oligosaccharides or oligosaccharide compositions.

### Example 2

Example 2 demonstrates the effect of certain oligosaccharide compositions, or the effect of one or more oligosaccharides, on single strain growth.

To determine the effect of an oligosaccharide composition, or the effect of one or more oligosaccharides, on specific microbial strain growth, selected microbes were grown in the presence of oligosaccharides as the sole carbon source and assayed for growth and metabolic output. Selected strains include microbes from the American Type Culture Collection and isolated from human specimens.

Selected strains were incubated in minimal media containing the following oligosaccharide compositions in a final concentration of 2% w/v: CLX101, CLX102, CLX103, CLX105, CLX108, CLX109, CLX110, CLX111, CLX112, CLX113 and CLX114.

Growth was monitored by measuring OD (600 nm) using microtiter plate-reader (Epoch 2, BioTek). FOS, GOS and inulin were included as controls. Specifically, fresh cultures were generated by transferring a colony (per replicate) into fresh liquid media and incubating in an anaerobic chamber (Anaerobic Chamber Vinyl Type B), using a mix of gas (carbon dioxide 5%, hydrogen 5%, nitrogen balance). De Man, Rogosa and Sharpe media (MRS) was used to propagate the following organisms: Lactobacillus crispatus, Lactobacillus rhamnosus, Bifidobacterium longum subsp. infantis, Bifidobacterium longum subsp. longum and Bifidobacterium pseudocatenulatum. Cells were harvested at late exponential phase and an experimental inoculum was prepared by washing once with phosphate-buffered saline (PBS). This was done by centrifuging the culture at 8000 g for 5 min, discarding the supernatant, resuspending in PBS and precipitating the cells once more before resuspending cells again fresh basal (no carbohydrate) media.

Minimal media containing 2% of each of the tested oligosaccharide compositions, 2% glucose or lactose (positive control) or no carbon source (negative control) were inoculated with 2% of the experimental inoculum. Growth in each media type was run 4 times (replicates) and media sterility was tested by incubating non-inoculated media. Growth was determined based on absorbance measurements at 600 nm. Minimal media used for Lactobacillus and Bifidobacteria was basal MRS previously described by Ruiz-Moyano, Totten et al. (2013). Measurements were exported using Gen5 2.0 software. Raw data from single strain growth was processed using an open-source tool designed to generate quantitative bacterial growth. Measurements were normalized by calculating the delta of OD₆₀₀ with a specific oligosaccharide composition vs negative control (media without carbohydrate), divided by the delta of OD₆₀₀ with the oligosaccharide composition vs positive control (2% glucose or lactose) and multiplied by 100.

Supernatants of single strain bacterial growths were derivatized with 2-nitrophenylhydrazine (2-NPH) for LC-MS/QqQ analysis. In brief, 20 μL of supernatant was added to 20 μL of N-(3-Dimethylaminopropyl)-N′-ethylcarbodiimide hydrochloride (1-EDC HCl) in 5% pyridine. Then 40 μL of 200 mM 2-NPH in 80% acetonitrile with 50 mM HCl was added and briefly vortexed prior to incubating for 30 minutes at 40° C. After incubation, samples were diluted with 400 μL of 10% acetonitrile and vortexed. The dilutions were centrifuged prior to LC-MS/QqQ analysis.

Short chain fatty acids were analyzed with a 1290 Infinity II LC (Agilent Technologies, Santa Clara, CA) equipped with a reverse phase column (Zorbax Eclipse C18 2.1×50 mm; Agilent Technologies, Santa Clara, CA) and 6490A triple quadrupole (QqQ) mass spectrometer (Agilent Technologies, Santa Clara, CA). LC separation was performed with 5% acetonitrile with 0.1% formic acid (solvent A) and 95% acetonitrile with 0.1% formic acid (solvent B). A separation gradient was as follows: 5% to 20% B for 2 minutes, then 20% to 55% B for 2 minutes, 55-100% B in 0.1 minute, hold at 100% B for 0.5 minutes, return to 5% B in 0.1 minute, and equilibrate at 5% B for 0.9 minutes. ESI-MS conditions were performed in positive mode and the dynamic multiple reaction monitoring (dMRM) mode was used to monitor the precursor and product ion transitions. Peak areas were quantitated using Agilent Quantitative Analysis software and areas were normalized to internal standards and compared to an external standard curve for quantitation.

Supernatants of fecal bacterial growths were diluted with acetonitrile (1:10 dilution) for LC-MS/QTOF analysis. Metabolites were analyzed with a 1290 Infinity II LC (Agilent Technologies, Santa Clara, CA) equipped with a HILIC column (InfinityLab Poroshell 120 HILIC-Z, 2.1×150 mm; Agilent Technologies, Santa Clara, CA) and 6530 LC-MS QTOF (Agilent Technologies, Santa Clara, CA). LC separation was performed with 10% 200 mM ammonium formate with 0.1% Formic Acid+90% Water (solvent A) and 10% 200 mM ammonium formate with 0.1% Formic Acid+90% Acetonitrile (solvent B). The MS conditions were set to positive mode with a scan range set at m/z 50-1700 at 1 spectra/see scan rate. Peak area was quantitated using Agilent Quantitative Analysis software and areas were normalized to internal standards for quantitation.

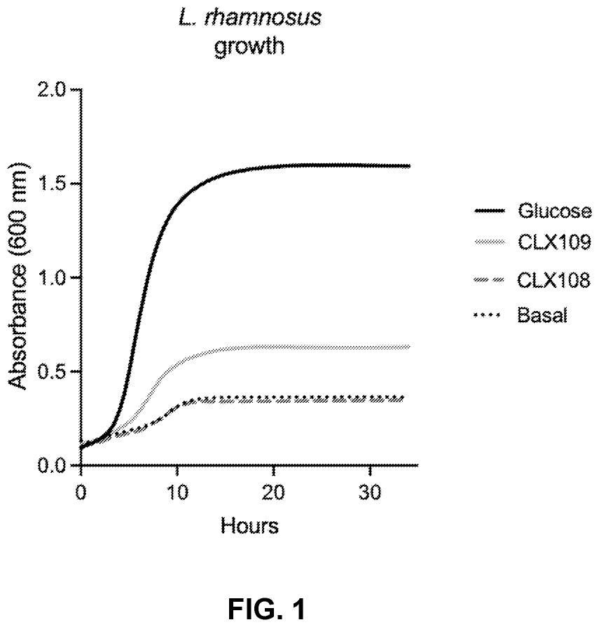

Differential growth of tested strains was observed due to the oligosaccharide composition used as a sole carbon source, and the number of strains they supported varies as a function of the oligosaccharide composition. L. crispatus growth was supported by CLX101, CLX102, CLX109, CLX110 and CLX112. L. rhamnosus growth was supported by CLX101, CLX109, CLX110, and CLX112. Bif. longum subsp. infantis growth was supported by CLX101, CLX105, CLX109. Bif. pseudocatenulatum growth was supported by CLX101, CLX102, CLX103, CLX105, CLX109, CLX110, CLX111, CLX114. Bif. longum subsp. longum growth was supported by CLX101, CLX105, CLX109, and CLX114. An example of selective growth is demonstrated by L. rhamnosus, in which some of the oligosaccharide compositions supported growth but not in others. The results are shown in Table Z and FIG. 1.

TABLE Z
Summary of growth of selected strains with oligosaccharide compositions.

| | Supported growth of selected strains | | | | | |
|---|---|---|---|---|---|---|
| Oligosaccharide | L. | L. | Bif. longum | Bif. | Bif. longum | F. |
| composition | crispatus | rhamnosus | subsp. infantis | pseudocatenatum | subsp. longum | prausnitzii |
| CLX101 | ++ | + | + | + | + | |
| CLX102 | + | − | − | + | − | |
| CLX103 | − | − | − | ++ | − | |
| CLX105 | − | − | + | + | ++ | |
| CLX108 | − | − | − | − | − | |
| CLX109 | + | + | + | ++ | ++ | |
| CLX110 | + | + | − | ++ | − | |
| CLX111 | − | − | − | + | − | |
| CLX112 | ++ | + | − | − | − | |
| CLX113 | − | − | − | − | − | |
| CLX114 | − | − | − | + | + | |

Key symbols:
−: <20,
+: 20-50;
++: >50

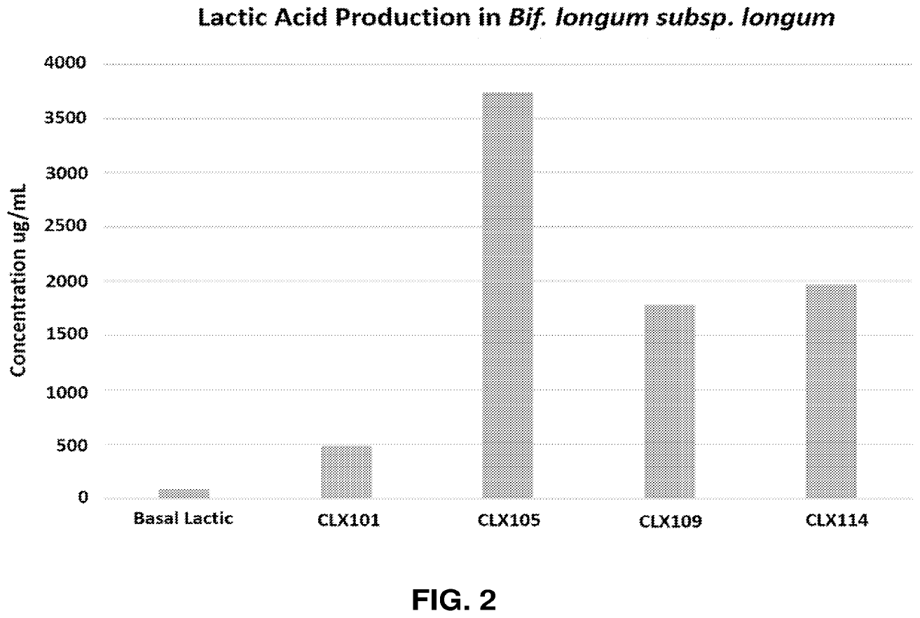

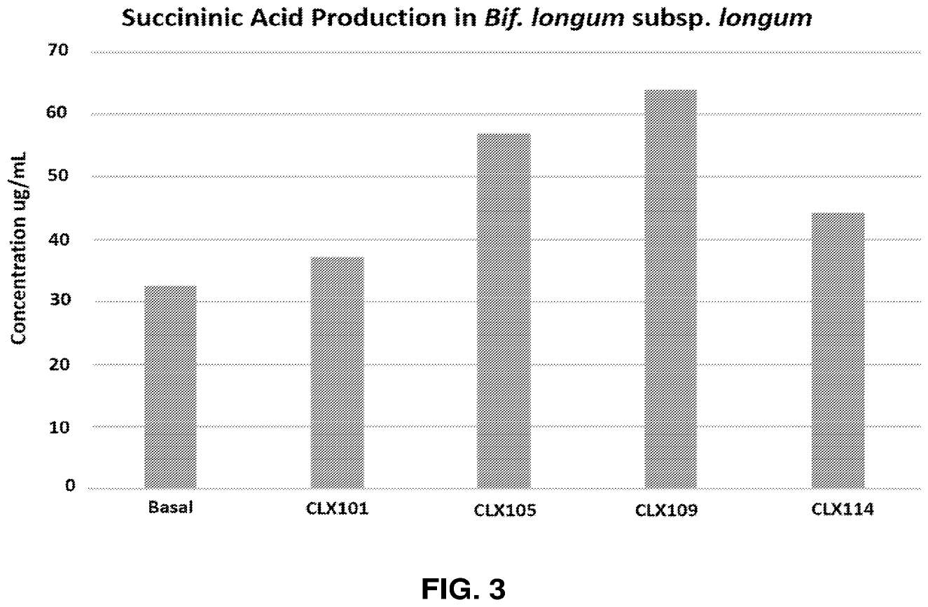

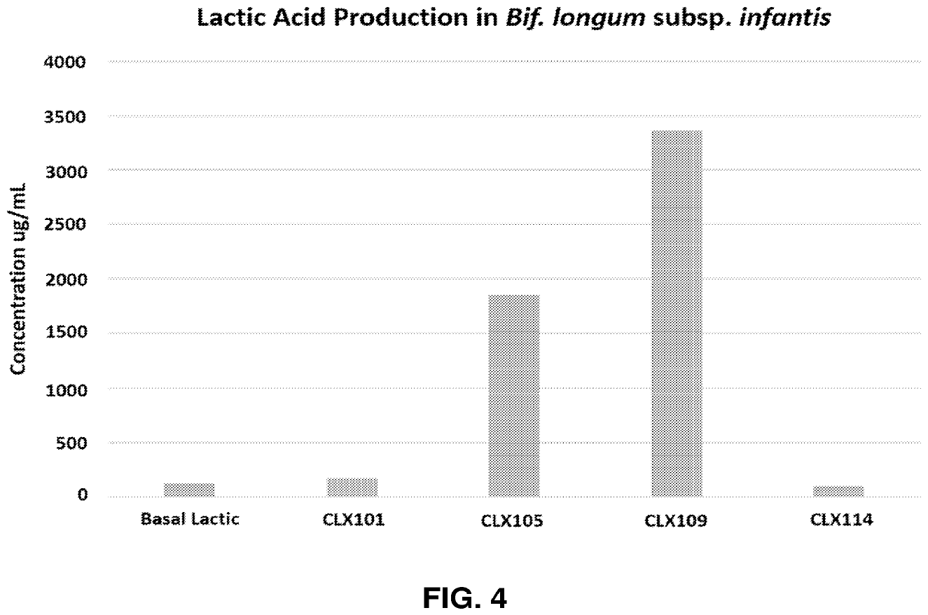

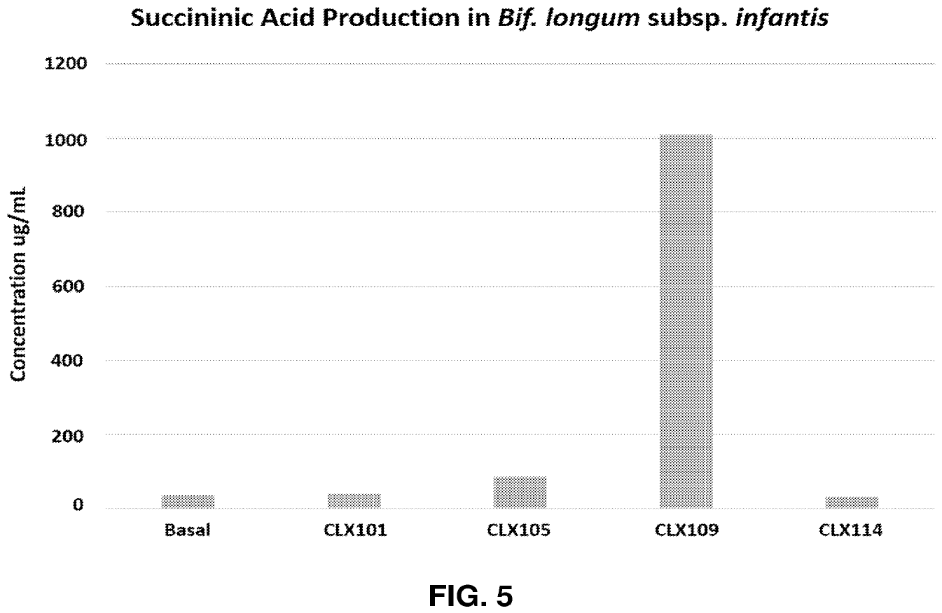

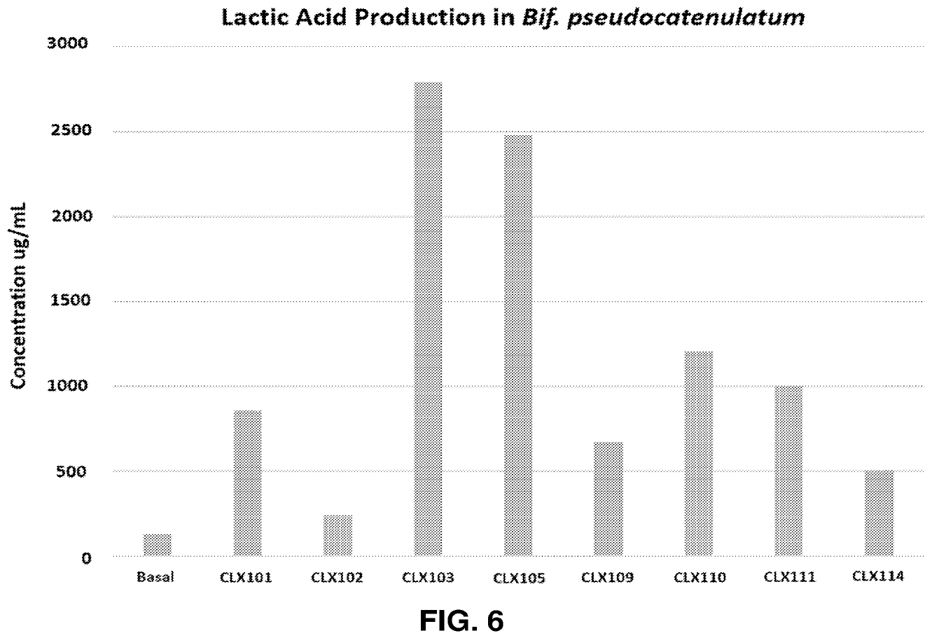

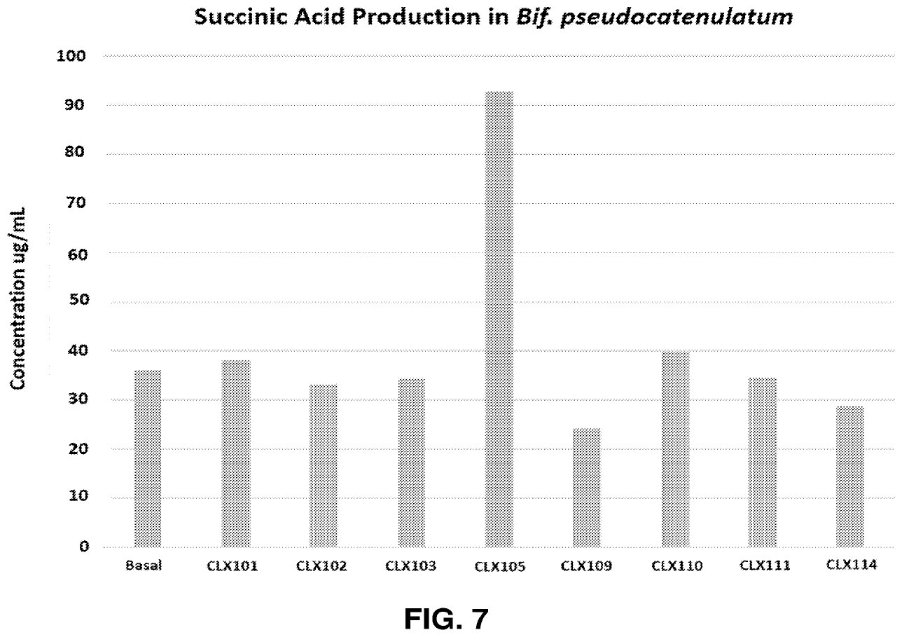

The selected microbial strains (Bif longum subsp. longum, Bif longum subsp. infantis, Bif. pseudocatenatum) showed preferential growth with specific oligosaccharide compositions. The growth resulted in the production of lactic and/or succinic acids above the levels of the basal samples. Bif longum subsp. longum produced lactic and succinic acids in CLX101, CLX105, CLX109 and CLX114 (FIGS. 2-3). Bif longum subsp. infantis produced lactic and succinic acids in CLX105 and CLX109, however the levels of both acids were comparable to the basal samples for CLX101 and CLX114 (FIGS. 4-5). Notably, Bif longum subsp. infantis produced 10 times more succinic acid in composition CLX109 and produced more than 15 times more lactic acid in compositions CLX105 and CLX109 than the other oligosaccharides. Bif. pseudocatenatum produced succinic acid in CLX105, however the other oligosaccharides CLX101, CLX102, CLX103, CLX109, CLX110, CLX111 and CLX114 were about the same levels as the basal sample (FIG. 7). Bif. pseudocatenulatum produced lactic acid in CLX101, CLX102, CLX103, CLX105, CLX109, CLX110, CLX111, and CLX114 above the levels of the basal samples. (FIG. 6). L. crispatus and L. rhamnosus produced lactic acid in CLX101, CLX110 and CLX112. Additionally, L. crispatus produced lactic acid in CLX102 and CLX109. L. crispatus produced succinic acid in CLX101, CLX102, CLX109, CLX110 and CLX112. However, L. rhamnosus did not produce any succinic acid levels above the basal samples.

These results indicate that, while many oligosaccharide compositions can support the growth of certain bacterial strains, they each uniquely modulate the metabolic (SCFA) outputs of that bacteria. This allows the optimization of certain symbiotic pairings that are focused on metabolite modulation.

### Example 3

Example 3 demonstrates the effect of certain oligosaccharide compositions, or the effect of one or more oligosaccharides, on single strain growth.

To determine the effect of an oligosaccharide composition, or the effect of one or more oligosaccharides, on specific microbial strain growth, selected microbe was grown in the presence of oligosaccharides as the sole carbon source and assayed for growth and metabolic output. Selected strain includes microbes from the American Type Culture Collection and isolated from human specimens.

Selected strains were incubated in minimal media containing the following oligosaccharide compositions in a final concentration of 2% w/v: CLX114, CLX115, CLX123, CLX125, CLX126 and CLX128. Growth was monitored by measuring OD (600 nm) using microtiter plate-reader (Epoch 2, BioTek). Specifically, fresh cultures were generated by transferring a colony (per replicate) into fresh liquid media and incubating in an anaerobic chamber (Anaerobic Chamber Vinyl Type B), using a mix of gas (carbon dioxide 5%, hydrogen 5%, nitrogen balance). De Man, Rogosa and Sharpe media (MRS) was used to propagate the Lactobacillus crispatus. Cells were harvested at late exponential phase and an experimental inoculum was prepared by washing once with phosphate-buffered saline (PBS). This was done by centrifuging the culture at 8000 g for 5 min, discarding the supernatant, resuspending in PBS and precipitating the cells once more before resuspending cells again fresh basal (no carbohydrate) media.

Minimal media containing 2% of each of the tested oligosaccharide compositions, 2% glucose or lactose (positive control) or no carbon source (negative control) were inoculated with 2% of the experimental inoculum. Growth in each media type was run 4 times (replicates) and media sterility was tested by incubating non-inoculated media. Growth was determined based on absorbance measurements at 600 nm. Minimal media used for Lactobacillus was basal MRS previously described by Ruiz-Moyano, Totten et al. (2013). Measurements were exported using Gen5 2.0 software. Raw data from single strain growth was processed using an open-source tool designed to generate quantitative bacterial growth. Measurements were normalized by calculating the delta of OD₆₀₀ with a specific oligosaccharide composition vs negative control (media without carbohydrate), divided by the delta of OD₆₀₀ with the oligosaccharide composition vs positive control (2% glucose or lactose) and multiplied by 100.

Supernatants of single strain bacterial growths were derivatized with 2-nitrophenylhydrazine (2-NPH) for LC-MS/QqQ analysis. In brief, 20 μL of supernatant was added to 20 μL of N-(3-Dimethylaminopropyl)-N′-ethylcarbodiimide hydrochloride (1-EDC HCl) in 5% pyridine. Then 40 μL of 200 mM 2-NPH in 80% acetonitrile with 50 mM HCl was added and briefly vortexed prior to incubating for 30 minutes at 40° C. After incubation, samples were diluted with 400 μL of 10% acetonitrile and vortexed. The dilutions were centrifuged prior to LC-MS/QqQ analysis.

Short chain fatty acids were analyzed with a 1290 Infinity II LC (Agilent Technologies, Santa Clara, CA) equipped with a reverse phase column (Zorbax Eclipse C18 2.1×50 mm; Agilent Technologies, Santa Clara, CA) and 6490A triple quadrupole (QqQ) mass spectrometer (Agilent Technologies, Santa Clara, CA). LC separation was performed with 5% acetonitrile with 0.1% formic acid (solvent A) and 95% acetonitrile with 0.1% formic acid (solvent B). A separation gradient was as follows: 5% to 20% B for 2 minutes, then 20% to 55% B for 2 minutes, 55-100% B in 0.1 minute, hold at 100% B for 0.5 minutes, return to 5% B in 0.1 minute, and equilibrate at 5% B for 0.9 minutes. ESI-MS conditions were performed in positive mode and the dynamic multiple reaction monitoring (dMRM) mode was used to monitor the precursor and production transitions. Peak areas were quantitated using Agilent Quantitative Analysis software and areas were normalized to internal standards and compared to an external standard curve for quantitation.

Supernatants of fecal bacterial growths were diluted with acetonitrile (1:10 dilution) for LC-MS/QTOF analysis. Metabolites were analyzed with a 1290 Infinity II LC (Agilent Technologies, Santa Clara, CA) equipped with a HILIC column (InfinityLab Poroshell 120 HILIC-Z, 2.1×150 mm; Agilent Technologies, Santa Clara, CA) and 6530 LC-MS QTOF (Agilent Technologies, Santa Clara, CA). LC separation was performed with 10% 200 mM ammonium formate with 0.1% Formic Acid+90% Water (solvent A) and 10% 200 mM ammonium formate with 0.1% Formic Acid+90% Acetonitrile (solvent B). The MS conditions were set to positive mode with a scan range set at m/z 50-1700 at 1 spectra/see scan rate. Peak area was quantitated using Agilent Quantitative Analysis software and areas were normalized to internal standards for quantitation.

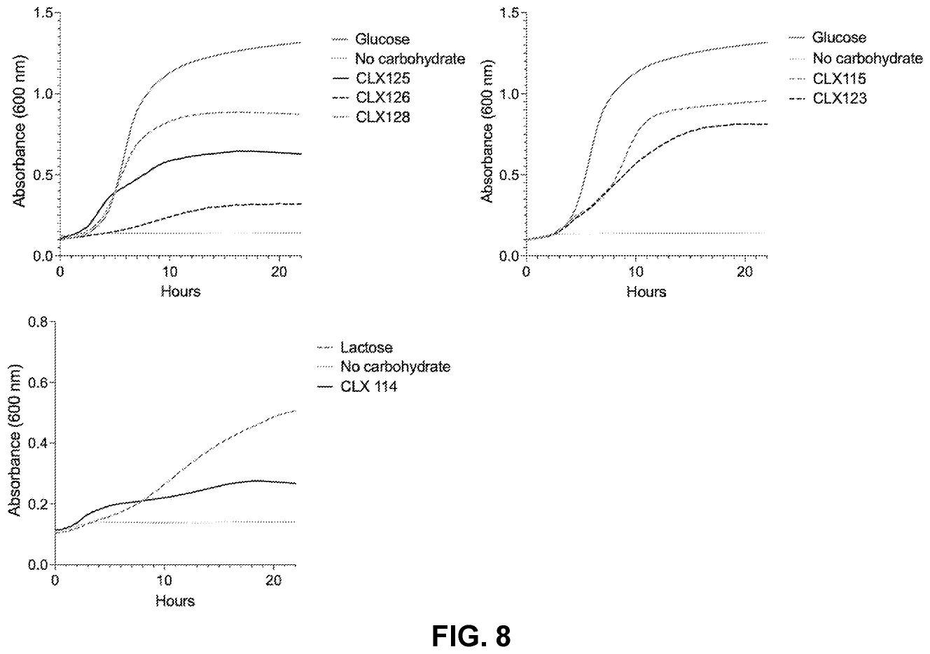

Differential growth of the tested strain was observed due to the oligosaccharide composition used as a sole carbon source. L. crispatus growth was supported by CLX114, CLX115, CLX123, CLX125, CLX126 and CLX128. The results are shown in FIG. 8.

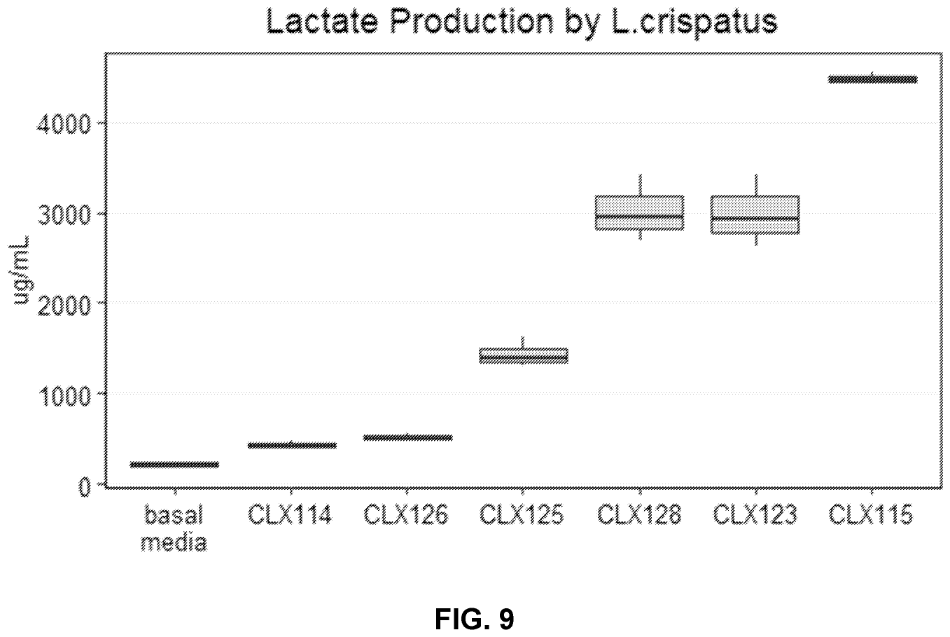

Growth of the selected microbial strains resulted in the production of lactic acid above the levels of the basal samples. The results are shown in FIG. 9.

These results indicate that, while many oligosaccharide compositions can support the growth of certain bacterial strains, they each uniquely modulate the metabolic (SCFA) outputs of that bacteria. This allows the optimization of certain symbiotic pairings that are focused on metabolite modulation.

### Example 4

Example 4 provides a method to depolymerize beta glucan and arabinan.

The use of iron-based and copper-based Fenton depolymerization has been demonstrated in several previous publications (WO2021097138A1, WO2018236917A1, WO2020247389A1, and WO2022241163A1). However, a thorough optimization of copper-based Fenton depolymerization has never been demonstrated. Here, we show a set of optimized parameters and provide instructions for the copper-based Fenton depolymerization of beta glucan and arabinan, particularly from cereals or legumes.

A solution containing 4% hydrogen peroxide with 43.4 mM, pH 5.5 ammonium acetate buffer is heated to 55° C. Beta Glucan or arabinan (or other) polysaccharides are stirred in gradually to a final concentration of 10%. To initiate the first reaction step, Copper (II) sulfate is added to a final concentration of 0.75 mM. The reaction is allowed to proceed for 2 hours at 55° C. then cooled to below 15° C. Next, to initiate the second reaction step, concentrated ammonium hydroxide is added to a final concentration of 0.67 M. The reaction is allowed to stir at 45° C. for 2 hours. The reaction is filtered by vacuum with a GD120 filter and Buchner funnel, then treated with MB 10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilize din minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

### Example 5

Example 5 provides a method to depolymerize beta glucan and arabinan.

The use of iron-based and copper-based Fenton depolymerization has been demonstrated in several previous publications (WO2021097138A1, WO2018236917A1, WO2020247389A1 and WO2022241163A1). Here, we show a set of optimized parameters for the iron-based Fenton depolymerization of beta glucan and arabinan, particularly from cereals and legumes.

A solution containing 7% hydrogen peroxide with 43.4 mM, pH 5.5 ammonium acetate buffer is heated to 55° C. Beta Glucan or arabinan (or other) polysaccharides are stirred in gradually to a final concentration of 5%. To initiate the first reaction step, Iron (II) sulfate is added to a final concentration of 1.15 mM. The reaction is allowed to proceed for 2 hours at 55° C. then cooled to below 15° C. Next, to initiate the second reaction step, concentrated ammonium hydroxide is added to a final concentration of 0.39 M. The reaction is allowed to stir at 45° C. for 2 hours. The reaction is filtered by vacuum with a GD120 filter and Buchner funnel, then treated with MB10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilized in minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

### Aspects of the Invention

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect A13) that references an aspect (e.g., Aspect A1) for which there are sub-aspects having the same top level number (e.g., Aspect A1a, A1b, A1c, and so forth) necessarily includes reference to those sub-aspects A1a, A1b, A1c, and so forth. In other words, if Aspect A13 refers to Aspect A1, and there are Aspects Ala and Alb present, then Aspect A13 refers to Aspects A1a or A1b. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect, . . . ” means that any aspect prior to aspect B13 is referenced, including aspects B1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y.

Aspect A1. A method of improving urogenital health in a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; and
wherein the administering step results in improving urogenital health in the female subject.

Aspect B1: A method of treating or preventing a vaginal infection in a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; and
wherein the administering step results in treating or preventing a vaginal infection in the female subject.

Aspect B2: The method of aspect B1, or any preceding aspect, wherein the vaginal infection comprises a bacterial infection.

Aspect B3: The method of aspect B2, or any preceding aspect, wherein the bacterial infection comprises bacterial vaginosis.

Aspect B4: The method of aspect B2, or any preceding aspect, wherein the bacterial infection comprises a urinary tract infection.

Aspect B5: The method of aspect B1, or any preceding aspect, wherein the vaginal infection comprises a fungal infection.

Aspect B6: The method of aspect B5, or any preceding aspect, wherein the fungal infection comprises a yeast infection.

Aspect C1: A method of modulating the microbiota of the urogenital tract of a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof;
wherein the administering step comprises contacting said microbiota of the urogenital tract with said one or more oligosaccharides; and wherein the administering step results in modulating the microbiota of the urogenital tract of the female subject.

Aspect C2: The method of aspect C1, or any preceding aspect, wherein the method results in increased abundance, e.g., a 10% increase, a 25% increase, a 50% increase, a 75% increase, a 100% increase, a 2× increase, a 3× increase, a 5× increase, a 10× increase, a 50× increase, a 100× increase, or a 1,000× increase, of Lactobacillus species in the urogenital tract of the female subject.

Aspect C3: The method of aspect C2, or any preceding aspect, wherein the Lactobacillus species comprise L. crispatus, L. gasseri, L. jensenii, L. iners, L. vaginalis, or any combination thereof.

Aspect D1: A method of lowering the pH of the urogenital tract of a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof;
wherein the administering step results in lowering the pH of the urogenital tract.

Aspect D2: The method of aspect D1, or any preceding aspect, wherein the method results in lowering the pH of the urogenital tract from a first pH of 4.5-7 (e.g., a first pH of 4.5, 4.75, 5, 5.25, 5.5, 5.75, 6, 6.25, 6.5, 6.75, or 7) to a second pH of less than 4.5 (for example, a second pH of 4.49. 4.25, 4, 3.75, 3.5, 3.25, or 3; optionally, not less than a pH of 2.5).

Aspect D3: The method of claim D2, or any preceding aspect, wherein the second pH is between 3.5-4.5 (for example, a pH of 3.5, 3.6, 3.7, 3.8, 3.9, 4, 4.1, 4.2, 4.3, 4.4, or 4.5).

Aspect E1: The method of any one aspects A1-D3, wherein the method results in enhanced microbial production of lactic acid in the urogenital tract of the female subject. For example, the method may result in about a 10% increase of lactic acid production, a 50% increase of lactic acid production, a 100% increase of lactic acid production, a 250% increase of lactic acid production, a 500% increase of lactic acid production, or a 1,000% increase of lactic acid production.

Aspect E2: The method of any one of aspects A1-E1, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, 8 to 30 subunits, 8 to 20 subunits, or preferably 3 to 30 subunits) wherein at least 5% (e.g., within the range of 5-100%, within the range of 10-100%, within the range of 15-100%, within the range of 20-100%, within the range of 50-100%, within the range of 75-100%, within the range of 85-100%, within the range of 95-100%, or 100%) of the subunits comprise a beta-1,3 glucose residue, a beta-1,4 glucose residue, or a combination thereof.

Aspect E2a: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, or 8 to 20 subunits) wherein at least 10% (e.g., within the range of 10-100%, within the range of 15-100%, within the range of 20-100%, within the range of 50-100%, within the range of 75-100%, within the range of 85-100%, within the range of 95-100%, or 100%) of the subunits comprise a beta-1,3 glucose residue, a beta-1,4 glucose residue, or a combination thereof.

Aspect E2b: The method of aspect E2, or any preceding aspect, wherein each of the one or more oligosaccharides independently contains about 3 to about 30 subunits (e.g., 3 to 30 subunits, 3 to 20 subunits, 3 to 10 subunits, or 5 to 30 subunits, 5 to 20 subunits, 5 to 10 subunits, 8 to 30 subunits, 8 to 20 subunits) wherein at least 20% (e.g., within the range of 20-100%, within the range of 50-100%, within the range of 75-100%, within the range of 85-100%, within the range of 95-100%, or 100%) of the subunits comprise a beta-1,3 glucose residue, a beta-1,4 glucose residue, or a combination thereof.

Aspect E2c: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, or 8 to 20 subunits) wherein at least 50% (e.g., within the range of 50-100%, within the range of 75-100%, within the range of 85-100%, within the range of 95-100%, or 100%) of the subunits comprise a beta-1,3 glucose residue, a beta-1,4 glucose residue, or a combination thereof.

Aspect E2d: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, or 8 to 20 subunits) wherein at least 10% (e.g., within the range of 10-100%, within the range of 15-100%, within the range of 20-100%, within the range of 50-100%, within the range of 75-100%, within the range of 85-100%, within the range of 95-100%, or 100%) of the subunits comprise a beta-1,4 glucose residue.

Aspect E2e: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, or 8 to 20 subunits) wherein at least 20% (e.g., within the range of 20-100%, within the range of 50-100%, within the range of 75-100%, within the range of 85-100%, within the range of 95-100%, or 100%) of the subunits comprise a beta-1,4 glucose residue.

Aspect E2c: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, or 8 to 20 subunits) wherein at least 50% (e.g., within the range of 50-100%, within the range of 75-100%, within the range of 85-100%, within the range of 95-100%, or 100%) of the subunits comprise a beta-1,4 glucose residue.

Aspect E2f: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, or 8 to 20 subunits) wherein at least 10% (e.g., within the range of 10-100%, within the range of 15-100%, within the range of 20-100%, within the range of 50-100%, within the range of 75-100%, within the range of 85-100%, within the range of 95-100%, or 100%) of the subunits comprise a beta-1,3 glucose residue.

Aspect E2g: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, or 8 to 20 subunits) wherein at least 20% (e.g., within the range of 20-100%, within the range of 50-100%, within the range of 75-100%, within the range of 85-100%, within the range of 95-100%, or 100%) of the subunits comprise a beta-1,3 glucose residue.

Aspect E2h: The method of aspect E2, or any preceding aspect, wherein each of the one or more oligosaccharides independently contains about 3 to about 30 subunits (e.g., 3 to 30 subunits, 3 to 20 subunits, 3 to 10 subunits, 5 to 30 subunits, 5 to 20 subunits, 5 to 10 subunits, 8 to 30 subunits, 8 to 20 subunits) wherein at least 50% (e.g., within the range of 50-100%, within the range of 75-100%, within the range of 85-100%, within the range of 95-100%, or 100%) of the subunits comprise a beta-1,3 glucose residue.

Aspect E3: The method of any one of aspects A1-E2h, wherein the one or more materials comprises beta glucan; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX115 set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX115 set forth in Table P; or
any combination thereof.

Aspect E4: The method of any one of aspects A1-E3, wherein the one or more materials comprises beta glucan.

Aspect E5: The method of aspect E4, or any preceding aspect wherein the one or more oligosaccharides contains beta-1,3 and beta-1,4 linked glucose residues.

Aspect E6: The method of aspect E4, or any preceding aspect, wherein the one or more oligosaccharides contains 3 to 50 subunits, wherein each subunit is a beta-1,3 glucose residue, a beta-1,4 glucose residue, or a combination thereof.

Aspect E7: The method of aspect E4, or any preceding aspect, wherein the one or more oligosaccharides comprises beta-1,3 linked glucose residues and beta-1,4 linked glucose residues, wherein the ratio of beta-1,3 linked residues:beta-1,4 linked residues ranges from 1:1 to 1:5 (e.g., 1:1, 1:1.5, 1:2, 1:2.5, 1:3, 1:3.5, 1:4, 1:4.5, or 1:5).

Aspect E8: The method of aspect E4, or any preceding aspect, wherein the one or more oligosaccharides has an average molecular weight (Mw) of less than 10,000 Da. For example, the one or more oligosaccharides has an average molecular weight (Mw) within the range of 1,000-10,000 Da, 2,000-10,000 Da, 2,5000-10,000 Da, 3,000-10,000 Da, 4,000-10,000 Da, 5,000-10,000 Da, 7,500-10,000 Da, or 8,000-10,000 Da.

Aspect E9: The method of aspect E4, or any preceding aspect, wherein the one or more oligosaccharides has an average molecular weight (Mw) of less than 8,000 Da. For example, the one or more oligosaccharides has an average molecular weight (Mw) within the range of 1,000-8,000 Da, 2,000-8,000 Da, 2,5000-8,000 Da, 3,000-8,000 Da, 4,000-8,000 Da, 5,000-8,000 Da, or 6,000-8,000 Da.

Aspect E10: The method of aspect E4, or any preceding aspect, wherein the one or more oligosaccharides has a dynamic viscosity ranging from 1 to 10 mPa*s at 100 mg/ml at 25° C. For example, the one or more oligosaccharides has a dynamic viscosity ranging from 1 to 10 mPa*s at 100 mg/ml at 25° C., 1.5 to 10 mPa*s at 100 mg/ml at 25° C., 1.8 to 10 mPa*s at 100 mg/ml at 25° C., 2 to 10 mPa*s at 100 mg/ml at 25° C., 2.5 to 10 mPa*s at 100 mg/ml at 25° C., 1 to 8 mPa*s at 100 mg/ml at 25° C., or 1.5 to 8 mPa*s at 100 mg/ml at 25° C.

Aspect E11: The method of aspect E4, or any preceding aspect, wherein at least 70% (e.g., within the range of 70-100%, within the range of 80-100%, or within the range of 90-100%) of the mass of the one or more oligosaccharides has a molecular mass of less than 100 kDa (e.g., within the range of 1-100 kDa, 1-80 kDa, 1-60 kDa, 1-50 kDa, 1-25 kDa, 5-100 kDa, 5-80 kDa, 10-100 kDa, or 10-80 kDa).

Aspect E12: The method of aspect E4, or any preceding aspect, wherein at least 60% (e.g., within the range of 60-100%, within the range of 70-100%, within the range of 80-100%, or within the range of 90-100%) of the mass of the one or more oligosaccharides has a molecular mass of less than 50 kDa (e.g., within the range of 1-50 kDa, 1-40 kDa, 1-25 kDa, 5-50 kDa, 5-25 kDa, or 10-50 kDa).

Aspect E13: The method of aspect E4, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 15 kDa (e.g., within the range of 1-15 kDa, 1-12 kDa, 1-10 kDa, 1-8 kDa, or 1-5 kDa).

Aspect E14: The method of aspect E4, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 5 kDa (e.g., within the range of 1-5 kDa, 1.2-5 kDa, 1.5-5 kDa, 1.8-5 kDa, or 2-5 kDa).

Aspect E15: The method of E4, or any preceding aspect, wherein the one or more materials comprises a cereal, a lichenan, a yeast, or any combination thereof.

Aspect E16: The method of aspect E4, or any preceding aspect, wherein the one or more materials comprises a cereal, and the cereal comprises a barley, a rye, a wheat, an oat, or any combination thereof.

Aspect E17: The method of aspect E4, or any preceding aspect, wherein at least 10 wt. % (e.g., within the range of 10-100 wt. %, within the range of 20-100 wt. %, within the range of 30-100 wt. %, within the range of 50-100 wt. %, within the range of 75-100 wt. %, or 100 wt. %) of the one or more oligosaccharides comprises a degree of polymerization between 2 and 30 (e.g., between 2 and 30, between 2 and 20, between 2 and 15, between 2 and 10, between 3 and 30, between 3 and 20, between 4 and 30, or between 4 and 20) based on total mass of the one or more oligosaccharides.

Aspect E18: The method of aspect E4, or any preceding aspect, wherein the one or more oligosaccharides contain at least 10%, (e.g., within the range of 10-100 wt. %, within the range of 20-100%, within the range of 30-100%, within the range of 50-100%, or within the range of 75-100%) glucose subunits.

Aspect E19: The method of aspect E4, or any preceding aspect, wherein the one or more oligosaccharides further comprises galactose subunits and mannose subunits, wherein the galactose subunits are present in an amount of at least 1% (e.g., within the range of 1-90%, within the range of 1-50%, within the range of 1-30%, within the range of 1-20%, within the range of 1-19%, within the range of 3-90%, within the range of 3-50%, within the range of 3-30%, within the range of 3-20%, or within the range of 5-90%), and wherein the mannose subunits are present in an amount of at least 1% (e.g., within the range of 1-90%, within the range of 1-50%, within the range of 1-30%, within the range of 1-20%, range of 3-90%, within the range of 3-50%, within the range of 3-30%, within the range of 3-20%, or within the range of 5-90%).

Aspect E20: The method of aspect E4, or any preceding aspect, wherein the one or more oligosaccharides comprises glucose subunits and galactose subunits, wherein the galactose:glucose ratio is between 0.05:1 to 2:1 (e.g., 0.05:1, 0.11:1, 0.2:1, 0.25:1, 0.5:1, 1:1, 1.1:1, 1.25:1, 1.5:1, or 2:1).

Aspect E21: The method of aspect E4, or any preceding aspect, wherein the one or more oligosaccharides comprises glucose subunits and mannose subunits, wherein the mannose:glucose ratio is between 0.1:1 to 2:1 (e.g., 0.1:1, 0.2:1, 0.25:1, 0.5:1, 1:1, 1.1:1, 1.25:1, 1.5:1, 2:1, or any preceding aspect).

Aspect E22: The method of aspect E4, or any preceding aspect, wherein the one or more oligosaccharides comprises mannose subunits, wherein the mannose subunits comprise 2-linkages, 3-linkages, and terminal linkages in a total amount of at least 10% (e.g., within the range of 10-100%, within the range of 20-100%, within the range of 30-100%, within the range of 50-100%, or within the range of 75-100%).

Aspect E23: The method of aspect E4, or any preceding aspect, wherein the glucose subunits comprise 3-linkages, 4-linkages, and terminal linkages in a total amount of at least 10% (e.g., within the range of 10-100%, within the range of 20-100%, within the range of 30-100%, within the range of 50-100%, or within the range of 75-100%).

Aspect E24: The method of aspect E4, or any preceding aspect, wherein the glucose subunits comprise a ratio of 3-linkages:4-linkages:terminal linkages of 1:0.5:1 to 3:2:1 (e.g., 1:0.5:1, 1.5:0.5:1, 2:0.5:1, 2.5:0.5:1, 3:0.5:1, 1:1:1, 1:1.5:1, 2:1:1, 1:2:1, 1.1:1.9:1, or any combination thereof).

Aspect E25: The method of aspect E4, or any preceding aspect, wherein the glucose subunits comprise a ratio of 3-linkages:4-linkages:terminal linkages of 0.4:1:1 to 0.7:2:1 (e.g., 0.4:1:1, 0.5:1:1, 0.6:1:1, 0.7:1:1, 0.4:1.5:1, 0.4:2: 1, 0.5:1.5:1, 0.5:2:1, 1:4:1, 2:4:1, 3:4:1, 1:5:1, 2:5:1, 1:6:1, 0.55:1.6:1, 1.8:5:1, or any combination thereof).

Aspect E26: The method of aspect E4, or any preceding aspect, wherein the glucose subunits comprise a ratio of 3-linkages:4-linkages of 0.1:1 to 0.8:1 (e.g., 0.1:1, 0.2:1, 0.3:1, 0.4:1, 0.5:1, 0.6:1, 0.7:1, 0.8:1, or any combination thereof).

Aspect E27: The method of aspect E4, or any preceding aspect, wherein the glucose subunits comprise a ratio of 3-linkages:4-linkages of 0.3:1 to 0.65:1 (e.g., 0.3:1, 0.35:1, 0.4:1, 0.5:1, 0.6:1, 0.65:1, or any combination thereof).

Aspect E28: The method of aspect E4, or any preceding aspect, wherein the glucose subunits comprise a ratio of 4-linkages:terminal linkages of 1:1 to 3:1 (e.g., 1:1, 1.1:1, 1.3:1, 1.5:1, 1.6:1, 1.8:1, 1.9:1, 2:1, 2.2:1, 2.5:1, 3:1, or any combination thereof).

Aspect E29: The method of aspect E4, or any preceding aspect, wherein the one or more oligosaccharides comprises glucose subunits and galactose subunits, wherein the one or more oligosaccharides comprises a ratio of glucose 4-linkages:galactose terminal linkages of 5:1 to 7:1 (e.g., 5:1, 5.5:1, 6:1, 6.1:1, 6.2:1, 6.5:1, 6.7:1, 7:1, or any combination thereof).

Aspect E30: The method of aspect E4, or any preceding aspect, wherein the one or more oligosaccharides comprises glucose subunits and galactose subunits, the one or more oligosaccharides comprises a ratio of glucose terminal linkages to galactose terminal linkages of 2:1 to 4:1 (e.g., 2:1, 2.5:1, 3:1, 3.1:1, 3.2:1, 3.3:1, 3.5:1, 3.7:1, 4:1, or any combination thereof).

Aspect E31: The method of aspect E4, or any preceding aspect, wherein the one or more oligosaccharides comprises glucose subunits having glucose 3-linkages, wherein the glucose 3-linkages are beta linked.

Aspect E32: The method of aspect E4, or any preceding aspect, wherein the one or more oligosaccharides comprises glucose subunits having glucose 4-linkages, wherein the glucose 4-linkages are beta linked.

Aspect E33: The method of aspect E4, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX110 set forth in Table F, CLX115 set forth in Table P, CLX115Cu set forth in Table V, CLX112 set forth in Table H, or any combination thereof.

Aspect E34: The method of aspect E4, or any preceding aspect, wherein the one or more oligosaccharides comprises at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table F, Table P, Table V, or Table H, based on total mass of the one or more oligosaccharides and total mass of the compounds set forth in Table F, Table P, Table V, or Table H, respectively.

Aspect E35: The method of aspect E4, or any preceding aspect, wherein the one or more oligosaccharides comprises one or more 2D NMR correlations within 30% (e.g., within 30%, or within 10%, within 15%, within 20%, or within 25%) of the 2D NMR correlations set forth in Table A for CLX110, CLX112, CLX115, or CLX115Cu.

Aspect E36: The method of aspect E4, or any preceding aspect, wherein the one or more oligosaccharides has a dynamic viscosity ranging from 0.6 to 1.2 mPa*s (e.g., 0.6 to 1.2 mPa*s, 0.8 to 1.2 mPa*s, or 1 to 1.2 mPa*s) at a concentration of 10 mg/ml in water at a temperature of 25° C.

Aspect E37: The method of aspect E4, or any preceding aspect, wherein the one or more oligosaccharides have a dynamic viscosity ranging from 0.7 to 3 mPa*s (e.g., 0.7 to 3 mPa*s, 1 to 3 mPa*s, 1.5 to 3 mPa*s, 2 to 3 mPa*s, 0.7 to 2.5 mPa*s, or 0.7 to 2 mPa*s) at a concentration of 50 mg/ml in water at a temperature of 25° C.

Aspect E38: The method of aspect E4, or any preceding aspect, wherein the one or more oligosaccharides have a dynamic viscosity ranging from 0.7 to 8 mPa*s (e.g., 0.7 to 8 mPa*s, 0.7 to 7 mPa*s, 0.7 to 5 mPa*s, 1 to 8 mPa*s, 2 to 8 mPa*s, or 0.7 to 6 mPa*s) at a concentration of 100 mg/ml in water at a temperature of 25° C.

Aspect E39: The method of aspect E4, or any preceding aspect, wherein the one or more oligosaccharides comprises CLX110, CLX112, CLX115, CLX115Cu, or a combination thereof.

Aspect E40: The method of aspect E4, or any preceding aspect wherein the one or more oligosaccharides comprises CLX115.

Aspect E41: The method of aspect E4, or any preceding aspect wherein the one or more oligosaccharides comprises CLX115Cu.

Aspect E42: The method of any one of aspects A1-E41, wherein the one or more materials comprises xanthan gum; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX123 set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX123 set forth in Table Q; or
any combination thereof.

Aspect E43: The method of any one of aspects A1-E41, wherein the one or more materials comprises xanthan gum.

Aspect E44: The method of aspect E43, or any preceding aspect, wherein the one or more oligosaccharides contains beta-1,4 linked glucose residues.

Aspect E45: The method of aspect E43, or any preceding aspect, wherein the one or more oligosaccharides has an average molecular weight (Mw) of less than 10,000 Da. For example, the one or more oligosaccharides has an average molecular weight (Mw) within the range of 1,000-10,000 Da, 2,000-10,000 Da, 2,5000-10,000 Da, 3,000-10,000 Da, 4,000-10,000 Da, 5,000-10,000 Da, 7,500-10,000 Da, or 8,000-10,000 Da.

Aspect E46: The method of aspect E43, or any preceding aspect, wherein the one or more oligosaccharides has an average molecular weight (Mw) of less than 8,000 Da. For example, the one or more oligosaccharides has an average molecular weight (Mw) within the range of 1,000-8,000 Da, 2,000-8,000 Da, 2,5000-8,000 Da, 3,000-8,000 Da, 4,000-8,000 Da, 5,000-8,000 Da, or 6,000-8,000 Da.

Aspect E47: The method of aspect E43, or any preceding aspect, wherein the one or more oligosaccharides has a dynamic viscosity ranging from 1 to 10 mPa*s at 100 mg/ml at 25° C. . . . For example, the one or more oligosaccharides has a dynamic viscosity ranging from 1 to 10 mPa*s at 100 mg/ml at 25° C., 1.5 to 10 mPa*s at 100 mg/ml at 25° C., 1.8 to 10 mPa*s at 100 mg/ml at 25° C., 2 to 10 mPa*s at 100 mg/ml at 25° C., 2.5 to 10 mPa*s at 100 mg/ml at 25° C., 1 to 8 mPa*s at 100 mg/ml at 25° C., or 1.5 to 8 mPa*s at 100 mg/ml at 25° C.

Aspect E48: The method of aspect E43, or any preceding aspect, wherein at least 70% (e.g., within the range of 70-100%, within the range of 80-100%, or within the range of 90-100%) of the mass of the one or more oligosaccharides has a molecular mass of less than 100 kDa (e.g., within the range of 1-100 kDa, 1-80 kDa, 1-60 kDa, 1-50 kDa, 1-25 kDa, 5-100 kDa, 5-80 kDa, 10-100 kDa, or 10-80 kDa).

Aspect E49: The method of aspect E43, or any preceding aspect, wherein at least 60% (e.g., within the range of 60-100%, within the range of 70-100%, within the range of 80-100%, or within the range of 90-100%) of the mass of the one or more oligosaccharides has a molecular mass of less than 50 kDa (e.g., within the range of 1-50 kDa, 1-40 kDa, 1-25 kDa, 5-50 kDa, 5-25 kDa, or 10-50 kDa).

Aspect E50: The method of aspect E43, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 15 kDa (e.g., within the range of 1-15 kDa, 1-12 kDa, 1-10 kDa, 1-8 kDa, or 1-5 kDa).

Aspect E51: The method of aspect E43, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 5 kDa (e.g., within the range of 1-5 kDa, 1.2-5 kDa, 1.5-5 kDa, 1.8-5 kDa, or 2-5 kDa).

Aspect E52: The method of aspect E43, or any preceding aspect, wherein the one or more materials comprises Xanthomonas campestris extract.

Aspect E53: The method of aspect E43, or any preceding aspect, wherein at least 10 wt. %, (e.g., within the range of 10-100 wt. %, within the range of 20-100 wt. %, within the range of 30-100 wt. %, within the range of 50-100 wt. %, within the range of 75-100 wt. %, or 100 wt. %) of the one or more oligosaccharides comprises a degree of polymerization between 2 and 30 (e.g., between 2 and 30, between 2 and 20, between 2 and 15, between 2 and 10, between 3 and 30, between 3 and 20, between 4 and 30, or between 4 and 20) based on total mass of the one or more oligosaccharides.

Aspect E54: The method of aspect E43, or any preceding aspect, wherein the one or more oligosaccharides contain at least 10% (e.g., within the range of 10-100%, within the range of 20-100%, within the range of 30-100%, within the range of 50-100%, or within the range of 75-100%) glucose subunits.

Aspect E55: The method of aspect E54, or any preceding aspect, wherein the one or more oligosaccharides further comprises galactose subunits, mannose subunits, glucuronic acid subunits, or a combination thereof.

Aspect E56: The method of aspect E55, or any preceding aspect, wherein the galactose subunits are present in an amount of at least 1% (e.g., within the range of 1-90%, within the range of 1-50%, within the range of 1-30%, within the range of 1-20%, within the range of 1-12%, within the range of 3-90%, within the range of 3-50%, within the range of 3-30%, within the range of 3-20%, or within the range of 5-90%), and wherein the mannose subunits are present in an amount of at least 1% (e.g., within the range of 1-90%, within the range of 1-50%, within the range of 1-31%, within the range of 1-30%, within the range of 1-20%, within the range of 1-12%, within the range of 3-90%, within the range of 3-50%, within the range of 3-30%, within the range of 3-20%, or within the range of 5-90%), and wherein the glucuronic acid subunits are present in an amount of at least 1% (e.g., within the range of within the range of 1-90%, within the range of 1-50%, within the range of 1-20%, within the range of 1-10%, within the range of 1-5%, or within the range of 1-4%)

Aspect E57: The method of aspect E43, or any preceding aspect, wherein the one or more oligosaccharides comprises glucose subunits and glucuronic acid subunits, wherein the glucuronic acid:glucose ratio is between 1:15 to 1:25 (e.g., 1:15, 1:16, 1:17, 1:18, 1:19, 1:20, 1:21, 1:22, 1:23, 1:24, 1:25, or any combination thereof).

Aspect E58: The method of aspect E43, or any preceding aspect, wherein the one or more oligosaccharides comprises glucose subunits and mannose subunits, wherein the mannose:glucose ratio is between 0.1:1 to 1:3 (e.g., 0.1:1, 0.5:1, 1:1, 1:1.5, 1:2, 1:2.5, 1:3, or any combination thereof).

Aspect E59: The method of aspect E43, or any preceding aspect, wherein the one or more oligosaccharides comprises mannose subunits, wherein the mannose subunits comprise 2-linkages, 4,6-linkages, and terminal linkages in a total amount of at least 10% (e.g., within the range of 10-100%, within the range of 20-100%, within the range of 30-100%, within the range of 50-100%, or within the range of 75-100%).

Aspect E60: The method of aspect E43, or any preceding aspect, wherein the one or more oligosaccharides comprises glucose subunits, wherein the glucose subunits comprise 3-linkages, 4-linkages, and terminal linkages in a total amount of at least 10% (e.g., within the range of 10-100%, within the range of 20-100%, within the range of 30-100%, within the range of 50-100%, or within the range of 75-100%).

Aspect E61: The method of aspect E43, or any preceding aspect, wherein the one or more oligosaccharides comprises glucuronic acid subunits, wherein the glucuronic acid subunits comprise 4-linkages in a total amount of at least 10% (e.g., within the range of 10-100%, within the range of 20-100%, within the range of 30-100%, within the range of 50-100%, or within the range of 75-100%).

Aspect E62: The method of aspect E43, or any preceding aspect, wherein the one or more oligosaccharides comprises galactose subunits, wherein the galactose subunits comprise 3,6-linkages in a total amount of at least 10% (e.g., within the range of 10-100%, within the range of 20-100%, within the range of 30-100%, within the range of 50-100%, or within the range of 75-100%).

Aspect E63: The method of aspect E60, or any preceding aspect, wherein the glucose subunits comprise a ratio of 3-linkages:4-linkages:terminal linkages of 1:10:4.

Aspect E64: The method of aspect E59, or any preceding aspect, wherein the mannose subunits comprise a ratio of 2-linkages:4,6-linkages:terminal linkages of 8:3:3.

Aspect E65: The method of aspect E43, or any preceding aspect, wherein the one or more oligosaccharides comprises glucose subunits, wherein the glucose subunits comprise a ratio of 4-linkages:terminal linkages of 5:2.

Aspect E66: The method of aspect E43, or any preceding aspect, wherein the one or more oligosaccharides comprises glucose subunits, wherein the glucose subunits comprise a ratio of 3-linkages:4-linkages of 1:10.

Aspect E67: The method of aspect E43, or any preceding aspect, wherein the one or more oligosaccharides comprises mannose subunits, wherein the mannose subunits comprise a ratio of 2-linkages:4,6-linkages of 8:3.

Aspect E68: The method of aspect E43, or any preceding aspect, wherein the one or more oligosaccharides comprises glucose subunits and mannose subunits, wherein the one or more oligosaccharides comprises a ratio of glucose 4-linkages:mannose 2-linkages of 15:8.

Aspect E69: The method of aspect E43, or any preceding aspect, wherein the one or more oligosaccharides comprises glucose subunits and galactose subunits, wherein the one or more oligosaccharides comprises a ratio of glucose 4-linkages:galactose 3,6-linkages of 5:2.

Aspect E70: The method of aspect E43, or any preceding aspect, wherein the one or more oligosaccharides comprises glucose subunits having glucose 3-linkages, wherein the glucose 3-linkages are beta linked.

Aspect E71: The method of aspect E43, or any preceding aspect, wherein the one or more oligosaccharides comprises glucose subunits having glucose 4-linkages, wherein the glucose 4-linkages are beta linked.

Aspect E72: The method of aspect E43, or any preceding aspect, wherein the one or more oligosaccharides comprises mannose subunits having mannose 2-linkages and mannose 4,6 linkages, wherein the mannose 2-linkages and mannose 4,6 linkages are alpha linked.

Aspect E73: The method of aspect E43, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX123 set forth in Table Q.

Aspect E74: The method of aspect E43, or any preceding aspect, wherein the one or more oligosaccharides comprises at least 30 wt. %, (e.g., within the range of 30-100 wt. %, within the range of 40-100 wt. %, within the range of 50-100 wt. %, within the range of 75-100 wt. %, or within the range of 85-100 wt. %) of the compounds set forth in Table Q based on total mass of the one or more oligosaccharides and total mass of the compounds set forth in Table Q.

Aspect E75: The method of aspect E43, or any preceding aspect, wherein the one or more oligosaccharides comprises one or more 2D NMR correlations within 30% (e.g., within 30%, or within 10%, within 15%, within 20%, or within 25%) of the 2D NMR correlations set forth in Table A for CLX123.

Aspect E76: The method of aspect E43, or any preceding aspect, wherein the one or more oligosaccharides has a dynamic viscosity ranging from 1 to 1.8 mPa*s at a concentration of 100 mg/ml in water at a temperature of 25° C. For example, the one or more oligosaccharides has a dynamic viscosity ranging from 1 to 1.8 mPa*s at 100 mg/ml at 25° C., 1 to 1.6 mPa*s at 100 mg/ml at 25° C., 1 to 1.5 mPa*s at 100 mg/ml at 25° C., or 1.2 to 1.8 mPa*s at 100 mg/ml at 25° C.

Aspect E77: The method of aspect E43, or any preceding aspect, wherein the one or more oligosaccharides have a dynamic viscosity of 1.6 mPa*s at a concentration of 100 mg/ml in water at a temperature of 25° C.

Aspect E78: The method of aspect E43, or any preceding aspect, wherein the one or more oligosaccharides comprises CLX123.

Aspect E79: The method of any one of aspect A1-E78, wherein the one or more materials comprises sugar cane; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX128 set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX128 set forth in Table T; or
any combination thereof.

Aspect E80: The method of any one of aspects A1-E79, wherein the one or more materials comprises sugar cane.

Aspect E81: The method of aspect E80, or any preceding aspect, wherein the one or more oligosaccharides contains 4-linked xylose residues.

Aspect E82: The method of aspect E80, or any preceding aspect, wherein the one or more oligosaccharides has an average molecular weight (Mw) of less than 10,000 Da. For example, the one or more oligosaccharides has an average molecular weight (Mw) within the range of 1,000-10,000 Da, 2,000-10,000 Da, 2,5000-10,000 Da, 3,000-10,000 Da, 4,000-10,000 Da, 5,000-10,000 Da, 7,500-10,000 Da, or 8,000-10,000 Da.

Aspect E83: The method of aspect E80, or any preceding aspect, wherein the one or more oligosaccharides has an average molecular weight (Mw) of less than 8,000 Da. For example, the one or more oligosaccharides has an average molecular weight (Mw) within the range of 1,000-8,000 Da, 2,000-8,000 Da, 2,5000-8,000 Da, 3,000-8,000 Da, 4,000-8,000 Da, 5,000-8,000 Da, or 6,000-8,000 Da.

Aspect E84: The method of aspect E80, or any preceding aspect, wherein the one or more oligosaccharides has a dynamic viscosity ranging from 1 to 10 mPa*s at 100 mg/ml at 25° C. For example, the one or more oligosaccharides has a dynamic viscosity ranging from 1 to 10 mPa*s at 100 mg/ml at 25° C., 1.5 to 10 mPa*s at 100 mg/ml at 25° C., 1.8 to 10 mPa*s at 100 mg/ml at 25° C., 2 to 10 mPa*s at 100 mg/ml at 25° C., 2.5 to 10 mPa*s at 100 mg/ml at 25° C., 1 to 8 mPa*s at 100 mg/ml at 25° C., or 1.5 to 8 mPa*s at 100 mg/ml at 25° C.

Aspect E85: The method of aspect E80, or any preceding aspect, wherein at least 70% (e.g., within the range of 70-100%, within the range of 80-100%, or within the range of 90-100%) of the mass of the one or more oligosaccharides has a molecular mass of less than 100 kDa (e.g., within the range of 1-100 kDa, 1-80 kDa, 1-60 kDa, 1-50 kDa, 1-25 kDa, 5-100 kDa, 5-80 kDa, 10-100 kDa, or 10-80 kDa).

Aspect E86: The method of aspect E80, or any preceding aspect, wherein at least 60% (e.g., within the range of 60-100%, within the range of 70-100%, within the range of 80-100%, or within the range of 90-100%) of the mass of the one or more oligosaccharides has a molecular mass of less than 50 kDa (e.g., within the range of 1-50 kDa, 1-40 kDa, 1-25 kDa, 5-50 kDa, 5-25 kDa, or 10-50 kDa).

Aspect E87: The method of aspect E80, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 15 kDa (e.g., within the range of 1-15 kDa, 1-12 kDa, 1-10 kDa, 1-8 kDa, or 1-5 kDa).

Aspect E88: The method of aspect E80, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 5 kDa (e.g., within the range of 1-5 kDa, 1.2-5 kDa, 1.5-5 kDa, 1.8-5 kDa, or 2-5 kDa).

Aspect E89: The method of aspect E80, or any preceding aspect, wherein at least 10 wt. % (e.g., within the range of 10-100 wt. %, within the range of 20-100 wt. %, within the range of 30-100 wt. %, within the range of 50-100 wt. %, within the range of 75-100 wt. %, or 100 wt. %) of the one or more oligosaccharides comprises a degree of polymerization between 2 and 30 (e.g., between 2 and 30, between 2 and 20, between 2 and 15, between 2 and 10, between 3 and 30, between 3 and 20, between 4 and 30, or between 4 and 20) based on total mass of the one or more oligosaccharides.

Aspect E90: The method of aspect E80, or any preceding aspect, wherein the one or more oligosaccharides contain at least 10% (e.g., within the range of 10-100%, within the range of 10-40%, within the range of 10-47%, within the range of 10-50%, within the range of 20-100%, within the range of 30-100%, within the range of 40-100%) xylose subunits.

Aspect E91: The method of aspect E80, or any preceding aspect, wherein the one or more oligosaccharides contain at least 10% (e.g., within the range of 10-100%, within the range of 10-35%, within the range of 10-50%, within the range of 20-100%, within the range of 30-100%, within the range of 35-100%) glucose subunits.

Aspect E92: The method of aspect E90, or any preceding aspect, wherein the one or more oligosaccharides further comprises galactose subunits, glucose subunits, arabinose subunits, or a combination thereof.

Aspect E93: The method of aspect E92, or any preceding aspect, wherein the galactose subunits are present in an amount of at least 1% (e.g., within the range of 1-90%, within the range of 1-50%, within the range of 1-30%, within the range of 1-20%, within the range of 1-10%, or within the range of 1-5%), and wherein the glucose subunits are present in an amount of at least 1% (e.g., within the range of 1-90%, within the range of 1-50%, within the range of 1-35%, within the range of 3-90%, within the range of 3-50%, within the range of 3-35%, or within the range of 5-90%), and wherein the arabinose subunits are present in an amount of at least 1% (e.g., within the range of 1-90%, within the range of 1-50%, within the range of 1-30%, within the range of 1-20%, within the range of 1-12%, range of 3-90%, within the range of 3-50%, within the range of 3-30%, within the range of 3-20%, or within the range of 5-90%).

Aspects E94: The method of aspect E80, or any preceding aspect, wherein the one or more oligosaccharides comprises xylose subunits and glucose subunits, wherein the xylose:glucose ratio is 10:7.

Aspect E95: The method of aspect E80, or any preceding aspect, wherein the one or more oligosaccharides comprises xylose subunits and arabinose subunits, wherein the xylose:arabinose ratio is 4:1.

Aspect E96: The method of aspect E90, or any preceding aspect, wherein the xylose subunits comprise 4-linkages, 3,4-linkages, and terminal linkages in a total amount of at least 10% (e.g., within the range of 10-100%, within the range of 20-100%, within the range of 30-100%, within the range of 50-100%, or within the range of 75-100%).

Aspect E97: The method of aspect E91, or any preceding aspect, wherein the glucose subunits comprise 3-linkages, 4-linkages, and terminal linkages in a total amount of at least 10% (e.g., within the range of 10-100%, within the range of 20-100%, within the range of 30-100%, within the range of 50-100%, or within the range of 75-100%).

Aspect E98: The method of aspect E80, or any preceding aspect, wherein the one or more oligosaccharides comprises arabinose subunits, wherein the arabinose subunits comprise 5-linkages and terminal linkages in a total amount of at least 10% (e.g., within the range of 10-100%, within the range of 20-100%, within the range of 30-100%, within the range of 50-100%, or within the range of 75-100%).

Aspect E99: The method of claim 103, wherein the xylose subunits comprise a ratio of 4-linkages:3,4-linkages:terminal linkages of 9:1.7:2.

Aspect E100: The method of aspect E80, or any preceding aspect, wherein the one or more oligosaccharides comprises arabinose subunits, wherein the arabinose subunits comprise a ratio of 5-linkages:terminal linkages of 7:8.

Aspect E101: The method of claim 104, wherein the glucose subunits comprise a ratio of 3-linkages:4-linkages:terminal linkages of 1:5:3.

Aspect E102: The method of claim 103, wherein the xylose subunits comprise a ratio of 4-linkages:3,4-linkages of 9:1.7.

Aspect E103: The method of aspect E90, or any preceding aspect, wherein the xylose subunits comprise a ratio of 4-linkages:terminal linkages of 9:2.

Aspect E104: The method of aspect E91, or any preceding aspect, wherein the glucose subunits comprise a ratio of 4-linkages:terminal linkages of 5:3.

Aspect E105: The method of aspect E91, or any preceding aspect, wherein the glucose subunits comprise a ratio of 4-linkages:3-linkages of 5:1.

Aspect E106: The method of aspect E80, or any preceding aspect, wherein the one or more oligosaccharides comprises xylose subunits and glucose subunits, wherein the one or more oligosaccharides comprises a ratio of xylose 4-linkages:glucose 4-linkages of 9:5.

Aspect E107: The method of aspect E80, or any preceding aspect, wherein the one or more oligosaccharides comprises xylose subunits having xylose 4-linkages, wherein the xylose 4-linkages are beta linked.

Aspect E108: The method of aspect E80, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX128 set forth in Table T.

Aspect E109: The method of aspect E80, or any preceding aspect, wherein the one or more oligosaccharides comprises at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table T based on total mass of the one or more oligosaccharides and total mass of the compounds set forth in Table T.

Aspect E110: The method of aspect E80, or any preceding aspect, wherein the one or more oligosaccharides comprises one or more 2D NMR correlations within 30% (e.g., within 30%, or within 10%, within 15%, within 20%, or within 25%) of the 2D NMR correlations set forth in Table A for CLX128.

Aspect E111: The method of aspect E80, or any preceding aspect, wherein the one or more oligosaccharides has a dynamic viscosity ranging from 1 to 2 mPa*s at a concentration of 100 mg/ml in water at a temperature of 25° C.

Aspect E112: The method of aspect E80, or any preceding aspect, wherein the one or more oligosaccharides has a dynamic viscosity of 1.8 mPa*s at a concentration of 100 mg/ml in water at a temperature of 25° C.

Aspect E113: The method of aspect E80, or any preceding aspect, wherein the one or more oligosaccharides comprises CLX128.

Aspect E114: The method of any one of aspects A1-E113, wherein the one or more materials comprises soy; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX126 set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX126 set forth in Table S; or
any combination thereof.

Aspect E115: The method of any one of aspects A1-E114, wherein the one or more materials comprises sugar beets, legumes, or a combination thereof.

Aspect E116: The method of aspect E115, or any preceding aspect, wherein the one or more materials comprises the legumes, and the legumes comprise soy, pea, tree nuts, or a combination thereof.

Aspect E117: The method of aspect E115, or any preceding aspect, wherein the one or more materials comprises legumes, wherein the legumes comprise soy.

Aspect E118: The method of aspect E117, or any preceding aspect, wherein the one or more oligosaccharides has an average molecular weight (Mw) of less than 10,000 Da. For example, the one or more oligosaccharides has an average molecular weight (Mw) within the range of 1,000-10,000 Da, 2,000-10,000 Da, 2,5000-10,000 Da, 3,000-10,000 Da, 4,000-10,000 Da, 5,000-10,000 Da, 7,500-10,000 Da, or 8,000-10,000 Da.

Aspect E119: The method of aspect E117, or any preceding aspect, wherein the one or more oligosaccharides has an average molecular weight (Mw) of less than 8,000 Da. For example, the one or more oligosaccharides has an average molecular weight (Mw) within the range of 1,000-8,000 Da, 2,000-8,000 Da, 2,5000-8,000 Da, 3,000-8,000 Da, 4,000-8,000 Da, 5,000-8,000 Da, or 6,000-8,000 Da.

Aspect E120: The method of aspect E117, or any preceding aspect, wherein the one or more oligosaccharides has a dynamic viscosity ranging from 1 to 10 mPa*s at 100 mg/ml at 25° C. For example, the one or more oligosaccharides has a dynamic viscosity ranging from 1 to 10 mPa*s at 100 mg/ml at 25° C., 1.5 to 10 mPa*s at 100 mg/ml at 25° C., 1.8 to 10 mPa*s at 100 mg/ml at 25° C., 2 to 10 mPa*s at 100 mg/ml at 25° C., 2.5 to 10 mPa*s at 100 mg/ml at 25° C., 1 to 8 mPa*s at 100 mg/ml at 25° C., or 1.5 to 8 mPa*s at 100 mg/ml at 25° C.

Aspect E121: The method of aspect E117, or any preceding aspect, wherein at least 70% (e.g., within the range of 70-100%, within the range of 80-100%, or within the range of 90-100%) of the mass of the one or more oligosaccharides has a molecular mass of less than 100 kDa (e.g., within the range of 1-100 kDa, 1-80 kDa, 1-60 kDa, 1-50 kDa, 1-25 kDa, 5-100 kDa, 5-80 kDa, 10-100 kDa, or 10-80 kDa).

Aspect E122: The method of aspect E117, or any preceding aspect, wherein at least 60% (e.g., within the range of 60-100%, within the range of 70-100%, within the range of 80-100%, or within the range of 90-100%) of the mass of the one or more oligosaccharides has a molecular mass of less than 50 kDa (e.g., within the range of 1-50 kDa, 1-40 kDa, 1-25 kDa, 5-50 kDa, 5-25 kDa, or 10-50 kDa).

Aspect E123: The method of aspect E117, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 15 kDa (e.g., within the range of 1-15 kDa, 1-12 kDa, 1-10 kDa, 1-8 kDa, or 1-5 kDa).

Aspect E124: The method of aspect E117, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 5 kDa (e.g., within the range of 1-5 kDa, 1.2-5 kDa, 1.5-5 kDa, 1.8-5 kDa, or 2-5 kDa).

Aspect E125: The method of aspect E117, or any preceding aspect, wherein the one or more oligosaccharides contains at least 30% (e.g., within the range of 30-100%, within the range of 30-80%, within the range of 30-60%, within the range of 30-50%, or within the range of 40-100%) galactose residues.

Aspect E126: The method of aspect E125, or any preceding aspect, wherein the one or more oligosaccharides further comprises arabinose subunits, xylose subunits, galacturonic acid subunits, glucose subunits, rhamnose subunits, fucose subunits, or a combination thereof.

Aspect E127: The method of aspect E126, or any preceding aspect, wherein the arabinose subunits are present in an amount of at least 1% (e.g., within the range of 1-50%, within the range of 1-35%, within the range of 1-34%, within the range of 3-50%, or within the range of 3-35%), and wherein the xylose subunits are present in an amount of at least 1% (e.g., within the range of 1-50%, within the range of 1-30%, within the range of 1-20%, within the range of 1-10%, or within the range of 1-5%), and wherein the galacturonic acid subunits are present in an amount of at least 1% (e.g., within the range of 1-50%, within the range of 1-30%, within the range of 1-20%, within the range of 1-10%, or within the range of 1-3%), and wherein the glucose subunits are present in an amount of at least 1%, and wherein the fucose subunits are present in an amount of at least 1% (e.g., within the range of 1-50%, within the range of 1-30%, within the range of 1-20%, within the range of 1-10%, or within the range of 1-2%), and wherein the rhamnose subunits are present in an amount of least 1% (e.g., within the range of 1-50%, within the range of 1-30%, within the range of 1-20%, within the range of 1-10%, or within the range of 1-2%).

Aspect E128: The method of aspect E117, or any preceding aspect, wherein the one or more oligosaccharides comprises galactose subunits and arabinose subunits, wherein the one or more oligosaccharides comprises a ratio of galactose 4-linkages:arabinose terminal linkages of 3:1.

Aspect E129: The method of aspect E117, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX126 set forth in Table S.

Aspect E130: The method of aspect E117, or any preceding aspect, wherein the one or more oligosaccharides comprises at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table S based on total mass of the one or more oligosaccharides and total mass of the compounds set forth in Table S.

Aspect E131: The method of aspect E117, or any preceding aspect, wherein the one or more oligosaccharides comprises one or more 2D NMR correlations within 30% (e.g., within 30%, or within 10%, within 15%, within 20%, or within 25%) of the 2D NMR correlations set forth in Table A for CLX126.

Aspect E132: The method of aspect E117, or any preceding aspect, wherein the one or more oligosaccharides have a dynamic viscosity of 4.4 mPa*s at a concentration of 100 mg/ml in water at a temperature of 25° C.

Aspect E133: The method of aspect E117, or any preceding aspect, wherein the one or more oligosaccharides comprises CLX126.

Aspect E134: The method of any one of aspects A1-E133, wherein the one or more materials contains at least 30% (e.g., within the range of 30-100%, within the range of 40-100%, within the range of 50-100%, within the range of 75-100%, or within the range of 85-100%) arabinose subunits.

Aspect E135: The method of aspect E134, or any preceding aspect, wherein the one or more materials further comprises galactose subunits, xylose subunits, glucose subunits, or any combination thereof.

Aspect E136: The method of aspect E134, or any preceding aspect, wherein the one or more oligosaccharides comprises arabinose subunits and galactose subunits, wherein the arabinose:galactose ratio is 4.5:1 to 7:1 (e.g., 4.5:1, 5:1, 5.5:1, 5.9:1, 6:1, 6.5:1, 7:1, or any combination thereof).

Aspect E137: The method of aspect E134, or any preceding aspect, wherein the one or more oligosaccharides comprises galactose subunits and xylose subunits, wherein the galactose:xylose ratio is 2.4:1 to 4.5:1 (e.g., 2.4:1, 2.7:1, 3:1, 3.1:1, 3.5:1, 4:1, 4.5:1, or any combination thereof).

Aspect E138: The method of aspect E134, or any preceding aspect, wherein the one or more oligosaccharides comprises xylose subunits and glucose subunits, wherein the xylose:glucose ratio is 0.5:1 to 3:1 (e.g., 0.5:1, 1:1, 1.2:1, 1.4:1, 2:1, 2.5:1, 3:1, or any combination thereof).

Aspect E139: The method of aspect E134, or any preceding aspect, wherein the one or more oligosaccharides comprises arabinose subunits, wherein the arabinose subunits comprise 5-linkages, 3,5-linkages, and terminal linkages in a total amount of at least 10% (e.g., within the range of 10-100%, within the range of 20-100%, within the range of 30-100%, within the range of 50-100%, or within the range of 75-100%).

Aspect E140: The method of aspect E134, or any preceding aspect, wherein the one or more oligosaccharides comprises arabinose subunits, wherein the arabinose subunits comprise a ratio of 3,5-linkages to 5-linkages of 1:1 to 2.3:1 (e.g., 1:1, 1.2:1, 1.4:1, 1.8:1, 2:1, 2.3:1, or any combination thereof).

Aspect E141: The method of aspect E134, or any preceding aspect, wherein the one or more oligosaccharides comprises arabinose subunits, wherein the arabinose subunits comprise a ratio of terminal linkages to 3,5-linkages of 0.5:1 to 3:1 (e.g., 0.5:1, 0.8:1, 1:1, 1.2:1, 1.3:1, 1.5:1, 2:1, 2.5:1, 3:1, or any combination thereof).

Aspect E142: The method of aspect E134, or any preceding aspect, wherein the one or more oligosaccharides comprises arabinose subunits and galactose subunits, wherein the arabinose subunits and the galactose subunits comprise a ratio of arabinose 3,5-linkages to galactose 4-linkages of 3:1 to 6:1 (e.g., 3:1, 3.5:1, 4:1, 4.5:1, 4.9:1, 5:1, 5.5:1, 6:1, or any combination thereof).

Aspect E143: The method of aspect E134, or any preceding aspect, wherein the one or more oligosaccharides comprises arabinose subunits and galactose subunits, wherein the arabinose subunits and the galactose subunits comprise a ratio of arabinose terminal linkages to galactose 4-linkages of 4:1 to 8.5:1 (e.g., 4:1, 4.5:1, 5:1, 5.5:1, 6:1, 6.5:1, 6.6:1, 7:1, 7.5:1, 8:1, 8.5:1, or any combination thereof).

Aspect E144: The method of aspect E134, or any preceding aspect, wherein the one or more oligosaccharides comprises CLX096.

Aspect E145: The method of any one of aspects A1-E144, wherein the one or more materials comprises gellan gum; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX125 set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX125 set forth in Table R; or
any combination thereof.

Aspect E146: The method of any one of aspects A1-E145, wherein the one or more materials comprises gellan gum.

Aspect E147: The method of aspect E146, or any preceding aspect, wherein the one or more oligosaccharides contains 4-linked glucose subunits, 3-linked glucose subunits, and 4-linked rhamnose subunits.

Aspect E148: The method of aspect E146, or any preceding aspect, wherein the one or more oligosaccharides has an average molecular weight (Mw) of less than 10,000 Da. For example, the one or more oligosaccharides has an average molecular weight (Mw) within the range of 1,000-10,000 Da, 2,000-10,000 Da, 2,5000-10,000 Da, 3,000-10,000 Da, 4,000-10,000 Da, 5,000-10,000 Da, 7,500-10,000 Da, or 8,000-10,000 Da.

Aspect E149: The method of aspect E146, or any preceding aspect, wherein the one or more oligosaccharides has an average molecular weight (Mw) of less than 8,000 Da. For example, the one or more oligosaccharides has an average molecular weight (Mw) within the range of 1,000-8,000 Da, 2,000-8,000 Da, 2,5000-8,000 Da, 3,000-8,000 Da, 4,000-8,000 Da, 5,000-8,000 Da, or 6,000-8,000 Da.

Aspect E150: The method of aspect E146, or any preceding aspect, wherein the one or more oligosaccharides has a dynamic viscosity ranging from 1 to 10 mPa*s at 100 mg/ml at 25° C. For example, the one or more oligosaccharides has a dynamic viscosity ranging from 1 to 10 mPa*s at 100 mg/ml at 25° C., 1.5 to 10 mPa*s at 100 mg/ml at 25° C., 1.8 to 10 mPa*s at 100 mg/ml at 25° C., 2 to 10 mPa*s at 100 mg/ml at 25° C., 2.5 to 10 mPa*s at 100 mg/ml at 25° C., 1 to 8 mPa*s at 100 mg/ml at 25° C., or 1.5 to 8 mPa*s at 100 mg/ml at 25° C.

Aspect E151: The method of aspect E146, or any preceding aspect, wherein at least 70% (e.g., within the range of 70-100%, within the range of 80-100%, or within the range of 90-100%) of the mass of the one or more oligosaccharides has a molecular mass of less than 100 kDa (e.g., within the range of 1-100 kDa, 1-80 kDa, 1-60 kDa, 1-50 kDa, 1-25 kDa, 5-100 kDa, 5-80 kDa, 10-100 kDa, or 10-80 kDa).

Aspect E152: The method of aspect E146, or any preceding aspect, wherein at least 60% (e.g., within the range of 60-100%, within the range of 70-100%, within the range of 80-100%, or within the range of 90-100%) of the mass of the one or more oligosaccharides has a molecular mass of less than 50 kDa (e.g., within the range of 1-50 kDa, 1-40 kDa, 1-25 kDa, 5-50 kDa, 5-25 kDa, or 10-50 kDa).

Aspect E153: The method of aspect E146, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 15 kDa (e.g., within the range of 1-15 kDa, 1-12 kDa, 1-10 kDa, 1-8 kDa, or 1-5 kDa).

Aspect E154: The method of aspect E146, or any preceding aspect, wherein at least 50% (e.g., within the range of 50-100%, within the range of 60-100%, within the range of 70-100%, within the range of 80-100%, or within the range of 90-100%) of the mass of the one or more oligosaccharides has a molecular mass of less than 5 kDa (e.g., within the range of 1-5 kDa, 1.2-5 kDa, 1.5-5 kDa, 1.8-5 kDa, or 2-5 kDa).

Aspect E155: The method of aspect E146, or any preceding aspect, wherein at least 10 wt. % (e.g., within the range of 10-100 wt. %, within the range of 20-100 wt. %, within the range of 30-100 wt. %, within the range of 50-100 wt. %, within the range of 75-100 wt. %, or 100 wt. %) of the one or more oligosaccharides comprises a degree of polymerization between 2 and 30 (e.g., between 2 and 30, between 2 and 20, between 2 and 15, between 2 and 10, between 3 and 30, between 3 and 20, between 4 and 30, or between 4 and 20) based on total mass of the one or more oligosaccharides.

Aspect E156: The method of aspect E146, or any preceding aspect, wherein the one or more oligosaccharides contain at least 10% (e.g., within the range of 10-100%, within the range of 10-80%, within the range of 10-60%, within the range of 10-50%, within the range of 10-44%, within the range of 40-100%, or within 44-100%) glucose subunits.

Aspect E157: The method of aspect E146, or any preceding aspect, wherein the one or more oligosaccharides contain at least 10% (e.g., within the range of 10-100%, within the range of 10-80%, within the range of 10-60%, within the range of 10-50%, within the range of 10-43%, within the range of 40-100%, or within 43-100%) rhamnose subunits.

Aspect E158: The method of aspect E146, or any preceding aspect, wherein the one or more oligosaccharides further comprises rhamnose subunits, glucuronic acid subunits, or a combination thereof.

Aspect E159: The method of aspect E158, or any preceding aspect, wherein the glucose subunits are present in an amount of at least 1% (e.g., within the range of 1-50%, within the range of 1-44%, within the range of 1-40%, within the range of 3-50%, within the range of 3-40%, or within the range of 3-44%), and wherein the rhamnose subunits are present in an amount of at least 1% (e.g., within the range of 1-50%, within the range of 1-43%, within the range of 1-40%, within the range of 3-50%, within the range of 3-40%, or within the range of 3-43%), and wherein the glucuronic acid subunits are present in an amount of at least 1% (e.g., within the range of 1-50%, within the range of 1-20%, within the range of 1-10%, within the range of 1-8%, within the range of 3-40%, within the range of 3-10%, or within the range of 3-8%).

Aspect E160: The method of aspect E146, or any preceding aspect, wherein the one or more oligosaccharides comprises glucose subunits and rhamnose subunits, wherein the glucose:rhamnose ratio is 1:1.

Aspect E161: The method of aspect E146, or any preceding aspect, wherein the one or more oligosaccharides comprises glucose subunits and glucuronic acid subunits, wherein the glucose:glucuronic acid ratio is 5.5:1.

Aspect E162: The method of aspect E146, or any preceding aspect, wherein the one or more oligosaccharides comprises rhamnose subunits and glucuronic acid subunits, wherein the rhamnose:glucuronic acid ratio is 5.4:1.

Aspect E163: The method of aspect E156, or any preceding aspect, wherein the glucose subunits comprise 4-linkages, 3-linkages, and terminal linkages in a total amount of at least 10% (e.g., within the range of 10-100%, within the range of 20-100%, within the range of 30-100%, within the range of 50-100%, or within the range of 75-100%).

Aspect E164: The method of E157, or any preceding aspect, wherein the rhamnose subunits comprise 4-linkages and terminal linkages in a total amount of at least 10% (e.g., within the range of 10-100%, within the range of 20-100%, within the range of 30-100%, within the range of 50-100%, or within the range of 75-100%).

Aspect E165: The method of aspect E146, or any preceding aspect, wherein the one or more oligosaccharides comprises glucuronic acid subunits, wherein the glucuronic acid subunits comprise 4-linkages and terminal linkages in a total amount of at least 10% (e.g., within the range of 10-100%, within the range of 20-100%, within the range of 30-100%, within the range of 50-100%, or within the range of 75-100%).

Aspect E166: The method of aspect E156, or any preceding aspect, wherein the glucose subunits comprise a ratio of 4-linkages:3-linkages:terminal linkages of 8:7:3.3.

Aspect E167: The method of aspect E157, or any preceding aspect, wherein the rhamnose subunits comprise a ratio of 4-linkages:terminal linkages of 3.1:1.

Aspect E168: The method of aspect E156, or any preceding aspect, wherein the glucose subunits comprise a ratio of 4-linkages:terminal linkages of 8:3.3.

Aspect E169: The method of aspect E157, or any preceding aspect, wherein the glucose subunits comprise a ratio of 4-linkages:3-linkages of 8:7.

Aspect E170: The method of aspect E156, wherein the glucose subunits comprise a ratio of 3-linkages:terminal linkages of 7:3.3.

Aspect E171: The method of aspect E146, or any preceding aspect, wherein the one or more oligosaccharides comprises rhamnose subunits and glucose subunits, wherein the one or more oligosaccharides comprises a ratio of rhamnose 4-linkages:glucose 4-linkages of 11:12.

Aspect E172: The method of aspect E146, or any preceding aspect, wherein the one or more oligosaccharides comprises rhamnose subunits having rhamnose 4-linkages, wherein the rhamnose 4-linkages are beta linked.

Aspect E173: The method of aspect E146, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX125 set forth in Table R.

Aspect E174: The method of aspect E146, or any preceding aspect, wherein the one or more oligosaccharides comprises at least 30 wt. %, (e.g., within the range of 30-100 wt. %, within the range of 40-100 wt. %, within the range of 50-100 wt. %, within the range of 75-100 wt. %, or within the range of 85-100 wt. %) of the compounds set forth in Table R based on total mass of the one or more oligosaccharides and total mass of the compounds set forth in Table R.

Aspect E175: The method of aspect E146, or any preceding aspect, wherein the one or more oligosaccharides comprises one or more 2D NMR correlations within 30% (e.g., within 30%, or within 10%, within 15%, within 20%, or within 25%) of the 2D NMR correlations set forth in Table A for CLX125.

Aspect E176: The method of aspect E146, or any preceding aspect, wherein the one or more oligosaccharides has a dynamic viscosity having a range of 1 to 2 mPa*s at a concentration of 100 mg/ml in water at a temperature of 25° C.

Aspect E177: The method of aspect E146, or any preceding aspect, wherein the one or more oligosaccharides has a dynamic viscosity of 1.5 mPa*s at a concentration of 100 mg/ml in water at a temperature of 25° C.

Aspect E178: The method of aspect E146, or any preceding aspect, wherein the one or more oligosaccharides comprises CLX125.

Aspect E179: The method of any one of aspects A1-E178, wherein the one or more oligosaccharides comprise at least one glycosidic bond comprising galactose-mannose (α1-6), xylose-xylose (β1-4), arabinose-xylose (α1-2/3), xylose-glucose (α1-6), galactose-xylose (β1-2), glucose-glucose (β1-4), or any combination thereof.

Aspect E180: The method of any one of aspects A1-E179, wherein the one or more oligosaccharides comprise at least one glycosidic bond that can be depolymerized via glycosyl hydrolase families GH 36 with EC 3.2.1.22 activity, GH 8 with EC 3.2.1.4 activity, GH 10 with EC 3.2.1.8 activity, GH 31 with EC 3.2.1. 1717 activity, GH 51 with EC 3.2.1.55 activity, GH 2 with EC 3.2.1. 23 activity, or any combination thereof.

Aspect E181: The method of any one of aspects A1-E180, wherein the one or more materials comprises homo-xylan, glucuronoxylan, glucuronoarabinoxylan, arabinoxylan, or any combination thereof; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX103 set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX103 set forth in Table E; or
any combination thereof.

Aspects E182: The method of any one of aspects A1-E181, wherein the one or more materials comprises at least one of homo-xylan, glucuronoxylan, or a combination thereof.

Aspects E183: The method of aspect E182, or any preceding aspect, wherein the one or more materials comprise wood, cell walls of seeds, Poaceae, or any combination thereof.

Aspect E184: The method of aspect E183, or any preceding aspect, wherein the one or more materials comprise wood, and the wood comprises hardwood, beechwood, or a combination thereof.

Aspect E185: The method of aspect E183, or any preceding aspect, wherein the one or more materials comprises cell walls of seeds, and the cell walls of seeds comprise cell walls of seeds from fruit seeds, vegetable seeds, apple seeds, pear seeds, peeper seeds, tomato seeds, peach seeds, cherry seeds, or any combination thereof.

Aspect E186: The method of aspect E183, or any preceding aspect, wherein the one or more materials comprises Poaceae, and the Poaceae comprises corn cob, sugar cane, bamboo, cereal brans, grasses, switchgrass, or any combination thereof.

Aspect E187: The method of aspect E182, or any preceding aspect, wherein the one or more oligosaccharides comprise at least 30% (e.g., within the range of 30-100%, within the range of 40-100%, within the range of 50-100%, within the range of 75-100%, or within the range of 85-100%) xylose subunits.

Aspect E188: The method of aspect E187, or any preceding aspect, wherein the xylose subunits comprise 4-linkages and terminal linkages in a total amount of at least 10% (e.g., within the range of 10-100%, within the range of 20-100%, within the range of 30-100%, within the range of 50-100%, or within the range of 75-100%).

Aspect E189: The method of aspect E188, or any preceding aspect, wherein the ratio of the 4-linkages to the terminal linkages is 6.5:1 to 8.5:1 (e.g., 6.5:1, 7:1, 7.5:1, 7.6:1, 8:1, 8.5:1, or any combination thereof).

Aspect E190: The method of aspect E188, or any preceding aspect, wherein the 4-linkages are beta linked.

Aspect E191: The method of aspect E182, or any preceding aspect, wherein at least 10 wt. %, (e.g., within the range of 10-100 wt. %, within the range of 20-100 wt. %, within the range of 30-100 wt. %, within the range of 50-100 wt. %, within the range of 75-100 wt. %, or 100 wt. %) of the one or more oligosaccharides comprises a degree of polymerization between 2 and 30 (e.g., between 2 and 30, between 2 and 20, between 2 and 15, between 2 and 10, between 3 and 30, between 3 and 20, between 4 and 30, or between 4 and 20) based on total mass of the one or more oligosaccharides.

Aspect E192: The method of aspect E182, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX103 set forth in Table E.

Aspect E193: The method of aspect E182, or any preceding aspect, wherein the one or more oligosaccharides comprises at least 30 wt. %, (e.g., within the range of 30-100 wt. %, within the range of 40-100 wt. %, within the range of 50-100 wt. %, within the range of 75-100 wt. %, or within the range of 85-100 wt. %) of the compounds set forth in Table E, based on total mass of the one or more oligosaccharides and total mass of the compounds set forth in Table E.

Aspect E194: The method of aspect E182, or any preceding aspect, wherein the one or more oligosaccharides comprise one or more of the 2D NMR correlations within 30% (e.g., within 30%, or within 10%, within 15%, within 20%, or within 25%) of the values set forth in Table A for CLX103.

Aspect E195: The method of aspect E182, or any preceding aspect, wherein the one or more oligosaccharides have a dynamic viscosity of between 0.6 and 1.2 mPa*s at a concentration of 10 mg/ml in water at a temperature of 25° C.

Aspect E196: The method of aspect E182, or any preceding aspect, wherein the one or more oligosaccharides have a dynamic viscosity of between 0.7 and 3 mPa*s (e.g., 0.7 and 3 mPa*s, 0.7 and 2 mPa*s, 1.2 and 3 mPa*s, or 0.7 and 1.5 mPa*s) at a concentration of 50 mg/ml in water at a temperature of 25° C.

Aspect E197: The method of aspect E182, or any preceding aspect, wherein the one or more oligosaccharides have a dynamic viscosity of between 0.7 and 8 mPa*s (e.g., 0.7 and 8 mPa*s, 1 and 8 mPa*s, 0.7 and 7 mPa*s, or 0.7 and 6 mPa*s) at a concentration of 100 mg/ml in water at a temperature of 25° C.

Aspect E198: The method of aspect E182, or any preceding aspect, wherein the one or more oligosaccharides comprises CLX103.

Aspect E199: The method of any one of aspects A1-E198, wherein the one or more materials comprises galactomannan.

Aspect E200: The method of aspect E199, or any preceding aspect, wherein the one or more materials comprises a seed mucilage, a cell wall of a mold, or a combination thereof.

Aspect E201: The method of any one of aspects A1-E200, wherein the one or more materials comprises pea; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX122 set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX122 set forth in Table U; or
any combination thereof.

Aspect E202: The method of any one of aspects A1-E201, wherein the one or more materials comprises pea; and
wherein each of the one or more oligosaccharides is independently characterized by at least one feature comprising:
1H-13C HSQC NMR correlations within 30% (e.g., within 30%, or within 10%, within 15%, within 20%, or within 25%) of the 1H-13C HSQC NMR correlations corresponding to one or more compounds of CLX122DSF set forth in Table A;
a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX122DSF set forth in Table W; or
any combination thereof.

Aspect E203: The method of any one of aspects A1-E202, wherein the one or more materials comprises pea.

Aspect E204: The method of aspect E203, or any preceding aspect, wherein the one or more oligosaccharides contain at least 10% (e.g., within the range of 10-100%, within the range of 30-100%, within the range of 50-100%, within the range of 77-100%, within the range of 80-100%, within the range of 10-90%, within the range of 10-80%, or within the range of 10-77%) arabinose subunits.

Aspect E205: The method of aspect E204, or any preceding aspect, wherein the one or more oligosaccharides further comprises galactose subunits, glucose subunits, galacturonic acid subunits, rhamnose subunits, or a combination thereof.

Aspect E206: The method of aspect E203, or any preceding aspect, wherein the galactose subunits are present in an amount of at least 1% (e.g., within the range of 1-50%, within the range of 1-20%, within the range of 1-10%, within the range of 1-8%, or within the range of 1-6%), and wherein the glucose subunits are present in an amount of at least 1% (e.g., within the range of 1-50%, within the range of 1-20%, within the range of 1-10%, within the range of 1-8%, within the range of 1-6%, or within 1-4%), and wherein the galacturonic acid subunits are present in an amount of at least 1% (e.g., within the range of 1-50%, within the range of 1-20%, within the range of 1-10%, within the range of 1-4%, or within the range of 1-3%), and wherein the rhamnose subunits are present in an amount of at least 1% (e.g., within the range of 1-50%, within the range of 1-20%, within the range of 1-10%, within the range of 1-5%, within the range of 1-3%, or within the range of 1-2%).

Aspect E207: The method of aspect E203, or any preceding aspect, wherein the one or more oligosaccharides comprises arabinose subunits and galactose subunits, wherein the arabinose:galactose ratio is 8:1.

Aspect E208: The method of aspect E203, or any preceding aspect, wherein the one or more oligosaccharides comprises arabinose subunits and glucose subunits, wherein the arabinose:glucose ratio is 7.7:1.

Aspect E209: The method of aspect E203, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX122 set forth in Table U, CLX122DSF set forth in Table W, or any combination thereof.

Aspect E210: The method of aspect E203, or any preceding aspect, wherein the one or more oligosaccharides comprises at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table U or Table W, based on total mass of the one or more oligosaccharides and total mass of the compounds set forth in Table U or Table W, respectively.

Aspect E211: The method of aspect E203, or any preceding aspect, wherein the one or more oligosaccharides comprises one or more 2D NMR correlations within 30% (e.g., within 30%, or within 10%, within 15%, within 20%, or within 25%) of the 2D NMR correlations set forth in Table A for CLX122 or CLX122DSF.

Aspect E212: The method of aspect E203, or any preceding aspect, wherein the one or more oligosaccharides has a dynamic viscosity ranging from 0.75 to 5 mPa*s (e.g., 0.75 and 5 mPa*s, 0.75 and 4 mPa*s, 0.75 and 3.5 mPa*s, 0.75 and 3 mPa*s, or 1 and 5 mPa*s) at a concentration of 100 mg/ml in water at a temperature of 25° C.

Aspect E213: The method of aspect E203, or any preceding aspect, wherein the one or more oligosaccharides have a dynamic viscosity ranging from 2 to 4 mPa*s (e.g., 2 and 4 mPa*s, 2 and 3.5 mPa*s, 2 and 3 mPa*s, or 2.5 and 3.5 mPa*s) at a concentration of 100 mg/ml in water at a temperature of 25° C.

Aspect E214: The method of aspect E203, or any preceding aspect, wherein the one or more oligosaccharides have a dynamic viscosity ranging from 0.75 to 3 mPa*s (e.g., 0.75 and 3 mPa*s, 0.75 and 2 mPa*s, 0.75 and 1.8 mPa*s, 1 and 3 mPa*s, or 1 and 2 mPa*s) at a concentration of 100 mg/ml in water at a temperature of 25° C.

Aspect E215: The method of aspect E203, or any preceding aspect, wherein the one or more oligosaccharides comprises CLX122, CLX122DSF, or a combination thereof.

Aspect E216: The method of aspect E203, or any preceding aspect, wherein the one or more oligosaccharides comprises CLX122.

Aspect E217: The method of aspect E203, or any preceding aspect, wherein the one or more oligosaccharides comprises CLX122DSF.

Aspect E218: The method of any one of aspects A1-E217, wherein the one or more materials comprises the seed mucilage, and the seed mucilage comprises a guar seed mucilage, a tara seed mucilage, a cassia seed mucilage, a carob seed mucilage, a fenugreek seed mucilage, or any combination thereof.

Aspect E219: The method of any one of aspects A1-E218, wherein the one or more materials comprises the cell wall of a mold, and the cell wall of a mold is derived from Aspergillus genus.

Aspect E220: The method of aspect E219, or any preceding aspect, wherein the one or more oligosaccharides comprise at least 10% (e.g., within the range of 10-100%, within the range of 20-100%, within the range of 30-100%, within the range of 50-100%, within the range of 60-100%, within the range of 70-100%, within the range of 80-100%, or within the range of 90-100%) galactose and mannose subunits.

Aspect E221: The method of aspect E220, or any preceding aspect, wherein the galactose:mannose ratio is within the range of 0.1-1 to 1:1.

Aspect E222: The method of aspect E220, or any preceding aspect, wherein the galactose:mannose ratio is 0.25:1.

Aspect E223: The method of aspect E220, or any preceding aspect, wherein the mannose subunits comprise 4-linkages, branched 4-6 linkages, and terminal linkages in a total amount of at least 10% (e.g., within the range of 10-100%, within the range of 20-100%, within the range of 30-100%, within the range of 50-100%, or within the range of 75-100%).

Aspect E224: The method of aspect E220, or any preceding aspect, wherein the mannose subunits comprise a ratio of the 4-linkages to the branched 4-6 linkages to the terminal linkages of 1.5:0.2:1 to 3.5:0.2:1, 1.5:0.2:1 to 1.5:0.5:1, 1.5:0.31:1 to 3.5:0.31:1, or any combination thereof.

Aspect E225: The method of aspect E220, or any preceding aspect, wherein the galactose subunits comprise terminal linkages in an amount of at least 10% (e.g., within the range of 10-100%, within the range of 20-100%, within the range of 30-100%, within the range of 50-100%, or within the range of 75-100%).

Aspect E226: The method of aspect E220, or any preceding aspect, wherein backbones of the one or more oligosaccharides comprises mannose that is beta 4-linked.

Aspect E227: The method of aspect E220, or any preceding aspect, wherein the mannose subunits comprise 6-linkages alpha branched to galactose subunits.

Aspect E228: The method of any one of aspects A1-E227, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX111 as set forth in Table G.

Aspect E229: The method of any one of aspects A1-E228, wherein the one or more oligosaccharides comprise at least 30% (e.g., within the range of 30-100 wt. %, within the range of 40-100 wt. %, within the range of 50-100 wt. %, within the range of 75-100 wt. %, or within the range of 85-100 wt. %) of the compounds set forth in Table G, based on total mass of the one or more oligosaccharides and total mass of the compounds set forth in Table G.

Aspect E230: The method of any one of aspects A1-E229, wherein the one or more oligosaccharides comprise one or more of the 2D NMR correlations within 30% (e.g., within 30%, or within 10%, within 15%, within 20%, or within 25%) of the 2D NMR correlations set forth in Table A for CLX111.

Aspect E231: The method of any one of aspects A1-E230, wherein the one or more oligosaccharides comprises CLX111.

Aspect E232: The method of any one of aspects A1-E231, wherein the one or more materials comprises a moringa plant or part thereof.

Aspect E233: The method of aspect E232, or any preceding aspect, wherein the moringa plant or part thereof comprises leaves, branches, trunks, roots, seeds, flowers, fruit, or any combination thereof.

Aspect E234: The method of aspect E232, or any preceding aspect, wherein the moringa plant or part thereof comprises the leaves.

Aspect E235: The method of aspect E232, or any preceding aspect, wherein the one or more oligosaccharides comprise at least 20% (e.g., within the range of 20-100%, within the range of 30-100%, within the range of 40-100%, within the range of 50-100%, within the range of 75-100%, or within the range of 85-100%) glucose subunits.

Aspect E236: The method of aspect E235, or any preceding aspect, wherein the one or more oligosaccharides further comprise galactose subunits, arabinose subunits, rhamnose subunits, or any combination thereof.

Aspect E237: The method of aspect E236, or any preceding aspect, wherein the glucose:galactose ratio is 1:1 to 3:1 (e.g., 1:1, 1.5:1, 1.9:1, 2:1, 2.5:1, 3:1, or any combination thereof).

Aspect E238: The method of aspect E236, or any preceding aspect, wherein the galactose:arabinose ratio is 1.5:1 to 4:1 (e.g., 1.5:1, 2:1, 2.5:1, 2.6:1, 3:1, 3.5:1, 4:1, or any combination thereof).

Aspect E239: The method of aspect E236, or any preceding aspect, wherein the arabinose:rhamnose ratio is between 0.5:1 to 2:1 (e.g., 0.5:1, 1:1, 1.2:1, 1.5:1, 2:1, or any combination thereof).

Aspect E240: The method of aspect E236, or any preceding aspect, wherein the glucose:rhamnose ratio is between 3:1 to 8:1 (e.g., 3:1, 3.5:1, 4:1, 4.5:1, 5:1, 5.5:1, 6:1, 7:1, 8:1, or any combination thereof).

Aspect E241: The method of aspect E236, or any preceding aspect, wherein the glucose subunits comprise 4-linkages and terminal linkages in a total amount of at least 10% (e.g., within the range of 10-100%, within the range of 20-100%, within the range of 30-100%, within the range of 50-100%, or within the range of 75-100%).

Aspect E242: The method of aspect E241, or any preceding aspect, wherein the glucose subunits comprise a ratio of the 4-linkages to the terminal linkages of 0.1:1 to 1.5:1 (e.g., 0.1:1, 0.3:1, 0.5:1 0.6:1, 0.64:1, 0.8:1, 1:1, 1.2:1, 1.5:1, or any combination thereof).

Aspect E243: The method of aspect E236, or any preceding aspect, wherein the galactose subunits are present and comprise 4-linkages, 6-linkages, and terminal linkages in a total amount of at least 10% (e.g., within the range of 10-100%, within the range of 20-100%, within the range of 30-100%, within the range of 50-100%, or within the range of 75-100%).

Aspect E244: The method of aspect E243, or any preceding aspect, wherein the galactose subunits comprise a ratio of the 4-linkages to the 6-linkages to the terminal linkages of 0.7:0.1:1 to 2:0.1:1, or 0.7:0.1:1 to 0.7:1:1, or 1.2:0.1:1 to 1.2:1:1 (e.g., 1.2:0.67:1), or any combination thereof).

Aspect E245: The method of aspect E236, or any preceding aspect, wherein the arabinose subunits are present and comprise 5-linkages and terminal linkages in a total amount of at least 5% (e.g., within the range of 5-100%, within the range of 10-100%, within the range of 20-100%, within the range of 30-100%, within the range of 50-100%, or within the range of 75-100%).

Aspect E246: The method of aspect E245, or any preceding aspect, wherein the arabinose subunits comprise a ratio of the 5-linkages to the terminal linkages of 0.1:1 to 0.8:1 (e.g., 0.1:1, 0.2:1, 0.46:1, 0.5:1, 0.8:1, or any combination thereof).

Aspect E247: The method of aspect E236, or any preceding aspect wherein the rhamnose subunits are present and comprise terminal linkages in a total amount of at least 1% (e.g., within the range of 1-50%, within the range of 1-30%, within the range of 1-20%, within the range of 1-15%, or within the range of 1-10%).

Aspect E248: The method of aspect E236, or any preceding aspect, wherein at least 10 wt. % (e.g., within the range of 10-100 wt. %, within the range of 20-100 wt. %, within the range of 30-100 wt. %, within the range of 50-100 wt. %, within the range of 75-100 wt. %, or 100 wt. %) of the one or more oligosaccharides comprises a degree of polymerization between 2 and 30 (e.g., between 2 and 30, between 2 and 20, between 2 and 15, between 2 and 10, between 3 and 30, between 3 and 20, between 4 and 30, or between 4 and 20) based on total mass of the one or more oligosaccharides.

Aspect E249: The method of aspect E232, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX097 as set forth in Table B.

Aspect E250: The method of aspect E232, or any preceding aspect, wherein the one or more oligosaccharides comprise at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table

B, based on total mass of the one or more oligosaccharides and total mass of the compounds set forth in Table B.

Aspect E251: The method of aspect E232, or any preceding aspect, wherein the one or more oligosaccharides comprises CLX097.

Aspect E252: The method of any one of aspect A1-E251, wherein the one or more materials comprises a Cucurbita species or part thereof.

Aspect E253: The method of aspect E252, or any preceding aspect, wherein the Cucurbita species or part thereof comprises flesh, skin, seeds, or any combination thereof of a squash, a gourd, a pumpkin, or any combination thereof.

Aspect E254: The method of aspect E252, or any preceding aspect, wherein the Cucurbita species or part thereof comprises the skin of a butternut squash.

Aspect E255: The method of aspect E252, or any preceding aspect wherein at least 30 wt. % (e.g., within the range of 30-100 wt. %, within the range of 40-100 wt. %, within the range of 50-100 wt. %, within the range of 75-100 wt. %, or within the range of 85-100 wt. %) of the one or more oligosaccharides comprises a degree of polymerization between 2 and 30 (e.g., between 2 and 30, between 2 and 20, between 2 and 15, between 2 and 10, between 3 and 30, between 3 and 20, between 4 and 30, or between 4 and 20) based on total mass of the one or more oligosaccharides.

Aspect E256: The method of aspect E252, or any preceding aspect, wherein the one or more oligosaccharides comprise at least 30% (e.g., within the range of 30-100%, within the range of 40-100%, within the range of 50-100%, within the range of 75-100%, or within the range of 85-100%) glucose subunits.

Aspect E257: The method of aspect E256, or any preceding aspect, wherein the one or more oligosaccharides further comprise galactose subunits, arabinose subunits, or a combination thereof.

Aspect E258: The method of aspect E257, or any preceding aspect, wherein the glucose:galactose ratio is 5:1 to 8:1 (e.g., 5:1, 5.5:1, 6:1, 6.5:1, 7:1, 8:1, or any combination thereof).

Aspect E259: The method of aspect E256, or any preceding aspect, wherein the galactose:arabinose ratio is 1:1 to 3:1 (e.g., 1:1, 1.5:1, 1.9:1, 2:1, 2.5:1, 3:1, or any combination thereof).

Aspect E260: The method of aspect E257, or any preceding aspect, wherein the glucose subunits comprise 3-linkages and terminal linkage in a total amount of at least 30% (e.g., within the range of 30-100%, within the range of 40-100%, within the range of 50-100%, within the range of 75-100%, or within the range of 85-100%).

Aspect E261: The method of aspect E260, or any preceding aspect, wherein the glucose subunits comprise a ratio of the terminal linkages to the 3-linkages of 30:1 to 40:1 (e.g., 30:1, 31:1, 32:1, 33:1, 34:1, 35:1, 36:1, 37:1, 38:1, 39:1, 40:1, or any combination thereof).

Aspect E262: The method of aspect E257, or any preceding aspect, wherein the galactose subunits are present and comprise 3-linkages in a total amount of at least 0.5% (e.g., within the range of 0.5-100%, within the range of 0.5-50%, within the range of 0.5-20%, within the range of 0.5-15%, within the range of 0.5-10%, within the range of 0.5-5%, or within the range of 0.5-2.5%).

Aspect E263: The method of claim E260, or any preceding aspect wherein the ratio of the terminal glucose linkages to the galactose 3-linkages is 30:1 to 40:1 (e.g., 30:1, 31:1, 32:1, 33:1, 34:1, 35:1, 36:1, 37:1, 38:1, 39:1, 40:1, or any combination thereof).

Aspect E264: The method of aspect E252, or any preceding aspect, wherein the one or more oligosaccharides comprises a mass, a retention time, an oligosaccharide weight percentage, and a retention factor within 30% (e.g., within 30%, or within 10%, within 15%, within 20%, or within 25%) of the mass, the retention time, the oligosaccharide weight percentage, and the retention factor corresponding to one or more compounds of CLX098 set forth in Table C.

Aspect E265: The method of aspect E252, or any preceding aspect, wherein the one or more oligosaccharides comprise at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table C, based on total mass of the one or more oligosaccharides and total mass of the compounds set forth in Table C.

Aspect E266: The method of aspect E252, or any preceding aspect, wherein the one or more oligosaccharides comprises CLX098.

Aspect E267: The method of any one of aspects A1-E266, wherein the one or more materials comprises spent distillers' grains.

Aspect E268: The method of any one of aspects A1-E267, wherein one or more materials comprises microbial curdlan.

Aspect E269: The method of any one of aspects A1-E268, wherein the one or more materials comprises glucomannan.

Aspect E270: The method of any one of aspects A1-E269, wherein the one or more materials comprises pectic galactan.

Aspect E271: The method of any one of aspects A1-E270, wherein the one or more oligosaccharides are preferentially metabolized by beneficial vaginal Lactobacillus species but not preferentially metabolized by vaginal dysbiosis-associated bacteria.

Aspect E272: The method of aspect E271, or any preceding aspect, wherein the vaginal dysbiosis-associated bacteria comprise Lactobacillus iners, Gardenerella vaginalis, Atopobium vaginae, Prevotella species, Mobiluncus species, or any combination thereof.

Aspect E273: The method of any one of aspects A1-E272, wherein the composition further comprises at least one probiotic.

Aspect E274: The method of aspect E273, or any preceding aspect, wherein the at least one probiotic comprises Lactobacillus species, Bifidobacterium species, or a combination thereof.

Aspect E275: The method of aspect E274, or any preceding aspect, wherein the at least one probiotic comprises the Lactobacillus species, and the Lactobacillus species comprises at least one of L. rhamnosus, L. acidophilus, L. fermentum, L. reuteri, L. crispatus, L. jensenii, L. gasseri, L. acidophilus, L. iners, L., plantarum, L. vaginalis, or any combination thereof.

Aspect E276: The method of aspect E274, or any preceding aspect, wherein the at least one probiotic comprises the Lactobacillus species, and the Lactobacillus species comprises L. reuteri RC-14, L. rhamnosus GR-1, L. fermentum LF15, or any combination thereof.

Aspect E277: The method of aspect E273, or any preceding aspect, wherein the at least one probiotic comprises a live biotherapeutic.

Aspect E278: The method of aspect E277, or any preceding aspect, wherein the live biotherapeutic comprises L. crispatus CTV-05.

Aspect E279: The method of aspect E274, or any preceding aspect, wherein the at least one probiotic comprises the Bifidobacterium species, and the Bifidobacterium species comprises B. longum, B. breve, B. adolescentis, B. pseudocatenulatum, B. animalis, B. animalis lactis, B. infantis, or any combination thereof.

Aspect E280: The method of any one of aspects A1-E279, wherein the therapeutically effective amount is between 50 mg and 10 g (e.g., between 50 mg and 10 g, between 50 mg and 7.5 g, between 50 mg and 5 g, between 50 mg and 2.5 g, between 50 mg and 1 g, between 100 mg and 10 g, 100 mg and 7.5 g, between 100 mg and 5 g, between 100 mg and 2.5 g, between 500 mg and 10 g, or between 500 mg and 7.5 g) of the one or more oligosaccharides.

Aspect E281: The method of any one of aspects A1-E280, wherein the therapeutically effective amount is between 50 mg and 10 g (e.g., between 50 mg and 10 g, between 50 mg and 7.5 g, between 50 mg and 5 g, between 50 mg and 2.5 g, between 50 mg and 1 g, between 100 mg and 10 g, 100 mg and 7.5 g, between 100 mg and 5 g, between 100 mg and 2.5 g, between 500 mg and 10 g, or between 500 mg and 7.5 g) per day of the one or more oligosaccharides.

Aspect E282: The method of any one of aspects A1-E281, wherein the method reduces a risk of bacterial vaginosis in the female subject, reduces a risk of recurrence of bacterial vaginosis in the female subject, reduces severity of bacterial vaginosis in the female subject, treats bacterial vaginosis in the female subject, reduces a risk of urinary tract infection when the female subject has bacterial vaginosis, reduces a risk of urinary tract infection when the female subject has had, but no longer has, bacterial vaginosis, or any combination thereof.

Aspect E283: The method of any one of aspects A1-E282, wherein the method reduces a risk of vulvovaginal candidiasis in the female subject, reduces a risk of recurrence of vulvovaginal candidiasis in the female subject, reduces severity of vulvovaginal candidiasis in the female subject, treats vulvovaginal candidiasis in the female subject, or any combination thereof.

Aspect F1: A pack for improving urogenital health in a female subject, the pack comprising at least 7 or at least 14 individual daily doses each of which comprising a therapeutically effective amount of one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect F2: The pack of aspect F1, or any preceding aspect, wherein the one or more materials or the one or more oligosaccharides is as described in any one of aspects E1-E283, or any preceding aspect.

Aspect G1: A formulation comprising one or more oligosaccharides as described in any one of aspects E1-E283, or any preceding aspect, or as derived from one or more materials as described in any one of aspects E1-E283, or any preceding aspect.

Aspect G2: A formulation comprising a composition comprising one or more oligosaccharides as described in any one of aspects E1-E283, or any preceding aspect.

Aspect G3: The formulation of aspect G1 or G2, or any preceding aspect, further comprising at least one microorganism.

Aspect G4: The formulation of aspect G3, or any preceding aspect, wherein the at least one microorganism comprises at least one of Bifidobacteria, Bifidobacterium pseudocatenulatum, Bifidobacterium animalis, Bacteroides, Bacteroides ovatus, Firmicutes, Clostridium butyricum, Ruminococcus, Ruminococcus gnavus, Ruminococcus torques, Blautia, Roseburia, Faecalibacterium, Lactobacillus crispatus, Lactobacillus rhamnosus, Lactobacillus Rhamnosus GG, Bifidobacterium longum subsp. longum, Bifidobacterium longum subsp. infantis, Ruminococcus torques, or any combination thereof.

Aspect G5: The formulation of aspect G4, or any preceding aspect, further comprising a pharmaceutically acceptable carrier.

Aspect G6: The formulation of aspect G1, or any preceding aspect, wherein the formulation is in the form of a food, a drink, a nutritional supplement, a medicine, an injection, a pill, a capsule, a cream, or a topical ointment.

Aspect H1: Use of a composition for improving urogenital health in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect I1: Use of a composition for treating or preventing a vaginal infection in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect J1: Use of a composition for modulating the microbiota of the urogenital tract in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect K1: Use of a composition for lowering the pH of the urogenital tract in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect L1: A medicament, for use in for improving urogenital health in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect M1: A medicament, for use in treating or preventing a vaginal infection in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect N1: A medicament, for use in modulating the microbiota of the urogenital tract in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect O1: A medicament, for use in lowering the pH of the urogenital tract in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

Aspect P1: A method of making a medicament for use in for improving urogenital health in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and
an optional carrier.

Aspect Q1: A method of making a medicament for use in treating or preventing a vaginal infection in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and
an optional carrier.

Aspect R1: A method of making a medicament for use in modulating the microbiota of the urogenital tract in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and
an optional carrier.

Aspect S1: A method of making a medicament for use in lowering the pH of the urogenital tract in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and
an optional carrier.

Aspect T1: The method of any one of aspects A1-S1, wherein the one or more oligosaccharides comprise at least 30% (e.g., within the range of 30-100%, within the range of 40-100%, within the range of 50-100%, within the range of 75-100%, or within the range of 85-100%) glucose subunits.

Aspect T2: The method of aspect T1, or any preceding aspect, wherein the one or more oligosaccharides further comprise galactose subunits, mannose subunits, arabinose subunits, xylose subunits, or any combination thereof.

Aspect T3: The method of aspect T2, or any preceding aspect, wherein the glucose: galactose ratio is between 12:1 to 25:1 (e.g., 12:1, 18:1, 25:1, or any combination thereof).

Aspect T4: The method of aspect T2, or any preceding aspect, wherein the galactose:mannose ratio is between 0.2:1 to 2:1 (e.g., 0.2:1, 0.72:1, 2:1, or any combination thereof).

Aspect T5: The method of any one of aspects T2-T4, or any preceding aspect, wherein the mannose: arabinose ratio is between 0.1:1 to 2:1 (e.g., 0.1:1, 0.61:1, 2:1, or any combination thereof).

Aspect T6: The method of any one of aspects T2-T5, or any preceding aspect, wherein the arabinose: xylose ratio is between 0.5:1 to 4:1 (e.g., 0.5:1, 1.5:1, 2:1, 3:1, 4:1, or any combination thereof).

Aspect T7: The method of any one aspects T2-T6, or any preceding aspect, wherein the glucose: xylose ratio is between 8:1 to 15:1 (e.g., 8:1, 12:1, 15:1, or any combination thereof).

Aspect T8: The method of any one of aspects T2-T7, or any preceding aspect, wherein the glucose subunits comprise 4-linkages, branched 4-6 linkages, and terminal linkages in a total amount of at least 40%, (e.g., within the range of 40-100%, within the range of 50-100%, within the range of 75-100%, or within the range of 85-100%)

Aspect T9: The method of aspect T8, or any preceding aspect, wherein the glucose subunits comprise a ratio of the 4-linkages to the terminal linkages of 1:1 to 6:1 (e.g., 1:1, 2:1, 3:1, 3.5:1, 4:1, 5:1, 6:1, or any combination thereof)

Aspect T10: The method of aspect T8, or any preceding aspect, wherein the glucose subunits comprise a ratio of the terminal linkages to the branched 4-6 linkages of 2:1 to 9:1 (e.g., 2:1, 3:1, 4:1, 5:1, 6:1, 6.7:1, 7:1, 8:1, 9:1, or any combination thereof).

Aspect T11: The method of any one of aspects T2-T10, or any preceding aspect, wherein the xylose subunits are present and comprise 4-linkages and branched 3-4 linkages in a total amount of at least 1%, at least 5%, at least 10%, or at least 15%, as determined by glycosidic linkage composition analysis.

Aspect T12: The method of aspect T11, or any preceding aspect, wherein the xylose subunits comprise a ratio of the 4-linkages to the branched 3-4 linkages of 0.5:1 to 2:1 (e.g., 0.5:1, 0.95:1, 1:1, 2:1, or any combination thereof).

Aspect T13: The method of any one of aspects T2-T12, or any preceding aspect, wherein the arabinose subunits are present and comprise terminal linkages in a total amount of at least 1% (e.g., within the range of 1-50%, within the range of 1-20%, within the range of 1-12%, within the range of 1-10%, within the range of 1-8%, or within the range of 1-6%).

Aspect T14: The method of any one of aspects T2-T13, or any preceding aspect, wherein the ratio of the terminal glucose linkages to the terminal arabinose linkages is 0.8:1 to 4:1 (e.g., 0.8:1, 1:1, 1.9:1, 2:1, 3:1, 4:1, or any combination thereof).

Aspect T15: The method of any one of aspects T1-T14, or any preceding aspect, wherein the one or more oligosaccharides comprise an oligosaccharide analysis within 30% (e.g., within 30%, or within 10%, within 15%, within 20%, or within 25%) of the values set forth in Table D.

Aspect T16: The method of any one of aspects T1-T15, or any preceding aspect, wherein the one or more oligosaccharides comprise at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table D, based on total mass of the one or more oligosaccharides and total mass of the compounds set forth in Table D.

Aspect T17: The method of any one aspects A1-T16, wherein the one or more oligosaccharides comprises CLX099.

Aspect T18: The method of any one of aspects A1-T17, wherein the one or more oligosaccharides comprise one or more of the compounds set forth in Table I.

Aspect T19: The method of any one of aspects A1-T18, wherein the one or more oligosaccharides comprise at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table I, based on total mass of the one or more oligosaccharides and total mass of the compounds set forth in Table I.

Aspect T20: The method of any one of aspects A1-T19, wherein the one or more oligosaccharides comprise one or more of the 2D NMR correlations described in Table A for CLX101.

Aspect T21: The method of any one of aspects A1-T20, wherein the one or more oligosaccharides comprises CLX101.

Aspect T22: The method of any one aspects A1-T21, wherein the one or more oligosaccharides comprise one or more of the compounds set forth in Table J.

Aspect T23: The method of any one aspects A1-T22, wherein the one or more oligosaccharides comprise at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table J, based on total mass of the one or more oligosaccharides and total mass of the compounds set forth in Table J.

Aspect T24: The method of any one aspects A1-T23, wherein the one or more oligosaccharides comprise one or more of the 2D NMR correlations described in Table A for CLX102.

Aspect T25: The method of any one of aspects A1-T24, wherein the one or more oligosaccharides comprises CLX102.

Aspect T26: The method of any one of aspects A1-T25, wherein the one or more oligosaccharides comprises one or more of the compounds set forth in Table M.

Aspect T27: The method of any one of aspects A1-T26, wherein the one or more oligosaccharides comprise at least 30 wt. % (e.g., within the range of 30-100 wt. %, within the range of 40-100 wt. %, within the range of 50-100 wt. %, within the range of 75-100 wt. %, or within the range of 85-100 wt. %) of the compounds set forth in Table M, based on total mass of the one or more oligosaccharides and total mass of the compounds set forth in Table M.

Aspect T28: The method of any one of aspects A1-T27, wherein the one or more oligosaccharides comprises CLX109.

Aspect U1: A method of improving urogenital health in a female subject, the method comprising:
administering to the female subject a therapeutically effective amount of a composition comprising one or more oligosaccharides;
wherein each of the one or more oligosaccharides independently contains 3 to 30 subunits (e.g., 3 to 30, 3 to 20, 3 to 15, 3 to 10, 4 to 30, 4 to 20, 4 to 15, or 4 to 10);
wherein at least 5% (e.g., within the range of 5-100%, within the range of 10-100%, within the range of 15-100%, within the range of 20-100%, within the range of 25-100%, within the range of 30-100%, within the range of 50-100%, within the range of 75-100%, or within the range of 85-100%) of the subunits comprise a beta-1,3 glucose residue, a beta-1,4 glucose residue, or a combination thereof; and
wherein the administering step results in improving urogenital health in the female subject.

Aspect V1: A method of treating or preventing a vaginal infection in a female subject, the method comprising:
administering to the female subject a therapeutically effective amount of a composition comprising one or more oligosaccharides;
wherein each of the one or more oligosaccharides independently contains 3 to 30 subunits (e.g., 3 to 30, 3 to 20, 3 to 15, 3 to 10, 4 to 30, 4 to 20, 4 to 15, or 4 to 10);
wherein at least 5% (e.g., within the range of 5-100%, within the range of 10-100%, within the range of 15-100%, within the range of 20-100%, within the range of 25-100%, within the range of 30-100%, within the range of 50-100%, within the range of 75-100%, or within the range of 85-100%) of the subunits comprise a beta-1,3 glucose residue, a beta-1,4 glucose residue, or a combination thereof; and
wherein the administering step results in treating or preventing a vaginal infection in the female subject.

Aspect W1: A method of modulating the microbiota of the urogenital tract of a female subject, the method comprising:
administering to the female subject a therapeutically effective amount of a composition comprising one or more oligosaccharides;
wherein each of the one or more oligosaccharides independently contains 3 to 30 subunits (e.g., 3 to 30, 3 to 20, 3 to 15, 3 to 10, 4 to 30, 4 to 20, 4 to 15, or 4 to 10);
wherein at least 5% (e.g., within the range of 5-100%, within the range of 10-100%, within the range of 15-100%, within the range of 20-100%, within the range of 25-100%, within the range of 30-100%, within the range of 50-100%, within the range of 75-100%, or within the range of 85-100%) of the subunits comprise a beta-1,3 glucose residue, a beta-1,4 glucose residue, or a combination thereof; and
wherein the administering step comprises contacting said microbiota of the urogenital tract with said one or more oligosaccharides; and
wherein the administering step results in modulating the microbiota of the urogenital tract of the female subject.

Aspect W2: The method of aspect W1, or any preceding aspect, wherein the method results in increased abundance of Lactobacillus species in the urogenital tract of the female subject.

Aspect W3: The method of aspect W2, or any preceding aspect, wherein the Lactobacillus species comprise L. crispatus, L. gasseri, L. jensenii, L. iners, L. vaginalis, or any combination thereof.

Aspect X1: A method of lowering the pH of the urogenital tract of a female subject, the method comprising:
administering to the female subject a therapeutically effective amount of a composition comprising one or more oligosaccharides;
wherein each of the one or more oligosaccharides independently contains 3 to 30 subunits (e.g., 3 to 30, 3 to 20, 3 to 15, 3 to 10, 4 to 30, 4 to 20, 4 to 15, or 4 to 10);
wherein at least 5% (e.g., within the range of 5-100%, within the range of 10-100%, within the range of 15-100%, within the range of 20-100%, within the range of 25-100%, within the range of 30-100%, within the range of 50-100%, within the range of 75-100%, or within the range of 85-100%) of the subunits comprise a beta-1,3 glucose residue, a beta-1,4 glucose residue, or a combination thereof; and
wherein the administering step results in lowering the pH of the urogenital tract.

Aspect X2: The method of aspect X1, or any preceding aspect, wherein the method results in lowering the pH of the urogenital tract from a first pH of 4.5-7 to a second pH of less than 4.5 (for example, a second pH of 4.49. 4.25, 4, 3.75, 3.5, 3.25, or 3; optionally, not less than a pH of 2.5).

Aspect X3: The method of aspect X2, or any preceding aspect, wherein the second pH is between 3.5-4.5 (for example, a pH of 3.5, 3.6, 3.7, 3.8, 3.9, 4, 4.1, 4.2, 4.3, 4.4, or 4.5).

Aspect Y1: The method of any one of aspects U1-X3, or any preceding aspect, wherein at least 10% (e.g., within the range of 10-100%, within the range of 15-100%, within the range of 20-100%, within the range of 25-100%, within the range of 30-100%, within the range of 50-100%, within the range of 75-100%, or within the range of 85-100%) of the one or more oligosaccharides contains 3 to 20 subunits (e.g., 3 to 20, 3 to 15, 3 to 10, 4 to 20, 4 to 15, or 4 to 10).

Aspect Y2: The method of any one of aspects U1-X3, or any preceding aspect, wherein at least 10% (e.g., within the range of 10-100%, within the range of 15-100%, within the range of 20-100%, within the range of 25-100%, within the range of 30-100%, within the range of 50-100%, within the range of 75-100%, or within the range of 85-100%) of the one or more oligosaccharides contains 3 to 10 subunits (e.g., 3 to 10 or 4 to 10)

Aspect Y3: The method of any one of aspects U1-Y2, or any preceding aspect, wherein at least 5% (e.g., within the range of 5-100%, within the range of 10-100%, within the range of 15-100%, within the range of 20-100%, within the range of 25-100%, within the range of 30-100%, within the range of 50-100%, within the range of 75-100%, or within the range of 85-100%) of the subunits comprise a beta-1,4 glucose residue.

Aspect Y4: The method of any one of aspects Ul-Y3, or any preceding aspect, wherein at least 5% (e.g., within the range of 5-100%, within the range of 10-100%, within the range of 15-100%, within the range of 20-100%, within the range of 25-100%, within the range of 30-100%, within the range of 50-100%, within the range of 75-100%, or within the range of 85-100%) of the subunits comprise a beta-1,3 glucose residue.

All references listed below, or anywhere else throughout this description, are hereby incorporated by reference herein in their entireties for all purposes to the extent not inconsistent with the description herein:
Al-Ghazzewi F. H., Tester R. F. Biotherapeutic agents and vaginal health. J Appl Microbiol. 2016, 121, 18-27.
Allsworth J E and Peipert J F. Prevalence of bacterial vaginosis: 2001-2004 National Health and Nutrition Examination Survey data. Obstet Gynecol 2007; 109(1):114-20.
Amicucci, M. J., Galermo, A. G., et al. (2019). “A rapid-throughput adaptable method for determining the monosaccharide composition of polysaccharides.” International Journal of Mass Spectrometry 438: 22-28.
Amicucci, M. J., Nandita, E., et al. (2019). “Function without Structures: The Need for In-Depth Analysis of Dietary Carbohydrates.” Journal of Agricultural and Food Chemistry 67(16): 4418-4424.
Amicucci, M. J., Park, D., et al. (2018). Production of bioactive oligosaccharides, WO 2018/236917.
Amicucci, M. J., Nandita, E., et al. (2020). “A nonenzymatic method for cleaving polysaccharides to yield oligosaccharides for structural analysis.” Nature Communications 11(1): 1-12.
Chew S. Y., Cheah Y. K., et al. (2015). “Probiotic Lactobacillus rhamnosus GR-1 and Lactobacillus reuteri RC-14 exhibit strong antifungal effects against vulvovaginal candidiasis-causing Candida glabrata isolates.” J. Appl. Microbiol. 118(5): 1180-1190.
Cohen C R, Lingappa J R, Baeten J M, et al. Bacterial vaginosis associated with increased risk of female-to-male HIV-1 transmission: a prospective cohort analysis among African couples. PLOS Med. 2012; 9(6):e1001251. doi: 10.1371/journal.pmed. 1001251. Epub 2012 Jun. 26.
Collins et al. (2018). “Promising Prebiotic Candidate Established by Evaluation of Lactitol, Lactulose, Raffinose, and Oligofructose for Maintenance of a Lactobacillus-Dominated Vaginal Microbiota.” Appl. Environ. Microbiol. doi: 10.1128/AEM.02200-17.
DiGiulio D. B., B. J. Callahan, P. J.McMurdie, E. K. Costello, D. J. Lyell, A. Robaczewska, C. L. Sun, D. S. A. Goltsman, R. J. Wong, G. Shaw, D. K. Stevenson, m S. P. Holmes, an D. A. Relman. Temporal and spatial variation of the human microbiota during pregnancy. Proc Natl Acad Sci USA 112: 11060-11065.
Dominguez, A. L., et al. (2014). “An overview of the recent developments on fructooligosaccharide production and applications.” Food and Bioprocess Technology 7(2): 324-337.
Fidel, P. L. Jr., Barousse M., Espinosa T., et al. (2004). “An intravaginal live Candida challenge in humans leads to new hypotheses for the immunopathogenesis of vulvovaginal candidiasis.” Infect Immun. 72(5): 2939-2946.
Galermo, A. G., Nandita, E., et al. (2018). “Liquid chromatography-tandem mass spectrometry approach for determining glycosidic linkages.” Analytical Chemistry 90(21): 13073-13080
Galermo, A. G., Nandita, E., et al. (2019). “Development of an extensive linkage library for characterization of carbohydrates.” Analytical Chemistry 91(20): 13022-13031.
Gosling, A., Stevens, G. W., et al. (2010). “Recent advances refining galactooligosaccharide production from lactose.” Food Chemistry 121(2): 307-318.
Guarner, F. and Malagelada, J. R. (2003). “Gut flora in health and disease.” The Lancet 361(9356): 512-519.
Hilbert D W, J. A. Schuyler, M. E. Adelson, E. Mordechai E., J D. Sobel, S E Gygax. Gardnerella vaginalis population dynamics in bacterial vaginosis. Eur J Clin Microbiol Infect Dis. 2017, 36 (7): 1269-1278.
LoCascio, R. G., Ninonuevo, M. R., et al. (2007). “Glycoprofiling of bifidobacterial consumption of human milk oligosaccharides demonstrates strain specific, preferential consumption of small chain glycans secreted in early human lactation.” Journal of Agricultural and Food Chemistry 55(22): 8914-8919.
Macklaim J M, Clemente J C, Knight R, Gloor G B, Reid G. 2015. Changes in vaginal microbiota following antimicrobial and probiotic therapy. Microb Ecol Health Dis 26:27799.
Mastromario P., Vitali B., Mosca, L; Bacterial vaginosis: A review on clinical trials with probiotics; New Microbiol, 2013, 36(3) 229-38.
Mendling W., A. Palmeira-de-Oliveira A., S. Biber, V. Prasauskas. An update on the role of Atopobium vaginae in bacterial vaginosis: what to consider when choosing a treatment? A mini review. Arch Gynecol Obstet 2019, 300 (1): 1-6.
Merighi, M., McCoy, J. M., et al. (2016). Biosynthesis of human milk oligosaccharides in engineered bacteria, WO 2012/112777.
Morais I. M. C., Cordeiro A. L., et al. (2017). “Biological and physicochemical properties of biosurfactants produced by Lactobacillus jensenii P6A and Lactobacillus gasseri P65.” Microb. Cell Fact. 16(1): 155.
Morrow, A. L., Ruiz-Palacios, G. M., et al. (2004). “Human milk oligosaccharides are associated with protection against diarrhea in breast-fed infants.” The Journal of Pediatrics 145(3): 297-303.
Osset J., Garcia E., et al. (2001). “Role of Lactobacillus as protector against vaginal candidiasis.” Med. Clin. 117(8): 285-288.
Rousseau et al. (2005). “Prebiotic effects of oligosaccharides on selected vaginal lactobacilli and pathogenic microorganisms.” Anaerobe, 11: 145-153.
Ruiz-Moyano S., Totten S., Garrido A., Smilowitz J., German B., Lebrilla C., Mills D. Variation in Consumption of Human Milk Oligosaccharides by Infant Gut-Associated Strains of Bifidobacterium breve; Appl Environ Microbiol, 2013, 79 (19): 6040-6049.
Smilowitz, J. T., Lebrilla, C. B., et al. (2014). “Breast milk oligosaccharides: structure-function relationships in the neonate.” Annual Review of Nutrition 34: 143-169.
Younes J. A., E. Lievens, R. Hummelen, R. V. van der Westen, G. Reid, M. I. Petrova. Women and their Microbes: The unexpected Friendship. Trends in Microbiology. 2018. 26:16-32.
Yu, S., Liu, J.-J., et al. (2018). “Production of a human milk oligosaccharide 2′-fucosyllactose by metabolically engineered Saccharomyces cerevisiae.” Microbial Cell Factories 17(1): 101.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties for all purposes, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of improving urogenital health in a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; and
wherein the administering step results in improving urogenital health in the female subject.

2. A method of treating or preventing a vaginal infection in a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof; and
wherein the administering step results in treating or preventing a vaginal infection in the female subject.

3. The method of claim 2, wherein the vaginal infection comprises a bacterial infection.

4. The method of claim 3, wherein the bacterial infection comprises bacterial vaginosis.

5. The method of claim 3, wherein the bacterial infection comprises a urinary tract infection.

6. The method of claim 2, wherein the vaginal infection comprises a fungal infection.

7. The method of claim 6, wherein the fungal infection comprises a yeast infection.

8. A method of modulating the microbiota of the urogenital tract of a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof;
wherein the administering step comprises contacting said microbiota of the urogenital tract with said one or more oligosaccharides; and
wherein the administering step results in modulating the microbiota of the urogenital tract of the female subject.

9. The method of claim 8, wherein the method results in increased abundance of Lactobacillus species in the urogenital tract of the female subject.

10. The method of claim 9, wherein the Lactobacillus species comprise L. crispatus, L. gasseri, L. jensenii, L. iners, L. vaginalis, or any combination thereof.

11. A method of lowering the pH of the urogenital tract of a female subject, the method comprising:
administering to the female subject a therapeutically effective amount of a composition comprising one or more oligosaccharides;
wherein the one or more oligosaccharides is derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof;
wherein the administering step results in lowering the pH of the urogenital tract.

12. The method of claim 11, wherein the method results in lowering the pH of the urogenital tract from a first pH of 4.5-7 to a second pH of less than 4.5.

13. The method of claim 12, wherein the second pH is between 3.5-4.5.

14. The method of any one of claims 1-13, wherein the method results in enhanced microbial production of lactic acid in the urogenital tract of the female subject.

15. The method of any one of claims 1-13, wherein each of the one or more oligosaccharides independently contains 3 to 50 subunits, wherein at least 10% of the subunits comprise a beta-1,3 glucose residue, a beta-1,4 glucose residue, or a combination thereof.

16. The method of any one of claims 1-13, wherein the one or more materials comprises beta glucan; and
wherein each of the one or more oligosaccharides is independently characterized by at least one feature comprising:
1H-13C HSQC NMR correlations within 30% of the 1H-13C HSQC NMR correlations corresponding to one or more compounds of CLX115 set forth in Table A;
a mass, a retention time, an oligosaccharide weight percentage, and a retention factor within 30% of the mass, the retention time, the oligosaccharide weight percentage, and the retention factor corresponding to one or more compounds of CLX115 set forth in Table P; or
any combination thereof.

17. The method of any one of claims 1-13, wherein the one or more materials comprises beta glucan.

18. The method of any one of claims 1-13, wherein the one or more materials comprises beta glucan and
wherein the one or more oligosaccharides contains beta-1,3 and beta-1,4 linked glucose residues.

19. The method any one of claims 1-13, wherein the one or more materials comprises beta glucan and wherein the one or more oligosaccharides contains 3 to 50 subunits, wherein each subunit is a beta-1,3 glucose residue, a beta-1,4 glucose residue, or a combination thereof.

20. The method any one of claims 1-13, wherein the one or more materials comprises beta glucan and wherein the one or more oligosaccharides comprises beta-1,3 linked glucose residues and beta-1,4 linked glucose residues, wherein the ratio of beta-1,3 linked residues:beta-1,4 linked residues ranges from 1:1 to 1:5.

21. The method any one of claims 1-13, wherein the one or more materials comprises beta glucan and, wherein the one or more oligosaccharides has an average molecular weight (Mw) of less than 10,000 Da.

22. The method any one of claims 1-13, wherein the one or more materials comprises beta glucan and, wherein the one or more oligosaccharides has an average molecular weight (Mw) of less than 8,000 Da.

23. The method of any one of claims 1-13, wherein the one or more materials comprises beta glucan and wherein the one or more oligosaccharides has a dynamic viscosity ranging from 1 to 10 mPa*s at 100 mg/ml at 25° C.

24. The method of any one of claims 1-13, wherein the one or more materials comprises beta glucan and wherein at least 70% of the mass of the one or more oligosaccharides has a molecular mass of less than 100 kDa.

25. The method of any one of claims 1-13, wherein the one or more materials comprises beta glucan and wherein at least 60% of the mass of the one or more oligosaccharides has a molecular mass of less than 50 kDa.

26. The method of any one of claims 1-13, wherein the one or more materials comprises beta glucan and wherein at least 50% of the mass of the one or more oligosaccharides has a molecular mass of less than 15 kDa.

27. The method of any one of claims 1-13, wherein the one or more materials comprises beta glucan and wherein at least 50% of the mass of the one or more oligosaccharides has a molecular mass of less than 5 kDa.

28. The method of any one of claims 1-13, wherein the one or more materials comprises beta glucan and wherein the one or more materials comprises a cereal, a lichenan, a yeast, or any combination thereof.

29. The method of any one of claims 1-13, wherein the one or more materials comprises beta glucan and wherein the one or more materials comprises a cereal, and the cereal comprises a barley, a rye, a wheat, an oat, or any combination thereof.

30. The method of any one of claims 1-13, wherein the one or more materials comprises beta glucan and wherein at least 10 wt. % of the one or more oligosaccharides comprises a degree of polymerization between 2 and 30 based on total mass of the one or more oligosaccharides.

31. The method of any one of claims 1-13, wherein the one or more materials comprises beta glucan and wherein the one or more oligosaccharides contain at least 10% glucose subunits.

32. The method of claim 31, wherein the one or more oligosaccharides further comprises galactose subunits and mannose subunits, wherein the galactose subunits are present in an amount of at least 1% and wherein the mannose subunits are present in an amount of at least 1%.

33. The method of any one of claims 1-13, wherein the one or more materials comprises beta glucan and wherein the one or more oligosaccharides comprises glucose subunits and galactose subunits, wherein the galactose: glucose ratio is between 0.05:1 to 2:1.

34. The method of any one of claims 1-13, wherein the one or more materials comprises beta glucan and wherein the one or more oligosaccharides comprises glucose subunits and mannose subunits, wherein the mannose: glucose ratio is between 0.1:1 to 2:1.

35. The method of any one of claims 1-13, wherein the one or more materials comprises beta glucan and wherein the one or more oligosaccharides comprises mannose subunits, wherein the mannose subunits comprise 2-linkages, 3-linkages, and terminal linkages in a total amount of at least 10%.

36. The method of claim 31, wherein the glucose subunits comprise 3-linkages, 4-linkages, and terminal linkages in a total amount of at least 10%.

37. The method of claim 31, wherein the glucose subunits comprise a ratio of 3-linkages:4-linkages:terminal linkages of 1:0.5:1 to 3:2:1.

38. The method of claim 31, wherein the glucose subunits comprise a ratio of 3-linkages:4-linkages:terminal linkages of 0.4:1:1 to 0.7:2:1.

39. The method of claim 31, wherein the glucose subunits comprise a ratio of 3-linkages:4-linkages of 0.1:1 to 0.8:1.

40. The method of claim 31, wherein the glucose subunits comprise a ratio of 3-linkages:4-linkages of 0.3:1 to 0.65:1.

41. The method of claim 31, wherein the glucose subunits comprise a ratio of 4-linkages:terminal linkages of 1:1 to 3:1.

42. The method of any one of claims 1-13, wherein the one or more materials comprises beta glucan and, wherein the one or more oligosaccharides comprises glucose subunits and galactose subunits, wherein the one or more oligosaccharides comprises a ratio of glucose 4-linkages:galactose terminal linkages of 5:1 to 7:1.

43. The method of any one of claims 1-13, wherein the one or more materials comprises beta glucan and wherein the one or more oligosaccharides comprises glucose subunits and galactose subunits, the one or more oligosaccharides comprises a ratio of glucose terminal linkages to galactose terminal linkages of 2:1 to 4:1.

44. The method of any one of claims 1-13, wherein the one or more materials comprises beta glucan and wherein the one or more oligosaccharides comprises glucose subunits having glucose 3-linkages, wherein the glucose 3-linkages are beta linked.

45. The method of any one of claims 1-13, wherein the one or more materials comprises beta glucan and wherein the one or more oligosaccharides comprises glucose subunits having glucose 4-linkages, wherein the glucose 4-linkages are beta linked.

46. The method of any one of claims 1-13, wherein the one or more materials comprises beta glucan and wherein the one or more oligosaccharides comprises a mass, a retention time, an oligosaccharide weight percentage, and a retention factor within 30% of the mass, the retention time, the oligosaccharide weight percentage, and the retention factor corresponding to one or more compounds of CLX110 set forth in Table F, CLX115 set forth in Table P, CLX115Cu set forth in Table V, CLX112 set forth in Table H, or any combination thereof.

47. The method of any one of claims 1-13, wherein the one or more materials comprises beta glucan and wherein the one or more oligosaccharides comprises at least 30 wt. %, of the compounds set forth in Table F, Table P, Table V, or Table H, based on total mass of the one or more oligosaccharides and total mass of the compounds set forth in Table F, Table P, Table V, or Table H, respectively.

48. The method of any one of claims 1-13, wherein the one or more materials comprises beta glucan and wherein the one or more oligosaccharides comprises one or more 2D NMR correlations within 30% of the 2D NMR correlations set forth in Table A for CLX110, CLX112, CLX115, or CLX115Cu.

49. The method of any one of claims 1-13, wherein the one or more materials comprises beta glucan and wherein the one or more oligosaccharides has a dynamic viscosity ranging from 0.6 to 1.2 mPa*s at a concentration of 10 mg/ml in water at a temperature of 25° C.

50. The method of any one of claims 1-13, wherein the one or more materials comprises beta glucan and wherein the one or more oligosaccharides have a dynamic viscosity ranging from 0.7 to 3 mPa*s at a concentration of 50 mg/ml in water at a temperature of 25° C.

51. The method of any one of claims 1-13, wherein the one or more materials comprises beta glucan and wherein the one or more oligosaccharides have a dynamic viscosity ranging from 0.7 to 8 mPa*s at a concentration of 100 mg/ml in water at a temperature of 25° C.

52. The method of any one of claims 1-13, wherein the one or more materials comprises beta glucan and wherein the one or more oligosaccharides comprises CLX110, CLX112, CLX115, CLX115Cu, or a combination thereof.

53. The method of any one of claims 1-13, wherein the one or more materials comprises beta glucan and wherein the one or more oligosaccharides comprises CLX115.

54. The method of any one of claims 1-13, wherein the one or more materials comprises xanthan gum; and
wherein each of the one or more oligosaccharides is independently characterized by at least one feature comprising:
1H-13C HSQC NMR correlations within 30% of the 1H-13C HSQC NMR correlations corresponding to one or more compounds of CLX123 set forth in Table A;
a mass, a retention time, an oligosaccharide weight percentage, and a retention factor within 30% of the mass, the retention time, the oligosaccharide weight percentage, and the retention factor corresponding to one or more compounds of CLX123 set forth in Table Q; or
any combination thereof.

55. The method of any one of claims 1-13, wherein the one or more materials comprises xanthan gum.

56. The method of any one of claims 1-13, wherein the one or more materials comprises xanthan gum and wherein the one or more oligosaccharides contains beta-1,4 linked glucose residues.

57. The method of any one of claims 1-13, wherein the one or more materials comprises xanthan gum and wherein the one or more oligosaccharides has an average molecular weight (Mw) of less than 10,000 Da.

58. The method of any one of claims 1-13, wherein the one or more materials comprises xanthan gum and wherein the one or more oligosaccharides has an average molecular weight (Mw) of less than 8,000 Da.

59. The method of any one of claims 1-13, wherein the one or more materials comprises xanthan gum and wherein the one or more oligosaccharides has a dynamic viscosity ranging from 1 to 10 mPa*s at 100 mg/ml at 25° C.

60. The method of any one of claims 1-13, wherein the one or more materials comprises xanthan gum and wherein at least 70% of the mass of the one or more oligosaccharides has a molecular mass of less than 100 kDa.

61. The method of any one of claims 1-13, wherein the one or more materials comprises xanthan gum and wherein at least 60% of the mass of the one or more oligosaccharides has a molecular mass of less than 50 kDa.

62. The method of any one of claims 1-13, wherein the one or more materials comprises xanthan gum and wherein at least 50% of the mass of the one or more oligosaccharides has a molecular mass of less than 15 kDa.

63. The method of any one of claims 1-13, wherein the one or more materials comprises xanthan gum and wherein at least 50% of the mass of the one or more oligosaccharides has a molecular mass of less than 5 kDa.

64. The method of any one of claims 1-13, wherein the one or more materials comprises xanthan gum and wherein the one or more materials comprises Xanthomonas campestris extract.

65. The method of any one of claims 1-13, wherein the one or more materials comprises xanthan gum and wherein at least 10 wt. % of the one or more oligosaccharides comprises a degree of polymerization between 2 and 30 based on total mass of the one or more oligosaccharides.

66. The method of any one of claims 1-13, wherein the one or more materials comprises xanthan gum and wherein the one or more oligosaccharides contain at least 10% glucose subunits.

67. The method of any one of claims 1-13, wherein the one or more materials comprises xanthan gum, wherein the one or more oligosaccharides contain at least 10% glucose subunits and wherein the one or more oligosaccharides further comprises galactose subunits, mannose subunits, glucuronic acid subunits, or a combination thereof.

68. The method of any one of claims 1-13, wherein the one or more materials comprises xanthan gum, wherein the one or more oligosaccharides contain at least 10% glucose subunits, wherein the one or more oligosaccharides further comprises galactose subunits, mannose subunits, glucuronic acid subunits, or a combination thereof and wherein the galactose subunits are present in an amount of at least 1%, wherein the mannose subunits are present in an amount of at least 1%, and wherein the glucuronic acid subunits are present in an amount of at least 1%.

69. The method of any one of claims 1-13, wherein the one or more materials comprises xanthan gum and wherein the one or more oligosaccharides comprises glucose subunits and glucuronic acid subunits, wherein the glucuronic acid: glucose ratio is between 1:15 to 1:25.

70. The method of any one of claims 1-13, wherein the one or more materials comprises xanthan gum, wherein the one or more oligosaccharides comprises glucose subunits and mannose subunits, and wherein the mannose: glucose ratio is between 0.1:1 to 1:3.

71. The method of any one of claims 1-13, wherein the one or more materials comprises xanthan gum, wherein the one or more oligosaccharides comprises mannose subunits, and wherein the mannose subunits comprise 2-linkages, 4,6-linkages, and terminal linkages in a total amount of at least 10%.

72. The method of any one of claims 1-13, wherein the one or more materials comprises xanthan gum, wherein the one or more oligosaccharides comprises glucose subunits, and wherein the glucose subunits comprise 3-linkages, 4-linkages, and terminal linkages in a total amount of at least 10%.

73. The method of any one of claims 1-13, wherein the one or more materials comprises xanthan gum, wherein the one or more oligosaccharides comprises glucuronic acid subunits, and wherein the glucuronic acid subunits comprise 4-linkages in a total amount of at least 10%.

74. The method of any one of claims 1-13, wherein the one or more materials comprises xanthan gum, wherein the one or more oligosaccharides comprises galactose subunits, and wherein the galactose subunits comprise 3,6-linkages in a total amount of at least 10%.

75. The method of any one of claims 1-13, wherein the one or more materials comprises xanthan gum, wherein the one or more oligosaccharides comprises glucose subunits, wherein the glucose subunits comprise 3-linkages, 4-linkages, and terminal linkages in a total amount of at least 10% and wherein the glucose subunits comprise a ratio of 3-linkages:4-linkages:terminal linkages of 1:10:4.

76. The method of any one of claims 1-13, wherein the one or more materials comprises xanthan gum, wherein the one or more oligosaccharides comprises mannose subunits, wherein the mannose subunits comprise 2-linkages, 4,6-linkages, and terminal linkages in a total amount of at least 10% and wherein the mannose subunits comprise a ratio of 2-linkages:4,6-linkages:terminal linkages of 8:3:3.

77. The method of any one of claims 1-13, wherein the one or more materials comprises xanthan gum, wherein the one or more oligosaccharides comprises glucose subunits, and wherein the glucose subunits comprise a ratio of 4-linkages:terminal linkages of 5:2.

78. The method of any one of claims 1-13, wherein the one or more materials comprises xanthan gum, wherein the one or more oligosaccharides comprises glucose subunits, and wherein the glucose subunits comprise a ratio of 3-linkages:4-linkages of 1:10.

79. The method of any one of claims 1-13, wherein the one or more materials comprises xanthan gum, wherein the one or more oligosaccharides comprises mannose subunits, and wherein the mannose subunits comprise a ratio of 2-linkages:4,6-linkages of 8:3.

80. The method of any one of claims 1-13, wherein the one or more materials comprises xanthan gum, wherein the one or more oligosaccharides comprises glucose subunits and mannose subunits, and wherein the one or more oligosaccharides comprises a ratio of glucose 4-linkages:mannose 2-linkages of 15:8.

81. The method of any one of claims 1-13, wherein the one or more materials comprises xanthan gum, wherein the one or more oligosaccharides comprises glucose subunits and galactose subunits, and wherein the one or more oligosaccharides comprises a ratio of glucose 4-linkages:galactose 3,6-linkages of 5:2.

82. The method of any one of claims 1-13, wherein the one or more materials comprises xanthan gum and, wherein the one or more oligosaccharides comprises glucose subunits having glucose 3-linkages, and wherein the glucose 3-linkages are beta linked.

83. The method of any one of claims 1-13, wherein the one or more materials comprises xanthan gum, wherein the one or more oligosaccharides comprises glucose subunits having glucose 4-linkages, and wherein the glucose 4-linkages are beta linked.

84. The method of any one of claims 1-13, wherein the one or more materials comprises xanthan gum, wherein the one or more oligosaccharides comprises mannose subunits having mannose 2-linkages and mannose 4,6 linkages, and wherein the mannose 2-linkages and mannose 4,6 linkages are alpha linked.

85. The method of any one of claims 1-13, wherein the one or more materials comprises xanthan gum, wherein the one or more oligosaccharides comprises a mass, a retention time, an oligosaccharide weight percentage, and a retention factor within 30% of the mass, the retention time, the oligosaccharide weight percentage, and the retention factor corresponding to one or more compounds of CLX123 set forth in Table Q.

86. The method of any one of claims 1-13, wherein the one or more materials comprises xanthan gum and wherein the one or more oligosaccharides comprises at least 30 wt. % of the compounds set forth in Table Q based on total mass of the one or more oligosaccharides and total mass of the compounds set forth in Table Q.

87. The method of any one of claims 1-13, wherein the one or more materials comprises xanthan gum and wherein the one or more oligosaccharides comprises one or more 2D NMR correlations within 30% of the 2D NMR correlations set forth in Table A for CLX123.

88. The method of any one of claims 1-13, wherein the one or more materials comprises xanthan gum and wherein the one or more oligosaccharides has a dynamic viscosity ranging from 1 to 1.8 mPa*s at a concentration of 100 mg/ml in water at a temperature of 25° C.

89. The method of any one of claims 1-13, wherein the one or more materials comprises xanthan gum and wherein the one or more oligosaccharides have a dynamic viscosity of 1.6 mPa*s at a concentration of 100 mg/ml in water at a temperature of 25° C.

90. The method of any one of claims 1-13, wherein the one or more materials comprises xanthan gum and wherein the one or more oligosaccharides comprises CLX123.

91. The method of any one of claims 1-13, wherein the one or more materials comprises sugar cane; and
wherein each of the one or more oligosaccharides is independently characterized by at least one feature comprising:
1H-13C HSQC NMR correlations within 30% of the 1H-13C HSQC NMR correlations corresponding to one or more compounds of CLX128 set forth in Table A;
a mass, a retention time, an oligosaccharide weight percentage, and a retention factor within 30% of the mass, the retention time, the oligosaccharide weight percentage, and the retention factor corresponding to one or more compounds of CLX128 set forth in Table T; or
any combination thereof.

92. The method of any one of claims 1-13, wherein the one or more materials comprises sugar cane.

93. The method of any one of claims 1-13, wherein the one or more materials comprises sugar cane and wherein the one or more oligosaccharides contains 4-linked xylose residues.

94. The method of any one of claims 1-13, wherein the one or more materials comprises sugar cane and wherein the one or more oligosaccharides has an average molecular weight (Mw) of less than 10,000 Da.

95. The method of any one of claims 1-13, wherein the one or more materials comprises sugar cane and wherein the one or more oligosaccharides has an average molecular weight (Mw) of less than 8,000 Da.

96. The method of any one of claims 1-13, wherein the one or more materials comprises sugar cane and wherein the one or more oligosaccharides has a dynamic viscosity ranging from 1 to 10 mPa*s at 100 mg/ml at 25° C.

97. The method of any one of claims 1-13, wherein the one or more materials comprises sugar cane and wherein at least 70% of the mass of the one or more oligosaccharides has a molecular mass of less than 100 kDa.

98. The method of any one of claims 1-13, wherein the one or more materials comprises sugar cane and wherein at least 60% of the mass of the one or more oligosaccharides has a molecular mass of less than 50 kDa.

99. The method of any one of claims 1-13, wherein the one or more materials comprises sugar cane and wherein at least 50% of the mass of the one or more oligosaccharides has a molecular mass of less than 15 kDa.

100. The method any one of claims 1-13, wherein the one or more materials comprises sugar cane and wherein at least 50% of the mass of the one or more oligosaccharides has a molecular mass of less than 5 kDa.

101. The method of any one of claims 1-13, wherein the one or more materials comprises sugar cane and, wherein the sugar cane comprises leaf (barbojo), sugar cane stalks (cane), raw sugarcane cylinders or cubes, sugar cane bagasse, fresh sugar cane juice, sugar cane molasses, sugar cane rapadura, sugar cane flour, or processed sugar cane.

102. The method of any one of claims 1-13, wherein the one or more materials comprises sugar cane and wherein at least 10 wt. % of the one or more oligosaccharides comprises a degree of polymerization between 2 and 30 based on total mass of the one or more oligosaccharides.

103. The method of any one of claims 1-13, wherein the one or more materials comprises sugar cane and wherein the one or more oligosaccharides contain at least 10% xylose subunits.

104. The method of any one of claims 1-13, wherein the one or more materials comprises sugar cane and wherein the one or more oligosaccharides contain at least 10% glucose subunits.

105. The method of any one of claims 1-13, wherein the one or more materials comprises sugar cane, wherein the one or more oligosaccharides contain at least 10% xylose subunits and wherein the one or more oligosaccharides further comprises galactose subunits, glucose subunits, arabinose subunits, or a combination thereof.

106. The method of any one of claims 1-13, wherein the one or more materials comprises sugar cane, wherein the one or more oligosaccharides contain at least 10% xylose subunits, wherein the one or more oligosaccharides further comprises galactose subunits, glucose subunits, arabinose subunits, or a combination thereof, wherein the galactose subunits are present in an amount of at least 1%, wherein the glucose subunits are present in an amount of at least 1%, and wherein the arabinose subunits are present in an amount of at least 1%.

107. The method of any one of claims 1-13, wherein the one or more materials comprises sugar cane, wherein the one or more oligosaccharides comprises xylose subunits and glucose subunits, and wherein the xylose: glucose ratio is 10:7.

108. The method of any one of claims 1-13, wherein the one or more materials comprises sugar cane, wherein the one or more oligosaccharides comprises xylose subunits and arabinose subunits, and wherein the xylose: arabinose ratio is 4:1.

109. The method of any one of claims 1-13, wherein the one or more materials comprises sugar cane, wherein the one or more oligosaccharides contain at least 10% xylose subunits and wherein the xylose subunits comprise 4-linkages, 3,4-linkages, and terminal linkages in a total amount of at least 10%.

110. The method of any one of claims 1-13, wherein the one or more materials comprises sugar cane, wherein the one or more oligosaccharides contain at least 10% glucose subunits and wherein the glucose subunits comprise 3-linkages, 4-linkages, and terminal linkages in a total amount of at least 10%.

111. The method of any one of claims 1-13, wherein the one or more materials comprises sugar cane, wherein the one or more oligosaccharides comprises arabinose subunits, and wherein the arabinose subunits comprise 5-linkages and terminal linkages in a total amount of at least 10%.

112. The method of any one of claims 1-13, wherein the one or more materials comprises sugar cane, wherein the one or more oligosaccharides contain at least 10% xylose subunits and wherein the xylose subunits comprise a ratio of 4-linkages:3,4-linkages:terminal linkages of 9:1.7:2.

113. The method of any one of claims 1-13, wherein the one or more materials comprises sugar cane, wherein the one or more oligosaccharides comprises arabinose subunits, and wherein the arabinose subunits comprise a ratio of 5-linkages:terminal linkages of 7:8.

114. The method of any one of claims 1-13, wherein the one or more materials comprises sugar cane, wherein the one or more oligosaccharides contain at least 10% glucose subunits and wherein the glucose subunits comprise a ratio of 3-linkages:4-linkages:terminal linkages of 1:5:3.

115. The method of any one of claims 1-13, wherein the one or more materials comprises sugar cane, wherein the one or more oligosaccharides contain at least 10% xylose subunits and wherein the xylose subunits comprise a ratio of 4-linkages:3,4-linkages of 9:1.7.

116. The method of any one of claims 1-13, wherein the one or more materials comprises sugar cane, wherein the one or more oligosaccharides contain at least 10% xylose subunits and wherein the xylose subunits comprise a ratio of 4-linkages:terminal linkages of 9:2.

117. The method of any one of claims 1-13, wherein the one or more materials comprises sugar cane and wherein the one or more oligosaccharides contain at least 10% glucose subunits and wherein the glucose subunits comprise a ratio of 4-linkages:terminal linkages of 5:3.

118. The method of any one of claims 1-13, wherein the one or more materials comprises sugar cane, wherein the one or more oligosaccharides contain at least 10% glucose subunits and wherein the glucose subunits comprise a ratio of 4-linkages:3-linkages of 5:1.

119. The method of any one of claims 1-13, wherein the one or more materials comprises sugar cane, wherein the one or more oligosaccharides comprises xylose subunits and glucose subunits, and wherein the one or more oligosaccharides comprises a ratio of xylose 4-linkages:glucose 4-linkages of 9:5.

120. The method of any one of claims 1-13, wherein the one or more materials comprises sugar cane wherein the one or more oligosaccharides comprises xylose subunits having xylose 4-linkages, and wherein the xylose 4-linkages are beta linked.

121. The method of any one of claims 1-13, wherein the one or more materials comprises sugar cane and wherein the one or more oligosaccharides comprises a mass, a retention time, an oligosaccharide weight percentage, and a retention factor within 30% of the mass, the retention time, the oligosaccharide weight percentage, and the retention factor corresponding to one or more compounds of CLX128 set forth in Table T.

122. The method of any one of claims 1-13, wherein the one or more materials comprises sugar cane and wherein the one or more oligosaccharides comprises at least 30 wt. %, of the compounds set forth in Table T based on total mass of the one or more oligosaccharides and total mass of the compounds set forth in Table T.

123. The method of any one of claims 1-13, wherein the one or more materials comprises sugar cane and wherein the one or more oligosaccharides comprises one or more 2D NMR correlations within 30% of the 2D NMR correlations set forth in Table A for CLX128.

124. The method of any one of claims 1-13, wherein the one or more materials comprises sugar cane and wherein the one or more oligosaccharides has a dynamic viscosity ranging from 1 to 2 mPa*s at a concentration of 100 mg/ml in water at a temperature of 25° C.

125. The method of any one of claims 1-13, wherein the one or more materials comprises sugar cane and wherein the one or more oligosaccharides has a dynamic viscosity of 1.8 mPa*s at a concentration of 100 mg/ml in water at a temperature of 25° C.

126. The method of any one of claims 1-13, wherein the one or more materials comprises sugar cane and wherein the one or more oligosaccharides comprises CLX128.

127. The method of any one of claims 1-13, wherein the one or more materials comprises soy; and
wherein each of the one or more oligosaccharides is independently characterized by at least one feature comprising:
1H-13C HSQC NMR correlations within 30% of the 1H-13C HSQC NMR correlations corresponding to one or more compounds of CLX126 set forth in Table A;
a mass, a retention time, an oligosaccharide weight percentage, and a retention factor within 30% of the mass, the retention time, the oligosaccharide weight percentage, and the retention factor corresponding to one or more compounds of CLX126 set forth in Table S; or
any combination thereof.

128. The method of any one of claims 1-13, wherein the one or more materials comprises sugar beets, legumes, or a combination thereof.

129. The method of any one of claims 1-13, wherein the one or more materials comprises legumes, and the legumes comprise soy, pea, tree nuts, or a combination thereof.

130. The method of any one of claims 1-13, wherein the one or more materials comprises legumes, wherein the legumes comprise soy.

131. The method of any one of claims 1-13, wherein the one or more materials comprises legumes, wherein the legumes comprise soy and wherein the one or more oligosaccharides has an average molecular weight (Mw) of less than 10,000 Da.

132. The method of any one of claims 1-13, wherein the one or more materials comprises legumes, wherein the legumes comprise soy and wherein the one or more oligosaccharides has an average molecular weight (Mw) of less than 8,000 Da.

133. The method of any one of claims 1-13, wherein the one or more materials comprises legumes, wherein the legumes comprise soy and wherein the one or more oligosaccharides has a dynamic viscosity ranging from 1 to 10 mPa*s at 100 mg/ml at 25° C.

134. The method of any one of claims 1-13, wherein the one or more materials comprises legumes, wherein the legumes comprise soy and wherein at least 70% of the mass of the one or more oligosaccharides has a molecular mass of less than 100 kDa.

135. The method of any one of claims 1-13, wherein the one or more materials comprises legumes, wherein the legumes comprise soy and wherein at least 60% of the mass of the one or more oligosaccharides has a molecular mass of less than 50 kDa.

136. The method of any one of claims 1-13, wherein the one or more materials comprises legumes, wherein the legumes comprise soy and wherein at least 50% of the mass of the one or more oligosaccharides has a molecular mass of less than 15 kDa.

137. The method of any one of claims 1-13, wherein the one or more materials comprises legumes, wherein the legumes comprise soy and wherein at least 50% of the mass of the one or more oligosaccharides has a molecular mass of less than 5 kDa.

138. The method of any one of claims 1-13, wherein the one or more materials comprises legumes, wherein the legumes comprise soy and wherein the one or more oligosaccharides contains at least 30% galactose residues.

139. The method of any one of claims 1-13, wherein the one or more materials comprises legumes, wherein the legumes comprise soy, wherein the one or more oligosaccharides contains at least 30% galactose residues and wherein the one or more oligosaccharides further comprises arabinose subunits, xylose subunits, galacturonic acid subunits, glucose subunits, rhamnose subunits, fucose subunits, or a combination thereof.

140. The method of any one of claims 1-13, wherein the one or more materials comprises legumes, wherein the legumes comprise soy, wherein the one or more oligosaccharides contains at least 30% galactose residues, wherein the one or more oligosaccharides further comprises arabinose subunits, xylose subunits, galacturonic acid subunits, glucose subunits, rhamnose subunits, fucose subunits, or a combination thereof, wherein the arabinose subunits are present in an amount of at least 1%, wherein the xylose subunits are present in an amount of at least 1%, wherein the galacturonic acid subunits are present in an amount of at least 1%, wherein the glucose subunits are present in an amount of at least 1%, wherein the fucose subunits are present in an amount of at least 1%, and wherein the rhamnose subunits are present in an amount of least 1%.

141. The method of any one of claims 1-13, wherein the one or more materials comprises legumes, wherein the legumes comprise soy, wherein the one or more oligosaccharides comprises galactose subunits and arabinose subunits, and wherein the one or more oligosaccharides comprises a ratio of galactose 4-linkages:arabinose terminal linkages of 3:1.

142. The method of any one of claims 1-13, wherein the one or more materials comprises legumes, wherein the legumes comprise soy and wherein the one or more oligosaccharides comprises a mass, a retention time, an oligosaccharide weight percentage, and a retention factor within 30% of the mass, the retention time, the oligosaccharide weight percentage, and the retention factor corresponding to one or more compounds of CLX126 set forth in Table S.

143. The method of any one of claims 1-13, wherein the one or more materials comprises legumes, wherein the legumes comprise soy and wherein the one or more oligosaccharides comprises at least 30 wt. % of the compounds set forth in Table S based on total mass of the one or more oligosaccharides and total mass of the compounds set forth in Table S.

144. The method of any one of claims 1-13, wherein the one or more materials comprises legumes, wherein the legumes comprise soy and wherein the one or more oligosaccharides comprises one or more 2D NMR correlations within 30% of the 2D NMR correlations set forth in Table A for CLX126.

145. The method of any one of claims 1-13, wherein the one or more materials comprises legumes, wherein the legumes comprise soy and wherein the one or more oligosaccharides have a dynamic viscosity of 4.4 mPa*s at a concentration of 100 mg/ml in water at a temperature of 25° C.

146. The method of any one of claims 1-13, wherein the one or more materials comprises legumes, wherein the legumes comprise soy and wherein the one or more oligosaccharides comprises CLX126.

147. The method of any one of claims 1-13, wherein the one or more materials comprises gellan gum; and
wherein each of the one or more oligosaccharides is independently characterized by at least one feature comprising:
1H-13C HSQC NMR correlations within 30% of the 1H-13C HSQC NMR correlations corresponding to one or more compounds of CLX125 set forth in Table A;
a mass, a retention time, an oligosaccharide weight percentage, and a retention factor within 30% of the mass, the retention time, the oligosaccharide weight percentage, and the retention factor corresponding to one or more compounds of CLX125 set forth in Table R; or
any combination thereof.

148. The method of any one of claims 1-13, wherein the one or more materials comprises gellan gum.

149. The method of any one of claims 1-13, wherein the one or more materials comprises gellan gum and wherein the one or more oligosaccharides contains 4-linked glucose subunits, 3-linked glucose subunits, and 4-linked rhamnose subunits.

150. The method of any one of claims 1-13, wherein the one or more materials comprises gellan gum and wherein the one or more oligosaccharides has an average molecular weight (Mw) of less than 10,000 Da.

151. The method of any one of claims 1-13, wherein the one or more materials comprises gellan gum and wherein the one or more oligosaccharides has an average molecular weight (Mw) of less than 8,000 Da.

152. The method of any one of claims 1-13, wherein the one or more materials comprises gellan gum and wherein the one or more oligosaccharides has a dynamic viscosity ranging from 1 to 10 mPa*s at 100 mg/ml at 25° C.

153. The method of any one of claims 1-13, wherein the one or more materials comprises gellan gum and wherein at least 70% of the mass of the one or more oligosaccharides has a molecular mass of less than 100 kDa.

154. The method of any one of claims 1-13, wherein the one or more materials comprises gellan gum and wherein at least 60% of the mass of the one or more oligosaccharides has a molecular mass of less than 50 kDa.

155. The method of any one of claims 1-13, wherein the one or more materials comprises gellan gum and wherein at least 50% of the mass of the one or more oligosaccharides has a molecular mass of less than 15 kDa.

156. The method of any one of claims 1-13, wherein the one or more materials comprises gellan gum and wherein at least 50% of the mass of the one or more oligosaccharides has a molecular mass of less than 5 kDa.

157. The method of any one of claims 1-13, wherein the one or more materials comprises gellan gum and wherein at least 10 wt. % of the one or more oligosaccharides comprises a degree of polymerization between 2 and 30 based on total mass of the one or more oligosaccharides.

158. The method of any one of claims 1-13, wherein the one or more materials comprises gellan gum and wherein the one or more oligosaccharides contain at least 10% glucose subunits.

159. The method of any one of claims 1-13, wherein the one or more materials comprises gellan gum and wherein the one or more oligosaccharides contain at least 10% rhamnose subunits.

160. The method of any one of claims 1-13, wherein the one or more materials comprises gellan gum, wherein the one or more oligosaccharides contain at least 10% glucose subunits and wherein the one or more oligosaccharides further comprises rhamnose subunits, glucuronic acid subunits, or a combination thereof.

161. The method of any one of claims 1-13, wherein the one or more materials comprises gellan gum, wherein the one or more oligosaccharides contain at least 10% glucose subunits, wherein the one or more oligosaccharides further comprises rhamnose subunits, glucuronic acid subunits, or a combination thereof wherein the glucose subunits are present in an amount of at least 1%, wherein the rhamnose subunits are present in an amount of at least 1%, and wherein the glucuronic acid subunits are present in an amount of at least 1%.

162. The method of any one of claims 1-13, wherein the one or more materials comprises gellan gum and wherein the one or more oligosaccharides comprises glucose subunits and rhamnose subunits, wherein the glucose:rhamnose ratio is 1:1.

163. The method of any one of claims 1-13, wherein the one or more materials comprises gellan gum and wherein the one or more oligosaccharides comprises glucose subunits and glucuronic acid subunits, wherein the glucose:glucuronic acid ratio is 5.5:1.

164. The method of any one of claims 1-13, wherein the one or more materials comprises gellan gum wherein the one or more oligosaccharides comprises rhamnose subunits and glucuronic acid subunits, and wherein the rhamnose: glucuronic acid ratio is 5.4:1.

165. The method of any one of claims 1-13, wherein the one or more materials comprises gellan gum, wherein the one or more oligosaccharides contain at least 10% glucose subunits and wherein the glucose subunits comprise 4-linkages, 3-linkages, and terminal linkages in a total amount of at least 10%.

166. The method of any one of claims 1-13, wherein the one or more materials comprises gellan gum, wherein the one or more oligosaccharides contain at least 10% rhamnose subunits and wherein the rhamnose subunits comprise 4-linkages and terminal linkages in a total amount of at least 10%.

167. The method of any one of claims 1-13, wherein the one or more materials comprises gellan gum, wherein the one or more oligosaccharides comprises glucuronic acid subunits, and wherein the glucuronic acid subunits comprise 4-linkages and terminal linkages in a total amount of at least 10%.

168. The method of any one of claims 1-13, wherein the one or more materials comprises gellan gum, wherein the one or more oligosaccharides contain at least 10% glucose subunits and wherein the glucose subunits comprise a ratio of 4-linkages:3-linkages:terminal linkages of 8:7:3.3.

169. The method of any one of claims 1-13, wherein the one or more materials comprises gellan gum, wherein the one or more oligosaccharides contain at least 10% rhamnose subunits and wherein the rhamnose subunits comprise a ratio of 4-linkages:terminal linkages of 3.1:1.

170. The method of any one of claims 1-13, wherein the one or more materials comprises gellan gum wherein the one or more oligosaccharides contain at least 10% glucose subunits and wherein the glucose subunits comprise a ratio of 4-linkages:terminal linkages of 8:3.3.

171. The method of any one of claims 1-13, wherein the one or more materials comprises gellan gum, wherein the one or more oligosaccharides contain at least 10% glucose subunits and wherein the glucose subunits comprise a ratio of 4-linkages:3-linkages of 8:7.

172. The method of any one of claims 1-13, wherein the one or more materials comprises gellan gum, wherein the one or more oligosaccharides contain at least 10% glucose subunits and wherein the glucose subunits comprise a ratio of 3-linkages:terminal linkages of 7:3.3.

173. The method of any one of claims 1-13, wherein the one or more materials comprises gellan gum, wherein the one or more oligosaccharides comprises rhamnose subunits and glucose subunits, and wherein the one or more oligosaccharides comprises a ratio of rhamnose 4-linkages:glucose 4-linkages of 11:12.

174. The method of any one of claims 1-13, wherein the one or more materials comprises gellan gum, wherein the one or more oligosaccharides comprises rhamnose subunits having rhamnose 4-linkages, and wherein the rhamnose 4-linkages are beta linked.

175. The method of any one of claims 1-13, wherein the one or more materials comprises gellan gum and wherein the one or more oligosaccharides comprises a mass, a retention time, an oligosaccharide weight percentage, and a retention factor within 30% of the mass, the retention time, the oligosaccharide weight percentage, and the retention factor corresponding to one or more compounds of CLX125 set forth in Table R.

176. The method of any one of claims 1-13, wherein the one or more materials comprises gellan gum and wherein the one or more oligosaccharides comprises at least 30 wt. %, of the compounds set forth in Table R based on total mass of the one or more oligosaccharides and total mass of the compounds set forth in Table R.

177. The method of any one of claims 1-13, wherein the one or more materials comprises gellan gum and wherein the one or more oligosaccharides comprises one or more 2D NMR correlations within 30% of the 2D NMR correlations set forth in Table A for CLX125.

178. The method of any one of claims 1-13, wherein the one or more materials comprises gellan gum and wherein the one or more oligosaccharides has a dynamic viscosity having a range of 1 to 2 mPa*s at a concentration of 100 mg/ml in water at a temperature of 25° C.

179. The method of any one of claims 1-13, wherein the one or more materials comprises gellan gum, and wherein the one or more oligosaccharides has a dynamic viscosity of 1.5 mPa*s at a concentration of 100 mg/ml in water at a temperature of 25° C.

180. The method of any one of claims 1-13, wherein the one or more oligosaccharides comprises CLX125.

181. The method of any one of claims 1-13, wherein the one or more oligosaccharides comprise at least one glycosidic bond comprising galactose-mannose (α1-6), xylose-xylose (β1-4), arabinose-xylose (α1-2/3), xylose-glucose (α1-6), galactose-xylose (β1-2), glucose-glucose (β1-4), or any combination thereof.

182. The method of any one of claims 1-13, wherein the one or more oligosaccharides comprise at least one glycosidic bond that can be depolymerized via glycosyl hydrolase families GH 36 with EC 3.2.1.22 activity, GH 8 with EC 3.2.1.4 activity, GH 10 with EC 3.2.1.8 activity, GH 31 with EC 3.2.1. 1717 activity, GH 51 with EC 3.2.1.55 activity, GH 2 with EC 3.2.1. 23 activity, or any combination thereof.

183. The method of any one of claims 1-13, wherein the one or more materials comprises homo-xylan, glucuronoxylan, glucuronoarabinoxylan, arabinoxylan, or any combination thereof; and
wherein each of the one or more oligosaccharides is independently characterized by at least one feature comprising:
1H-13C HSQC NMR correlations within 30% of the 1H-13C HSQC NMR correlations corresponding to one or more compounds of CLX103 set forth in Table A;
a mass, a retention time, an oligosaccharide weight percentage, and a retention factor within 30% of the mass, the retention time, the oligosaccharide weight percentage, and the retention factor corresponding to one or more compounds of CLX103 set forth in Table E; or
any combination thereof.

184. The method of any one of claims 1-13, wherein the one or more materials comprises at least one of homo-xylan, glucuronoxylan, or a combination thereof.

185. The method of any one of claims 1-13, wherein the one or more materials comprises at least one of homo-xylan, glucuronoxylan, or a combination thereof and wherein the one or more oligosaccharides comprises CLX103.

186. The method of any one of claims 1-13, wherein the one or more materials comprises galactomannan.

187. The method of any one of claims 1-13, wherein the one or more materials comprises galactomannan and wherein the one or more materials comprises a seed mucilage, a cell wall of a mold, or a combination thereof.

188. The method of any one of claims 1-13, wherein the one or more materials comprises pea; and
wherein each of the one or more oligosaccharides is independently characterized by at least one feature comprising:
1H-13C HSQC NMR correlations within 30% of the 1H-13C HSQC NMR correlations corresponding to one or more compounds of CLX122 set forth in Table A;
a mass, a retention time, an oligosaccharide weight percentage, and a retention factor within 30% of the mass, the retention time, the oligosaccharide weight percentage, and the retention factor corresponding to one or more compounds of CLX122 set forth in Table U; or
any combination thereof.

189. The method of any one of claims 1-13, wherein the one or more materials comprises pea; and
wherein each of the one or more oligosaccharides is independently characterized by at least one feature comprising:
1H-13C HSQC NMR correlations within 30% of the 1H-13C HSQC NMR correlations corresponding to one or more compounds of CLX122DSF set forth in Table A;
a mass, a retention time, an oligosaccharide weight percentage, and a retention factor within 30% of the mass, the retention time, the oligosaccharide weight percentage, and the retention factor corresponding to one or more compounds of CLX122DSF set forth in Table W; or
any combination thereof.

190. The method of any one of claims 1-13, wherein the one or more materials comprises pea.

191. The method of any one of claims 1-13, wherein the one or more materials comprises pea and wherein the one or more oligosaccharides contain at least 10% arabinose subunits.

192. The method of any one of claims 1-13, wherein the one or more materials comprises pea, wherein the one or more oligosaccharides contains at least 10% arabinose subunits and wherein the one or more oligosaccharides further comprises galactose subunits, glucose subunits, galacturonic acid subunits, rhamnose subunits, or a combination thereof.

193. The method of any one of claims 1-13, wherein the one or more materials comprises pea, wherein the one or more oligosaccharides contains at least 10% arabinose subunits, wherein the one or more oligosaccharides further comprises galactose subunits, glucose subunits, galacturonic acid subunits, rhamnose subunits, or a combination thereof, wherein the galactose subunits are present in an amount of at least 1%, wherein the glucose subunits are present in an amount of at least 1%, wherein the galacturonic acid subunits are present in an amount of at least 1%, and wherein the rhamnose subunits are present in an amount of at least 1%.

194. The method of any one of claims 1-13, wherein the one or more materials comprises pea, wherein the one or more oligosaccharides comprises arabinose subunits and galactose subunits, and wherein the arabinose: galactose ratio is 8:1.

195. The method of any one of claims 1-13, wherein the one or more materials comprises pea, wherein the one or more oligosaccharides comprises arabinose subunits and glucose subunits, and wherein the arabinose: glucose ratio is 7.7:1.

196. The method of any one of claims 1-13, wherein the one or more materials comprises pea, and wherein the one or more oligosaccharides comprises a mass, a retention time, an oligosaccharide weight percentage, and a retention factor within 30% of the mass, the retention time, the oligosaccharide weight percentage, and the retention factor corresponding to one or more compounds of CLX122 set forth in Table U, CLX122DSF set forth in Table W, or any combination thereof.

197. The method of any one of claims 1-13, wherein the one or more materials comprises pea and wherein the one or more oligosaccharides comprises at least 30 wt. % of the compounds set forth in Table U or Table W, based on total mass of the one or more oligosaccharides and total mass of the compounds set forth in Table U or Table W, respectively.

198. The method of any one of claims 1-13, wherein the one or more materials comprises pea and wherein the one or more oligosaccharides comprises one or more 2D NMR correlations within 30% of the 2D NMR correlations set forth in Table A for CLX122 or CLX122DSF.

199. The method of any one of claims 1-13, wherein the one or more materials comprises pea and wherein the one or more oligosaccharides has a dynamic viscosity ranging from 0.75 to 5 mPa*s at a concentration of 100 mg/ml in water at a temperature of 25° C.

200. The method of any one of claims 1-13, wherein the one or more materials comprises pea and wherein the one or more oligosaccharides have a dynamic viscosity ranging from 2 to 4 mPa*s at a concentration of 100 mg/ml in water at a temperature of 25° C.

201. The method of any one of claims 1-13, wherein the one or more materials comprises pea and wherein the one or more oligosaccharides have a dynamic viscosity ranging from 0.75 to 3 mPa*s at a concentration of 100 mg/ml in water at a temperature of 25° C.

202. The method of any one of claims 1-13, wherein the one or more materials comprises pea and wherein the one or more oligosaccharides comprises CLX122, CLX122DSF, or a combination thereof.

203. The method of any one of claims 1-13, wherein the one or more materials comprises pea and wherein the one or more oligosaccharides comprises CLX122.

204. The method of any one of claims 1-13, wherein the one or more materials comprises pea and wherein the one or more oligosaccharides comprises CLX122DSF.

205. The method of any one of claims 1-13, wherein the one or more oligosaccharides comprises a mass, a retention time, an oligosaccharide weight percentage, and a retention factor within 30% of the mass, the retention time, the oligosaccharide weight percentage, and the retention factor corresponding to one or more compounds of CLX111 as set forth in Table G.

206. The method of any one of claims 1-13, wherein the one or more oligosaccharides comprise at least 30 wt. % of the compounds set forth in Table G, based on total mass of the one or more oligosaccharides and total mass of the compounds set forth in Table G.

207. The method of any one of claims 1-13, wherein the one or more oligosaccharides comprise one or more of the 2D NMR correlations within 30% of the 2D NMR correlations set forth in Table A for CLX111.

208. The method of any one of claims 1-13, wherein the one or more materials comprises pea, wherein the one or more oligosaccharides comprises one or more 2D NMR correlations within 30% of the 2D NMR correlations set forth in Table A for CLX122 or CLX122DSF and wherein the one or more oligosaccharides have a dynamic viscosity of between 0.6 and 1.2 mPa*s at a concentration of 10 mg/ml in water at a temperature of 25° C.

209. The method of any one of claims 1-13, wherein the one or more oligosaccharides comprises CLX111.

210. The method of any one of claims 1-13, wherein the one or more materials comprises arabinan.

211. The method of any one of claims 1-13, wherein the one or more materials comprises a moringa plant or part thereof.

212. The method of any one of claims 1-13, wherein the one or more materials comprises a moringa plant or part thereof and wherein the moringa plant or part thereof comprises leaves, branches, trunks, roots, seeds, flowers, fruit, or any combination thereof.

213. The method of any one of claims 1-13, wherein the one or more materials comprises a moringa plant or part thereof and wherein the moringa plant or part thereof comprises leaves.

214. The method of any one of claims 1-13, wherein the one or more materials comprises a Cucurbita species or part thereof.

215. The method of any one of claims 1-13, wherein the one or more materials comprises a Cucurbita species or part thereof and wherein the Cucurbita species or part thereof comprises flesh, skin, seeds, or any combination thereof of a squash, a gourd, a pumpkin, or any combination thereof.

216. The method of any one of claims 1-13, wherein the one or more materials comprises a Cucurbita species or part thereof and wherein the Cucurbita species or part thereof comprises the skin of a butternut squash.

217. The method of any one of claims 1-13, wherein the one or more materials comprises spent distillers' grains.

218. The method of any one of claims 1-13, wherein one or more materials comprises microbial curdlan.

219. The method of any one of claims 1-13, wherein the one or more materials comprises glucomannan.

220. The method of any one of claims 1-13, wherein the one or more materials comprises pectic galactan.

221. The method of any one of claims 1-13, wherein the one or more oligosaccharides are preferentially metabolized by beneficial vaginal Lactobacillus species but not preferentially metabolized by vaginal dysbiosis-associated bacteria.

222. The method of any one of claims 1-13, wherein the one or more oligosaccharides are preferentially metabolized by beneficial vaginal Lactobacillus species, but not preferentially metabolized by vaginal dysbiosis-associated bacteria and wherein the vaginal dysbiosis-associated bacteria comprise Lactobacillus iners, Gardenerella vaginalis, Atopobium vaginae, Prevotella species, Mobiluncus species, or any combination thereof.

223. The method of any one of claims 1-13, wherein the composition further comprises at least one probiotic.

224. The method of any one of claims 1-13, wherein the composition further comprises at least one probiotic and wherein the at least one probiotic comprises Lactobacillus species, Bifidobacterium species, or a combination thereof.

225. The method of any one of claims 1-13, wherein the composition further comprises at least one probiotic, wherein the at least one probiotic comprises Lactobacillus species, Bifidobacterium species, or a combination thereof and wherein the at least one probiotic comprises the Lactobacillus species, and the Lactobacillus species comprises at least one of L. rhamnosus, L. acidophilus, L. fermentum, L. reuteri, L. crispatus, L. jensenii, L. gasseri, L. acidophilus, L. iners, L., plantarum, L. vaginalis, or any combination thereof.

226. The method of any one of claims 1-13, wherein the composition further comprises at least one probiotic, wherein the at least one probiotic comprises Lactobacillus species, Bifidobacterium species, or a combination thereof and wherein the at least one probiotic comprises the Lactobacillus species, and the Lactobacillus species comprises L. reuteri RC-14, L. rhamnosus GR-1, L. fermentum LF15, or any combination thereof.

227. The method of any one of claims 1-13, wherein the composition further comprises at least one probiotic and wherein the at least one probiotic comprises a live biotherapeutic.

228. The method of any one of claims 1-13, wherein the composition further comprises at least one probiotic, wherein the at least one probiotic comprises a live biotherapeutic and wherein the live biotherapeutic comprises L. crispatus CTV-05.

229. The method of any one of claims 1-13, wherein the composition further comprises at least one probiotic, wherein the at least one probiotic comprises Lactobacillus species, Bifidobacterium species, or a combination thereof and wherein the at least one probiotic comprises the Bifidobacterium species, and the Bifidobacterium species comprises B. longum, B. breve, B. adolescentis, B. pseudocatenulatum, B. animalis, B. animalis lactis, B. infantis, or any combination thereof.

230. The method of any one of claims 1-13, wherein the therapeutically effective amount is between 50 mg and 10 g of the one or more oligosaccharides.

231. The method of any one of claims 1-13, wherein the therapeutically effective amount is between 50 mg and 10 g per day of the one or more oligosaccharides.

232. The method of any one of claims 1-13, wherein the method reduces a risk of bacterial vaginosis in the female subject, reduces a risk of recurrence of bacterial vaginosis in the female subject, reduces severity of bacterial vaginosis in the female subject, treats bacterial vaginosis in the female subject, reduces a risk of urinary tract infection when the female subject has bacterial vaginosis, reduces a risk of urinary tract infection when the female subject has had, but no longer has, bacterial vaginosis, or any combination thereof.

233. The method of any one of claims 1-13, wherein the method reduces a risk of vulvovaginal candidiasis in the female subject, reduces a risk of recurrence of vulvovaginal candidiasis in the female subject, reduces severity of vulvovaginal candidiasis in the female subject, treats vulvovaginal candidiasis in the female subject, or any combination thereof.

234. A pack for improving urogenital health in a female subject, the pack comprising at least 7 or at least 14 individual daily doses each of which comprising a therapeutically effective amount of one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

235. The pack of claim 234, wherein the one or more materials or the one or more oligosaccharides is as described in any one of claims 15-220.

236. A formulation comprising one or more oligosaccharides as described in any one of claims 15-220 or as derived from one or more materials as described in any one of claims 15-220.

237. A formulation comprising a composition comprising one or more oligosaccharides as described in any one of claims 15-230.

238. The formulation of claim 236 or 237, further comprising at least one microorganism.

239. The formulation of claim 236 or 237, further comprising at least one microorganism, wherein the at least one microorganism comprises at least one of Bifidobacteria, Bifidobacterium pseudocatenulatum, Bifidobacterium animalis, Bacteroides, Bacteroides ovatus, Firmicutes, Clostridium butyricum, Ruminococcus, Ruminococcus gnavus, Ruminococcus torques, Blautia, Roseburia, Faecalibacterium, Lactobacillus crispatus, Lactobacillus rhamnosus, Lactobacillus Rhamnosus GG, Bifidobacterium longum subsp. longum, Bifidobacterium longum subsp. infantis, Ruminococcus torques, or any combination thereof.

240. The formulation of any one of claims 236-239, further comprising a pharmaceutically acceptable carrier.

241. The formulation of any one of claims 236-240, wherein the formulation is in the form of a food, a drink, a nutritional supplement, a medicine, an injection, a pill, a capsule, a cream, or a topical ointment.

242. Use of a composition for improving urogenital health in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

243. Use of a composition for treating or preventing a vaginal infection in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

244. Use of a composition for modulating the microbiota of the urogenital tract in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

245. Use of a composition for lowering the pH of the urogenital tract in a female subject, wherein the composition comprises one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

246. A medicament, for use in for improving urogenital health in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

247. A medicament, for use in treating or preventing a vaginal infection in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

248. A medicament, for use in modulating the microbiota of the urogenital tract in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

249. A medicament, for use in lowering the pH of the urogenital tract in a female subject, comprising an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof.

250. A method of making a medicament for use in for improving urogenital health in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and
an optional carrier.

251. A method of making a medicament for use in treating or preventing a vaginal infection in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and
an optional carrier.

252. A method of making a medicament for use in modulating the microbiota of the urogenital tract in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and an optional carrier.

253. A method of making a medicament for use in lowering the pH of the urogenital tract in a female subject, the method comprising combining:
an effective amount of a composition having one or more oligosaccharides derived from one or more materials comprising galactomannan, β-glucan, homo-xylan, glucuronoxylan, glucuronoarabinoxylan, arabinoxylan, arabinan, arabinogalactan, xyloglucan, microbial curdlan, glucomannan, pectic galactan, a moringa plant or part thereof, a Cucurbita species plant or part thereof, spent distillers' grains, gellan gum, xanthan gum, legumes, soy, pea, sugar cane or any combination thereof, and an optional carrier.

254. A method of improving urogenital health in a female subject, the method comprising:
administering to the female subject a therapeutically effective amount of a composition comprising one or more oligosaccharides;
wherein each of the one or more oligosaccharides independently contains 3 to 30 subunits;
wherein at least 5% of the subunits comprise a beta-1,3 glucose residue, a beta-1,4 glucose residue, or a combination thereof; and
wherein the administering step results in improving urogenital health in the female subject.

255. A method of treating or preventing a vaginal infection in a female subject, the method comprising:
administering to the female subject a therapeutically effective amount of a composition comprising one or more oligosaccharides;
wherein each of the one or more oligosaccharides independently contains 3 to 30 subunits;
wherein at least 5% of the subunits comprise a beta-1,3 glucose residue, a beta-1,4 glucose residue, or a combination thereof; and
wherein the administering step results in treating or preventing a vaginal infection in the female subject.

256. A method of modulating the microbiota of the urogenital tract of a female subject, the method comprising:
administering to the female subject a therapeutically effective amount of a composition comprising one or more oligosaccharides;
wherein each of the one or more oligosaccharides independently contains 3 to 30 subunits;
wherein at least 5% of the subunits comprise a beta-1,3 glucose residue, a beta-1,4 glucose residue, or a combination thereof; and
wherein the administering step comprises contacting said microbiota of the urogenital tract with said one or more oligosaccharides; and
wherein the administering step results in modulating the microbiota of the urogenital tract of the female subject.

257. The method of claim 256, wherein the method results in increased abundance of Lactobacillus species in the urogenital tract of the female subject.

258. The method of claim 257, wherein the Lactobacillus species comprise L. crispatus, L. gasseri, L. jensenii, L. iners, L. vaginalis, or any combination thereof.

259. A method of lowering the pH of the urogenital tract of a female subject, the method comprising:
administering to the female subject a therapeutically effective amount of a composition comprising one or more oligosaccharides;
wherein each of the one or more oligosaccharides independently contains 3 to 30 subunits;
wherein at least 5% of the subunits comprise a beta-1,3 glucose residue, a beta-1,4 glucose residue, or a combination thereof; and
wherein the administering step results in lowering the pH of the urogenital tract.

260. The method of claim 259, wherein the method results in lowering the pH of the urogenital tract from a first pH of 4.5-7 to a second pH of less than 4.5.

261. The method of claim 260, wherein the second pH is between 3.5-4.5.

262. The method of any one of claims 254-261, wherein at least 10% of the one or more oligosaccharides contains 3 to 20 subunits.

263. The method of any one of claims 254-261, wherein at least 10% of the one or more oligosaccharides contains 3 to 10 subunits.

264. The method of any one of claims 254-261, wherein at least 5% of the subunits comprise a beta-1,4 glucose residue.

265. The method of any one of claims 254-261, wherein at least 5% of the subunits comprise a beta-1,3 glucose residue.
